SEQUENCE LISTING (317 sequences)

SEQ ID NO: 1 - protein, 1425 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDAALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 2 - protein, 1497 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVREDKRPAATKKAGQAKKKK

SEQ ID NO: 3 - DNA, 24 bp, synthetic construct
  source 1..24: mol_type = other DNA
ggttctgggtacttttatctgtcc

SEQ ID NO: 4 - DNA, 24 bp, synthetic construct
  source 1..24: mol_type = other DNA
gtctgcctaacaggaggtgggggt

SEQ ID NO: 5 - DNA, 24 bp, synthetic construct
  source 1..24: mol_type = other DNA
gaatatcaggagactaggaaggag

SEQ ID NO: 6 - DNA, 22 bp, synthetic construct
  source 1..22: mol_type = other DNA
gcctccctgcagggctgctccc

SEQ ID NO: 7 - DNA, 24 bp, synthetic construct
  source 1..24: mol_type = other DNA
gagctagtcttcttcctccaaccc

SEQ ID NO: 8 - DNA, 24 bp, synthetic construct
  source 1..24: mol_type = other DNA
gatctgtcccctccaccccacagt

SEQ ID NO: 9 - DNA, 24 bp, synthetic construct
  source 1..24: mol_type = other DNA
ggcccaaatgaaaggagtgagagg

SEQ ID NO: 10 - DNA, 24 bp, synthetic construct
  source 1..24: mol_type = other DNA
gcatcctcttgctttctttgcctg

SEQ ID NO: 11 - DNA, 24 bp, synthetic construct
  source 1..24: mol_type = other DNA
ggagtcgccagaggccggtggtgg

SEQ ID NO: 12 - DNA, 24 bp, synthetic construct
  source 1..24: mol_type = other DNA
gcccagcggccggatatcagctgc

SEQ ID NO: 13 - DNA, 23 bp, synthetic construct
  source 1..23: mol_type = other DNA
ggaagggaacatattactattgc

SEQ ID NO: 14 - DNA, 24 bp, synthetic construct
  source 1..24: mol_type = other DNA
gtggagtggcctgctatcagctac

SEQ ID NO: 15 - DNA, 24 bp, synthetic construct
  source 1..24: mol_type = other DNA
gaggaagggaacatattactattg

SEQ ID NO: 16 - DNA, 24 bp, synthetic construct
  source 1..24: mol_type = other DNA
gtgaattctcatcagctaaaatgc

SEQ ID NO: 17 - DNA, 24 bp, synthetic construct
  source 1..24: mol_type = other DNA
gctcactcacccacacagacacac

SEQ ID NO: 18 - DNA, 24 bp, synthetic construct
  source 1..24: mol_type = other DNA
ggaagaatttcattctgttctcag

SEQ ID NO: 19 - DNA, 24 bp, synthetic construct
  source 1..24: mol_type = other DNA
gctcagttttcctggattatgcct

SEQ ID NO: 20 - DNA, 24 bp, synthetic construct
  source 1..24: mol_type = other DNA
gcgttggagcggggagaaggccag

SEQ ID NO: 21 - DNA, 24 bp, synthetic construct
  source 1..24: mol_type = other DNA
gggccgcggagatagctgcagggc

SEQ ID NO: 22 - DNA, 24 bp, synthetic construct
  source 1..24: mol_type = other DNA
gcccacccggcggcgcctccctgc

SEQ ID NO: 23 - DNA, 24 bp, synthetic construct
  source 1..24: mol_type = other DNA
gcgtggcagctgatatccggccgc

SEQ ID NO: 24 - DNA, 24 bp, synthetic construct
  source 1..24: mol_type = other DNA
gccgcggcgcgacgtggagccagc

SEQ ID NO: 25 - DNA, 24 bp, synthetic construct
  source 1..24: mol_type = other DNA
gtgctccccagcccaaaccgccgc

SEQ ID NO: 26 - DNA, 24 bp, synthetic construct
  source 1..24: mol_type = other DNA
gtcagattggcttgctcggaattg

SEQ ID NO: 27 - DNA, 24 bp, synthetic construct
  source 1..24: mol_type = other DNA
gctgggtgaatggagcgagcagcg

SEQ ID NO: 28 - DNA, 24 bp, synthetic construct
  source 1..24: mol_type = other DNA
gtcctggagtgacccctggccttc

SEQ ID NO: 29 - DNA, 24 bp, synthetic construct
  source 1..24: mol_type = other DNA
gatcctggagtgacccctggcctt

SEQ ID NO: 30 - DNA, 24 bp, synthetic construct
  source 1..24: mol_type = other DNA
gtgtgtccctctccccacccgtcc

SEQ ID NO: 31 - DNA, 24 bp, synthetic construct
  source 1..24: mol_type = other DNA
gttggagcggggagaaggccaggg

SEQ ID NO: 32 - DNA, 24 bp, synthetic construct
  source 1..24: mol_type = other DNA
gtaccctccaataatttggctggc

SEQ ID NO: 33 - DNA, 24 bp, synthetic construct
  source 1..24: mol_type = other DNA
gataatttggctggcaattccgag

SEQ ID NO: 34 - DNA, 24 bp, synthetic construct
  source 1..24: mol_type = other DNA
gcaggggccaggtgtccttctctg

SEQ ID NO: 35 - DNA, 24 bp, synthetic construct
  source 1..24: mol_type = other DNA
gaatggcaggcggaggttgtactg

SEQ ID NO: 36 - DNA, 24 bp, synthetic construct
  source 1..24: mol_type = other DNA
gagtgagagagtgagagagagaca

SEQ ID NO: 37 - DNA, 24 bp, synthetic construct
  source 1..24: mol_type = other DNA
gtgagcaggcacctgtgccaacat

SEQ ID NO: 38 - DNA, 24 bp, synthetic construct
  source 1..24: mol_type = other DNA
gcgtgggggctccgtgccccacgc

SEQ ID NO: 39 - DNA, 24 bp, synthetic construct
  source 1..24: mol_type = other DNA
gcatgggcaggggctggggtgcac

SEQ ID NO: 40 - DNA, 24 bp, synthetic construct
  source 1..24: mol_type = other DNA
gaaaattgtgatttccagatccac

SEQ ID NO: 41 - DNA, 23 bp, synthetic construct
  source 1..23: mol_type = other DNA
gagcagaaaaaattgtgatttcc

SEQ ID NO: 42 - DNA, 19 bp, synthetic construct
  source 1..19: mol_type = other DNA
tggcttagcacctctccat

SEQ ID NO: 43 - DNA, 25 bp, synthetic construct
  source 1..25: mol_type = other DNA
agaggagccttctgactgctgcaga

SEQ ID NO: 44 - DNA, 18 bp, synthetic construct
  source 1..18: mol_type = other DNA
tccgtcttcctccactcc

SEQ ID NO: 45 - DNA, 19 bp, synthetic construct
  source 1..19: mol_type = other DNA
tagagaactgggtagtgtg

SEQ ID NO: 46 - DNA, 24 bp, synthetic construct
  source 1..24: mol_type = other DNA
gtacatgaagcaactccagtccca

SEQ ID NO: 47 - DNA, 24 bp, synthetic construct
  source 1..24: mol_type = other DNA
tggtgattatgggagaactggagc

SEQ ID NO: 48 - DNA, 21 bp, synthetic construct
  source 1..21: mol_type = other DNA
ggcataaggaaatcgaaggtc

SEQ ID NO: 49 - DNA, 23 bp, synthetic construct
  source 1..23: mol_type = other DNA
acacgggcagcatgggaatagtc

SEQ ID NO: 50 - DNA, 22 bp, synthetic construct
  source 1..22: mol_type = other DNA
cctgtgtggctttgctttggtc

SEQ ID NO: 51 - DNA, 23 bp, synthetic construct
  source 1..23: mol_type = other DNA
ggaggaagagtagctcgccgagg

SEQ ID NO: 52 - DNA, 24 bp, synthetic construct
  source 1..24: mol_type = other DNA
agggagagggaagtgtggggaagg

SEQ ID NO: 53 - DNA, 23 bp, synthetic construct
  source 1..23: mol_type = other DNA
gttgggggctctaagttatgtat

SEQ ID NO: 54 - DNA, 24 bp, synthetic construct
  source 1..24: mol_type = other DNA
gtggtttctgctctcaccgggagg

SEQ ID NO: 55 - DNA, 24 bp, synthetic construct
  source 1..24: mol_type = other DNA
tggtttctgctctcaccgggaggg

SEQ ID NO: 56 - DNA, 26 bp, synthetic construct
  source 1..26: mol_type = other DNA
tctgctctcaccgggaggggctccgg

SEQ ID NO: 57 - DNA, 24 bp, synthetic construct
  source 1..24: mol_type = other DNA
aggtggtttctgctctcaccggga

SEQ ID NO: 58 - DNA, 24 bp, synthetic construct
  source 1..24: mol_type = other DNA
ggtttctgctctcaccgggagggg

SEQ ID NO: 59 - DNA, 24 bp, synthetic construct
  source 1..24: mol_type = other DNA
ggcctcagcttttcacttcctgcc

SEQ ID NO: 60 - DNA, 24 bp, synthetic construct
  source 1..24: mol_type = other DNA
cttttcacttcctgccggggcgtt

SEQ ID NO: 61 - DNA, 24 bp, synthetic construct
  source 1..24: mol_type = other DNA
tttcacttcctgccggggcgtttg

SEQ ID NO: 62 - DNA, 24 bp, synthetic construct
  source 1..24: mol_type = other DNA
ttcacttcctgccggggcgtttga

SEQ ID NO: 63 - DNA, 24 bp, synthetic construct
  source 1..24: mol_type = other DNA
ccacactccccggctttcagcccc

SEQ ID NO: 64 - DNA, 24 bp, synthetic construct
  source 1..24: mol_type = other DNA
ggctttcagccccgtttcttggga

SEQ ID NO: 65 - DNA, 24 bp, synthetic construct
  source 1..24: mol_type = other DNA
gctttcagccccgtttcttgggaa

SEQ ID NO: 66 - DNA, 24 bp, synthetic construct
  source 1..24: mol_type = other DNA
cgggtcttgcacttcttgatgggg

SEQ ID NO: 67 - DNA, 24 bp, synthetic construct
  source 1..24: mol_type = other DNA
tcttgcacttcttgatggggagta

SEQ ID NO: 68 - DNA, 24 bp, synthetic construct
  source 1..24: mol_type = other DNA
ttgcacttcttgatggggagtacg

SEQ ID NO: 69 - DNA, 22 bp, synthetic construct
  source 1..22: mol_type = other DNA
tgcacttcttgatggggagtac

SEQ ID NO: 70 - DNA, 23 bp, synthetic construct
  source 1..23: mol_type = other DNA
gtcacctgcctcgtggaatacgg

SEQ ID NO: 71 - DNA, 23 bp, synthetic construct
  source 1..23: mol_type = other DNA
gcacctgcctcgtggaatacggt

SEQ ID NO: 72 - DNA, 22 bp, synthetic construct
  source 1..22: mol_type = other DNA
gttcagcgtgtccggctttggc

SEQ ID NO: 73 - DNA, 24 bp, synthetic construct
  source 1..24: mol_type = other DNA
gtggtgagcaagggcgaggagctg

SEQ ID NO: 74 - DNA, 24 bp, synthetic construct
  source 1..24: mol_type = other DNA
gggcgaggagctgttcaccggggt

SEQ ID NO: 75 - DNA, 24 bp, synthetic construct
  source 1..24: mol_type = other DNA
gtgaacttgtggccgtttacgtcg

SEQ ID NO: 76 - DNA, 24 bp, synthetic construct
  source 1..24: mol_type = other DNA
gcgtccagctcgaccaggatgggc

SEQ ID NO: 77 - DNA, 24 bp, synthetic construct
  source 1..24: mol_type = other DNA
gcggtgaacagctcctcgcccttg

SEQ ID NO: 78 - DNA, 24 bp, synthetic construct
  source 1..24: mol_type = other DNA
gggcaccaccccggtgaacagctc

SEQ ID NO: 79 - DNA, 24 bp, synthetic construct
  source 1..24: mol_type = other DNA
ggcaccaccccggtgaacagctcc

SEQ ID NO: 80 - DNA, 24 bp, synthetic construct
  source 1..24: mol_type = other DNA
gggatgggcaccaccccggtgaac

SEQ ID NO: 81 - DNA, 24 bp, synthetic construct
  source 1..24: mol_type = other DNA
gcgtgtccggctttggcgagacaa

SEQ ID NO: 82 - DNA, 24 bp, synthetic construct
  source 1..24: mol_type = other DNA
gtccggctttggcgagacaaatca

SEQ ID NO: 83 - DNA, 24 bp, synthetic construct
  source 1..24: mol_type = other DNA
gatcacctgcctcgtggaatacgg

SEQ ID NO: 84 - DNA, 24 bp, synthetic construct
  source 1..24: mol_type = other DNA
gacgctgaacttgtggccgtttac

SEQ ID NO: 85 - DNA, 24 bp, synthetic construct
  source 1..24: mol_type = other DNA
gccaaagccggacacgctgaactt

SEQ ID NO: 86 - DNA, 23 bp, synthetic construct
  source 1..23: mol_type = other DNA
ggaacttgtggccgtttacgtcg

SEQ ID NO: 87 - DNA, 22 bp, synthetic construct
  source 1..22: mol_type = other DNA
gaacttgtggccgtttacgtcg

SEQ ID NO: 88 - DNA, 21 bp, synthetic construct
  source 1..21: mol_type = other DNA
gacttgtggccgtttacgtcg

SEQ ID NO: 89 - DNA, 20 bp, synthetic construct
  source 1..20: mol_type = other DNA
gcttgtggccgtttacgtcg

SEQ ID NO: 90 - DNA, 19 bp, synthetic construct
  source 1..19: mol_type = other DNA
gttgtggccgtttacgtcg

SEQ ID NO: 91 - DNA, 22 bp, synthetic construct
  source 1..22: mol_type = other DNA
gcacctgcctcgtggaatacgg

SEQ ID NO: 92 - DNA, 21 bp, synthetic construct
  source 1..21: mol_type = other DNA
gacctgcctcgtggaatacgg

SEQ ID NO: 93 - DNA, 20 bp, synthetic construct
  source 1..20: mol_type = other DNA
gcctgcctcgtggaatacgg

SEQ ID NO: 94 - DNA, 20 bp, synthetic construct
  source 1..20: mol_type = other DNA
gggccaggtgtccttctctg

SEQ ID NO: 95 - DNA, 20 bp, synthetic construct
  source 1..20: mol_type = other DNA
ggcaggcggaggttgtactg

SEQ ID NO: 96 - DNA, 20 bp, synthetic construct
  source 1..20: mol_type = other DNA
gagagagtgagagagagaca

SEQ ID NO: 97 - DNA, 20 bp, synthetic construct
  source 1..20: mol_type = other DNA
gcaggcacctgtgccaacat

SEQ ID NO: 98 - DNA, 20 bp, synthetic construct
  source 1..20: mol_type = other DNA
gggggctccgtgccccacgc

SEQ ID NO: 99 - DNA, 20 bp, synthetic construct
  source 1..20: mol_type = other DNA
gggcaggggctggggtgcac

SEQ ID NO: 100 - DNA, 23 bp, synthetic construct
  source 1..23: mol_type = other DNA
cctgtgtggctttgctttggtcg

SEQ ID NO: 101 - DNA, 24 bp, synthetic construct
  source 1..24: mol_type = other DNA
agaactcaggaccaacttattctg

SEQ ID NO: 102 - DNA, 23 bp, synthetic construct
  source 1..23: mol_type = other DNA
atgacagacacaaccagagggca

SEQ ID NO: 103 - DNA, 20 bp, synthetic construct
  source 1..20: mol_type = other DNA
taggaaggaggaggcctaag

SEQ ID NO: 104 - DNA, 19 bp, synthetic construct
  source 1..19: mol_type = other DNA
ccaatattgcatgggatgg

SEQ ID NO: 105 - DNA, 22 bp, synthetic construct
  source 1..22: mol_type = other DNA
atcaaattccagcaccgagcgc

SEQ ID NO: 106 - DNA, 23 bp, synthetic construct
  source 1..23: mol_type = other DNA
accattgaggacgtttgtctcac

SEQ ID NO: 107 - DNA, 23 bp, synthetic construct
  source 1..23: mol_type = other DNA
catgtcctcaagtcaagaacaag

SEQ ID NO: 108 - DNA, 24 bp, synthetic construct
  source 1..24: mol_type = other DNA
gctaggggagagtcccactgtcca

SEQ ID NO: 109 - DNA, 24 bp, synthetic construct
  source 1..24: mol_type = other DNA
gtagggtgtgatgggaggctaagc

SEQ ID NO: 110 - DNA, 24 bp, synthetic construct
  source 1..24: mol_type = other DNA
agaccgagtggcagtgacagcaag

SEQ ID NO: 111 - DNA, 24 bp, synthetic construct
  source 1..24: mol_type = other DNA
gtcttcctgctctgtgcgcacgac

SEQ ID NO: 112 - DNA, 60 bp, synthetic construct
  source 1..60: mol_type = other DNA
tagcggccgctcatgcgcggcgcattacctttacnnnnnnnnnnggatcctctagagtcg

SEQ ID NO: 113 - DNA, 58 bp, synthetic construct
  source 1..58: mol_type = other DNA
acaggaaacagctatgaccatgaaagcttgcatgcctgcaggtcgactctagaggatc

SEQ ID NO: 114 - DNA, 41 bp, synthetic construct
  source 1..41: mol_type = other DNA
ctacacgacgctcttccgatctcctggagcgtgtacgttgg

SEQ ID NO: 115 - DNA, 42 bp, synthetic construct
  source 1..42: mol_type = other DNA
ctacacgacgctcttccgatctcctgtggtcccagctacttg

SEQ ID NO: 116 - DNA, 41 bp, synthetic construct
  source 1..41: mol_type = other DNA
ctacacgacgctcttccgatctatctgcgatgtcctcgagg

SEQ ID NO: 117 - DNA, 40 bp, synthetic construct
  source 1..40: mol_type = other DNA
ctacacgacgctcttccgatcttggtgtgcgcctctaacg

SEQ ID NO: 118 - DNA, 45 bp, synthetic construct
  source 1..45: mol_type = other DNA
ctacacgacgctcttccgatctggagtcttgctttgtcactcaga

SEQ ID NO: 119 - DNA, 41 bp, synthetic construct
  source 1..41: mol_type = other DNA
ctacacgacgctcttccgatctagcctagacccagtcccat

SEQ ID NO: 120 - DNA, 42 bp, synthetic construct
  source 1..42: mol_type = other DNA
ctacacgacgctcttccgatctgctgggcatagtagtggact

SEQ ID NO: 121 - DNA, 41 bp, synthetic construct
  source 1..41: mol_type = other DNA
ctacacgacgctcttccgatcttggggaggctgagacacga

SEQ ID NO: 122 - DNA, 41 bp, synthetic construct
  source 1..41: mol_type = other DNA
ctacacgacgctcttccgatctcttgggaggctgaggcaag

SEQ ID NO: 123 - DNA, 40 bp, synthetic construct
  source 1..40: mol_type = other DNA
agacgtgtgctcttccgatctcaggaggatgagagccagg

SEQ ID NO: 124 - DNA, 43 bp, synthetic construct
  source 1..43: mol_type = other DNA
agacgtgtgctcttccgatctcagggtctcactctatcaccca

SEQ ID NO: 125 - DNA, 42 bp, synthetic construct
  source 1..42: mol_type = other DNA
agacgtgtgctcttccgatctactgaatgggttgaacttggc

SEQ ID NO: 126 - DNA, 45 bp, synthetic construct
  source 1..45: mol_type = other DNA
agacgtgtgctcttccgatctgagacagaatcttgctctgtctcc

SEQ ID NO: 127 - DNA, 40 bp, synthetic construct
  source 1..40: mol_type = other DNA
agacgtgtgctcttccgatcttcccagctacttgggaggc

SEQ ID NO: 128 - DNA, 42 bp, synthetic construct
  source 1..42: mol_type = other DNA
agacgtgtgctcttccgatctcctgcccaaatagggaagcag

SEQ ID NO: 129 - DNA, 42 bp, synthetic construct
  source 1..42: mol_type = other DNA
agacgtgtgctcttccgatcttggcgccttagtctctgctac

SEQ ID NO: 130 - DNA, 44 bp, synthetic construct
  source 1..44: mol_type = other DNA
agacgtgtgctcttccgatctgcatgagacacagtttcactctg

SEQ ID NO: 131 - DNA, 42 bp, synthetic construct
  source 1..42: mol_type = other DNA
agacgtgtgctcttccgatctgagagagtctcactgcgttgc

SEQ ID NO: 132 - DNA, 41 bp, synthetic construct
  source 1..41: mol_type = other DNA
ctacacgacgctcttccgatcttctctcacccactgggcac

SEQ ID NO: 133 - DNA, 40 bp, synthetic construct
  source 1..40: mol_type = other DNA
agacgtgtgctcttccgatctgcttccagacgagtgcaga

SEQ ID NO: 134 - DNA, 49 bp, synthetic construct
  source 1..49: mol_type = other DNA
ctacacgacgctcttccgatctaagttttcaaaccagaagaactacgac

SEQ ID NO: 135 - DNA, 42 bp, synthetic construct
  source 1..42: mol_type = other DNA
ctacacgacgctcttccgatctccggtataagtcctggagcg

SEQ ID NO: 136 - DNA, 40 bp, synthetic construct
  source 1..40: mol_type = other DNA
ctacacgacgctcttccgatctgccagggagcaatggcag

SEQ ID NO: 137 - DNA, 41 bp, synthetic construct
  source 1..41: mol_type = other DNA
ctacacgacgctcttccgatctcctcgaattccacggggtt

SEQ ID NO: 138 - DNA, 42 bp, synthetic construct
  source 1..42: mol_type = other DNA
ctacacgacgctcttccgatctgttggtgggagggaagtgag

SEQ ID NO: 139 - DNA, 40 bp, synthetic construct
  source 1..40: mol_type = other DNA
ctacacgacgctcttccgatctgatggcggttgtagcggc

SEQ ID NO: 140 - DNA, 46 bp, synthetic construct
  source 1..46: mol_type = other DNA
ctacacgacgctcttccgatctcacataaacctatgtttcagcaga

SEQ ID NO: 141 - DNA, 44 bp, synthetic construct
  source 1..44: mol_type = other DNA
ctacacgacgctcttccgatctgctagttggattgaagcagggt

SEQ ID NO: 142 - DNA, 40 bp, synthetic construct
  source 1..40: mol_type = other DNA
ctacacgacgctcttccgatctttgagtgcggcagcttcc

SEQ ID NO: 143 - DNA, 43 bp, synthetic construct
  source 1..43: mol_type = other DNA
ctacacgacgctcttccgatctataaccctcccaggcaaagtc

SEQ ID NO: 144 - DNA, 41 bp, synthetic construct
  source 1..41: mol_type = other DNA
ctacacgacgctcttccgatctagcctgcacatctgagctc

SEQ ID NO: 145 - DNA, 41 bp, synthetic construct
  source 1..41: mol_type = other DNA
ctacacgacgctcttccgatctggagcattgaagtgcctgg

SEQ ID NO: 146 - DNA, 38 bp, synthetic construct
  source 1..38: mol_type = other DNA
agacgtgtgctcttccgatctcagcctgggaccactga

SEQ ID NO: 147 - DNA, 39 bp, synthetic construct
  source 1..39: mol_type = other DNA
agacgtgtgctcttccgatctcatcctcgacagtcgcgg

SEQ ID NO: 148 - DNA, 47 bp, synthetic construct
  source 1..47: mol_type = other DNA
agacgtgtgctcttccgatctgactgatcaagtagaatactcatggg

SEQ ID NO: 149 - DNA, 39 bp, synthetic construct
  source 1..39: mol_type = other DNA
agacgtgtgctcttccgatctccctgccagcactgaagc

SEQ ID NO: 150 - DNA, 47 bp, synthetic construct
  source 1..47: mol_type = other DNA
agacgtgtgctcttccgatctggttcctatctttctagaccaggagt

SEQ ID NO: 151 - DNA, 39 bp, synthetic construct
  source 1..39: mol_type = other DNA
agacgtgtgctcttccgatctagtgtggagggctcaggg

SEQ ID NO: 152 - DNA, 40 bp, synthetic construct
  source 1..40: mol_type = other DNA
agacgtgtgctcttccgatctgatgggcagaggaaggcaa

SEQ ID NO: 153 - DNA, 41 bp, synthetic construct
  source 1..41: mol_type = other DNA
agacgtgtgctcttccgatcttcactctcatgagcgtccca

SEQ ID NO: 154 - DNA, 41 bp, synthetic construct
  source 1..41: mol_type = other DNA
ctacacgacgctcttccgatctaaggttccttgcggttcgc

SEQ ID NO: 155 - DNA, 38 bp, synthetic construct
  source 1..38: mol_type = other DNA
agacgtgtgctcttccgatctcgctgccattgctccct

SEQ ID NO: 156 - DNA, 43 bp, synthetic construct
  source 1..43: mol_type = other DNA
ctacacgacgctcttccgatcttctcgcacattcttcacgtcc

SEQ ID NO: 157 - DNA, 42 bp, synthetic construct
  source 1..42: mol_type = other DNA
agacgtgtgctcttccgatctaggaaccttcccgacttaggg

SEQ ID NO: 158 - DNA, 44 bp, synthetic construct
  source 1..44: mol_type = other DNA
ctacacgacgctcttccgatctcccgcccatcttctagaaagac

SEQ ID NO: 159 - DNA, 40 bp, synthetic construct
  source 1..40: mol_type = other DNA
ctacacgacgctcttccgatcttgccaggtgagggactgg

SEQ ID NO: 160 - DNA, 41 bp, synthetic construct
  source 1..41: mol_type = other DNA
agacgtgtgctcttccgatcttctgggagttctctgctgcc

SEQ ID NO: 161 - DNA, 41 bp, synthetic construct
  source 1..41: mol_type = other DNA
agacgtgtgctcttccgatcttgcccaaccttagcaaggag

SEQ ID NO: 162 - DNA, 40 bp, synthetic construct
  source 1..40: mol_type = other DNA
ctacacgacgctcttccgatcttaccttggagcaacggcg

SEQ ID NO: 163 - DNA, 39 bp, synthetic construct
  source 1..39: mol_type = other DNA
agacgtgtgctcttccgatctcccaggacgaggatggag

SEQ ID NO: 164 - DNA, 20 bp, synthetic construct
  source 1..20: mol_type = other DNA
gatagtcactccaggggttg

SEQ ID NO: 165 - DNA, 20 bp, synthetic construct
  source 1..20: mol_type = other DNA
gtggtgaaccaatcagtcct

SEQ ID NO: 166 - protein, 1140 aa, synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRGTGGPKKKRKVYPYDVPDYAGYPYDVPDYAGSYPYDVPDYAGSAAPAAKKKKLDFESG

SEQ ID NO: 167 - protein, 1168 aa, synthetic construct
MVPKKKRKVEDKRPAATKKAGQAKKKKMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVREDKRPAATKKAGQAKKKKYPYDVPDYAGYPYDVPDYAGSYPYDVPDYAAAPAAKKKKLD

SEQ ID NO: 168 - protein, 1135 aa, synthetic construct
PKKKRKVNAMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRGGGGSGGGGSGGGGSPAAKKKKLDGGGSKRPAATKKAGQAKKKK

SEQ ID NO: 169 - DNA, 25 bp, synthetic construct
  source 1..25: mol_type = other DNA
ggtagggtgtgatgggaggctaagc

SEQ ID NO: 170 - DNA, 23 bp, synthetic construct
  source 1..23: mol_type = other DNA
cttcatctgtatcttcaggatca

SEQ ID NO: 171 - protein, 6 aa, synthetic construct
HAHAHA

SEQ ID NO: 172 - protein, 6 aa, synthetic construct
HHHHHH

SEQ ID NO: 173 - DNA, 30 bp, synthetic construct
  source 1..30: mol_type = other DNA
gcggtctaggtgccctcgtagggtcggccc

SEQ ID NO: 174 - DNA, 24 bp, synthetic construct
  source 1..24: mol_type = other DNA
actcactcacccacacagacacac

SEQ ID NO: 175 - DNA, 15 bp, synthetic construct
  source 1..15: mol_type = other DNA
cacacacgtcctcac

SEQ ID NO: 176 - DNA, 39 bp, synthetic construct
  source 1..39: mol_type = other DNA
gtgtgtgagtgagtgggtgtgtctgtgtgtgcaggagtg

SEQ ID NO: 177 - RNA, 29 nt, synthetic construct
  source 1..29: mol_type = other RNA
  ncRNA 1..29: ncRNA_class = sgRNA
gctcactcacccacacagacacacgttgt

SEQ ID NO: 178 - DNA, 30 bp, synthetic construct
  source 1..30: mol_type = other DNA
ctcactcacccacacagacacacacgtcct

SEQ ID NO: 179 - DNA, 30 bp, synthetic construct
  source 1..30: mol_type = other DNA
tccagctcgaccaggatgggcaccaccccg

SEQ ID NO: 180 - DNA, 30 bp, synthetic construct
  source 1..30: mol_type = other DNA
ttcagctcgaccaggatgggcaccaccccg

SEQ ID NO: 181 - DNA, 30 bp, synthetic construct
  source 1..30: mol_type = other DNA
tccagcttgaccaggatgggcaccaccccg

SEQ ID NO: 182 - DNA, 30 bp, synthetic construct
  source 1..30: mol_type = other DNA
ttcagcttgaccaggatgggcaccaccccg

SEQ ID NO: 183 - DNA, 30 bp, synthetic construct
  source 1..30: mol_type = other DNA
ggtcgagctggacggcgacgtaaacggcca

SEQ ID NO: 184 - DNA, 30 bp, synthetic construct
  source 1..30: mol_type = other DNA
ggttgagctggacggcgacgtaaacggcca

SEQ ID NO: 185 - DNA, 30 bp, synthetic construct
  source 1..30: mol_type = other DNA
agttcatctgcaccaccggcaagctgcccg

SEQ ID NO: 186 - DNA, 30 bp, synthetic construct
  source 1..30: mol_type = other DNA
agtttatctgcaccaccggcaagctgcccg

SEQ ID NO: 187 - DNA, 30 bp, synthetic construct
  source 1..30: mol_type = other DNA
agttcatttgcaccaccggcaagctgcccg

SEQ ID NO: 188 - DNA, 30 bp, synthetic construct
  source 1..30: mol_type = other DNA
cgtgccctggcccaccctcgtgaccaccct

SEQ ID NO: 189 - DNA, 30 bp, synthetic construct
  source 1..30: mol_type = other DNA
cgtgccttggcccaccctcgtgaccaccct

SEQ ID NO: 190 - DNA, 40 bp, synthetic construct
  source 1..40: mol_type = other DNA
aacgactggagcagtaatgcctggtggcccagcttcctct

SEQ ID NO: 191 - DNA, 40 bp, synthetic construct
  source 1..40: mol_type = other DNA
aacgactggagcggtaatgcctggtggcccagcttcctct

SEQ ID NO: 192 - RNA, 24 nt, synthetic construct
  source 1..24: mol_type = other RNA
  ncRNA 1..24: ncRNA_class = sgRNA
gtgaacttgtggccgtttacgtcc

SEQ ID NO: 193 - RNA, 24 nt, synthetic construct
  source 1..24: mol_type = other RNA
  ncRNA 1..24: ncRNA_class = sgRNA
gtgaacttgtggccgtttacgtgg

SEQ ID NO: 194 - RNA, 24 nt, synthetic construct
  source 1..24: mol_type = other RNA
  ncRNA 1..24: ncRNA_class = sgRNA
gtgaacttgtggccgtttacgacg

SEQ ID NO: 195 - RNA, 24 nt, synthetic construct
  source 1..24: mol_type = other RNA
  ncRNA 1..24: ncRNA_class = sgRNA
gtgaacttgtggccgtttacctcg

SEQ ID NO: 196 - RNA, 24 nt, synthetic construct
  source 1..24: mol_type = other RNA
  ncRNA 1..24: ncRNA_class = sgRNA
gtgaacttgtggccgtttaggtcg

SEQ ID NO: 197 - RNA, 24 nt, synthetic construct
  source 1..24: mol_type = other RNA
  ncRNA 1..24: ncRNA_class = sgRNA
gtgaacttgtggccgttttcgtcg

SEQ ID NO: 198 - RNA, 24 nt, synthetic construct
  source 1..24: mol_type = other RNA
  ncRNA 1..24: ncRNA_class = sgRNA
gtgaacttgtggccgttaacgtcg

SEQ ID NO: 199 - RNA, 24 nt, synthetic construct
  source 1..24: mol_type = other RNA
  ncRNA 1..24: ncRNA_class = sgRNA
gtgaacttgtggccgtatacgtcg

SEQ ID NO: 200 - RNA, 24 nt, synthetic construct
  source 1..24: mol_type = other RNA
  ncRNA 1..24: ncRNA_class = sgRNA
gtgaacttgtggccgattacgtcg

SEQ ID NO: 201 - RNA, 24 nt, synthetic construct
  source 1..24: mol_type = other RNA
  ncRNA 1..24: ncRNA_class = sgRNA
gtgaacttgtggccctttacgtcg

SEQ ID NO: 202 - RNA, 24 nt, synthetic construct
  source 1..24: mol_type = other RNA
  ncRNA 1..24: ncRNA_class = sgRNA
gtgaacttgtggcggtttacgtcg

SEQ ID NO: 203 - RNA, 24 nt, synthetic construct
  source 1..24: mol_type = other RNA
  ncRNA 1..24: ncRNA_class = sgRNA
gtgaacttgtgggcgtttacgtcg

SEQ ID NO: 204 - RNA, 24 nt, synthetic construct
  source 1..24: mol_type = other RNA
  ncRNA 1..24: ncRNA_class = sgRNA
gtgaacttgtgcccgtttacgtcg

SEQ ID NO: 205 - RNA, 24 nt, synthetic construct
  source 1..24: mol_type = other RNA
  ncRNA 1..24: ncRNA_class = sgRNA
gtgaacttgtcgccgtttacgtcg

SEQ ID NO: 206 - RNA, 24 nt, synthetic construct
  source 1..24: mol_type = other RNA
  ncRNA 1..24: ncRNA_class = sgRNA
gtgaacttgaggccgtttacgtcg

SEQ ID NO: 207 - RNA, 24 nt, synthetic construct
  source 1..24: mol_type = other RNA
  ncRNA 1..24: ncRNA_class = sgRNA
gtgaacttctggccgtttacgtcg

SEQ ID NO: 208 - RNA, 24 nt, synthetic construct
  source 1..24: mol_type = other RNA
  ncRNA 1..24: ncRNA_class = sgRNA
gtgaactagtggccgtttacgtcg

SEQ ID NO: 209 - RNA, 24 nt, synthetic construct
  source 1..24: mol_type = other RNA
  ncRNA 1..24: ncRNA_class = sgRNA
gtgaacatgtggccgtttacgtcg

SEQ ID NO: 210 - RNA, 24 nt, synthetic construct
  source 1..24: mol_type = other RNA
  ncRNA 1..24: ncRNA_class = sgRNA
gtgaagttgtggccgtttacgtcg

SEQ ID NO: 211 - RNA, 24 nt, synthetic construct
  source 1..24: mol_type = other RNA
  ncRNA 1..24: ncRNA_class = sgRNA
gtgatcttgtggccgtttacgtcg

SEQ ID NO: 212 - RNA, 24 nt, synthetic construct
  source 1..24: mol_type = other RNA
  ncRNA 1..24: ncRNA_class = sgRNA
gtgtacttgtggccgtttacgtcg

SEQ ID NO: 213 - RNA, 24 nt, synthetic construct
  source 1..24: mol_type = other RNA
  ncRNA 1..24: ncRNA_class = sgRNA
gtcaacttgtggccgtttacgtcg

SEQ ID NO: 214 - RNA, 24 nt, synthetic construct
  source 1..24: mol_type = other RNA
  ncRNA 1..24: ncRNA_class = sgRNA
gagaacttgtggccgtttacgtcg

SEQ ID NO: 215 - DNA, 31 bp, synthetic construct
  source 1..31: mol_type = other DNA
cacacacacccacacagacacaccccccccc

SEQ ID NO: 216 - DNA, 30 bp, synthetic construct
  source 1..30: mol_type = other DNA
ctccctcacacacacagacacacacctccc

SEQ ID NO: 217 - DNA, 30 bp, synthetic construct
  source 1..30: mol_type = other DNA
cacacacacacacacagacacacacacccc

SEQ ID NO: 218 - DNA, 31 bp, synthetic construct
  source 1..31: mol_type = other DNA
gtgtgtccctctccccacccgtccctgtccg

SEQ ID NO: 219 - DNA, 31 bp, synthetic construct
  source 1..31: mol_type = other DNA
ttgtctccctgtccccacccgtccccttcag

SEQ ID NO: 220 - DNA, 31 bp, synthetic construct
  source 1..31: mol_type = other DNA
ctgcctccctctgcccacccgtccttcccac

SEQ ID NO: 221 - DNA, 31 bp, synthetic construct
  source 1..31: mol_type = other DNA
ctgtgcctctctccccacccttccacaccct

SEQ ID NO: 222 - DNA, 31 bp, synthetic construct
  source 1..31: mol_type = other DNA
gctcatccccctccccacccgtcctcgcccg

SEQ ID NO: 223 - DNA, 31 bp, synthetic construct
  source 1..31: mol_type = other DNA
tgaggaccgccctgggcctgggagaatccct

SEQ ID NO: 224 - DNA, 31 bp, synthetic construct
  source 1..31: mol_type = other DNA
gaaggaccaccctaggcctgggagactccct

SEQ ID NO: 225 - protein, 11 aa, synthetic construct
KQERRPPSGRG

SEQ ID NO: 226 - protein, 10 aa, synthetic construct
KQERSPSGRG

SEQ ID NO: 227 - DNA, 33 bp, synthetic construct
  source 1..33: mol_type = other DNA
aaagacgagagtggccctccccgagggagaggg

SEQ ID NO: 228 - DNA, 33 bp, synthetic construct
  source 1..33: mol_type = other DNA
tttctgctctcaccgggaggggctccctctccc

SEQ ID NO: 229 - DNA, 36 bp, synthetic construct
  source 1..36: mol_type = other DNA
gttttacatgaatgtccgaggtcatcacgagtcagg

SEQ ID NO: 230 - DNA, 36 bp, synthetic construct
  source 1..36: mol_type = other DNA
caaaatgtacttacaggctccagtagtgctcagtcc

SEQ ID NO: 231 - DNA, 23 bp, synthetic construct
  source 1..23: mol_type = other DNA
cgcaaagctgcatccaccccccg

SEQ ID NO: 232 - DNA, 24 bp, synthetic construct
  source 1..24: mol_type = other DNA
tgaggaccgccctgggcctgggag

SEQ ID NO: 233 - DNA, 30 bp, synthetic construct
  source 1..30: mol_type = other DNA
aacgactggagcagtaatgcctggtggccc

SEQ ID NO: 234 - DNA, 20 bp, synthetic construct
  source 1..20: mol_type = other DNA
ctggaccccacgagacatcc

SEQ ID NO: 235 - DNA, 30 bp, synthetic construct
  source 1..30: mol_type = other DNA
ggcggctggagcaggattgcctggagttcc

SEQ ID NO: 236 - DNA, 30 bp, synthetic construct
  source 1..30: mol_type = other DNA
caggattggaacagtaatgactggcaaacc

SEQ ID NO: 237 - DNA, 33 bp, synthetic construct
  source 1..33: mol_type = other DNA
ggccgaccctacgagggcacctagaccgccaag

SEQ ID NO: 238 - DNA, 33 bp, synthetic construct
  source 1..33: mol_type = other DNA
ccggctgggatgctcccgtggatctggcggttc

SEQ ID NO: 239 - protein, 9 aa, synthetic construct
GRPYETTAK

SEQ ID NO: 240 - DNA, 24 bp, synthetic construct
  source 1..24: mol_type = other DNA
gcggtctaggtgccctcgtagggt

SEQ ID NO: 241 - DNA, 24 bp, synthetic construct
  source 1..24: mol_type = other DNA
gcggtctgggtgccctcgtagggt

SEQ ID NO: 242 - RNA, 98 nt, synthetic construct
  source 1..98: mol_type = other RNA
  ncRNA 1..98: ncRNA_class = guide_RNA
  mRNA 1..98: note = Guide mRNA
cctgtctcaggcggtgctcctagcagacatccttcccacccttgtcatcaaactcctggcgcctcttggccttcacttgctgccttacaggtctgggt

SEQ ID NO: 243 - RNA, 98 nt, synthetic construct
  source 1..98: mol_type = other RNA
  mRNA 1..98: note = Guide mRNA
  ncRNA 1..98: ncRNA_class = guide_RNA
ggacagagtccgccacgaggatcgtctgtaggaagggtgggaacagtagtttgaggaccgcggagaaccggaagtgaacgacggaatgtccagaccca

SEQ ID NO: 244 - RNA, 98 nt, synthetic construct
  source 1..98: mol_type = other RNA
  mRNA 1..98: note = Guide mRNA
  ncRNA 1..98: ncRNA_class = guide_RNA
cctgtcccaggctgtgctcctggcggacatcctccccacactcgtcatcaaattgttggctcctcttggccttcacctgctgccctacaggtctgggt

SEQ ID NO: 245 - RNA, 98 nt, synthetic construct
  source 1..98: mol_type = other RNA
  mRNA 1..98: note = Guide mRNA
  ncRNA 1..98: ncRNA_class = guide_RNA
ggacagggtccgacacgaggaccgcctgtaggaggggtgtgagcagtagtttaacaaccgaggagaaccggaagtggacgacgggatgtccagaccca

SEQ ID NO: 246 - DNA, 30 bp, Mus musculus
  source 1..30: mol_type = other DNA
tgtctcaggcggtgctcctagcagacatcc

SEQ ID NO: 247 - DNA, 30 bp, Homo sapiens
  source 1..30: mol_type = other DNA
tgtcccaggctgtgctcctggcggacatcc

SEQ ID NO: 248 - DNA, 30 bp, Mus musculus
  source 1..30: mol_type = other DNA
cccagacctgtaaggcagcaagtgaaggcc

SEQ ID NO: 249 - DNA, 30 bp, Homo sapiens
  source 1..30: mol_type = other DNA
cccagacctgtagggcagcaggtgaaggcc

SEQ ID NO: 250 - DNA, 30 bp, synthetic construct
  source 1..30: mol_type = other DNA
ccacagctcccccgggtgctcgtcagtgga

SEQ ID NO: 251 - DNA, 35 bp, synthetic construct
  source 1..35: mol_type = other DNA
aactccatctccacagctcccccgggtgctcgtca

SEQ ID NO: 252 - protein, 13 aa, synthetic construct
GRGSPPRREQKPP

SEQ ID NO: 253 - protein, 12 aa, synthetic construct
GRGSPSREQKPP

SEQ ID NO: 254 - DNA, 40 bp, synthetic construct
  source 1..40: mol_type = other DNA
gggagagggagcccctcccggtgagaagcagaaaccacct

SEQ ID NO: 255 - DNA, 39 bp, synthetic construct
  source 1..39: mol_type = other DNA
ccctctccctcggggagggccactctcgtctttggtgga

SEQ ID NO: 256 - DNA, 30 bp, synthetic construct
  source 1..30: mol_type = other DNA
tctgctctcaccgggaggggctccctctcc

SEQ ID NO: 257 - DNA, 30 bp, synthetic construct
  source 1..30: mol_type = other DNA
gtggtttctgctctcaccgggaggggctcc

SEQ ID NO: 258 - protein, 12 aa, synthetic construct
QETAPPTKKRKT

SEQ ID NO: 259 - protein, 12 aa, synthetic construct
QETVLPIKKCKT

SEQ ID NO: 260 - DNA, 36 bp, synthetic construct
  source 1..36: mol_type = other DNA
caggagaccgtactccccatcaagaagtgcaagacc

SEQ ID NO: 261 - DNA, 36 bp, synthetic construct
  source 1..36: mol_type = other DNA
gtcctctggcatgaggggtagttcttcacgttctgg

SEQ ID NO: 262 - DNA, 30 bp, synthetic construct
  source 1..30: mol_type = other DNA
ttgcacttcttgatggggagtacggtctcc

SEQ ID NO: 263 - protein, 12 aa, synthetic construct
MVIKRPGRKRKA

SEQ ID NO: 264 - protein, 11 aa, synthetic construct
MVIKRPGRKKA

SEQ ID NO: 265 - DNA, 36 bp, synthetic construct
  source 1..36: mol_type = other DNA
atggtgatcaaacgccccggcaggaagtgaaaagct

SEQ ID NO: 266 - DNA, 36 bp, synthetic construct
  source 1..36: mol_type = other DNA
taccactagtttgcggggccgtccttcacttttcga

SEQ ID NO: 267 - DNA, 30 bp, synthetic construct
  source 1..30: mol_type = other DNA
tttcacttcctgccggggcgtttgatcacc

SEQ ID NO: 268 - protein, 10 aa, synthetic construct
  VARIANT 3: note = I can be replaced by T
QAIPKKRGRK

SEQ ID NO: 269 - protein, 9 aa, synthetic construct
QAIPKKRGK

SEQ ID NO: 270 - DNA, 30 bp, synthetic construct
  source 1..30: mol_type = other DNA
caggccattcccaagaaacggggctgaaag

SEQ ID NO: 271 - DNA, 30 bp, synthetic construct
  source 1..30: mol_type = other DNA
gtccggtaagggttctttgccccgactttc

SEQ ID NO: 272 - DNA, 30 bp, synthetic construct
  source 1..30: mol_type = other DNA
ggctttcagccccgtttcttgggaatggcc

SEQ ID NO: 273 - DNA, 30 bp, synthetic construct
  source 1..30: mol_type = other DNA
tgaggaccgccctgggcctgggagaatccc

SEQ ID NO: 274 - DNA, 11 bp, synthetic construct
  source 1..11: mol_type = other DNA
gaaaaaatccc

SEQ ID NO: 275 - RNA, 30 nt, synthetic construct
  source 1..30: mol_type = other RNA
ggcgtctaggtgccctcgtagggtcggccc

SEQ ID NO: 276 - RNA, 30 nt, synthetic construct
  source 1..30: mol_type = other RNA
gcgcactaggtgccctcgtagggtcggccc

SEQ ID NO: 277 - RNA, 30 nt, synthetic construct
  source 1..30: mol_type = other RNA
gcggtgaaggtgccctcgtagggtcggccc

SEQ ID NO: 278 - RNA, 30 nt, synthetic construct
  source 1..30: mol_type = other RNA
ggcgtcttcgtgccctcgtagggtcggccc

SEQ ID NO: 279 - RNA, 30 nt, synthetic construct
  source 1..30: mol_type = other RNA
gcggtctagcagccctcgtagggtcggccc

SEQ ID NO: 280 - RNA, 30 nt, synthetic construct
  source 1..30: mol_type = other RNA
gcggtctaggtcgcctcgtagggtcggccc

SEQ ID NO: 281 - RNA, 30 nt, synthetic construct
  source 1..30: mol_type = other RNA
gcggtctaggtgcggtcgtagggtcggccc

SEQ ID NO: 282 - RNA, 30 nt, synthetic construct
  source 1..30: mol_type = other RNA
gcggtctaggtgcccaggtagggtcggccc

SEQ ID NO: 283 - RNA, 30 nt, synthetic construct
  source 1..30: mol_type = other RNA
gcggtctaggtgccctccaagggtcggccc

SEQ ID NO: 284 - DNA, 29 bp, synthetic construct
  source 1..29: mol_type = other DNA
ccgcaaagctgcatccaccccccaggacc

SEQ ID NO: 285 - DNA, 32 bp, synthetic construct
  source 1..32: mol_type = other DNA
ccgcaaagctgcatccaccccccgagaggacc

SEQ ID NO: 286 - DNA, 30 bp, synthetic construct
  source 1..30: mol_type = other DNA
tgacttcacggtaactgggagagggagccc

SEQ ID NO: 287 - DNA, 30 bp, synthetic construct
  source 1..30: mol_type = other DNA
atgctggtgtctaggagatacacctccacc

SEQ ID NO: 288 - DNA, 30 bp, synthetic construct
  source 1..30: mol_type = other DNA
ccgcaaagctgcatccaccccccgaggacc

SEQ ID NO: 289 - DNA, 30 bp, synthetic construct
  source 1..30: mol_type = other DNA
cgcaaagctgcatccaccccccgaggacca

SEQ ID NO: 290 - DNA, 30 bp, synthetic construct
  source 1..30: mol_type = other DNA
gctgcatccaccccccgaggaccagcccct

SEQ ID NO: 291 - DNA, 30 bp, synthetic construct
  source 1..30: mol_type = other DNA
tgcatccaccccccgaggaccagcccctcg

SEQ ID NO: 292 - DNA, 39 bp, synthetic construct
  source 1..39: mol_type = other DNA
gggagagggagcccctcccggtgagagcagaaaccacct

SEQ ID NO: 293 - DNA, 30 bp, synthetic construct
  source 1..30: mol_type = other DNA
ggtttctgctctcaccgggaggggctccct

SEQ ID NO: 294 - DNA, 29 bp, synthetic construct
  source 1..29: mol_type = other DNA
tcttgcacttcttgatggggagtcggtct

SEQ ID NO: 295 - DNA, 30 bp, synthetic construct
  source 1..30: mol_type = other DNA
ttcacttcctgccggggcgtttgatcacca

SEQ ID NO: 296 - DNA, 30 bp, synthetic construct
  source 1..30: mol_type = other DNA
gctttcagccccgtttcttgggaatggcct

SEQ ID NO: 297 - DNA, 40 bp, synthetic construct
  source 1..40: mol_type = other DNA
tgcacttttcttaaaggaaagtaatggaccagtgaaggtg

SEQ ID NO: 298 - DNA, 36 bp, synthetic construct
  source 1..36: mol_type = other DNA
  modified_base 13: mod_base = OTHER; note = uracil
tcgtgttgtgggtggacgacataatagaggtttgag

SEQ ID NO: 299 - DNA, 32 bp, synthetic construct
  source 1..32: mol_type = other DNA
gggaagcattaaaagactgactgaaggcctgc

SEQ ID NO: 300 - protein, 1129 aa, synthetic construct
KRTADGSEFESPKKKRKVEDMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 301 - DNA, 3390 bp, synthetic construct
  source 1..3390: mol_type = other DNA
aaacggacagccgacggaagcgagttcgagtcaccaaagaagaagcggaaagtcgaagatatggccgccttcaagcctaacccaatcaattacatcctgggactggacatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtctaa

SEQ ID NO: 302 - protein, 1172 aa, synthetic construct
PKKKRKVEDKRPAATKKAGQAKKKKMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKKRPAATKKAGQAKKKKYPYDVPDYAGYPYDVPDYAGSYPYDVPDYAAAPAAKKKKLD

SEQ ID NO: 303 - DNA, 3519 bp, synthetic construct
  source 1..3519: mol_type = other DNA
cctaagaagaagagaaaggtggaagataaacgcccagcagctacaaagaaggcaggtcaagccaagaaaaagaaaatggccgccttcaagcctaacccaatcaattacatcctgggactggacatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaatggcgatatggtgcgggtggacgtgtacaccaaagctggcaaaaactacctggttcctgtgtacgtgtggcaagtggctcagggcatcctgccaaatagagccgtgaccagcggaaaatccgaagccgactgggacctgatcgacgagagcttcgagttcaagttctccctgagcagaggcgatctggttgaaatgatcagcaacaagggaaggatctttggctactacaacggcctggacagagcgaatggcagcataggaatcagagagcacgacctggaaaagagcaaaggcaaagatggcgtgcatagagtaggcgtgaaaaccgccaccgccttcaacaagtaccacgtggaccctctgggcaaggaaatccacagatgcagcagcgagcctagacctaccctcaaaatcaagtccaagaaaaagcggcccgcagcaaccaagaaggcaggacaggccaagaagaagaagtacccctatgacgtgcctgactacgccgggtatccctacgacgtgcctgattacgccgggtcctatccctacgacgtgccagattacgctgcagctccagcagcgaagaaaaagaagctggattaa

SEQ ID NO: 304 - protein, 1335 aa, synthetic construct
KRTADGSEFESPKKKRKVEDMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGGSGGSGGSGGSGGSGGSGGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGSSGSETPGTSESATPESSGGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 305 - DNA, 4008 bp, synthetic construct
  source 1..4008: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtctaa

SEQ ID NO: 306 - protein, 1335 aa, synthetic construct
KRTADGSEFESPKKKRKVEDMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKEGGSGGSGGSGGSGGSGGSGGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGSSGSETPGTSESATPESSGTGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 307 - DNA, 4008 bp, synthetic construct
  source 1..4008: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtctaa

SEQ ID NO: 308 - protein, 1335 aa, synthetic construct
KRTADGSEFESPKKKRKVEDMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPGGSGGSGGSGGSGGSGGSGGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINGSSGSETPGTSESATPESSGEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 309 - DNA, 4008 bp, synthetic construct
  source 1..4008: mol_type = other DNA
aaacggacagccgacggaagcgagttcgagtcaccaaagaagaagcggaaagtcgaagatatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctggcggatcaggaggctctggcggttcaggtggatcaggcggtagcggaggttcaggtggttctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaacggctcctctggctctgagacacctggcacaagcgagagcgcaacacctgaaagcagcggcgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtctaa

SEQ ID NO: 310 - protein, 1337 aa, synthetic construct
KRTADGSEFESPKKKRKVGGSGGGSGGGSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSMAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNATMVRVDVYTKAGKNYLVPVYVWQVAQGILPNRAVTSGKSEADWDLIDESFEFKFSLSRGDLVEMISNKGRIFGYYNGLDRANGSIGIREHDLEKSKGKDGVHRVGVKTATAFNKYHVDPLGKEIHRCSSEPRPTLKIKSKKEDKRTADGSEFEPKKKRKV

SEQ ID NO: 311 - DNA, 4014 bp, synthetic construct
  source 1..4014: mol_type = other DNA
aaacggacagccgacggaagcgagttcgagtcaccaaagaagaagcggaaagtcggcggtagcggcggaggcagcggtggcggcagcggctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacagcaaacggggcgccgcaggctccctgatgaacgtgctgaactaccccggcatgaatcaccgcgtcgaaattaccgagggaatcctggcagatgaatgtgccgccctgctgtgcgacttctaccggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcaatggccgccttcaagcctaacccaatcaattacatcctgggactggccatcggaatcgcatccgtgggatgggctatggtggagatcgacgaggaggagaatcctatccggctgatcgatctgggcgtgagagtgtttgagagggccgaggtgccaaagaccggcgattctctggctatggcccggagactggcacggagcgtgaggcgcctgacacggagaagggcacacaggctgctgagggcacgccggctgctgaagagagagggcgtgctgcaggcagcagacttcgatgagaatggcctgatcaagagcctgccaaacaccccctggcagctgagagcagccgccctggacaggaagctgacaccactggagtggtctgccgtgctgctgcacctgatcaagcaccgcggctacctgagccagcggaagaacgagggagagacagcagacaaggagctgggcgccctgctgaagggagtggccaacaatgcccacgccctgcagaccggcgatttcaggacacctgccgagctggccctgaataagtttgagaaggagtccggccacatcagaaaccagaggggcgactatagccacaccttctcccgcaaggatctgcaggccgagctgatcctgctgttcgagaagcagaaggagtttggcaatccacacgtgagcggaggcctgaaggagggaatcgagaccctgctgatgacacagaggcctgccctgtccggcgacgcagtgcagaagatgctgggacactgcaccttcgagcctgcagagccaaaggccgccaagaacacctacacagccgagcggtttatctggctgacaaagctgaacaatctgagaatcctggagcagggatccgagaggccactgaccgacacagagagggccaccctgatggatgagccttaccggaagtctaagctgacatatgcccaggccagaaagctgctgggcctggaggacaccgccttctttaagggcctgagatacggcaaggataatgccgaggcctccacactgatggagatgaaggcctatcacgccatctctcgcgccctggagaaggagggcctgaaggacaagaagtcccccctgaacctgagctccgagctgcaggatgagatcggcaccgccttctctctgtttaagaccgacgaggatatcacaggccgcctgaaggacagggtgcagcctgagatcctggaggccctgctgaagcacatctctttcgataagtttgtgcagatcagcctgaaggccctgagaaggatcgtgccactgatggagcagggcaagcggtacgacgaggcctgcgccgagatctacggcgatcactatggcaagaagaacacagaggagaagatctatctgccccctatccctgccgacgagatcagaaatcctgtggtgctgagggccctgtcccaggcaagaaaagtgatcaacggagtggtgcgccggtacggatctccagcccggatccacatcgagaccgccagagaagtgggcaagagcttcaaggaccggaaggagatcgagaagagacaggaggagaatcgcaaggatcgggagaaggccgccgccaagtttagggagtacttccctaactttgtgggcgagccaaagtctaaggacatcctgaagctgcgcctgtacgagcagcagcacggcaagtgtctgtatagcggcaaggagatcaatctggtgcggctgaacgagaagggctatgtggagatcgatcacgccctgcctttctccagaacctgggacgattcttttaacaataaggtgctggtgctgggcagcgagaaccagaataagggcaatcagacaccatacgagtatttcaatggcaaggacaactccagggagtggcaggagttcaaggcccgcgtggagacctctagatttcccaggagcaagaagcagcggatcctgctgcagaagttcgacgaggatggctttaaggagtgcaacctgaatgacaccagatacgtgaaccggttcctgtgccagtttgtggccgatcacatcctgctgaccggcaagggcaagagaagggtgttcgcctctaatggccagatcacaaacctgctgaggggattttggggactgaggaaggtgcgggcagagaatgacagacaccacgcactggatgcagtggtggtggcatgcagcaccgtggcaatgcagcagaagatcacaagattcgtgaggtataaggagatgaacgcctttgacggcaagaccatcgataaggagacaggcaaggtgctgcaccagaagacccacttcccccagccttgggagttctttgcccaggaagtgatgatccgggtgttcggcaagccagacggcaagcctgagtttgaggaggccgataccccagagaagctgaggacactgctggcagagaagctgtctagcaggccagaggcagtgcacgagtacgtgaccccactgttcgtgtccagggcacccaatcggaagatgtctggcgcccacaaggacacactgagaagcgccaagaggtttgtgaagcacaacgagaagatctccgtgaagagagtgtggctgaccgagatcaagctggccgatctggagaacatggtgaattacaagaacggcagggagatcgagctgtatgaggccctgaaggcaaggctggaggcctacggaggaaatgccaagcaggccttcgacccaaaggataaccccttttataagaagggaggacagctggtgaaggccgtgcgggtggagaagacccaggagagcggcgtgctgctgaataagaagaacgcctacacaatcgccgacaacgccaccatggtgcgggtggacgtgtacaccaaggccggcaagaactacctggttcctgtgtacgtgtggcaggtggcccagggcatcttacccaaccgcgccgtgaccagcggcaagtccgaggctgactgggacctgatcgatgagagcttcgagttcaagttctctctgtcccggggagatctcgtggaaatgatctccaacaagggcagaatcttcggctactacaacggcctggacagagccaacggctctattggaattagagagcacgacctagagaagagcaagggcaaagacggcgtgcatagagtgggagtgaaaacagctacagcatttaacaagtaccacgtggatcccctgggcaaagagatccacagatgcagcagcgaacccagacctacactgaaaatcaagtctaagaaggaggataaaagaaccgccgacggcagcgaattcgagcccaagaagaagaggaaagtctaa

SEQ ID NO: 312 - DNA, 240 bp, synthetic construct
  source 1..240: mol_type = other DNA
ccggctgggttgggaaggttgtctccgggaaggtctaggctctcactgtgttctcctgtctcaggcggtgctcctagcagacatccttcccacccttgtcatcaaactcctggcgcctcttggccttcacttgctgccttacaggtctgggtcggggtggcaggagggaggcagggtgggaggaagcgggagtctggagagtctcgcatggaggtggccttctgagttcctcctcatctc

SEQ ID NO: 313 - DNA, 240 bp, synthetic construct
  source 1..240: mol_type = other DNA
ggccgacccaacccttccaacagaggcccttccagatccgagagtgacacaagaggacagagtccgccacgaggatcgtctgtaggaagggtgggaacagtagtttgaggaccgcggagaaccggaagtgaacgacggaatgtccagacccagccccaccgtcctccctccgtcccaccctccttcgccctcagacctctcagagcgtacctccaccggaagactcaaggaggagtagag

SEQ ID NO: 314 - DNA, 30 bp, synthetic construct
  source 1..30: mol_type = other DNA
gcggtcttcgtgccctcgtagggtcggccc

SEQ ID NO: 315 - DNA, 30 bp, synthetic construct
  source 1..30: mol_type = other DNA
gcggtctaggtgccctcgttcggtcggccc

SEQ ID NO: 316 - DNA, 30 bp, synthetic construct
  source 1..30: mol_type = other DNA
gcggtctaggtgccctcgtagcctcggccc

SEQ ID NO: 317 - RNA, 24 nt, synthetic construct
  source 1..24: mol_type = other RNA
  ncRNA 1..24: ncRNA_class = sgRNA
gtgaacttgtggccgtttacgtcg

(19) United States
(12) Patent Application Publication
(10) Pub. No.: US 2024/0408238 A1
(43) Pub. Date: Dec. 12, 2024

(54) NME2CAS9 INLAID DOMAIN FUSION PROTEINS

(71) Applicant: UNIVERSITY OF MASSACHUSETTS, Boston, MA (US)

(72) Inventors: Erik Joseph SONTHEIMER, Auburndale, MA (US); Wen XUE, Natick, MA (US); Han ZHANG, Worcester, MA (US); Nathan BAMIDELE, Shrewsbury, MA (US); Xiaolong DONG, Worcester, MA (US)

(21) Appl. No.: 18/649,101

(22) Filed: Apr. 29, 2024

Related U.S. Application Data
(63) Continuation of application No. PCT/US2022/048261, filed on Oct. 28, 2022.
(60) Provisional application No. 63/274,667, filed on Nov. 2, 2021.

(51) Int. Cl. A61K 48/00; C12N 9/22; C12N 9/78; C12N 15/11

(52) U.S. Cl. CPC A61K 48/0058; C12N 9/22; C12N 9/78; C12N 15/11; C12Y 305/04004; C12N 2310/20; C12N 2750/14143

(57) ABSTRACT

The present invention is related to the field of gene editing. In particular, the gene editing is directed toward single nucleotide base editing. For example, such single nucleotide base editing results in a conversion of a mutated base pair to a wild type base pair. The high accuracy and precision of the presently disclosed single nucleotide base gene editor is accomplished by a fusion protein including an NmeCas9 nuclease and an inlaid nucleotide deaminase protein domain. The Nme2Cas9 protospacer interacting domain may be replaced with an SmuCas9 protospacer interacting domain.

### RELATED APPLICATIONS

This application is a continuation of International Patent Application No. PCT/US2022/048261, filed Oct. 28, 2022, which claims priority to U.S. Provisional Patent Application Ser. No. 63/274,667, filed Nov. 2, 2021, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Aug. 28, 2024, is named 753930_UM9-293PCCON_ST26.xml, and is 332,866 bytes in size.

### FIELD OF THE INVENTION

The present invention is related to the field of gene editing. In particular, the gene editing is directed toward single nucleotide base editing. For example, such single nucleotide base editing results in a conversion of a mutated base pair to a wild type base pair. The high accuracy and precision of the presently disclosed single nucleotide base gene editor is accomplished by a fusion protein including an NmeCas9 nuclease and an inlaid nucleotide deaminase protein domain. The Nme2Cas9 protospacer interacting domain may be replaced with an SmuCas9 protospacer interacting domain.

### BACKGROUND

Many human diseases arise due to the mutation of a single base. The ability to correct such genetic aberrations is paramount in treating these genetic disorders. Clustered regularly interspaced short palindromic repeats (CRISPR) along with CRISPR associated (Cas) proteins comprise an RNA-guided adaptive immune system in archaea and bacteria. These systems provide immunity by targeting and inactivating nucleic acids that originate from foreign genetic elements.

SpyCas9 base editing platforms cannot be used to target all single-base mutations due to their limited editing windows. The editing window is constrained in part by the requirement for an NGG PAM and by the requirement that the edited base(s) be a very precise distance from the PAM. SpyCas9 is also intrinsically associated with high off-targeting effects in genome editing.

Gene editing using CRISPR-Cas9 technologies has advanced genetic research and promises to revolutionize gene therapy. Cytosine and adenine base editors (CBEs and ABEs) were developed as a way to precisely correct point mutations without inducing double-strand breaks or requiring a DNA donor. Base editors are comprised of a catalytically impaired Cas9 domain that is completely inactive or cleaves only one strand (a.k.a. dead/dCas9 or nickase/nCas9, respectively) fused to one or more cytosine deaminase (CBE) or adenine deaminase (ABE) domains. For efficient base editing to occur, the Cas9 base editor fusion must recognize a short sequence motif, called a PAM, adjacent to the target site, and a target adenine within an “editing window” upstream of PAM. The PAM and editing window are defined by the Cas domain, deaminase, and the type of fusion between the two effectors.

What is needed in the art is a highly accurate Cas9 single base editing platform having a programmable target specificity due to recognition of a diverse population of PAM sites and a superior gene editing efficiency to permit consistent clinical genetic mutation reversion to a wild type genotype.

### SUMMARY OF THE INVENTION

The present invention is related to the field of gene editing. In particular, the gene editing is directed toward single nucleotide base editing. For example, such single nucleotide base editing results in a conversion of a C·G base pair to a T·A base pair or an A·T base pair to a G·C base pair.. The high accuracy and precision of the presently disclosed single nucleotide base gene editor is accomplished by a fusion protein including an NmeCas9 nuclease and an inlaid nucleotide deaminase protein domain. The Nme2Cas9 protospacer interacting domain may be replaced with an SmuCas9 protospacer interacting domain.

In one embodiment, the present invention contemplates a fusion protein comprising an Nme2Cas9 protein and an inlaid nucleotide base editor (NBE) domain. In one embodiment, the inlaid NBE domain is an adenine base editor (ABE) domain. In one embodiment, the inlaid ABE domain is an inlaid adenosine deaminase protein domain. In one embodiment, the inlaid adenosine deaminase protein domain is an adenosine deaminase8e protein domain (ABE8e). In one embodiment, the inlaid NBE domain is a cytidine base editor (CBE) domain. In one embodiment, the inlaid CBE domain is an inlaid cytosine deaminase protein domain. In one embodiment, the cytosine deaminase protein domain includes, but is not limited to, evoFERNY or rAPOBEC1 In one embodiment, the Nme2Cas9 protein comprises a protospacer adjacent motif interacting domain (PID) having a peptide that hybridizes with an N₄CC nucleotide sequence or an N₄C nucleotide sequence. In one embodiment, the PID is an Nme2Cas9 PID or an SmuCas9 PID. In one embodiment, said fusion protein further comprises a nuclear localization signal protein that includes, but is not limited to, nucleoplasmin (NLS) and/or SV40 NLS and/or C-myc NLS. In one embodiment, the protein further comprises a uracil glycosylase inhibitor. In one embodiment, the Nme2Cas9 protein further comprises a mutation. In one embodiment, said mutation is a D16A mutation. In one embodiment, the Nme2Cas9 (D16A) is a nickase. In one embodiment, said Nme2Cas9 protein further comprises a linker that flanks at least one inlaid domain protein. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates an adeno-associated virus (AAV) comprising a vector encoding a fusion protein comprising an Nme2Cas9 protein and an inlaid nucleotide base editor (NBE) domain. In one embodiment, the inlaid NBE domain is an adenine base editor (ABE) domain. In one embodiment, the inlaid ABE domain is an inlaid adenosine deaminase protein domain. In one embodiment, the inlaid adenosine deaminase protein domain is an adenosine deaminase8e protein (ABE8e). In one embodiment, the inlaid NBE domain is a cytidine base editor (CBE) domain. In one embodiment, the inlaid CBE protein domain is an inlaid cytosine deaminase protein domain. In one embodiment, the cytosine deaminase protein includes, but is not limited to, evoFERNY or rAPOBEC1. In one embodiment, the Nme2Cas9 protein comprises a protospacer adjacent motif interacting domain (PID) having a peptide that hybridizes with an N₄CC nucleotide sequence or an N₄C nucleotide sequence. In one embodiment, the PID is an Nme2Cas9 PID or an SmuCas9 PID. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid domain protein. In one embodiment, the linker is approximately 73 amino acids in length. In one embodiment, the liker is approximately 20 amino acids in length. In one embodiment, said linker is a 3xHA-tag (SEQ ID NO:171).

In one embodiment, the present invention contemplates a method, comprising: a) providing; i) a patient exhibiting at least one symptom of a genetic disease; and ii) an adeno-associated virus (AAV) comprising a vector encoding a fusion protein comprising an Nme2Cas9 protein and an inlaid nucleotide base editor (NBE) domain; b) treating said patient with said adeno-associated virus under conditions such that said at least one symptom of the genetic disease is reduced. In one embodiment, the genetic disease is caused by a gene with a mutated single base, wherein said gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to the N₄CC nucleotide sequence or N₄C nucleotide sequence. In one embodiment, the treating replaces said mutated single base with a wild type single base. In one embodiment, the inlaid NBE domain is an adenine base editor (ABE) domain. In one embodiment, the inlaid ABE domain is an inlaid adenosine deaminase protein domain. In one embodiment, the inlaid adenosine deaminase protein domain is an adenosine deaminase8e protein (ABE8e). In one embodiment, the inlaid NBE domain is a cytidine base editor (CBE) domain. In one embodiment, the inlaid CBE protein domain is an inlaid cytosine deaminase protein domain. In one embodiment, the cytosine deaminase protein includes, but is not limited to, evoFERNY or rAPOBEC1. In one embodiment, the Nme2Cas9 protein comprises a protospacer adjacent motif interacting domain (PID) having a peptide that hybridizes with an N₄CC nucleotide sequence or an N₄C nucleotide sequence. In one embodiment, the PID is an Nme2Cas9 PID or an SmuCas9 PID. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid domain protein. In one embodiment, the linker is approximately 73 amino acids in length. In one embodiment, the linker is approximately 20 amino acids in length. In one embodiment, said linker is a 3xHA-tag (SEQ ID NO:171). In one embodiment, the genetic disease includes, but is not limited to tyrosinemia, muscular dystrophy, Rett syndrome, Batten disease or amyotrophic lateral sclerosis (ALS).

In one embodiment, the present invention contemplates a method, comprising: a) providing; i) a patient exhibiting at least one symptom of tyrosinemia; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said at least one symptom of tyrosinemia is reduced. In one embodiment, the patient further comprises a Fah gene with a mutated single base, wherein said Fah gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to the N₄CC nucleotide sequence or N4C nucleotide sequence. In one embodiment, the PID is an Nme2Cas9 PID or an SmuCas9 PID. In one embodiment, the treating replaces said mutated single base with a wild type single base. In one embodiment, the inlaid nucleotide deaminase protein domain is an inlaid adenosine deaminase protein domain. In one embodiment, the inlaid adenosine deaminase protein domain is an inlaid adenosine deaminase8e protein (ABE8e). In one embodiment, the inlaid nucleotide deaminase protein domain is an inlaid cytosine deaminase protein domain. In one embodiment, the cytosine deaminase protein domain includes, but is not limited to, evoFERNY or rAPOBEC1, In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, said linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient exhibiting at least one symptom of muscular dystrophy; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said at least one symptom of muscular dystrophy is reduced. In one embodiment, the patient further comprises a Dmd gene with a mutated single base, wherein said Dmd gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to said N₄CC nucleotide sequence or said N₄C nucleotide sequence. In one embodiment, the PID is an Nme2Cas9 PID or an SmuCas9 PID. In one embodiment, the treating replaces said mutated single base with a wild type single base. In one embodiment, the inlaid nucleotide deaminase protein domain is an inlaid adenosine deaminase protein domain. In one embodiment, the inlaid adenosine deaminase protein domain is an inlaid adenosine deaminase8e protein (ABE8e). In one embodiment, the inlaid nucleotide deaminase protein domain is an inlaid cytosine nucleotide deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient exhibiting at least one symptom of Rett's syndrome; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said at least one symptom of Rett's syndrome is reduced. In one embodiment, the patient further comprises a MeCP2 gene with a mutated single base, wherein said MeCP2 gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to said N₄CC nucleotide sequence or the N₄C nucleotide sequence. In one embodiment, the PID is an Nme2Cas9 PID or an SmuCas9 PID. In one embodiment, the treating replaces said mutated single base with a wild type single base. In one embodiment, the inlaid nucleotide deaminase protein domain is an inlaid adenosine deaminase protein domain. In one embodiment, the inlaid adenosine deaminase protein domain is an adenosine deaminase8e protein (ABE8e). In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient exhibiting at least one symptom of Batten disease; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said at least one symptom of Batten disease is reduced. In one embodiment, the patient further comprises a CLN3 gene with a mutated single base, wherein said CLN3 gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to said N₄CC nucleotide sequence or the N₄C nucleotide sequence. In one embodiment, the PID is an Nme2Cas9 PID or an SmuCas9 PID. In one embodiment, the treating replaces said mutated single base with a wild type single base. In one embodiment, the inlaid nucleotide deaminase protein domain is an inlaid adenosine deaminase protein domain. In one embodiment, the inlaid adenosine deaminase protein domain is an adenosine deaminase8e protein (ABE8e). In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient exhibiting at least one symptom of amyotrophic lateral sclerosis (ALS); and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said at least one symptom of ALS is reduced. In one embodiment, the patient further comprises a SOD1 gene with a mutated single base, wherein said SOD1 gene is flanked by an N₄CC nucleotide sequence or an N₄C nucleotide sequence. In one embodiment, the Nme2Cas9 protein comprises a protospacer adjacent motif interacting domain (PID) having a peptide that hybridizes to said N₄CC nucleotide sequence or the N₄C nucleotide sequence. In one embodiment, the PID is an Nme2Cas9 PID or an SmuCas9 PID. In one embodiment, the treating replaces said mutated single base with a wild type single base. In one embodiment, the inlaid nucleotide deaminase protein domain is an inlaid adenosine deaminase protein domain. In one embodiment, the inlaid adenosine deaminase protein domain is an adenosine deaminase8e protein (ABE8e). In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient comprising a gene with a mutated single base, wherein said gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and a genetic disease does not develop. In one embodiment, the genetic disease includes, but is not limited to tyrosinemia, muscular dystrophy, Rett syndrome, Batten disease or ALS. In one embodiment, the Nme2Cas9 protein comprises a protospacer adjacent motif interacting domain (PID) having a peptide that hybridizes to said N₄CC nucleotide sequence or the N₄C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1, In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag. (SEQ ID NO:171). In one embodiment, the gene includes, but is not limited to, a Fah gene, a Dmd gene, a MeCP2 gene, a CLN3 gene and an SOD1 gene.

In one embodiment, the present invention contemplates a method, comprising: a) providing; i) a patient comprising a Fah gene with a mutated single base, wherein said mutated Fah gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and tyrosinemia does not develop. In one embodiment, the mutated Fah gene causes tyrosinemia. In one embodiment, the Nme2Cas9 protein comprises a protospacer adjacent motif interacting domain (PID) having a peptide that hybridizes to said N₄CC nucleotide sequence or the N₄C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient comprising a Dmd gene with a mutated single base, wherein said mutated Dmd gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and muscular dystrophy does not develop. In one embodiment, the mutated Dmd gene causes muscular dystrophy. In one embodiment, the Nme2Cas9 protein comprises a protospacer adjacent motif interacting domain (PID) having a peptide that hybridizes to said N₄CC nucleotide sequence or N₄C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length In one embodiment, the linker is approximately 20 amino acids in length. In one embodiment, said linker is a 3xHA-tag (SEQ ID NO:171).

In one embodiment, the present invention contemplates a method, comprising: a) providing; i) a patient comprising a MeCP2 gene with a mutated single base, wherein said mutated MeCP2 gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and Rett's syndrome does not develop. In one embodiment, the mutated MeCP2 gene causes Rett's syndrome. In one embodiment, the Nme2Cas9 protein comprises a protospacer adjacent motif interacting domain (PID) having a peptide that hybridizes to said N₄CC nucleotide sequence or the N4C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient comprising a CLN3 gene with a mutated single base, wherein said mutated CLN3 gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and Batten disease does not develop. In one embodiment, the mutated CLN3 gene causes Batten disease. In one embodiment, the Nme2Cas9 protein comprises a protospacer adjacent motif interacting domain (PID) having a peptide that hybridizes to said N₄CC nucleotide sequence or the N4C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

In one embodiment, the present invention contemplates a method, comprising: a) providing; i) a patient comprising a SOD1 gene with a mutated single base, wherein said mutated SOD1 gene is flanked by an N₄CC nucleotide sequence or an N₄C nucleotide sequence; and ii) an adeno-associated virus (AAV) comprising a vector encoding a fusion protein comprising an Nme2Cas9 protein and an inlaid nucleotide deaminase protein domain; b) treating said patient with said adeno-associated virus under conditions such that said mutated single base is replaced with a wild type single base and amyotrophic lateral sclerosis (ALS) does not develop. In one embodiment, the mutated MeCP2 gene causes Rett's syndrome. In one embodiment, the Nme2Cas9 protein comprises a protospacer adjacent motif interacting domain (PID) having a peptide that hybridizes to said N₄CC nucleotide sequence or the N4C nucleotide sequence. In one embodiment, the PID is an Nme2Cas9 PID or an SmuCas9 PID. In one embodiment, the inlaid nucleotide deaminase protein domain is an inlaid adenosine deaminase protein domain. In one embodiment, the inlaid adenosine deaminase protein domain is an adenosine deaminase8e protein (ABE8e) domain. In one embodiment, the inlaid nucleotide deaminase protein domain is an inlaid cytosine deaminase protein domain. In one embodiment, the cytosine deaminase protein domain includes, but is not limited to, evoFERNY or rAPOBEC1. In one embodiment, said AAV is an adeno-associated virus 8. In one embodiment, said AAV is an adeno-associated virus 6. In one embodiment, said fusion protein further comprises a nuclear localization signal protein including, but not limited to, nucleoplasmin (NLS) and/or SV40 NLS and/or C-myc NLS. In one embodiment, the fusion protein further comprises a uracil glycosylase inhibitor. In one embodiment, said Nme2Cas9 protein further comprises a mutation. In one embodiment, said mutation is a D16A mutation. In one embodiment, said Nme2Cas9 (D16A) protein is a nickase. In one embodiment, said Nme2Cas9 protein further comprises a linker that flanks at least one inlaid protein domain. In one embodiment, the linker is approximately 73 amino acids in length. In one embodiment, the linker is approximately 20 amino acids in length. In one embodiment, said linker is a 3xHA-tag (SEQ ID NO:171).

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target. Such a specific genomic target includes, but is not limited to, a chromosomal region, a gene, a promoter, an open reading frame or any nucleic acid sequence.

As used herein, the term “single base” refers to one, and only one, nucleotide within a nucleic acid sequence. When used in the context of single base editing, it is meant that the base at a specific position within the nucleic acid sequence is replaced with a different base. This replacement may occur by many mechanisms, including but not limited to, substitution or modification.

As used herein, the term “target” or “target site” refers to a pre-identified nucleic acid sequence of any composition and/or length. Such target sites include, but is not limited to, a chromosomal region, a gene, a promoter, an open reading frame or any nucleic acid sequence. In some embodiments, the present invention interrogates these specific genomic target sequences with complementary sequences of gRNA.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “viral vector” encompasses any nucleic acid construct derived from a virus genome capable of incorporating heterologous nucleic acid sequences for expression in a host organism. For example, such viral vectors may include, but are not limited to, adeno-associated viral vectors, lentiviral vectors, SV40 viral vectors, retroviral vectors, adenoviral vectors. Although viral vectors are occasionally created from pathogenic viruses, they may be modified in such a way as to minimize their overall health risk. This usually involves the deletion of a part of the viral genome involved with viral replication. Such a virus can efficiently infect cells but, once the infection has taken place, the virus may require a helper virus to provide the missing proteins for production of new virions. Preferably, viral vectors should have a minimal effect on the physiology of the cell it infects and exhibit genetically stable properties (e.g., do not undergo spontaneous genome rearrangement). Most viral vectors are engineered to infect as wide a range of cell types as possible. Even so, a viral receptor can be modified to target the virus to a specific kind of cell. Viruses modified in this manner are said to be pseudotyped. Viral vectors are often engineered to incorporate certain genes that help identify which cells took up the viral genes. These genes are called marker genes. For example, a common marker gene confers antibiotic resistance to a certain antibiotic.

As used herein, the term “genetic disease” refers to any medical condition having a primary causative factor of a mutated gene. The gene mutation may comprise a nucleic acid sequence wherein at least one, if not more, nucleotides are not wild type.

As used herein the term “Dmd gene” refers to a genetic locus that, when mutated, is believed to result in symptoms of muscular dystrophy.

As used herein the term “Fah gene” refers to a genetic locus that, when mutated, is believed to result in symptoms of tyrosinemia.

As used herein, the term “MeCP2 gene” refers to a genetic locus that, when mutated, is believed to result in symptoms of Rett's syndrome.

As used herein the “ROSA26 gene” or “Rosa26 gene” refers to a human or mouse (respectively) locus that is widely used for achieving generalized expression in the mouse. Targeting to the ROSA26 locus may be achieved by introducing a desired gene into the first intron of the locus, at a unique XbaI site approximately 248 bp upstream of the original gene trap line. A construct may be constructed using an adenovirus splice acceptor followed by a gene of interest and a polyadenylation site inserted at the unique XbaI site. A neomycin resistance cassette may also be included in the targeting vector.

As used herein the “PCSK9 gene” or “Pcsk9 gene” refers to a human or mouse (respectively) locus that encodes a PCSK9 protein. The PCSK9 gene resides on chromosome 1 at the band 1p32.3 and includes 13 exons. This gene may produce at least two isoforms through alternative splicing.

The term “proprotein convertase subtilisin/kexin type 9” and “PCSK9” refers to a protein encoded by a gene that modulates low density lipoprotein levels. Proprotein convertase subtilisin/kexin type 9, also known as PCSK9, is an enzyme that in humans is encoded by the PCSK9 gene. Seidah et al., “The secretory proprotein convertase neural apoptosis-regulated convertase 1 (NARC-1): liver regeneration and neuronal differentiation” Proc. Natl. Acad. Sci. U.S.A. 100 (3): 928-933 (2003). Similar genes (orthologs) are found across many species. Many enzymes, including PSCK9, are inactive when they are first synthesized, because they have a section of peptide chains that blocks their activity; proprotein convertases remove that section to activate the enzyme. PSCK9 is believed to play a regulatory role in cholesterol homeostasis. For example, PCSK9 can bind to the epidermal growth factor-like repeat A (EGF-A) domain of the low-density lipoprotein receptor (LDL-R) resulting in LDL-R internalization and degradation. Clearly, it would be expected that reduced LDL-R levels result in decreased metabolism of LDL-C, which could lead to hypercholesterolemia.

The term “hypercholesterolemia” as used herein, refers to any medical condition wherein blood cholesterol levels are elevated above the clinically recommended levels. For example, if cholesterol is measured using low density lipoproteins (LDLs), hypercholesterolemia may exist if the measured LDL levels are above, for example, approximately 70 mg/dl. Alternatively, if cholesterol is measured using free plasma cholesterol, hypercholesterolemia may exist if the measured free cholesterol levels are above, for example, approximately 200-220 mg/dl.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer DNA”. The spacers are short segments of DNA from a virus and may serve as a ‘memory’ of past exposures to facilitate an adaptive defense against future invasions.

As used herein, the term “Cas” or “CRISPR-associated (cas)” refers to genes often associated with CRISPR repeat-spacer arrays.

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence.

As used herein, the term “N-terminal domain” refers to the fusion of a first peptide or protein at the N-terminal end of a second peptide or protein. For example, a nucleotide deaminase protein may be “N-terminally” fused to the last amino acid of a Cas9 nuclease protein.

As used herein, the term “inlaid domain” refers to the fusion of a first peptide or protein between the C-terminal and N-terminal ends of a second peptide or protein. For example, a nucleotide deaminase protein is an “inlaid domain” when inserted between the C-terminal and N-terminal ends of a Cas9 nuclease protein.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “fluorescent protein” refers to a protein domain that comprises at least one organic compound moiety that emits fluorescent light in response to the appropriate wavelengths. For example, fluorescent proteins may emit red, blue and/or green light. Such proteins are readily commercially available including, but not limited to: i) mCherry (Clonetech Laboratories): excitation: 556/20 nm (wavelength/bandwidth); emission: 630/91 nm; ii) sfGFP (Invitrogen): excitation: 470/28 nm; emission: 512/23 nm; iii) TagBFP (Evrogen): excitation 387/11 nm; emission 464/23 nm.

As used herein, the term “sgRNA” refers to single guide RNA used in conjunction with CRISPR associated systems (Cas). sgRNAs contains nucleotides of sequence complementary to the desired target site. Watson-crick pairing of the sgRNA with the target site recruits the nuclease-deficient Cas9 to bind the DNA at that locus.

As used herein, the term “orthogonal” refers targets that are non-overlapping, uncorrelated, or independent. For example, if two orthogonal nuclease-deficient Cas9 gene fused to different effector domains were implemented, the sgRNAs coded for each would not cross-talk or overlap. Not all nuclease-deficient Cas9 genes operate the same, which enables the use of orthogonal nuclease-deficient Cas9 gene fused to a different effector domains provided the appropriate orthogonal sgRNAs.

As used herein, the term “phenotypic change” or “phenotype” refers to the composite of an organism's observable characteristics or traits, such as its morphology, development, biochemical or physiological properties, phenology, behavior, and products of behavior. Phenotypes result from the expression of an organism's genes as well as the influence of environmental factors and the interactions between the two.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed to a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

Low stringency conditions comprise conditions equivalent to binding or hybridization at 42° C. in a solution consisting of 5×SSPE (43.8 g/l NaCl, 6.9 g/l NaH₂PO₄·H₂O and 1.85 g/l EDTA, pH adjusted to 7.4 with NaOH), 0.1% SDS, 5×Denhardt's reagent {50×Denhardt's contains per 500 ml: 5 g Ficoll (Type 400, Pharmacia), 5 g BSA (Fraction V; Sigma)} and 100 g/ml denatured salmon sperm DNA followed by washing in a solution comprising 5×SSPE, 0.1% SDS at 42° C. when a probe of about 500 nucleotides in length is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Co t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E present an illustrative approach by which to design inlaid domains within a Ca9 protein.

FIG. 1A: Different views of Nme1Cas9 are presented as a ternary complex (PDB:6JDV). Black spheres=N/C-termini. Colored spheres=Domain insertion sites. Legend: Inlaid domain locations within the Nme1Cas9 protein.

FIG. 1B: (SEQ ID NO:173) Exemplary gene editing activities of Nme2-ABE8e constructs in HEK293T cells using an ABE mCherry reporter system activated upon A-to-G editing by plasmid transfection and measured by flow cytometry. Protospacer with target adenine (red), and PAM (bold, underlined). n=3 biological replicates, data represent mean±SD.

FIG. 1C: Exemplary gene editing efficiency in HEK293T cells at eight (8) dual PAM genomic target sites. A-to-G editing at PAM-matched endogenous HEK293T genomic loci between four (4) Nme2-ABE8e constructs, each having a different inlaid domain position, was compared to an SpyCas9-ABE8e fusion protein. Maximally edited adenine for each target was plotted. Gene editing activities were measured by amplicon sequencing. n=3 biological replicates, data represent mean±SD.

FIG. 1D: Exemplary data showing mean gene editing window and associated efficiency in HEK293T cells using eight (8) dual PAM genomic target sites. The data is presented as a summary of mean A-to-G editing activities and editing windows for Spy- and Nme2-ABE8e constructs. Each position in the protospacer represents the mean A-to-G editing efficiency across eight (8) PAM-matched endogenous target sites measured via amplicon sequencing. Crossed out boxes represent no adenine in the targets tested. n=3 biological replicates.

FIG. 1E: Exemplary data showing a measured maximum gene editing efficiency in HEK293T cells using eight (8) dual PAM genomic target sites. Each data point represents the maximum A-to-G editing rate of an individual dual PAM target site in accordance with the Spy and Nme1 constructs presented in FIG. 1C as measured by amplicon sequencing. n=3 biological replicates. Data represent mean±SEM.

FIGS. 2A-2C present exemplary embodiments of specific inlaid domain Cas9 constructs

FIG. 2A: Schematic representation of eight (8) representative Nme2^{D16A}Cas9-ABE constructs and their respective inlaid domain insertion sites.

FIG. 2B: Exemplary data showing a mean gen editing window and associated efficiency in HEK293T cells using fifteen (15) genomic target sites. The data is presented as a summary of mean A-to-G editing activities and editing windows for the eight (8) Nme2-ABE8e constructs depicted in FIG. 2A. Each position in the protospacer represents the mean A-to-G editing efficiency across fifteen (15) endogenous target sites in HEK293T cells as measured via amplicon sequencing. n=3 biological replicates.

FIG. 2C: Exemplary data showing a scatter plot of the maximum A-to-G editing rate observed for an individual target site as measured by amplicon sequencing. The line and error bars represents the mean and SEM of efficiency observed across all target sites. Fold improvement relative to the n-terminal fusion shown below. n=3 biological replicates. Two-way ANOVA analysis: ns, p>0.05; **p<0.01; ***p<0.001 ****p<0.0001

FIGS. 3A-3B illustrate embodiments of an NmeCas9 deaminase fusion protein single base editor.

FIG. 3A: An YE1-BE3-nNme2Cas9 (D16A)-UGI construct.

FIG. 3B: An ABE7.10 nNme2Cas9 (D16A) construct.

FIGS. 4A-4C present exemplary data of the electroporation of HEK293T cells with DNA plasmids comprising a YE1-BE3-nNme2Cas9 (D16A)-UGI fusion protein.

FIG. 4A: (SEQ ID NO:174-177) The TS25 endogenous target site. GN23 sgRNA base-pairs with the target DNA strand, leaving the displaced DNA strand for cytidine deaminase to edit.

FIG. 4B: (SEQ ID NO:178) Sequencing data showing a doublet nucleotide peak (7^{th} position from 5′ end; arrow) demonstrating the successful single base editing of a cytidine to a thymidine (e.g., a C·G base pair conversion to a T·A base pair).

FIG. 4C: A quantitation of the data shown in FIG. 2B plotting the percent conversion of C→T single base editing. The percentage of C converted to T is about 40% in the base editor- and sgRNA-treated sample (p-value=6.88×10-6). The “no sgRNA” control displays the background noise due to Sanger sequencing. EditR (Kluesner et al., 2018) was used to perform the analysis.

FIGS. 5A-5D present specific UGI target sites that were respectively integrated into YE1-BE3-nNme2Cas9/D16A mutant fusion proteins and co-expressed with enhanced green fluorescent protein (EGFP) in a stable K562-derived cell line. Converted bases are highlighted in orange color. Background signals were filtered using negative control samples (YE1-BE3-nNme2Cas9 nucleofected K562 cells without sgRNA constructs). N₄CC PAMs are boxed. The percentage of total reads exhibiting mutations in base-editor-targeted sites is shown in the right column.

FIG. 5A (SEQ ID NO:179-182): EGFP-Site 1

FIG. 5B (SEQ ID NO:183-184): EGFP-Site 2

FIG. 5C(SEQ ID NO:185-187): EGFP-Site 3

FIG. 5D (SEQ ID NO:188-189): EGFP-Site 4

FIG. 6 (SEQ ID NO:190-191) presents an exemplary alignment of the wildtype Fah gene with the tyrosinemia Fah mutant gene showing an A-G single base gene editing target site (position 9). The respective SpyCas9 single PAM site and NmeCas9 double PAM sites are indicated for demonstrating the suboptimal targeting window relative to the SpyCas9 PAM site.

FIGS. 7A-7E illustrate that the closely related Neisseria meningitidis 1, 2 and 3 Cas9 orthologs that have distinct PAMs.

FIG. 7A shows an exemplary schematic showing mutated residues (orange spheres) between Nme2Cas9 (left) and Nme3Cas9 (right) mapped onto the predicted structure of Nme1Cas9, revealing the cluster of mutations in the PID (black).

FIG. 7B shows an exemplary experimental workflow of the in vitro PAM discovery assay with a 10-bp randomized PAM region. Following in vitro digestion, adapters were ligated to cleaved products for library construction and sequencing.

FIG. 7C shows exemplary sequence logos resulting from in vitro PAM discovery reveal the enrichment of a N₄GATT PAM for Nme1Cas9, consistent with its previously established specificity.

FIG. 7D shows exemplary sequence logos indicating that Nme1Cas9 with its PID swapped with that of Nme2Cas9 (left) or Nme3Cas9 (right) requires a C at PAM position 5. The remaining nucleotides were not determined with high confidence due to the modest cleavage efficiency of the PID-swapped protein chimeras (see FIG. 6C).

FIG. 7E shows an exemplary sequence logo showing that full-length Nme2Cas9 recognizes an N₄CC PAM, based on efficient substrate cleavage of a target pool with a fixed C at PAM position 5, and with PAM nts 1-4 and 6-8 randomized.

FIGS. 8A-8D present a characterization of Neisseria meningitidis Cas9 orthologs with rapidly-evolving PIDs, as related to FIG. 7.

FIG. 8A shows an exemplary unrooted phylogenetic tree of NmeCas9 orthologs that are >80% identical to Nme1Cas9. Three distinct branches emerged, with the majority of mutations clustered in the PID. Groups 1 (blue), 2 (orange), and 3 (green) have PIDs with >98%, ˜52%, and ˜86% identity to Nme1Cas9, respectively. Three representative Cas9 orthologs (one from each group) (Nme1Cas9, Nme2Cas9 and Nme3Cas9) are indicated.

FIG. 8B shows an exemplary schematic showing the CRISPR-cas loci of the strains encoding the three Cas9 orthologs (Nme1Cas9, Nme2Cas9, and Nme3Cas9) from (A). Percent identities of each CRISPR-Cas component with N. meningitidis 8013 (encoding Nme1Cas9) are shown. Blue and red arrows denote pre-crRNA and tracrRNA transcription initiation sites, respectively.

FIG. 8C shows an exemplary normalized read counts (% of total reads) from cleaved DNAs from the in vitro assays for intact Nme1Cas9 (grey), for chimeras with Nme1Cas9's PID swapped with those of Nme2Cas9 and Nme3Cas9 (mixed colors), and for full-length Nme2Cas9 (orange), are plotted. The reduced normalized read counts indicate lower cleavage efficiencies in the chimeras.

FIG. 8D shows an exemplary sequence logos from the in vitro PAM discovery assay on an NNNNCNNN PAM pool by Nme1Cas9 with its PID swapped with those of Nme2Cas9 (left) or Nme3Cas9 (right).

FIGS. 9A-9D present exemplary data showing that Nme2Cas9 uses a 22-24 nt spacer to edit sites adjacent to an N₄CC PAM. All experiments were done in triplicate, and error bars represent the standard error of the mean (s.e.m.).

FIG. 9A shows an exemplary schematic diagram depicting transient transfection and editing of HEK293T TLR2.0 cells, with mCherry+ cells detected by flow cytometry 72 hours after transfection.

FIG. 9B shows an exemplary Nme2Cas9 editing of the TLR2.0 reporter. Sites with N₄CC PAMs were targeted with varying efficiencies, while no Nme2Cas9 targeting was observed at an N₄GATT PAM or in the absence of sgRNA. SpyCas9 (targeting a previously validated site with an NGG PAM) and Nme1Cas9 (targeting N₄GATT) were used as positive controls.

FIG. 9C shows an exemplary effect of spacer length on the efficiency of Nme2Cas9 editing. An sgRNA targeting a single TLR2.0 site, with spacer lengths varying from 24 to 20 nts (including the 5′-terminal G required by the U6 promoter), indicate that highest editing efficiencies are obtained with 22-24 nt spacers.

FIG. 9D shows an exemplary An Nme2Cas9 dual nickase can be used in tandem to generate NHEJ- and HDR-based edits in TLR2.0. Nme2Cas9- and sgRNA-expressing plasmids, along with an 800-bp dsDNA donor for homologous repair, were electroporated into HEK293T TLR2.0 cells, and both NHEJ (mCherry+) and HDR (GFP+) outcomes were scored by flow cytometry. HNH nickase, Nme2Cas9^{D16A}; RuvC nickase, Nme2Cas9^{H588A} Cleavage sites 32 bp and 64 bp apart were targeted using either nickase. The HNH nickase (Nme2Cas9^{D16A}) yielded efficient editing, particularly with the cleavage sites that were separated by 32 bp, whereas the RuvC nickase (Nme2Cas9^{H588A}) was not effective. Wildtype Nme2Cas9 was used as a control.

FIGS. 10A-10D present exemplary data showing PAM, spacer, and seed requirements for Nme2Cas9 targeting in mammalian cells, as related to FIG. 7. All experiments were done in triplicate and error bars represent s.e.m.

FIG. 10A shows an exemplary Nme2Cas9 targeting at N₄CD sites in TLR2.0, with editing estimated based on mCherry+ cells. Four sites for each non-C nucleotide at the tested position (N₄CA, N₄CT and N₄CG) were examined, and an N₄CC site was used as a positive control.

FIG. 10B shows an exemplary Nme2Cas9 targeting at N₄DC sites in TLR2.0 [similar to (A)].

FIG. 10C shows exemplary guide truncations on a TLR2.0 site with a N₄CCA PAM, revealing similar length requirements as those observed at the other site.

FIG. 10D (SEQ ID NO: 317,192-214) shows exemplary Nme2Cas9 targeting efficiency is differentially sensitive to single-nucleotide mismatches in the seed region of the sgRNA. Data show the effects of walking single-nucleotide sgRNA mismatches along the 23-nt spacer in a TLR2.0 target site.

FIGS. 11A-11B present exemplary data showing Nme2Cas9 genome editing at endogenous loci in mammalian cells via multiple delivery methods. All results represent 3 independent biological replicates, and error bars represent s.e.m.

FIG. 11A shows an exemplary Nme2Cas9 genome editing of endogenous human sites in HEK293T cells following transient transfection of Nme2Cas9- and sgRNA-expressing plasmids. 40 sites were screened initially (Table 1); the 14 sites shown (selected to include representatives of varying editing efficiencies, as measured by TIDE) were then re-analyzed in triplicate. An Nme1Cas9 target site (with an N₄GATT PAM) was used as a negative control.

FIG. 11B shows exemplary data charts: Left panel: Transient transfection of a single plasmid expressing both Nme2Cas9 and sgRNA (targeting the Pcsk9 and Rosa26 loci) enables editing in Hepa1-6 mouse cells, as detected by TIDE. Right panel: Electroporation of sgRNA plasmids into K562 cells stably expressing Nme2Cas9 from a lentivector results in efficient indel formation.

FIG. 11C shows exemplary Nme2Cas9 can be electroporated as an RNP complex to induce genome editing. 40 picomoles Cas9 along with 50 picomoles of in vitro-transcribed sgRNAs targeting three different loci were electroporated into HEK293T cells. Indels were measured after 72 h using TIDE.

FIGS. 12A-12B present exemplary data showing dose dependence and segmental deletions by Nme2Cas9, as related to FIG. 9.

FIG. 12A shows exemplary increasing the dose of electroporated Nme2Cas9 plasmid (500 ng, vs. 200 ng in FIG. 3A) improves editing efficiency at two sites (TS16 and TS6). Data provided in yellow are re-used from FIG. 11A.

FIG. 12B shows exemplary Nme2Cas9 can be used to create precise segmental deletions. Two TLR2.0 targets with cleavage sites 32 bp apart were targeted simultaneously with Nme2Cas9. The majority of lesions created were deletions of exactly 32 bp (blue).

FIGS. 13A-13C present exemplary data showing that Nme2Cas9 is subject to inhibition by a subset of type II-C anti-CRISPR families in vitro and in cells. All experiments were done in triplicate and error bars represent s.e.m.

FIG. 13A: In vitro cleavage assay of Nme1Cas9 and Nme2Cas9 in the presence of five previously characterized anti-CRISPR proteins (10:1 ratio of Acr:Cas9). Top: Nme1Cas9 efficiently cleaves a fragment containing a protospacer with an N₄GATT PAM in the absence of an Acr or in the presence of a negative control Acr (AcrE2). All five previously characterized type II-C Acr families inhibited Nme1Cas9, as expected. Bottom: Nme2Cas9 inhibition mirrors that of Nme1Cas9, except for the lack of inhibition by AcrIIC5_{Smu}.

FIG. 13B: Genome editing in the presence of the five previously described anti-CRISPR families. Plasmids expressing Nme2Cas9 (200 ng), sgRNA (100 ng) and each respective Acr (200 ng) were co-transfected into HEK293T cells, and genome editing was measured using Tracking of Indels by Decomposition (TIDE) 72 hr post transfection. Consistent with our in vitro analyses, all type II-C anti-CRISPRs except AcrIIC5_{Smu} inhibited genome editing, albeit with different efficiencies.

FIG. 13C: Acr inhibition of Nme2Cas9 is dose-dependent with distinct apparent potencies. Nme2Cas9 is fully inhibited by AcrIIC1_{Nme} and AcrIIC4_{Hpa} at 2:1 and 1:1 mass ratios of cotransfected Acr and Nme2Cas9 plasmids, respectively.

FIG. 14 presents exemplary data showing that a Nme2Cas9 PID swap renders Nme1Cas9 insensitive to AcrIIC5_{Smu} inhibition, as related to FIG. 11. In vitro cleavage by the Nme1Cas9-Nme2Cas9PID chimera in the presence of previously characterized Acr proteins (10 uM Cas9-sgRNA+100 uM Acr).

FIGS. 15A-15E present exemplary data showing orthogonality and relative accuracy of Nme2Cas9 and SpyCas9 at dual target sites, as related to FIG. 12.

FIG. 15A shows exemplary Nme2Cas9 and SpyCas9 guides are orthogonal. TIDE results show the frequencies of indels created by both nucleases targeting DS2 with either their cognate sgRNAs or with the sgRNAs of the other ortholog.

FIG. 15B shows exemplary Nme2Cas9 and SpyCas9 exhibiting comparable on-target editing efficiencies as assessed by GUIDE-seq. Bars indicate on-target read counts from GUIDE-Seq at the three dual sites targeted by each ortholog. Orange bars represent Nme2Cas9 and black bars represent SpyCas9.

FIG. 15C shows an exemplary SpyCas9's on-target vs. off-target read counts for each site. Orange bars represent the on-target reads while black bars represent off-targets.

FIG. 15D shows exemplary Nme2Cas9's on-target vs. off-target reads for each site.

FIG. 15E (SEQ ID NO:178, 215-222) bar graphs showing exemplary indel efficiencies (measured by TIDE) at potential off-target sites predicted by CRISPRSeek. On- and off-target site sequences are shown on the left, with the PAM region underlined and sgRNA mismatches and non-consensus PAM nucleotides given in red.

FIGS. 16A-16E present exemplary data showing that Nme2Cas9 exhibits little or no detectable off-targeting in mammalian cells.

FIG. 16A shows an exemplary schematic depicting dual sites (DSs) targetable by both SpyCas9 and Nme2Cas9 by virtue of their non-overlapping PAMs. The Nme2Cas9 PAM (orange) and SpyCas9 PAM (blue) are highlighted. A 24 nt Nme2Cas9 guide sequence is indicated in yellow; the corresponding guide sequence for SpyCas9 would be 4 nt shorter at the 5′ end.

FIG. 16B shows an exemplary Nme2Cas9 and SpyCas9 that both induce indels at DSs. Six DSs in VEGFA (with GN₃GN₁₉NGGNCC sequences) were selected for direct comparisons of editing by the two orthologs. Plasmids expressing each Cas9 (with the same promoter, linkers, tags and NLSs) and its cognate guide were transfected into HEK293T cells. Indel efficiencies were determined by TIDE 72 hrs post transfection. Nme2Cas9 editing was detectable at all six sites and was marginally or significantly more efficient than SpyCas9 at two sites (DS2 and DS6, respectively). SpyCas9 edited four out of the six sites (DS1, DS2, DS4 and DS6), with two sites showing significantly higher editing efficiencies than Nme2Cas9 (DS1 and DS4). DS2, DS4 and DS6 were selected for GUIDE-Seq analysis as Nme2Cas9 was equally efficient, less efficient and more efficient than SpyCas9, respectively, at these sites.

FIG. 16C shows exemplary Nme2Cas9 genome editing that is highly accurate in human cells. Numbers of off-target sites detected by GUIDE-Seq for each nuclease at individual target sites are shown. In addition to dual sites, we analyzed TS6 (because of its high on-target editing efficiency) and Pcsk9 and Rosa26 sites in mouse Hepa1-6 cells (to measure accuracy in another cell type).

FIG. 16D shows an exemplary targeted deep sequencing to detect indels in edited cells confirms the high Nme2Cas9 accuracy indicated by GUIDE-seq.

FIG. 16E (SEQ ID NO:223 and 224) shows an exemplary sequence for the validated off-target site of the Rosa26 guide, showing the PAM region (underlined), the consensus CC PAM dinucleotide (bold), and three mismatches in the PAM-distal portion of the spacer (red).

FIGS. 17A-17C present exemplary data showing Nme2Cas9 genome editing in vivo via all-in-one AAV delivery.

FIG. 17A shows exemplary workflow for delivery of AAV8.sgRNA.Nme2Cas9 to lower cholesterol levels in mice by targeting Pcsk9. Top: schematic of the all-in-one AAV vector expressing Nme2Cas9 and the sgRNA (individual genome elements not to scale). BGH, bovine growth hormone poly(A) site; HA, epitope tag; NLS, nuclear localization sequence; h, human-codon-optimized. Bottom: Timeline for AAV8.sgRNA.Nme2Cas9 tail-vein injections (4×10¹¹ GCs), followed by cholesterol measurements at day 14 and indel, histology and cholesterol analyses at day 28 post-injection.

FIG. 17B shows an exemplary TIDE analysis to measure indels in DNA extracted from livers of mice injected with AAV8.Nme2Cas9+ sgRNA targeting Pcsk9 and Rosa26 (control) loci. Indel efficiency at the lone off-target site identified by GUIDE-seq for these two sgRNAs (Rosa26|OT1) were also assessed by TIDE.

FIG. 17C shows an exemplary reduced serum cholesterol levels in mice injected with the Pcsk9-targeting guide compared to the Rosa26-targeting controls. P values are calculated by unpaired two-tailed t-test.

FIGS. 18A-18B present exemplary data showing PCSK9 knockdown and liver histology following Nme2Cas9 AAV delivery and editing, related to FIG. 15.

FIG. 18A shows exemplary Western blotting using anti-PCSK9 antibody reveals strongly reduced levels of PCSK9 in the livers of mice treated with sgPcsk9, compared to mice treated with sgRosa26. 2 ng of recombinant PCSK9 was used as a mobility standard (left-most lane), and a cross-reacting band in the liver samples is indicated by an asterisk. GAPDH was used as loading control (bottom panel).

FIG. 18B shows exemplary H&E staining from livers of mice injected with AAV8.Nme2Cas9+sgRosa26 (left) or AAV8.Nme2Cas9+sgPcsk9 (right) vectors. Scale bars, 25 μm.

FIGS. 19A-19C present exemplary data showing Tyr editing ex vivo in mouse zygotes, related to FIG. 18.

FIG. 19A shows an exemplary two sites in Tyr, each with N₄CC PAMs, were tested for editing in Hepa1-6 cells. The sgTyr2 guide exhibited higher editing efficiency and was selected for further testing.

FIG. 19B shows an exemplary seven mice that survived post-natal development, and each exhibited coat color phenotypes as well as on-target editing, as assayed by TIDE.

FIG. 19C shows an exemplary Indel spectra from tail DNA of each mouse from (B), as well as an unedited C57BL/6NJ mouse, as indicated by TIDE analysis. Efficiencies of insertions (positive) and deletions (negative) of various sizes are indicated.

FIGS. 20A-20C present exemplary data showing Nme2Cas9 genome editing ex vivo via all-in-one AAV delivery.

FIG. 20A shows an exemplary workflow for single-AAV Nme2Cas9 editing ex vivo to generate albino C57BL/6NJ mice by targeting the Tyr gene. Zygotes are cultured in KSOM containing AAV6.Nme2Cas9:sgTyr for 5-6 hours, rinsed in M2, and cultured for a day before being transferred to the oviduct of pseudo-pregnant recipients.

FIG. 20B shows exemplary albino (left) and chinchilla or variegated (middle) mice generated by 3×10⁹ GCs, and chinchilla or variegated mice (right) generated by 3×10⁸ GCs of zygotes with AAV6.Nme2Cas9:sgTyr.

FIG. 20C shows an exemplary summary of Nme2Cas9.sgTyr single-AAV ex vivo Tyr editing experiments at two AAV doses.

FIGS. 21A-21E present exemplary data showing gene editing differences between fusion proteins of NmeCas9 and SpyCas9 nuclease with an N-terminally fused adenine deaminase domain.

FIG. 21A: Schematic representation of the ABE reporter cell line.

FIG. 21B: Schematic representation of the Nme2Cas9-ABE constructs.

FIG. 21C: Comparison of Nme2Cas9-ABE editing efficiency to those of SpyCas9-ABEs in the ABE reporter cell line (n=3 biological replicates).

FIG. 21D: Summary of editing windows and comparison of editing efficiencies for the ABEs at endogenous genomic loci. Each data point represents the mean A-to-G editing efficiency at the indicated position in the spacer measured by amplicon deep sequencing across 12 Nme2Cas9 target sites and 8 target SpyCas9 target sites, respectively. (n=3 biological replicates).

FIG. 21E: Comparison of Nme2Cas9-ABE8e mismatch tolerance to that of SpyCas9-ABE8e in the ABE reporter cell line. The activities of the effectors with the mismatched guides are normalized to that of the perfectly complementary (WT) guide. Red, mismatched nucleotides; green, PAM sequence (n=3 biological replicates).

FIGS. 22A-22C present exemplary data showing a summary of the individual A-to-G conversion efficiency at twelve target sites for Nme2Cas9-ABE8e, which include eight dual-target sites (DS 2-28) and four Nme2Cas9-specific target sites (Nm2 1-4), and eight dual-target sites for SpCas9-ABE7.10 and SpyCas9 ABE8e. Each data point represents the A-to-G conversion efficiency at the indicated nucleotide position measured by amplicon deep sequencing (n=3 biological replicates).

FIGS. 23A-23D present exemplary data showing single base mutation reversion and exon skipping strategy by a fusion protein comprising an Nme2Cas9-ABE8e construct in MeCP2 and Dmd genes.

FIG. 23A (SEQ ID NO:225-228): Schematic representation of a nonsense mutation in the human MeCP2 gene (c. 502 C>T; p.R168X) that causes Rett Syndrome. The black underline denotes the target sequence of an Nme2Cas9-ABE8e guide for reverting the mutant A to G (wildtype) position 10 (red, bold). The PAM region is underlined in green. A bystander edit at position 16 (orange) can generate a missense mutation (c. 496 T>C; p.S166P).

FIG. 23B: Amplicon deep sequencing quantifying the editing efficiency in Rett patient-derived fibroblasts transfected with the Nme2Cas9-ABE8e mRNA and the synthetic sgRNA_MeCP2 noted in a (n=3 biological replicates).

FIG. 23C(SEQ ID NO:229 and 230): Schematic representation of the exon skipping strategy that restores the reading frame of the mouse Dmd transcript. Deletion of exon 51 (ΔEx51) can alter the reading frame and generate a premature stop codon in exon 52 (red). Adenine base editing at the splice site of exon 50 (red) by Nme2Cas9-ABE8e can cause exon 50 skipping (gray) and restore the Dmd reading frame. The PAM region is underlined in green.

FIG. 23D: Amplicon deep sequencing quantifying the editing efficiency at the target site in the mouse N2a cells transfected with the Nme2Cas9-ABE8e and sgRNA_Dmd expression plasmid (n=3 biological replicates).

FIGS. 24A-24C present exemplary data evaluating nuclear localization signal protein delivery of fusion proteins comprising an N-terminally fused Nme2Cas9-ABE8e protein.

FIG. 24A: Schematic representation of single AAV constructs with different NLS configurations.

FIG. 24B: Comparison of different NLS configurations by plasmid transfection in the ABE reporter cell line.

FIG. 24C (SEQ ID NO:231 and 232): Comparison of U6 and miniU6 promoters for sgRNA expression in the 2xBP_SV40 NLS construct targeting the ABE reporter site (left) or endogenous human (middle) and mouse (right) genomic sites by plasmid transfection in cultured cells followed by amplicon deep sequencing (n=3 biological replicates).

FIGS. 25A-25C present exemplary data confirming Fah gene mutation reversion with the AAV delivery plasmid/vectors.

FIG. 25A: Schematic representation of U6 or miniU6 AAV N-terminally fused Nme2Cas9-ABE8e constructs.

FIG. 25B: Anti-FAH IHC staining showing FAH⁺ hepatocytes, before NEBC withdrawal, in the Fah^{PM/PM} mouse injected with AAV9 expressing Nme2Cas9-ABE8e with a sgRNA targeting either the Fah gene, or the Rosa26 gene that serves as a negative control. Scale bar, 500 μm. The bar graph quantifies the percentage of FAH⁺ hepatocytes detected by IHC (n=4 mice per group).

FIG. 25C: Quantification of the editing efficiency by amplicon deep-sequencing of the genomic DNA extracted from AAV9 injected mouse livers harvested before NTBC withdrawal (n=4 mice per group).

FIGS. 26A-26I present exemplary data showing single base mutation reversion and exon 8 skipping strategy in the Fah gene.

FIG. 26A (SEQ ID NO:233): Illustration of the pathogenic point mutation in the FahPM/PM mouse model that causes exon 8 skipping of the Fah gene, and the guide design for Nme2Cas9-ABE8e to correct the point mutation. Red and bold, target adenine; orange, other bystander adenines; green and underlined, PAM.

FIG. 26B: Illustration of constructs of the single AAV vector plasmids used in in vivo studies.

FIG. 26C: Editing efficiencies at the Fah mutant site by AAV plasmid electroporation in MEF cells derived from the FahPM/PM mouse. Data are from amplicon deep sequencing (n=2 biological replicates).

FIG. 26D: Anti-FAH immunohistochemistry (IHC) staining showing FAH+ hepatocytes, before NTBC withdrawal, in the FahPM/PM mouse hydrodynamically injected with the indicated plasmid. The bar graph quantifies the percentage of FAH+ hepatocytes detected by IHC. Scale bars, 500 μm.

FIG. 26E: Body weight plot of mice injected with the single-AAV vector plasmid showing gradual weight gain over a month after NTBC withdrawal.

FIG. 26F: RT-PCR analysis of the plasmid- or PBS-injected mouse livers using primers that span exons 5 and 9. The wild-type amplicon is 405 bp and exon 8 skipped amplicon is 305 bp.

FIG. 26G (SEQ ID NO:234): Representative Sanger sequencing trace of the 405 bp RT-PCR band.

FIG. 26H: Anti-FAH IHC staining showing expansion of FAH+ hepatocytes 40 days post NTBC withdrawal. Scale bars, 500 μm.

FIG. 26I: Quantification of the editing efficiency by amplicon deep sequencing of genomic DNA of the treated mouse livers harvested 40 days post NTBC withdrawal. (d-i, n=2 mice per group).

FIG. 27 presents exemplary data showing flow cytometry gating strategy for the ABE reporter cell line.

FIGS. 28A-28B present exemplary data validating N-terminally fused ABE domain construct stability subsequent to AAV delivery at a target site.

FIG. 28A: Alkaline gel electrophoresis of AAV9 genomic DNAs targeted to the Rosa26 locus.

FIG. 28B (SEQ ID NO:232): Quantification of the gene editing at the Rosa26 locus by amplicon deep sequencing using mouse livers injected with indicated AAV9. (n=3 mice per group).

FIGS. 29A-29B present an off-target analysis for the N-terminally fused Nme2Cas9-ABE8e domain construct at the Fah gene.

FIG. 29A (SEQ ID NO:233, 235 and 236): Sequence of the Fah on-target site and two top-rated Cas-OFFinder predicted off-target sites for Nme2Cas9-ABE8e. Bases that are different from the on-target site are labeled in red. PAM, green, underlined.

FIG. 29B: Representative amplicon deep sequencing reads at the predicted off-target sites in mouse injected with AAV9 expressing Nme2Cas9-ABE8e and sgRNA-Fah.

FIG. 30 presents an illustrative three-dimensional representation of an induced separation of an inlaid nucleotide deaminase protein domain and the N-terminus of a Cas9 protein.

FIGS. 31A-31C present one embodiment of a Cas9 protein with an inlaid nucleotide deaminase domain.

FIG. 31A: One embodiment of an inlaid nucleotide deaminase domain insert. Orange: Nucleotide Deaminase protein; Purple: N-terminal linker; Blue: C-terminal linker.

FIG. 31B: An illustration of several candidate inlaid nucleotide deaminase domain insertion sites in the NmeCas9 protein (as indicated by colored lines). TadA8e Deaminase was inserted into regions of a RUV—C Nme2Cas9 (D16A) nickase. The insertion sites based on the criteria in red, and were based on NmeCas9 crystal structures (PDB: 6jDV; Sun et al. Mol Cell. 2019).

FIG. 31C: Proposed three-dimensional locations within the NmeCas9 PDB 6JDV of the inlaid nucleotide deaminase protein domains illustrated in FIG. 30B (color matched). Site 1: Q291-RECII (red); Site 2: D328-RECII (orange); Site 3: K339-RECII (taupe); Site 4: R643-HNH (green); Site 5: E659-Linker 2 (light blue); Site 6: V715-RUVCIII (dark blue); Site 7: E761-RUVCIII (purple); and Site 8: P795-RUVCIII (pink).

FIGS. 32A-32B present exemplary data showing gene editing activity for the ABE inlaid domain Cas9 locations defined in FIG. 30.

FIG. 32A (SEQ ID NO:237-239): A schematic of the ABE mCherry reporter system for identifying gene editing activity. The ABE reporter is stably integrated into the genome of HEK293T cells

FIG. 32B: Representative photomicrographs of gene editing activity at various ABE inlaid domain Cas9 locations as indicated by the red fluorescence intensity. Fluorescent images of ABE reporter cells 72 hrs post transfection with plasmids that express Nme2Cas9-ABE and the guide RNA to correct the mCherry stop codon.

FIG. 33 (SEQ ID NO:240 and 241) presents exemplary data of Sanger sequencing of ABE mCherry reporter data in FIG. 32 after editing with Nme2Cas9-ABE8e variants. The positive control is N-term fused Nme2Cas9-ABE8e. The dashed black line represents the target adenine base.

FIGS. 34A-34B present exemplary data showing estimated gene editing data based upon the mCherry system. Quantification of editing rates for the inlaid Nme2Cas9-ABE variants is compared to the N-terminal fused Nme2Cas9-ABE8e as a positive control (gray bar).

FIG. 34A: Flow cytometry of ABE mCherry reporter cells 72 hrs post transfection with an ABE effector and guide RNA.

FIG. 34B: Amplicon sequencing of the targeted mCherry locus, showing % reads with an A to G conversion at the target adenine.

FIGS. 35A-35C present exemplary data showing gene editing activity of three endogenous loci using the eight ABE inlaid domain location described in FIG. 30. Editing Rates of Nme2Cas9-ABE variants at three endogenous genomic loci 72 hrs post transient transfection. Data analyzed by sanger sequencing and EditR tool that quantifies nucleotide frequency in a pool of PCR amplicons. The spacer sequence is on the X-axis and the observed nucleotide frequency is on the Y-Axis.

FIG. 35A: LINC01588-DS12

FIG. 35B: FANCF-DS28

FIG. 35C: MECP2-G2

FIG. 36 presents exemplary data comparing N-terminal domain Nme2-ABE with inlaid domain Nme2-ABE gene editing of DMD mutations. Sanger sequencing quantifies the editing efficiency at the target site in mouse N2a cells transfected with Nme2-ABE8e-nt, −i1, −i7 and −i8 expressed in an sgRNA_Dmd expression plasmid (n=2 biological replicates, 2 technical replicates).

FIGS. 37A-37B present illustrative guide RNA sequences with slice donors and acceptors that target CLN3 exon 5 used with Nme2-ABE constructs to treat Batten disease

FIG. 37A (SEQ ID NO:242 and 243): Guide mRNA sequence targeting mouse CLN3 exon 5.

FIG. 37B (SEQ ID NO:244 and 245): Guide mRNA sequence targeting human CLN3 exon 5.

FIG. 38 (SEQ ID NO:246-249) presents illustrative splice donor and splice acceptor target sequences in CLN3 exon 5 to treat Batten disease. for human and mouse. Target sequences are from 5′ to 3′ with target adenine (red), and PAM (bold, underlined). Measured via amplicon sequencing (n=3).

FIGS. 39A-39B present exemplary data showing CLN3 exon 5 gene editing efficiency with the Nme2-ABE-i1 construct.

FIG. 39A: A-to-G editing with Nme2-ABE8-i1 effectors at the splice donor or acceptor of mouse CLN3 in N2A cells with single AAV vector plasmid. Measured via sanger sequencing (n=3). Exon Skipping validated by RT-PCR.

FIG. 39B: A-to-G editing with Nme2-ABE8e-nt, i1 and −i1^{V106W} constructs at the splice donor or acceptor of human CLN3 with vector plasmids expressing effector or guide. Measured via amplicon sequencing (n=3).

FIGS. 40A-40D present exemplary data showing mouse CLN3 gene targeting with Nme2-iABE8e_1 to generate exon 5 skipping in cultured N2a cells.

FIG. 40A (SEQ ID NO:312 and 313): Illustration of mouse CLN3 exon 5 sequence alignment with splice acceptor/donor positions and three Nme2-Cas9 guides.

FIG. 40B: Exemplary data of CLN3 exon 5 mutation conversion with Nme2-Cas9-ABE administered using different guide constructs depicted in FIG. 22(5)A.

FIG. 40C: A representative gel electrophotograph showing RT-PCR on mCLN3 transcript from transfected N2a cells. NC: negative control; 1: Nme2-iABE_1-mCLN3_G1; 2: Nme2-iABE_1-mCLN3_G4.

FIG. 40D (SEQ ID NO:250): Exemplary Sanger sequencing base calling data showing CLN3 exon 5 skipping subsequent to Nme2-Caso-ABE gene editing by the adjacent location of CLN3 exon 4 and exon 6.

FIGS. 41A-41C present exemplary data showing single AAV delivery of Nme2-iABE_1-sgRNA targeting mouse brain CLN3 genes.

FIG. 41A: Exemplary data of CLN3 exon 5 editing efficiency in mouse cortex, striatum, hippocampus and thalamus using the different Nme2-Cas9 guide constructs in accordance with FIG. 22(5).

FIG. 41B: Exemplary data showing total RNA (RT-PCR) in mouse striatal tissue subsequent to a high dose AAV delivery of Nme2-iABE_1-sgRNA and Nme2-iABE_4-sgRNA.

FIG. 41C(SEQ ID NO:251): Exemplary Sanger sequencing base calling data showing CLN3 exon 5 skipping subsequent to Nme2-Caso-ABE gene editing by the adjacent location of CLN3 exon 4 and exon 6.

FIGS. 42A-42E present exemplary mouse exon 5 Nme2-ABE editing data comparing plasmid injections into neonatal intracerebral ventricles (ICV) with adult intrastriatial (IS).

FIG. 42A: Illustrative AAV plasmid constructs.

FIG. 42B: Adult B6 mouse IS, 8×10⁹ GC/mouse, 8 weeks incubation, deep sequencing (target A-to-G).

FIG. 42C: P1 B6 mouse neonate ICV, 1.5×10¹⁰ GC/mouse, 4 weeks incubation, deep sequencing (target A-to-G).

FIG. 42D: Exemplary data showing gene editing in mouse striatum FIG. 42E: Exemplary data showing gene editing in mouse liver.

FIGS. 43A-43C present exemplary photomicrographs showing brain regional Nme2-1ABE8e_1 mRNA transcript expression in transverse mouse brain slices.

FIG. 43A: Adult IS injection of AAV9-Nme2-iABE8e_1-sgRNA 8-week mouse, bilateral IS injection, 9×10⁹ vg per side.

FIG. 43B: Neonatal ICV injection of AAV9-Nme2-iABE8e_1-sgRNA P1 mouse, bilateral ICV injection, 3×10¹⁰ vg per mouse.

FIG. 43C: Phosphate buffered saline control injection.

FIGS. 44A-44B present exemplary data showing Nme2Cas9-ABE conversion of Rett Syndrome: c.502 C>T mutation.

FIG. 44A (SEQ ID NO:252-255): A schematic illustration of a portion of Mecp2 exon 4 in Rett patient-derived fibroblasts (PDFs). Nonsense mutation: c.502 C>T; p.R168X (red); Potential bystander edits (orange).

FIG. 44B (SEQ ID NO:256-257): Exemplary data of A-to-G editing of Mecp2 c.502 C>T in the RETT-PDF cell line in accordance with FIG. 22(1+)A, with Nme2-ABE8e effectors delivered as mRNA with synthetic sgRNAs. Protospacer with target adenine (red), bystander adenine (orange), and PAM (bold, underlined). n=3 biological replicates, data represent mean±SD.

FIGS. 45A-45B present exemplary data showing Nme2Cas9-ABE conversion of Rett Syndrome c.916 C>T mutation.

FIG. 45A (SEQ ID NO:258-261): A schematic illustration of a portion of Mecp2 exon 4 in RETT patient derived fibroblasts. Missense mutation: c.916 C>T; p.R306C (red). Potential bystander edits (orange).

FIG. 45B (SEQ ID NO:262): Exemplary data of A-to-G editing of Mecp2 916C>T in RETT-PDF cell line in accordance with FIG. 22(2+)A. with Nme2-ABE8e effectors delivered as mRNA with synthetic sgRNAs. Protospacer with target adenine (red), bystander adenine (orange), and PAM (bold, underlined). n=3 biological replicates, data represent mean±SD.

FIGS. 46A-46B present exemplary data showing Nme2Cas9-ABE conversion of Rett Syndrome c.763C>T mutation.

FIG. 46A (SEQ ID NO:263-266): A schematic illustration of a portion of Mecp2 exon 4 in RETT piggyBac cells, Missense mutation: c.763 C>T; p.R255X (red).

FIG. 46B (SEQ ID NO:267): Exemplary data of A-to-G editing of Mecp2 763C>T in RETT-PiggyBac cell line in accordance with FIG. 22(3+)A with Nme2-ABE8e effectors and sgRNA delivered as plasmids. Protospacer with target adenine (red), and PAM (bold, underlined). n=2 biological replicates, data represent mean±SD.

FIGS. 47A-47B present exemplary data showing Nme2Cas9-ABE conversion of Rett Syndrome c.808C>T mutation.

FIG. 47A (SEQ ID NO:268-271): A schematic illustration of a portion of Mecp2 exon 4 in RETT piggyBac cells, Missense mutation: c.808C>T; p.R270X (red) and potential bystander edits. (orange).

FIG. 47B (SEQ ID NO:272): Exemplary data of A-to-G editing of Mecp2: c.808C>T in RETT-PiggyBac cell line in accordance with FIG. 22(4+)A with Nme2-ABE8e effectors and sgRNA delivered as mRNA with synthetic sgRNA. Protospacer with target adenine (red), and PAM (bold, underlined). n=2 biological replicates, data represent mean±SD.

FIGS. 48A-48C present exemplary data showing in vivo gene editing with AAV9 comparing: i) an Nme2-ABE8e-terminal ABE domain (nt) construct; ii) an Nme2-ABE8e-inlaid ABE domain (i1) construct; and iii) an Nme2-ABE8e-mutated inlaid ABE domain (i1^{V106W}) construct.

FIG. 48A: Illustrative schematics of the Nme2-ABE8e-nt, −i1 and i1^{V106W} AAV constructs.

FIG. 48B: Exemplary data showing in vivo gene editing with the −nt, i1 and i1^{V106W} AAV Nme2-ABE constructs in mouse liver (left panel) and striatum (right panel). Liver data is expressed as a quantification of the editing efficiency at the Rosa26 locus as measured by amplicon deep sequencing using liver genomic DNA from tail-vein injected mice at 4×10¹¹ vg/mouse (n=3 mice per group). Striatal data is expressed as a quantification of the editing efficiency at the Rosa26 locus as measured by amplicon deep sequencing from intrastriatally injected mice at 1×10¹⁰ vg/side (n=3 mice per group). Data is represented mean±SD and analyzed using a two-way ANOVA analysis: ns, p>0.05; ***p<0.05; **p<0.01; ***p<0.001 ****p<0.0001.

FIG. 48C(SEQ ID NO:273 and 274): Exemplary data showing in vivo off-target gene editing with the −nt, i1 and i1^{V106W} AAV Nme2-ABE constructs in mouse liver relative to a Rosa26 on-target site protospacer and a previously validated Nme2-ABE8e off-target site (OT1). Upper Panel: A Rosa26 protospacer sequence annotated with: i) target adenines (red); ii) OT1 mismatches (orange); and iii) PAMs (bold, underlined). Lower Pane: A bar graph showing representative data of the quantification of A-to-G editing as measured by amplicon deep sequencing reads at the Rosa26-OT1 site by mice tail vein AAV injection. Data represent mean±SD. two-way ANOVA analysis: ns, p>0.05; ****p<0.0001.

FIGS. 49A-49E present exemplary data showing sensitivity of guide-dependent ABE domain Cas9 constructs.

FIG. 49A (SEQ ID NO: 173): Upper Panel: A 5′ to 3′ overlapping target sequence. Red: target adenine; Bold/Underline=PAM. Lower Panel: Exemplary bar graph data showing a comparison of on-target activity of Spy-ABE8e, Nme2-ABE8e-nt and Nme2-ABE8e-i1 constructs using the ABE mCherry reporter system by plasmid transfection and flow cytometry.

FIG. 49B (SEQ ID NO:173,275-283): Exemplary sequences show Spy-ABE8e construct mismatch (mm) nucleotide (orange) tolerance in ABE mCherry reporter cells with the overlapping target site depicted in FIG. 27(2+)A. Bar Graph: Single guide RNAs with mismatched (mm) nucleotides (orange) are normalized to the activity of fully complementary guides. n=3 biological replicates, Bars: Mean±SD.

FIG. 49C: (TOP TO BOTTOM: SEQ ID NO:173,275-277,314,279-283,315-316)Nme2-ABE8e-nt construct mismatch tolerance in ABE mCherry reporter cells as in FIG. 27(2)B at the overlapping target site as in FIG. 27(2)A.

FIG. 49D: (TOP TO BOTTOM: SEQ ID NO:173,275-277,314,279-283,315-316) Nme2-ABE8e-i1 construct mismatch tolerance in ABE mCherry reporter cells as in FIG. 27(2)B at the overlapping target site as in FIG. 27(2)A.

FIG. 49E: Ratios of on-target/off-target editing for the Spy, Nme2-nt, Nme2-i1 and Nme2-i1^{V106W} ABE constructs tested at the overlapping Linc01588 target site (S2A) and the orthogonal SaCas9 R-loops (S2B). On-target efficiency for Spy-ABE8e is derived from the mean editing within its editing window as to not skew the ratio when compared to the wider on-target editing window of the three (3) Nme2-ABE8e constructs. n=3 biological replicates, data represent mean±SD. two-way ANOVA analysis: ns, p>0.05; *p<0.05; **p<0.01; ***p<0.001 ****p<0.0001.

FIGS. 50A-50B present exemplary data showing sensitivity of guide-independent ABE Cas9 constructs.

FIG. 50A: Exemplary data showing orthogonal R-Loop off-Target activity of guide-independent DNA A-to-G editing at orthogonal SaCas9 R-loops with Spy, Nme2-nt, Nme-i1 and Nme-i1^{V106W} ABE constructs as measured via amplicon sequencing. HNH nicking of the SaCas9 protein increased editing sensitivity at the orthogonal R-loops. n=3 biological replicates, data represent mean±SD.

50B (SEQ ID NO:284): Exemplary data showing on-target activity at a dual PAM Site (DS12) at the indicated target site (Upper Panel). Lower Panel: On-target activity of the Spy, Nme2-nt, Nme-i1 and Nme-i1^{V106W} ABE constructs were tested for the R-loop activity at a target site with overlapping PAMs as measured via amplicon sequencing. Box: Spy-ABE8e editing window. Overlapping target site sequence from 5′ to 3′ with adenines (red), and Spy- and Nme2-PAMs bold and underlined. n=3 biological replicates per off-target R-loop, data represent mean±SD.

FIGS. 51A-51C present representative Nme2Cas9 embodiments of inlaid TadA7.10 deaminase domains (e.g., ABE7.10).

FIG. 51A (SEQ ID NO:285): Upper Panel: 5′-3′ target sequence. Targeted adenine's (red). PAM (bold, underlined). Lower Panel: Exemplary data of A-to-G editing at an endogenous HEK293T Linc01588 loci with Nme2-ABE8e-i1, Nme2-ABE7-i1, Nme2-ABE7-i7 and Nme2-ABE7-i8 constructs. Measured via amplicon sequencing (n=3).

FIG. 51B (SEQ ID NO:286): Upper Panel: 5′-3′ target sequence. Targeted adenine's (red). PAM (bold, underlined). Lower Panel: Exemplary data of A-to-G editing at an endogenous HEK293T MeCP2 loci with Nme2-ABE8e-i1, Nme2-ABE7-i1, Nme2-ABE7-i7 and Nme2-ABE7-i8 constructs. Measured via amplicon sequencing (n=3).

FIG. 51C(SEQ ID NO:287): Upper Panel: 5′-3′ target sequence. Targeted adenine's (red). PAM (bold, underlined). Lower Panel: Exemplary data of A-to-G editing at an endogenous HEK293T Pcsk9 loci with Nme2-ABE8e-i1, Nme2-ABE7-i1, Nme2-ABE7-i7 and Nme2-ABE7-i8 constructs. Measured via amplicon sequencing (n=3).

FIGS. 52A-52C illustrate various embodiments of an Nme2Cas9 cytidine base editor (CBE) domain constructs.

FIG. 52A: Schematic representation of exemplary Nme2Cas9-CBE constructs: i) Nme2Cas9-CBE-(nt) N-terminal domain; ii) Nme2Cas9-CBE-(i1) inlaid domain; iii) Nme2Cas9-CBE-(i7) inlaid domain; and iv) Nme2Cas9-CBE-(i8) inlaid domain. The CBE may be a cytidine deaminase including, but not limited to, evoFERNY or rAPOBEC1.

FIG. 52B: Exemplary data of C-to-T editing at endogenous HEK293T genomic loci with Nme2-evoFERNY-nt, i1, i7 and i8 constructs (top) and Nme2-rAPOBEC1-nt, i1, i7 and i8 constructs (bottom) as measured by amplicon sequencing. n=3 biological replicates, data represent mean±SD.

FIG. 52C: Exemplary data showing rAPOBEC1 editing window and editing efficiency in HEK293T cells using three (3) genomic target sites. Data is expressed as a summary of mean C-to-T editing at the three endogenous HEK293T genomic target with Nme2-rAPOBEC1-nt, −i1, i7 and i8 constructs. Crossed out boxes denote no cytidine at the position within the target's tested. Measured via amplicon sequencing. n=3 biological replicates.

FIGS. 53A-53E present exemplary data showing PID Chimera's expand the targeting scope of Nme2Cas9 base editors.

FIG. 53A: Exemplary data showing expanded PAM scope of PID chimeric Nme2Cas9 nucleases.

FIG. 53B: Cartoon schematic of chimeric Nme2-ABE8e effectors with SmuCas9 PAM interacting domains

FIG. 53C: Examples of A-to-G editing with PID chimeric Nme2-ABE8e effectors at endogenous HEK293T genomic loci with N4CN PAMs by plasmid transfection and measured by amplicon sequencing. n=3 biological replicates, data represent mean±SD.

FIG. 53D: Each data point represents the mean A-to-G editing rates of an individual target site, measured by amplicon sequencing. The line represents the mean efficiency observed across all target sites. n=3 biological replicates.

FIG. 53E (SEQ ID NO:288-291): A-to-G editing with PID chimeric Nme2-ABE8e effectors at Linc01588 endogenous HEK293T genomic loci with N4CN PAMs by plasmid transfection and measured by amplicon sequencing. Target spacers 5′ to 3′ with PAM bold underlined. n=3 biological replicates, data represent mean±SD.

FIGS. 54A-54B present exemplary data showing conversion of the c.502 C>T (RETT-PDF) mutation with a chimeric Nme2Cas9Smu construct.

FIG. 54A (SEQ ID NO: 252,253,292,255): Schematic of a portion of Mecp2 exon 4, highlighting the (c.502 C>T; p.R168X) nonsense mutation (red) and potential bystander edits (orange), in RETT patient derived fibroblasts,

FIG. 54B (SEQ ID NO:256,257,293): A-to-G editing of Mecp2 502C>T in RETT patient fibroblasts in (A), with Nme2-ABE8e effectors delivered as mRNA with synthetic sgRNAs. Protospacer with target adenine (red), bystander adenine (orange), and PAM (bold, underlined). n=3 biological replicates, data represent mean±SD.

FIGS. 55A-55B present exemplary data showing conversion of the c.916 C>T (RETT-PDF) mutation with a chimeric Nme2Cas9Smu construct.

FIG. 55A (SEQ ID NO: 258-261): Schematic of a portion of Mecp2 exon 4, highlighting the (c.916 C>T; p.R306C) missense mutation (red) and potential bystander edits (orange), in RETT patient derived fibroblasts.

FIG. 55B (SEQ ID NO:294 and 262): A-to-G editing of Mecp2 916C>T in RETT-PDF cell line in (A), with Nme2-ABE8e effectors delivered as mRNA with synthetic sgRNAs. Protospacer with target adenine (red), bystander adenine (orange), and PAM (bold, underlined). n=3 biological replicates, data represent mean±SD.

FIGS. 56A-56B present exemplary data showing conversion of the c.763C>T (RETT-PiggyBac) mutation with a chimeric Nme2Cas9Smu construct.

FIG. 56A (SEQ ID NO:263-266): Schematic of a portion of Mecp2 exon 4, highlighting the (c.763 C>T; p.R255X) missense mutation (red) in RETT piggyBac cells.

FIG. 56B (SEQ ID NO: 267 and 295): A-to-G editing of Mecp2 763C>T in RETT-PiggyBac cell line in (A), with Nme2-ABE8e effectors and sgRNA delivered as plasmids. Protospacer with target adenine (red), and PAM (bold, underlined). n=2 biological replicates, data represent mean±SD.

FIGS. 57A-57B present exemplary data showing conversion of the c.808C>T (RETT-PiggyBac) mutation with a chimeric Nme2Cas9Smu construct.

FIG. 57A (SEQ ID NO:268-271): Schematic of a portion of Mecp2 exon 4, highlighting the (c.808C>T; p.R270X) missense mutation (red) in RETT PiggyBac cells and potential bystander edits. (orange).

FIG. 57B (SEQ ID NO:272 and 296): A-to-G editing of Mecp2 808C>T in RETT-PiggyBac cell line in (A), with Nme2-ABE8e effectors delivered as mRNA with synthetic sgRNAs. Protospacer with target adenine (red), bystander adenines (orange) and PAM (bold, underlined). n=2 biological replicates, data represent mean±SD.

FIGS. 58A-58C illustrate the expanded PAM scope and increased candidate targets provided by chimeric Cas9^{Smu} nucleases having single C PAMs.

FIG. 58A: A summary of reported respective target scope comparing dinucleotide C PAMs to single C PAMs.

FIG. 58B: An illustration of reported change in Cas9 nuclease PAM preference as a result of a PID swap.

FIG. 58C: A reported characterization of the SmuCas9 PID.

FIGS. 59A-59C present exemplary data showing in vivo ameliation of ALS symptoms following inlaid domain Nme2Cas9-ABE administration.

FIG. 59A: Dual AAV9 vector design.

FIG. 59B: Survival curve.

FIG. 59C: Representative L5 ventral root cross sections in P110 mice (e.g., lifespan midpoint) showing ALS-mediated cell breakdown reversal subsequent to gene editing.

FIGS. 60A-60B (SEQ ID NO:297 and 298) present a representative illustration of SOD1 exon 2 skipping by editing splicing sites. Intron residues are in lower case.

FIG. 60A: Gene editing of the intron 1 splice acceptor. The N₄CC PAM places the target A at A15. The single-C PAM places the target B at A15.

FIG. 60B: Gene editing the intron 2 splice donor. Additional G residues (for single-C PAMs) are highlighted.

FIG. 61 (SEQ ID NO:299) presents a portion of the SOD1 exon 2 loci to identify potential Nme2Cas9-ABE fusion proteins and their respective guide RNAs to correct an SOD1_{G37R} mutation. Missense bystander nucleotides are in blue. Frameshift mutation nucleotides are in red.

FIGS. 62A-62B present an exemplary Nme2Cas9^{Smu} construct: (FIG. 62A) (SEQ ID NO:300)amino acid sequence; (FIG. 62B) (SEQ ID NO: 301) nucleic acid sequence; BPSV40-NLS (purple), Nme2Cas9-delta PID (blue), SmuCas9-PID (orange), Linkers (black).

FIGS. 63A-63B present an exemplary Nme2Cas9^{Smu} construct: (FIG. 63A) (SEQ ID NO:302)amino acid sequence; (FIG. 63B) (SEQ ID NO:303) nucleic acid sequence; SV40-NLS (purple), nucleoplasmin-NLS (green), Nme2Cas9-delta PID (blue), SmuCas9 PID (orange), 3xHA (SEQ ID NO:171) (italicized), unlabeled-NLS (red), Linkers (black).

FIGS. 64A-64B present an exemplary Nme2^{Smu}-ABE8e-i1 inlaid domain construct. (FIG. 64A) (SEQ ID NO:304) amino acid sequence; (FIG. 64B) (SEQ ID NO:305) nucleic acid sequence; BPSV40-NLS (purple), Nme2Cas9-delta PID (blue), TadA8e (red), SmuCas9 PID (orange), Linkers (black).

FIGS. 65A-65B present an exemplary Nme2^{Smu}-ABE8e-i7 inlaid domain construct. (FIG. 65A) (SEQ ID NO:306)amino acid sequence; (FIG. 65B) (SEQ ID NO:307) nucleic acid sequence; BPSV40-NLS (purple), Nme2Cas9-delta PID (blue), TadA8e (red), SmuCas9 PID (orange), Linkers (black).

FIGS. 66A-66B presents an exemplary Nme2^{Smu}-ABE8e-i8 inlaid domain construct. (FIG. 66A) (SEQ ID NO:308)amino acid sequence; (FIG. 66B) (SEQ ID NO:309) nucleic acid sequence; BPSV40-NLS (purple), Nme2Cas9-delta PID (blue), TadA8e (red), SmuCas9 PID (orange), Linkers (black).

FIGS. 67A-67B presents an exemplary Nme2^{Smu}-ABE8e-nt N-terminal domain construct. (FIG. 67A) (SEQ ID NO:310) amino acid sequence; (FIG. 67B) (SEQ ID NO:311) nucleic acid sequence; BPSV40-NLS (purple), Nme2Cas9-delta PID (blue), TadA8e (red), SmuCas9 PID (orange), Linkers (black).

### DETAILED DESCRIPTION OF THE INVENTION

The present invention is related to the field of gene editing. In particular, the gene editing is directed toward single nucleotide base editing. For example, such single nucleotide base editing results in a conversion of a mutated base pair to a wild type base pair. The high accuracy and precision of the presently disclosed single nucleotide base gene editor is accomplished by a fusion protein including an NmeCas9 nuclease and an inlaid nucleotide deaminase protein domain. The Nme2Cas9 protospacer interacting domain may be replaced with an SmuCas9 protospacer interacting domain.

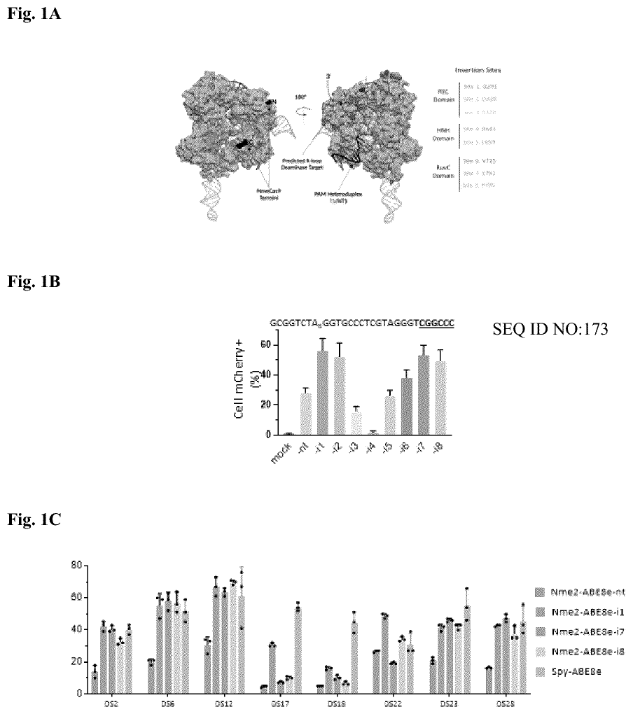

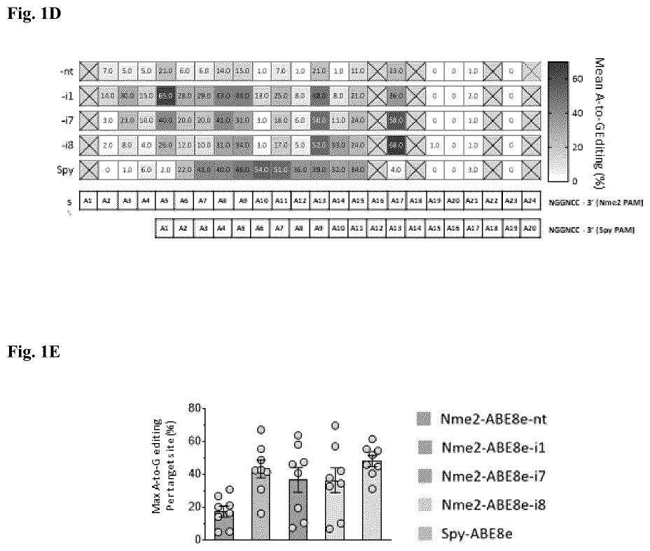

In one embodiment, the present invention contemplates a Cas9 protein contemplating an exogenous inlaid domain. In one embodiment, the exogenous inlaid domain is inserted in the Cas9 REC domain. In one embodiment, the exogenous inlaid domain is inserted in the Cas9 HNH domain. In one embodiment, the exogenous inlaid domain is inserted in the Cas9 RuvC domain. In one embodiment, the exogenous inlaid domain is a nucleotide base editor. In one embodiment, the nucleotide base editor is an adenine base editor (ABE). In one embodiment, the nucleotide base editor is a cytidine base editor (CBE). For example, an inlaid domain Nme2Cas9-ABE fusion protein comprises greater gene editing efficiency as compared to a N-terminal domain NmwCas9-ABE fusion protein. See, FIGS. 1A-D.

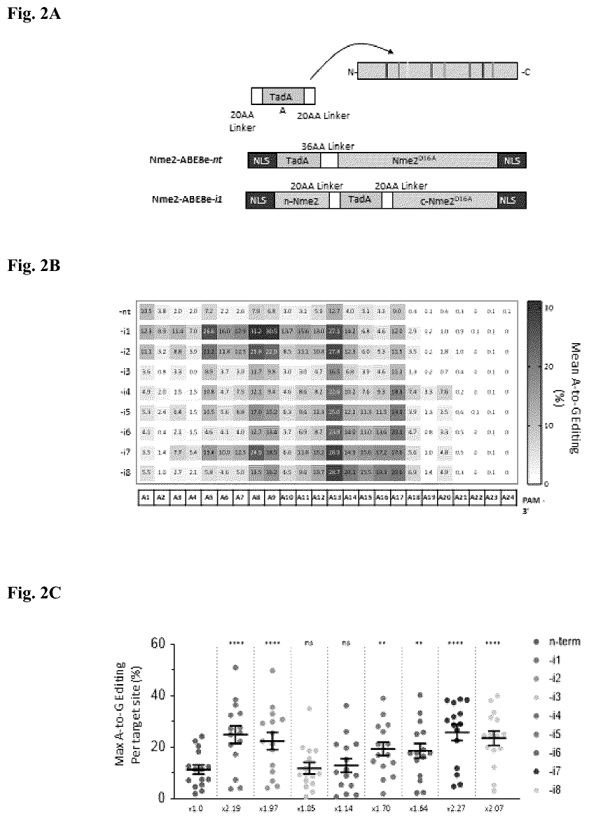

The insertion of an inlaid domain may be placed in a variety of positions within the Cas9 protein, each of which has superior gene editing activity as compared to the N-terminal domain construct. See, FIGS. 2A-C.

### I. CRISPR Cas9 Gene Editors

### A. N-Terminal Cas9 Deaminase Fusion Proteins

Fusion proteins have been reported comprising an Nme2Cas9 and an N-terminal deaminase protein. See, FIG. 3A and FIG. 3B. For example, the deaminase protein is Apobec1 (YE1-BE3). Kim et al., “Increasing the genome-targeting scope and precision of base editing with engineered Cas9-cytidine deaminase fusions”. Nature Biotechnology 35 (2017). The YE1-BE3-nNme2Cas9 (D16A)-UGI construct has the sequence of:

(SEQ ID NO: 1)
MSSETGPVAVDPTLRRRIEPHEFEVFFDPRELRKETCLLYEINWGGRHSIWRHTSQNTNKH
VEVNFIEKFTTERYFCPNTRCSITWFLSYSPCGECSRAITEFLSRYPHVTLFIYIARLYHHAD
PENRQGLRDLISSGVTIQIMTEQESGYCWRNFVNYSPSNEAHWPRYPHLWVRLYVLELYC
IILGLPPCLNILRRKQPQLTFFTIALQSCHYQRLPPHILWATGLKSGSETPGTSESATPESMA
AFKPNPINYILGLAIGIASVGWAMVEIDEEENPIRLIDLGVRVFERAEVPKTGDSLAMARRLARSV
RRLTRRRAHRLLRARRLLKREGVLQAADFDENGLIKSLPNTPWQLRAAALDRKLTPLEWSAVLL
HLIKHRGYLSQRKNEGETADKELGALLKGVANNAHALQTGDFRTPAELALNKFEKESGHIRNQ
RGDYSHTFSRKDLQAELILLFEKQKEFGNPHVSGGLKEGIETLLMTQRPALSGDAVQKMLGHC
TFEPAEPKAAKNTYTAERFIWLTKLNNLRILEQGSERPLTDTERATLMDEPYRKSKLTYAQARKL
LGLEDTAFFKGLRYGKDNAEASTLMEMKAYHAISRALEKEGLKDKKSPLNLSSELQDEIGTAFS
LFKTDEDITGRLKDRVQPEILEALLKHISFDKFVQISLKALRRIVPLMEQGKRYDEACAEIYGDH
YGKKNTEEKIYLPPIPADEIRNPVVLRALSQARKVINGVVRRYGSPARIHIETAREVGKSFKDRKEI
EKRQEENRKDREKAAAKFREYFPNFVGEPKSKDILKLRLYEQQHGKCLYSGKEINLVRLNEKGY
VEIDAALPFSRTWDDSFNNKVLVLGSENQNKGNQTPYEYFNGKDNSREWQEFKARVETSRFPR
SKKQRILLQKFDEDGFKECNLNDTRYVNRFLCQFVADHILLTGKGKRRVFASNGQITNLLRGF
WGLRKVRAENDRHHALDAVVVACSTVAMQQKITRFVRYKEMNAFDGKTIDKETGKVLHQKTH
FPQPWEFFAQEVMIRVFGKPDGKPEFEEADTPEKLRTLLAEKLSSRPEAVHEYVTPLFVSRAPN
RKMSGAHKDTLRSAKRFVKHNEKISVKRVWLTEIKLADLENMVNYKNGREIELYEALKARLEAY
GGNAKQAFDPKDNPFYKKGGQLVKAVRVEKTQESGVLLNKKNAYTIADNGDMVRVDVFCKV
DKKGKNQYFIVPIYAWQVAENILPDIDCKGYRIDDSYTFCFSLHKYDLIAFQKDEKSKVEFAYYI
NCDSSNGRFYLAWHDKGSKEQQFRISTQNLVLIQKYQVNELGKEIRPCRLKKRPPVRSGGSTNL
SDIIEKETGKQLVIQESILMLPEEVEEVIGNKPESDILVHTAYDESTDENVMLLTSDAP
EYKPWALVIQDSNGENKIKMLSGGSPKKKRKV*

YE1-BE3 (underlined); linker (bold), nNme2Cas9 (italics), UGI (bold/underlined), SV40 NLS (unannotated*).

Another example comprises an Nme2Cas9 and a terminal TadA adenine deaminase protein (e.g., ABE7.10). The ABE7.10-nNme2Cas9 (D16A) construct has the following sequence:

(SEQ ID NO: 2)
MSEVEFSHEYWMRHALTLAKRAWDEREVPVGAVLVHNNRVIGEGWNRPIGRHDPTAHA
EIMALRQGGLVMQNYRLIDATLYVTLEPCVMCAGAMIHSRIGRVVEGARDAKTGAAGSL
MDVLHHPGMNHRVEITEGILADECAALLSDFFRMRRQEIKAQKKAQSSTDSGGSSGGSSG
SETPGTSESATPESSGGSSGGSSEVEFSHEYWMRHALTLAKRARDEREVPVGAVLVLN
NRVIGEGWNRAIGLHDPTAHAEIMALRQGGLVMQNYRLIDATLYVTFEPCVMCAGA
MIHSRIGRVVFGVRNAKTGAAGSLMDVLHYPGMNHRVEITEGILADECAALLCYFFR
MPRQVFNAQKKAQSSTDSGGSSGGSSGSETPGTSESATPESSGGSSGGSMAAFKPNPINYILG
LAIGIASVGWAMVEIDEEENPIRLIDLGVRVFERAEVPKTGDSLAMARRLARSVRRLTRRRAHRLL
RARRLLKREGVLQAADFDENGLIKSLPNTPWQLRAAALDRKLTPLEWSAVLLHLIKHRGYLSQR
KNEGETADKELGALLKGVANNAHALQTGDFRTPAELALNKFEKESGHIRNQRGDYSHTFSRKD
LQAELILLFEKQKEFGNPHVSGGLKEGIETLLMTQRPALSGDAVQKMLGHCTFEPAEPKAAKN
TYTAERFIWLTKLNNLRILEQGSERPLTDTERATLMDEPYRKSKLTYAQARKLLGLEDTAFFKGL
RYGKDNAEASTLMEMKAYHAISRALEKEGLKDKKSPLNLSSELQDEIGTAFSLFKTDEDITGRLK
DRVQPEILEALLKHISFDKFVQISLKALRRIVPLMEQGKRYDEACAEIYGDHYGKKNTEEKIYLP
PIPADEIRNPVVLRALSQARKVINGVVRRYGSPARIHIETAREVGKSFKDRKEIEKRQEENRKDRE
KAAAKFREYFPNFVGEPKSKDILKLRLYEQQHGKCLYSGKEINLVRLNEKGYVEIDHALPFSRT
WDDSFNNKVLVLGSENQNKGNQTPYEYFNGKDNSREWQEFKARVETSRFPRSKKQRILLQKF
DEDGFKECNLNDTRYVNRFLCQFVADHILLTGKGKRRVFASNGQITNLLRGFWGLRKVRAEND
RHHALDAVVVACSTVAMQQKITRFVRYKEMNAFDGKTIDKETGKVLHQKTHFPQPWEFFAQE
VMIRVFGKPDGKPEFEEADTPEKLRTLLAEKLSSRPEAVHEYVTPLFVSRAPNRKMSGAHKDTL
RSAKRFVKHNEKISVKRVWLTEIKLADLENMVNYKNGREIELYEALKARLEAYGGNAKQAFDPK
DNPFYKKGGQLVKAVRVEKTQESGVLLNKKNAYTIADNGDMVRVDVFCKVDKKGKNQYFIVPI
YAWQVAENILPDIDCKGYRIDDSYTFCFSLHKYDLIAFQKDEKSKVEFAYYINCDSSNGRFYLAW
HDKGSKEQQFRISTQNLVLIQKYQVNELGKEIRPCRLKKRPPVREDKRPAATKKAGQAKKKK*

TadA (underlined), TadA 7.10 (underlined/bold), linker (bold), nNme2Cas9 (italics), Nucleoplasmin NLS (unannotated*).

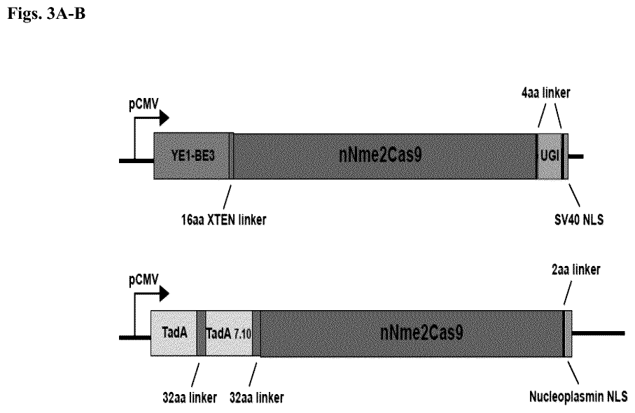

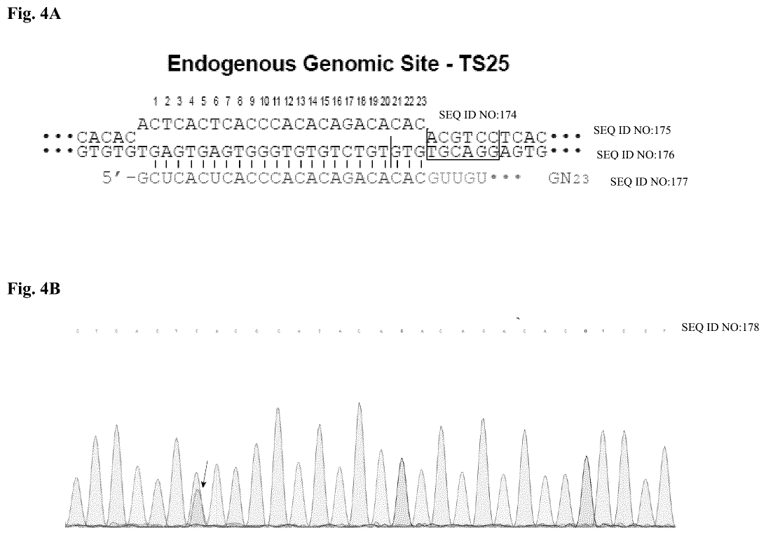

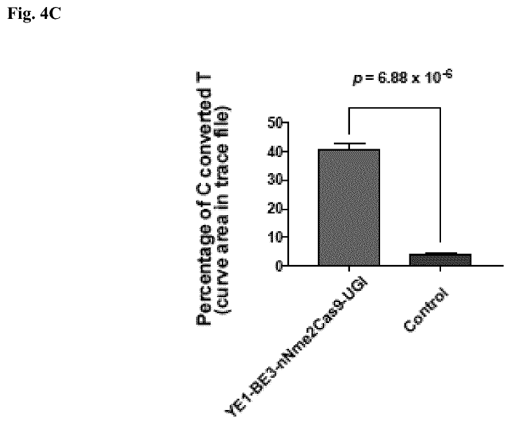

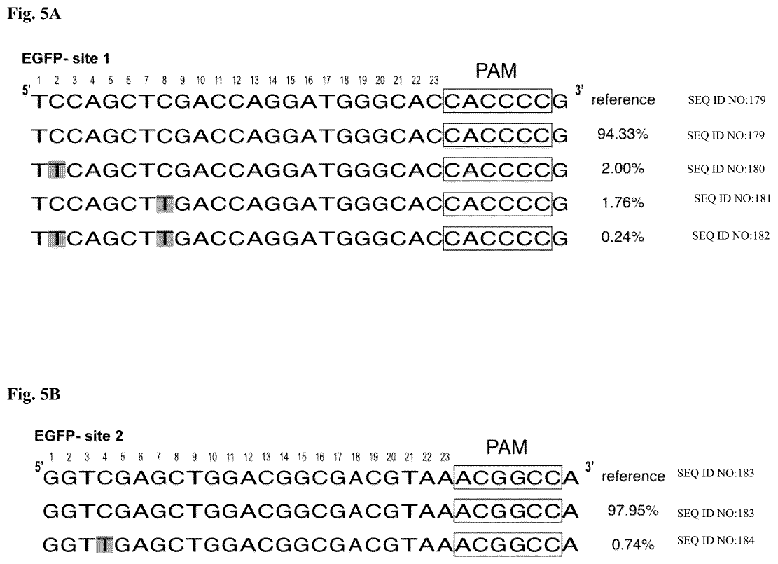

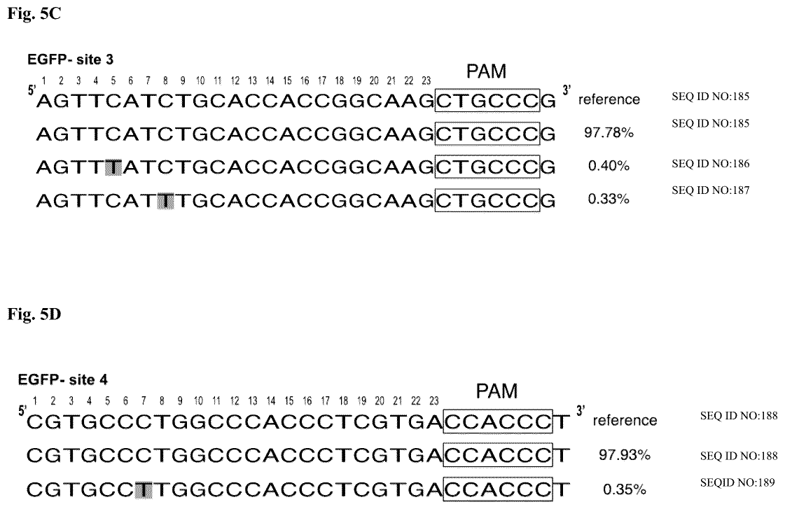

Electroporation of HEK293T cells with DNA plasmids comprising a YE1-BE3-Nme2Cas9 with a terminally fused nucleotide deaminase protein demonstrated single-base editing of a C·G base pair to a T·A base pair at an endogenous target site (TS25). See, FIGS. 4A-C. Four other YE1-BE3-nNme2Cas9/D16A mutant N-terminal fusion proteins were co-expressed with enhanced green fluorescent protein (EGFP) in a stable K562-derived cell line. Each YE1-BE3-nNme2Cas9/D16A mutant N-terminal fusion protein had a specific UGI target site. See, FIGS. 5A-D. Deep-sequencing analysis indicates YE1-BE3-nNme2Cas9 converts C residues to T residues at each of the four EGFP target sites. The percentage of editing ranged from 0.24% to 2%. The potential base editing window is from nucleotides 2-8 in the displaced DNA strand, counting the nucleotide at the 5′ (PAM-distal) end as nucleotide #1.

The expression of an ABE7.10-nNme2Cas9 (D16A) N-terminal fusion protein for base editing may be an effective treatment for tyrosinemia by reversing a G-to-A point mutation in the Fah gene. G-to-A mutation (red) at the last nucleotide of exon 8 in Fah gene, causing exon skipping. Exon skipping provides benefit by eliminating a pathogenic mutation from a mature mRNA, restoring reading frame to compensate for a disease-causing frameshift mutation, or inactivating the expression of a gene that contributes to disease (by inducing an out-of-frame splicing event, or deleting an essential gene region from the mature mRNA, or both). The latter allows ABEs to induce gene knockouts, given that ABEs cannot be used to introduce nonsense mutations—the ABE precursor to either G-containing stop codon (UAG or UGA) is UAA, which is already a stop codon. ABEs can convert AG splice acceptor site dinucleotides to GG and can also convert GU splice donor site dinucleotides (AC on the opposite strand) to GC.

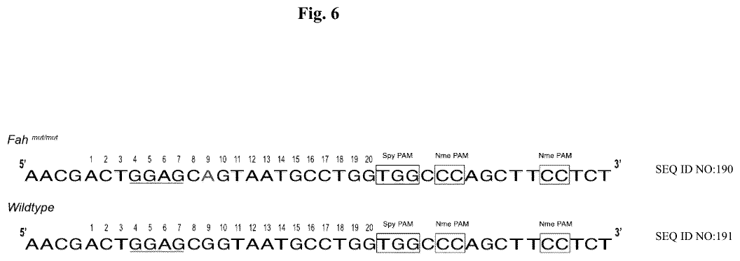

FAH deficiency leads to toxin accumulation and severe liver damage. The position of a SpyCas9 PAM (black rectangular box) downstream of the mutation is not optimal for designing the sgRNA since the A mutation is out of the efficient base editing window of ABE7.10, which is 4-7th nt at the 5′ (PAM-distal) end (underlined) (Gaudelli et al., 2017). However, there are two Nme2Cas9 PAMs (red rectangular box) in the downstream sequences that can potentially correct the mutation and revert DNA sequence to wildtype via ABE7.10-nNme2Cas9 (D16A). This figure serves as a potential example of a site where Nme2Cas9 with N-terminal adenine deaminase proteins could overcome limitations of existing base editors. See, FIG. 6. It is further believed that Nme2Cas9 base editors can perform precise base editing that cannot be achieved with conventional SpyCas9-derived base editors due to a suboptimal base editing window relative to available PAMs nearby.

### B. Nme2Cas9 PAM Interacting Domains

Protospacer adjacent motif (PAM) recognition by Cas9 orthologs occurs predominantly through protein-DNA interactions between the PAM Interacting Domain (PID) and the nucleotides adjacent to the protospacer (Jiang and Doudna, 2017). PAM mutations often enable phage escape from type II CRISPR immunity (Paez-Espino et al., 2015), placing these systems under selective pressure not only to acquire new CRISPR spacers, but also to evolve new PAM specificities via PID mutations. In addition, some phages and MGEs express anti-CRISPR (Acr) proteins that inhibit Cas9 (Pawluk et al., 2016; Hynes et al., 2017; Rauch et al., 2017). PID binding is an effective inhibitory mechanism adopted by some Acrs (Dong et al., 2017; Shin et al., 2017; Yang and Patel, 2017), suggesting that PID variation may also be driven by selective pressure to escape Acr inhibition. Cas9 PIDs can evolve such that closely-related orthologs recognize distinct PAMs, as illustrated recently in two species of Geobacillus. The Cas9 encoded by G. stearothermophilus recognizes a N₄CRAA PAM, but when its PID was swapped with that of strain LC300's Cas9, its PAM requirement changed to N₄GMAA (Harrington et al., 2017b).

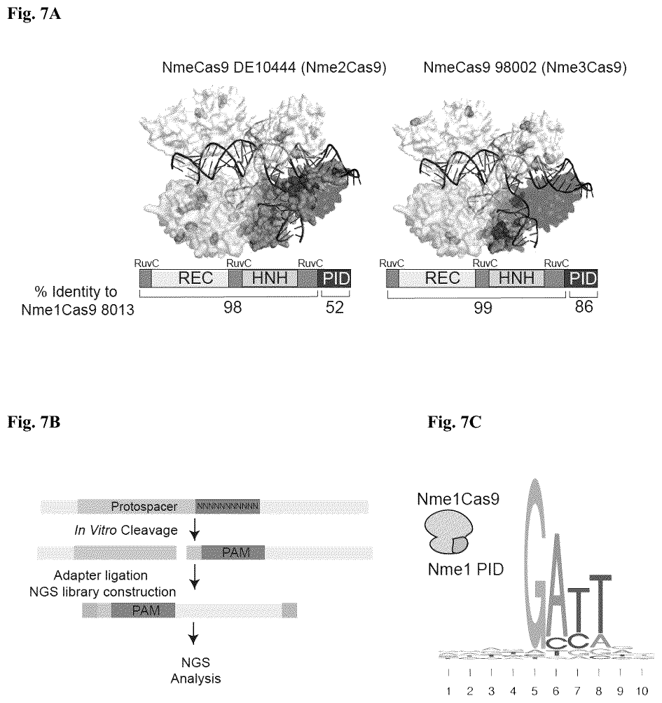

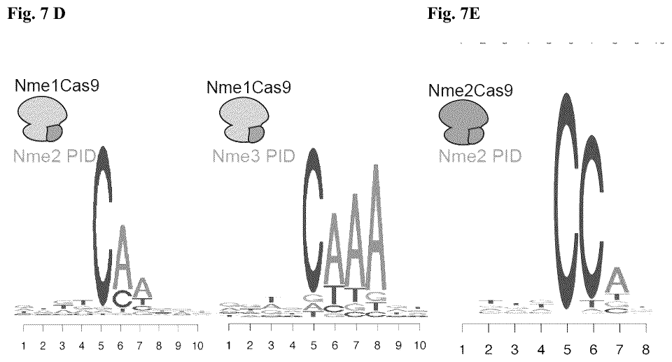

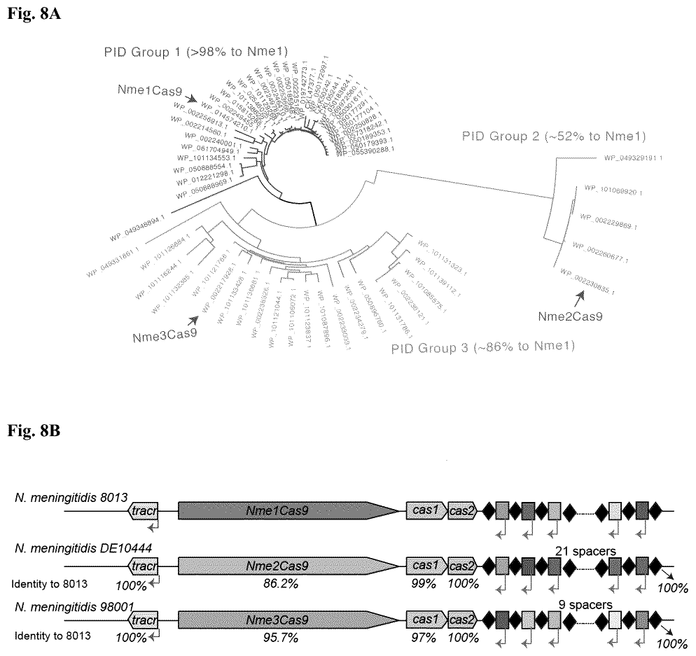

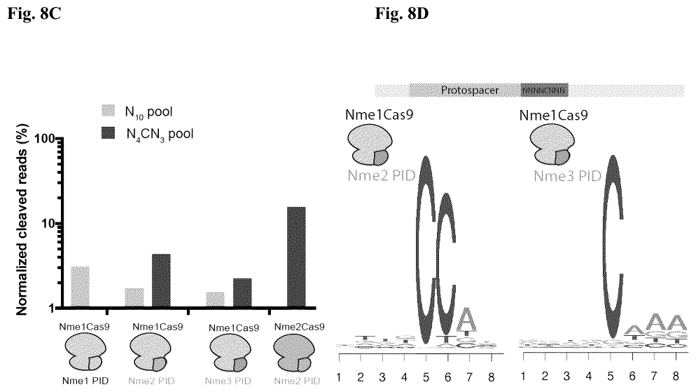

In one embodiment, the present invention contemplates a plurality of N. meningitidis Cas9 orthologs with divergent PIDs that recognize different PAMs. In one embodiment, the present invention contemplates a Cas9 protein with a high sequence identity (>80% along their entire lengths) to that of NmeCas9 strain 8013 (Nme1Cas9) (Zhang et al., 2013). Nme1Cas9 also has a small size and naturally high accuracy as discussed above. (Lee et al., 2016; Amrani et al., 2018). Alignments revealed three clades of meningococcal Cas9 orthologs, each with >98% identity in the N-terminal ˜820 amino acid (aa) residues, which includes all regions of the protein other than the PID. See, FIG. 7A and FIG. 8A.

All of these Cas9 orthologs are 1,078-1,082 aa in length. The first clade (group 1) includes orthologs in which the >98% aa sequence identity with Nme1Cas9 extends through the PID. In contrast, the other two groups had PIDs that were significantly diverged from that of Nme1Cas9, with group 2 and group 3 orthologs averaging ˜52% and ˜86% PID sequence identity with Nme1Cas9, respectively. One meningococcal strain was selected from each group: i) De11444 from group 2; and ii) 98002 from group 3 for detailed analysis, which are referred to herein as Nme2Cas9 (1,082 aa) and Nme3Cas9 (1,081 aa), respectively. The CRISPR-cas loci from these two strains have repeat sequences and spacer lengths that are identical to those of strain 8013. See, FIG. 8B. This strongly suggested that their mature crRNAs also have 24 nt guide sequences and 24 nt repeat sequences (Zhang et al., 2013). Similarly, the tracrRNA sequences of De11444 and 98002 were 100% identical to the 8013 tracrRNA. See, FIG. 8B. These observations imply that the same sgRNA sequence scaffold can guide DNA cleavage by all three Cas9s.

To determine whether these Cas9 orthologs have distinct PAMs, the PID of Nme1Cas9 was replaced with that of either Nme2Cas9 or Nme3Cas9. To identify the corresponding PAM requirements, these protein chimeras were expressed in Escherichia coli, purified, and used for in vitro PAM identification (Karvelis et al., 2015; Ran et al., 2015; Kim et al., 2017). Briefly, a pool of DNA fragments containing a protospacer followed by a 10-nt randomized sequence was cleaved in vitro using recombinant Cas9 and a cognate, in vitro-transcribed sgRNA. See, FIG. 7B. Only those DNAs containing a Cas9 PAM sequence were expected to be cleaved. Cleavage products were then sequenced to identify the PAMs. See, FIGS. 7C-D.

The expected N₄GATT PAM consensus was validated in the recovered full-length Nme1Cas9. See, FIG. 7C. Chimeric PID-swapped derivatives exhibited a strong preference for a C residue in the 5^{th} position in place of the G recognized by Nme1Cas9. See, FIG. 7D. Any remaining PAM nucleotides could not be confidently assigned due to the low cleavage efficiencies of the chimeric proteins under the conditions used. See, FIG. 8C. To further resolve the PAMs, in vitro assays were performed on a library with a 7-nt randomized sequence possessing an invariant C at the 5^{th} PAM position (e.g., 5′-NNNNCNNN-3′ on the sgRNA-noncomplementary strand). This strategy yielded a much higher cleavage efficiency and the results indicated that the Nme2Cas9 and Nme3Cas9 PIDs recognize NNNNCC (A) and NNNNCAAA PAMs, respectively. See, FIGS. 8C-D. The Nme3Cas9 consensus is similar to that of GeoCas9 (Harrington et al., 2017b).

These tests were repeated using a full-length Nme2Cas9 (rather than a PID-swapped chimera) with the NNNNCNNN DNA pool, and again a NNNNCC (A) consensus was recovered. See, FIG. 7E. It was noted that this test had more efficient cleavage. See, FIG. 8C. These data suggest that one or more of the 15 amino acid changes in Nme2Cas9 (relative to Nme1Cas9) outside of the PID support efficient DNA cleavage activity. See, FIG. 8C. Because the unique, 2-3 nt PAM of Nme2Cas9 affords a higher density of potential target sites than the previously described compact Cas9 orthologs, it was selected for further analyses.

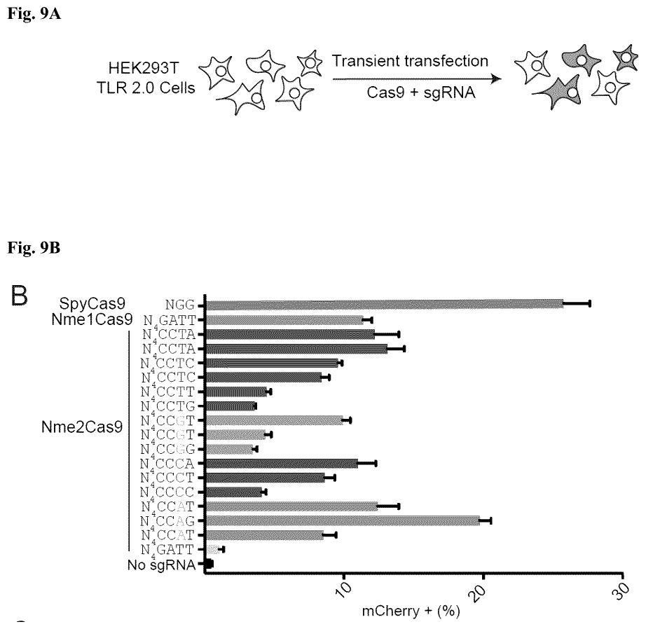

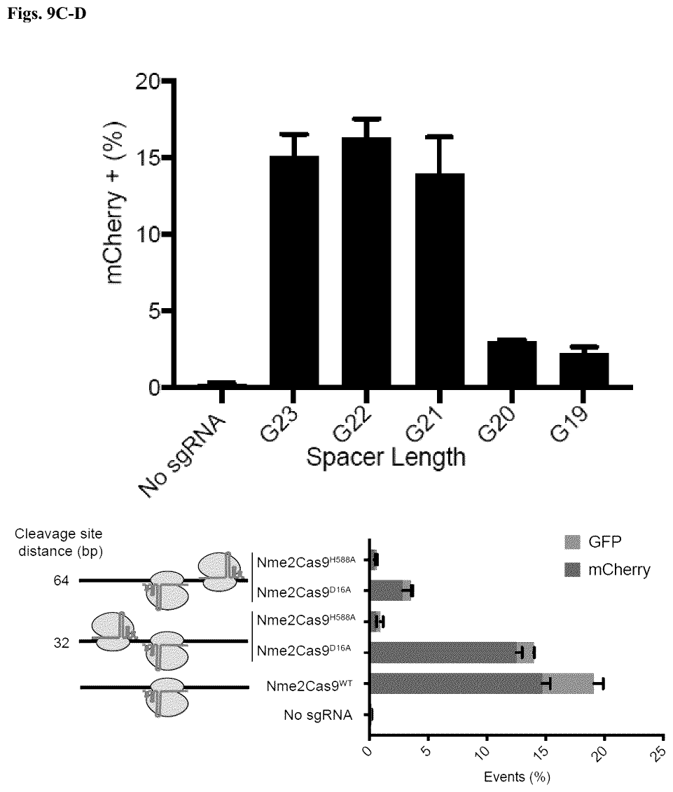

To test the efficacy of Nme2Cas9 in human genome editing, a full-length (e.g., not PID-swapped) human-codon-optimized Nme2Cas9 construct was cloned into a mammalian expression plasmid with appended nuclear localization signals (NLSs) and linkers validated previously for Nme1Cas9 (Amrani et al., 2018). For initial tests, a modified, fluorescence-based Traffic Light Reporter (TLR2.0) was used (Certo et al., 2011). Briefly, a disrupted GFP is followed by an out-of-frame T2A peptide and mCherry cassette. When DNA double-strand breaks (DSBs) are introduced in the broken-GFP cassette, a subset of non-homologous end joining (NHEJ) repair events leave +1-frameshifted indels, placing mCherry in frame and yielding red fluorescence that can be easily quantified by flow cytometry See, FIG. 9A. Homology-directed repair (HDR) outcomes can also be scored simultaneously by including a DNA donor that restores the functional GFP sequence, yielding a green fluorescence (Certo et al., 2011). Because some indels do not introduce a +1 frameshift, the fluorescence readout generally provides an underestimate of the true editing efficiency. Nonetheless, the speed, simplicity, and low cost of the assay makes it useful as an initial, semi-quantitative measure of genome editing in HEK293T cells carrying a single TLR2.0 locus incorporated via lentivector.

For initial tests, Nme2Cas9 plasmid was transiently co-transfected with one of fifteen sgRNA plasmids carrying spacers that target TLR2.0 sites with N₄CC PAMs. No HDR donor was included, so only NHEJ-based editing (mCherry) was scored. Most sgRNAs were in a G23 format (i.e. a 5′-terminal G to facilitate transcription, followed by a 23 nt guide sequence), as used routinely for Nme1Cas9 (Lee et al., 2016; Pawluk et al., 2016; Amrani et al., 2018; Ibraheim et al., 2018). No sgRNA and an sgRNA targeting an N₄GATT PAM were used as negative controls, and SpyCas9+ sgRNA and Nme1Cas9+ sgRNA co-transfections (targeting NGG and N₄GATT protospacers, respectively) were included as positive controls. Editing by SpyCas9 and Nme1Cas9 was readily detectable (˜28% and 10% mCherry, respectively). See, FIG. 7B.

For Nme2Cas9, all 15 targets with N₄CC PAMs were functional, though to various extents ranging from 4% to 20% mCherry. These fifteen sites include examples with each of the four possible nucleotides in the 7^{th} PAM position (e.g., after the CC dinucleotide), indicating that a slight preference for an A residue was observed in vitro (FIG. 7E) does not reflect a PAM requirement for editing applications in human cells. The N₄GATT PAM control yielded mCherry signal similar to no-sgRNA control. See, FIG. 9B.

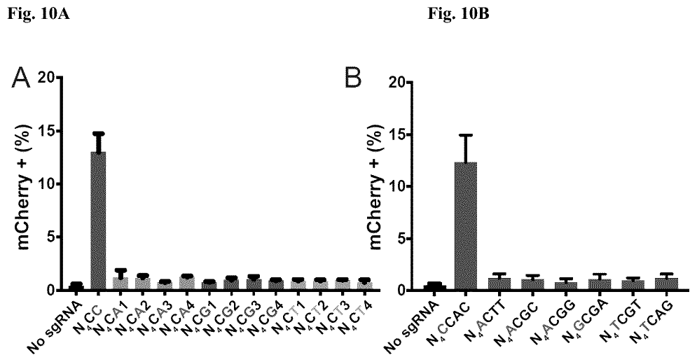

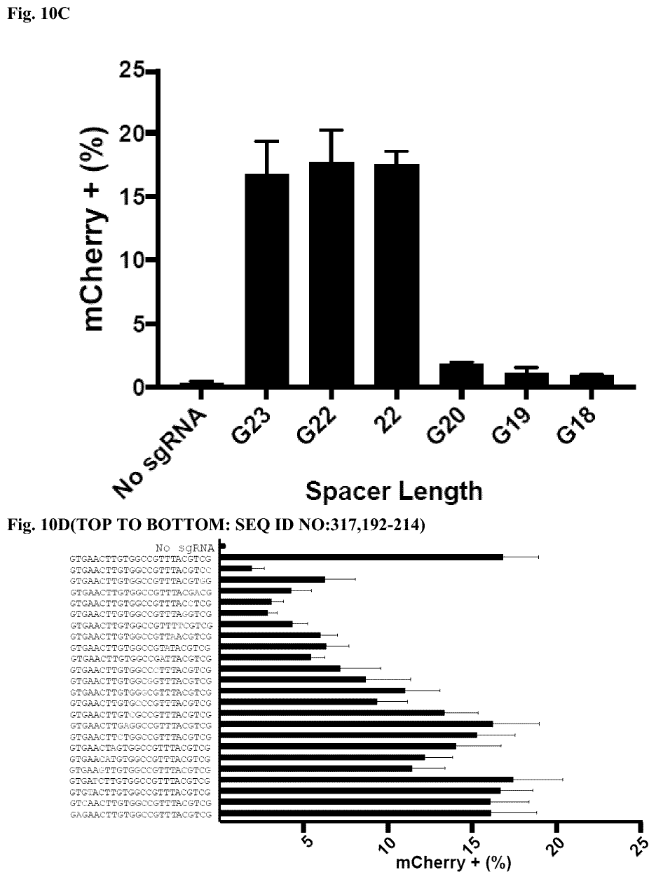

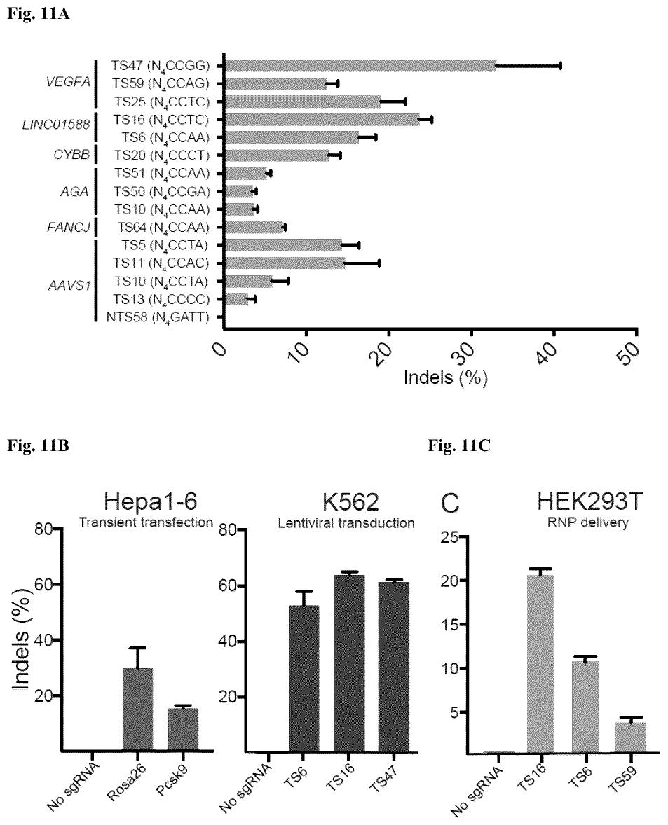

To determine whether both C residues in the N₄CC PAM are involved in editing, a series of N₄DC (D=A, T, G) and N₄CD PAM sites were tested in TLR2.0 reporter cells. See, FIGS. 10A and 10B. No detectable editing was found at any of these sites, providing an initial indication that both C residues of the N₄CC PAM consensus are required for efficient Nme2Cas9 activity.

The length of the spacer in the crRNA differs among Cas9 orthologs and can affect on-vs. off-target activity (Cho et al., 2014; Fu et al., 2014). SpyCas9's optimal spacer length is 20 nts, with truncations down to 17 nts tolerated (Fu et al., 2014). In contrast, Nme1Cas9 usually has 24-nt spacers (Hou et al., 2013; Zhang et al., 2013), and tolerates truncations down to 18-20 nts (Lee et al., 2016; Amrani et al., 2018). To test spacer length requirements for Nme2Cas9, guide RNA plasmids were created for each targeted single TLR2.0 site, but with varying spacer lengths. See, FIG. 9C and FIG. 10C. Comparable activities were observed with G23, G22 and G21 guides, but significantly decreased activity upon further truncation to G20 and G19 lengths. See, FIG. 9C. These results validate Nme2Cas9 as a genome editing platform, with 22-24 nt guide sequences, at N₄CC PAM sites in cultured human cells.

### B. HDR And HNH Cas9 Nickases

Cas9 enzymes use their HNH and RuvC domains to cleave the guide-complementary and non-complementary strand of the target DNA, respectively. Cas9 nickases (nCas9s), in which either the HNH or RuvC domain is mutationally inactivated, have been used to induce homology-directed repair (HDR) and to improve genome editing specificity via DSB induction by dual nickases (Mali et al., 2013a; Ran et al., 2013).

To test the efficacy of Nme2Cas9 as a nickase, a Nme2Cas9^{D16A} (HNH nickase) and Nme2Cas9^{H588A} (RuvC nickase) were created, which possess alanine mutations in catalytic residues of the RuvC and HNH domains, respectively (Esvelt et al., 2013; Hou et al., 2013; Zhang et al., 2013). TLR2.0 cells, along with a GFP donor dsDNA, were used to determine whether Nme2Cas9-induced nicks can induce precise edits via HDR. Target sites within TLR2.0 were used to test the functionality of each nickase using guides targeting cleavage sites spaced 32 bp and 64 bp apart. See, FIG. 9D. Wildtype Nme2Cas9 targeting a single site showed efficient editing, with both NHEJ and HDR as outcomes of repair. For nickases, cleavage sites 32 bp and 64 bp apart showed editing using the Nme2Cas9^{D16A} (HNH nickase), but neither target pair worked with Nme2Cas9^{H588A}. These results suggest that Nme2Cas9 HNH nickase can be used for efficient genome editing, as long as the sites are in close proximity.

Studies in previously characterized Cas9s have identified a specific region proximal to the PAM where Cas9 activity is highly sensitive to sequence mismatches. This 8 to 12-nt region is known as the seed sequence and has been observed among all Cas9s characterized to date (Gorski et al., 2017). To determine whether Nme2Cas9 also possesses a seed sequence, a series of transient transfections was performed, each targeting the same locus in TLR2.0, but with a single-nucleotide mismatch at different positions of the guide. See, FIG. 10D. A significant decrease in the number of mCherry-positive cells was observed for mismatches in the first 10-12 nts proximal to the PAM, suggesting that Nme2Cas9 possesses a seed sequence in this region.

### C. Cas9 Plasmid/Vector Cell Transfection

Nme2Cas9's ability to function in different mammalian cell lines was tested using various delivery methods. As an initial test, forty (40) different sites (29 with a N₄CC PAM, and 11 sites were tested with a N₄CD PAM). Several loci were selected (AAVS1, VEGFA, etc.), and target sites with N₄CC PAMs were randomly chosen for editing with Nme2Cas9. Editing (%) was determined by transiently transfecting 150 ng of Nme2Cas9 along with 150 ng of sgRNA plasmids followed by TIDE analysis 72 hours post-transfection. A subset of target sites and their respective TIDE primer sets exhibiting a range of editing efficiencies in this initial screen was selected for repeat analyses in triplicate. See, FIG. 11A; Table 1 and Table 2.

TABLE 1
Exemplary Endogenous human genome editing sites targeted by Nme2Cas9 (bolded nts).

| | Site | SEQ ID | | | | |
|---|---|---|---|---|---|---|
| No. | Name | NO | Spacer Seq | PAM | Locus | Editing (%) |
| 1 | TS1 | 3 | GGTTCTG | CCTCCACC | AAVS1 | ND |
| | | | GGTACTT | | | |
| | | | TTATCTGT | | | |
| | | | CC | | | |
| 2 | TS4 | 4 | GTCTGCC | TAGACGAA | AAVS1 | 11 |
| | | | TAACAGG | | | |
| | | | AGGTGGG | | | |
| | | | GGT | | | |
| 3 | TS5 | 5 | GAATATC | GAGGCCTA | AAVS1 | 15 |
| | | | AGGAGAC | | | |
| | | | TAGGAAG | | | |
| | | | GAG | | | |
| 4 | TS6 | 6 | GCCTCCC | CAGCCCAA | LINC01588 | 20 |
| | | | TGCAGGG | | | |
| | | | CTGCTCC | | | |
| | | | C | | | |
| 5 | TS10 | 7 | GAGCTAG | GGGCCCTA | AAVS1 | 3.5 |
| | | | TCTTCTTC | | | |
| | | | CTCCAAC | | | |
| | | | CC | | | |
| 6 | TS11 | 8 | GATCTGT | GGGGCCAC | AAVS1 | 9 |
| | | | CCCCTCC | | | |
| | | | ACCCCAC | | | |
| | | | AGT | | | |
| 7 | TS12 | 9 | GGCCCAA | TGACCCGA | AAVS1 | 10 |
| | | | ATGAAAG | | | |
| | | | GAGTGAG | | | |
| | | | AGG | | | |
| 8 | TS13 | 10 | GCATCCT | GACACCCC | AVS1 | 2 |
| | | | CTTGCTTT | A | | |
| | | | CTTTGCCT | | | |
| | | | G | | | |
| 9 | TS16 | 11 | GGAGTCG | ATTTCCTC | LINC01588 | 28 |
| | | | CCAGAGG | | | |
| | | | CCGGTGG | | | |
| | | | TGG | | | |
| 10 | TS17 | 12 | GCCCAGC | CACGCCCG | LINC01588 | ND |
| | | | GGCCGGA | | | |
| | | | TATCAGC | | | |
| | | | TGC | | | |
| 11 | TS18 | 13 | GGAAGGG | TTTCCCTC | CYBB | 1 |
| | | | AACATAT | | | |
| | | | TACTATT | | | |
| | | | GC | | | |
| 12 | TS19 | 14 | GTGGAGT | CTATCCAA | CYBB | 6 |
| | | | GGCCTGC | | | |
| | | | TATCAGC | | | |
| | | | TAC | | | |
| 13 | TS20 | 15 | GAGGAAG | CTTTCCCT | CYBB | 11.2 |
| | | | GGAACAT | | | |
| | | | ATTACTA | | | |
| | | | TTG | | | |
| 14 | TS21 | 16 | GTGAATT | CAAGCCTT | CYBB | 1 |
| | | | CTCATCA | | | |
| | | | GCTAAAA | | | |
| | | | TGC | | | |
| 15 | TS25 | 17 | GCTCACT | ACGTCCTC | VEGFA | 15.6 |
| | | | CACCCAC | | | |
| | | | ACAGACA | | | |
| | | | CAC | | | |
| 16 | TS26 | 18 | GGAAGAA | TTTTCCTG | CFTR | 2 |
| | | | TTTCATTC | | | |
| | | | TGTTCTC | | | |
| | | | AG | | | |
| 17 | TS27 | 19 | GCTCAGT | GGCACCAT | CFTR | 4 |
| | | | TTTCCTG | | | |
| | | | GATTATG | | | |
| | | | CCT | | | |
| 18 | TS31 | 20 | GCGTTGG | GGGTCACT | VEGFA | 9 |
| | | | AGCGGGG | | | |
| | | | AGAAGGC | | | |
| | | | CAG | | | |
| 19 | TS34 | 21 | GGGCCGC | GGGGCCCC | LINC01588 | ND |
| | | | GGAGATA | | | |
| | | | GCTGCAG | | | |
| | | | GGC | | | |
| 20 | TS35 | 22 | GCCCACC | AGGGCTGC | LINC01588 | ND |
| | | | CGGCGGC | | | |
| | | | GCCTCCC | | | |
| | | | TGC | | | |
| 21 | TS36 | 23 | GCGTGGC | TGGGCGTC | LINC01588 | ND |
| | | | AGCTGAT | | | |
| | | | ATCCGGC | | | |
| | | | CGC | | | |
| 22 | TS37 | 24 | GCCGCGG | CCCGCAAA | LINC01588 | ND |
| | | | CGCGACG | | | |
| | | | TGGAGCC | | | |
| | | | AGC | | | |
| 23 | TS38 | 25 | GTGCTCC | GGCGCGAC | LINC01588 | 2 |
| | | | CCAGCCC | | | |
| | | | AAACCGC | | | |
| | | | CGC | | | |
| 24 | TS41 | 26 | GTCAGAT | CCAGCCAA | AGA | 3 |
| | | | TGGCTTG | | | |
| | | | CTCGGAA | | | |
| | | | TTG | | | |
| 25 | TS44 | 27 | GCTGGGT | TCTTCGAG | VEGFA | 3 |
| | | | GAATGGA | | | |
| | | | GCGAGCA | | | |
| | | | GCG | | | |
| 26 | TS45 | 28 | GTCCTGG | TCCCCGCT | VEGFA | 7.4 |
| | | | AGTGACC | | | |
| | | | CCTGGCC | | | |
| | | | TTC | | | |
| 27 | TS46 | 29 | GATCCTG | CTCCCCGC | VEGFA | 6 |
| | | | GAGTGAC | | | |
| | | | CCCTGGC | | | |
| | | | CTT | | | |
| 28 | TS47 | 30 | GTGTGTC | CTGTCCGG | VEGFA | 23.1 |
| | | | CCTCTCC | | | |
| | | | CCACCCG | | | |
| | | | TCC | | | |
| 29 | TS48 | 31 | GTTGGAG | GTCACTCC | VEGFA | 2 |
| | | | CGGGGAG | | | |
| | | | AAGGCCA | | | |
| | | | GGG | | | |
| 30 | TS49 | 20 | GCGTTGG | GGGTCACT | VEGFA | 4 |
| | | | AGCGGGG | | | |
| | | | AGAAGGC | | | |
| | | | CAG | | | |
| 31 | TS50 | 32 | GTACCCT | AATTCCGA | AGA | 6 |
| | | | CCAATAA | | | |
| | | | TTTGGCT | | | |
| | | | GGC | | | |
| 32 | TS51 | 33 | GATAATT | CAAGCCAA | AGA | 4.5 |
| | | | TGGCTGG | | | |
| | | | CAATTCC | | | |
| | | | GAG | | | |
| 33 | TS58 | 34 | GCAGGGG | GGGGCCTC | VEGFA | 5 |
| | (DS1) | | CCAGGTG | | | |
| | | | TCCTTCTC | | | |
| | | | TG | | | |
| 34 | TS59 | 35 | GAATGGC | GGGGCCAG | VEGFA | 11.5 |
| | (DS2) | | AGGCGGA | | | |
| | | | GGTTGTA | | | |
| | | | CTG | | | |
| 35 | TS60 | 36 | GAGTGAG | CGGGCCAG | VEGFA | 3 |
| | (DS3) | | AGAGTGA | | | |
| | | | GAGAGAG | | | |
| | | | ACA | | | |
| 36 | TS61 | 37 | GTGAGCA | GGGCCCGC | VEGFA | 3.5 |
| | (DS4) | | GGCACCT | | | |
| | | | GTGCCAA | | | |
| | | | CAT | | | |
| 37 | TS62 | 38 | GCGTGGG | GGGTCCAT | VEGFA | 3.4 |
| | (DS5) | | GGCTCCG | | | |
| | | | TGCCCCA | | | |
| | | | CGC | | | |
| 38 | TS63 | 39 | GCATGGG | AGGCCCAG | VEGFA | 16 |
| | (DS6) | | CAGGGGC | | | |
| | | | TGGGGTG | | | |
| | | | CAC | | | |
| 39 | TS64 | 40 | GAAAATT | AAGCCCAA | FANCJ | 7 |
| | | | GTGATTT | | | |
| | | | CCAGATC | | | |
| | | | CAC | | | |
| 40 | TS65 | 41 | GAGCAGA | AGATCCAC | FANCJ | ND |
| | | | AAAAATT | | | |
| | | | GTGATTT | | | |
| | | | CC | | | |

TABLE 2
Exemplary TIDE Primers

| | Target | Primer | SEQ ID | | SEQ ID | |
|---|---|---|---|---|---|---|
| No. | Site | name | NO | Forward primer | NO | Reverse primer |
| 1 | TS1 | AAVS1_ | 42 | TGGCTTAGCACCT | 101 | AGAACTCAGGACCAACTTATTC |
| | | TIDE1 | | CTCCAT | | TG |
| 2 | TS4 | AAVS1_ | 42 | TGGCTTAGCACCT | 101 | AGAACTCAGGACCAACTTATTC |
| | | TIDE1 | | CTCCAT | | TG |
| 3 | TS5 | AAVS1_ | 42 | TGGCTTAGCACCT | 101 | AGAACTCAGGACCAACTTATTC |
| | | TIDE1 | | CTCCAT | | TG |
| 4 | TS6 | LINC01588_ | 43 | AGAGGAGCCTTCT | 102 | ATGACAGACACAACCAGAGGG |
| | | TIDE | | GACTGCTGCAGA | | CA |
| 5 | TS10 | AAVS1_ | 42 | TGGCTTAGCACCT | 101 | AGAACTCAGGACCAACTTATTC |
| | | TIDE1 | | CTCCAT | | TG |
| 6 | TS11 | AAVS1_ | 42 | TGGCTTAGCACCT | 101 | AGAACTCAGGACCAACTTATTC |
| | | TIDE1 | | CTCCAT | | TG |
| 7 | TS12 | AAVS1_ | 44 | TCCGTCTTCCTCC | 103 | TAGGAAGGAGGAGGCCTAAG |
| | | TIDE2 | | ACTCC | | |
| 8 | TS13 | AAVS1_ | 44 | TCCGTCTTCCTCC | 103 | TAGGAAGGAGGAGGCCTAAG |
| | | TIDE2 | | ACTCC | | |
| 9 | TS16 | LINC01588_ | 43 | AGAGGAGCCTTCT | 102 | ATGACAGACACAACCAGAGGG |
| | | TIDE | | GACTGCTGCAGA | | CA |
| 10 | TS17 | LINC01588_ | 43 | AGAGGAGCCTTCT | 102 | ATGACAGACACAACCAGAGGG |
| | | TIDE | | GACTGCTGCAGA | | CA |
| 11 | TS18 | NTS55_ | 45 | TAGAGAACTGGGT | 104 | CCAATATTGCATGGGATGG |
| | | TIDE | | AGTGTG | | |
| 12 | TS19 | NTS55_ | 45 | TAGAGAACTGGGT | 104 | CCAATATTGCATGGGATGG |
| | | TIDE | | AGTGTG | | |
| 13 | TS20 | NTS55_ | 45 | TAGAGAACTGGGT | 104 | CCAATATTGCATGGGATGG |
| | | TIDE | | AGTGTG | | |
| 14 | TS21 | NTS55_ | 45 | TAGAGAACTGGGT | 104 | CCAATATTGCATGGGATGG |
| | | TIDE | | AGTGTG | | |
| 15 | TS25 | VEGF_ | 46 | GTACATGAAGCAA | 105 | ATCAAATTCCAGCACCGAGCG |
| | | TIDE3 | | CTCCAGTCCCA | | C |
| 16 | TS26 | hCFTR_ | 47 | TGGTGATTATGGG | 106 | ACCATTGAGGACGTTTGTCTCA |
| | | TIDE1 | | AGAACTGGAGC | | C |
| 17 | TS27 | hCFTR_ | 47 | TGGTGATTATGGG | 106 | ACCATTGAGGACGTTTGTCTCA |
| | | TIDE1 | | AGAACTGGAGC | | C |
| 18 | TS31 | VEGF_ | 46 | GTACATGAAGCAA | 105 | ATCAAATTCCAGCACCGAGCG |
| | | TIDE3 | | CTCCAGTCCCA | | C |
| 19 | TS34 | LINC01588_ | 43 | AGAGGAGCCTTCT | 102 | ATGACAGACACAACCAGAGGG |
| | | TIDE | | GACTGCTGCAGA | | CA |
| 20 | TS35 | LINC01588_ | 43 | AGAGGAGCCTTCT | 102 | ATGACAGACACAACCAGAGGG |
| | | TIDE | | GACTGCTGCAGA | | CA |
| 21 | TS36 | LINC01588_ | 43 | AGAGGAGCCTTCT | 102 | ATGACAGACACAACCAGAGGG |
| | | TIDE | | GACTGCTGCAGA | | CA |
| 22 | TS37 | LINC01588_ | 43 | AGAGGAGCCTTCT | 102 | ATGACAGACACAACCAGAGGG |
| | | TIDE | | GACTGCTGCAGA | | CA |
| 23 | TS38 | LINC01588_ | 43 | AGAGGAGCCTTCT | 102 | ATGACAGACACAACCAGAGGG |
| | | TIDE | | GACTGCTGCAGA | | CA |
| 24 | TS41 | AGA_ | 48 | GGCATAAGGAAAT | 107 | CATGTCCTCAAGTCAAGAACA |
| | | TIDE1 | | CGAAGGTC | | AG |
| 25 | TS44 | VEGF_ | 46 | GTACATGAAGCAA | 105 | ATCAAATTCCAGCACCGAGCG |
| | | TIDE3 | | CTCCAGTCCCA | | C |
| 26 | TS45 | VEGF_ | 46 | GTACATGAAGCAA | 105 | ATCAAATTCCAGCACCGAGCG |
| | | TIDE3 | | CTCCAGTCCCA | | C |
| 27 | TS46 | VEGF_ | 46 | GTACATGAAGCAA | 105 | ATCAAATTCCAGCACCGAGCG |
| | | TIDE3 | | CTCCAGTCCCA | | C |
| 28 | TS47 | VEGF_ | 46 | GTACATGAAGCAA | 105 | ATCAAATTCCAGCACCGAGCG |
| | | TIDE3 | | CTCCAGTCCCA | | C |
| 29 | TS48 | VEGF_ | 46 | GTACATGAAGCAA | 105 | ATCAAATTCCAGCACCGAGCG |
| | | TIDE3 | | CTCCAGTCCCA | | C |
| 30 | TS49 | VEGF_ | 46 | GTACATGAAGCAA | 105 | ATCAAATTCCAGCACCGAGCG |
| | | TIDE3 | | CTCCAGTCCCA | | C |
| 31 | TS50 | AGA_ | 48 | GGCATAAGGAAAT | 107 | CATGTCCTCAAGTCAAGAACA |
| | | TIDE1 | | CGAAGGTC | | AG |
| 32 | TS51 | AGA_ | 48 | GGCATAAGGAAAT | 107 | CATGTCCTCAAGTCAAGAACA |
| | | TIDE1 | | CGAAGGTC | | AG |
| 33 | TS58 | VEGF_ | 49 | ACACGGGCAGCAT | 108 | GCTAGGGGAGAGTCCCACTGT |
| | (DS1) | TIDE4 | | GGGAATAGTC | | CCA |
| 34 | TS59 | VEGF_ | 50 | CCTGTGTGGCTTT | 169 | GGTAGGGTGTGATGGGAGGCT |
| | (DS2) | TIDE5 | | GCTTTGGTC | | AAGC |
| 35 | TS60 | VEGF_ | 50 | CCTGTGTGGCTTT | 169 | GGTAGGGTGTGATGGGAGGCT |
| | (DS3) | TIDE5 | | GCTTTGGTC | | AAGC |
| 36 | TS61 | VEGF_ | 50 | CCTGTGTGGCTTT | 169 | GGTAGGGTGTGATGGGAGGCT |
| | (DS4) | TIDE5 | | GCTTTGGTC | | AAGC |
| 37 | TS62 | VEGF_ | 51 | GGAGGAAGAGTA | 110 | AGACCGAGTGGCAGTGACAGC |
| | (DS5) | TIDE6 | | GCTCGCCGAGG | | AAG |
| 38 | TS63 | VEGF_ | 52 | AGGGAGAGGGAA | 111 | GTCTTCCTGCTCTGTGCGCACG |
| | (DS6) | TIDE7 | | GTGTGGGGAAGG | | AC |
| 39 | TS64 | FancJ_ | 53 | GTTGGGGGCTCTA | 170 | CTTCATCTGTATCTTCAGGATC |
| | | TIDE5 | | AGTTATGTAT | | A |
| 40 | TS65 | FancJ_ | 53 | GTTGGGGGCTCTA | 170 | CTTCATCTGTATCTTCAGGATC |
| | | TIDE5 | | AGTTATGTAT | | A |

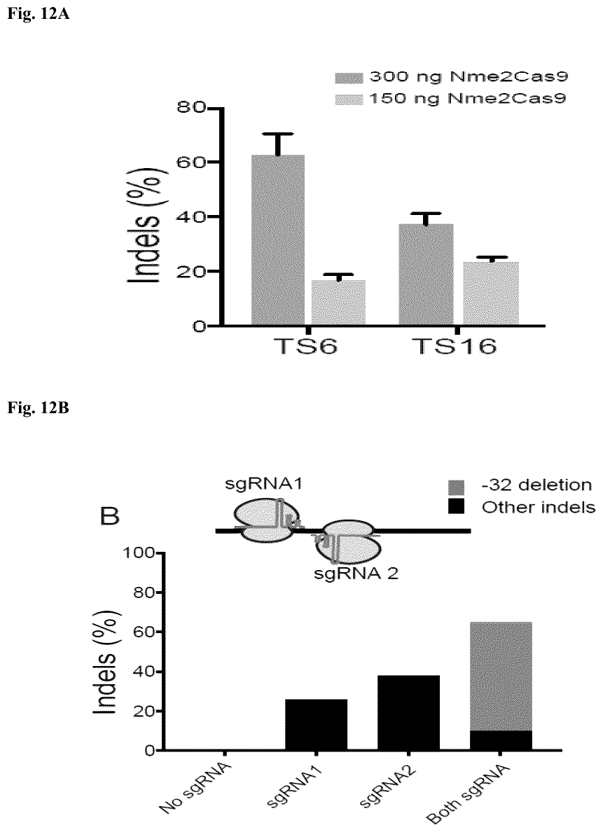

HEK293T cells were used to support transient transfections and at 72-hours post transfection the cells were harvested, followed by genomic DNA extraction and selective amplification of the targeted locus. TIDE analysis was used to measure indel efficiency at each locus (Brinkman et al., 2014). Nme2Cas9 editing was detectable at most of these sites, even though efficiencies varied depending on the target sequence. Interestingly, Nme2Cas9 induced indels at several genomic sites with N₄CD PAMs, albeit less consistently and at lower levels. Table 1. Fourteen (14) sites with N₄CC PAMs were analyzed in triplicate, and consistent editing was observed. See, FIG. 11A. In addition, editing efficiency could be improved significantly by increasing the quantity of the Nme2Cas9 plasmid delivered, and this high efficiency could be extended to precise segmental deletion with two guides. See, FIGS. 12A and 12B.

The ability of Nme2Cas9 to function was tested in mouse Hepa1-6 cells (hepatoma-derived). For Hepa1-6 cells, a single plasmid encoding both Nme2Cas9 and an sgRNA (targeting either Rosa26 or Pcsk9) was transiently transfected and indels were measured after 72 hrs. Editing was readily observed at both sites. See, FIG. 11B, left. Nme2Cas9's functionality was also tested when stably expressed in human leukemia K562 cells. To this end, a lentiviral construct was created expressing Nme2Cas9 and transduced cells to stably express Nme2Cas9 under the control of the SFFV promoter. This stable cell line did not show any visible differences with respect to growth and morphology in comparison to untransduced cells, suggesting that Nme2Cas9 is not toxic when stably expressed. These cells were transiently electroporated with plasmids expressing sgRNAs and analyzed by TIDE after 72 hours to measure indel efficiencies. Efficient (>50%) editing was observed at all three sites tested, validating Nme2Cas9's ability to function upon lentiviral delivery in K562 cells. See, FIG. 11B.

Ribonucleoprotein (RNP) delivery of Cas9 and its sgRNA is also useful for some genome editing applications, and the greater transience of Cas9's presence can minimize off-target editing (Kim et al., 2014; Zuris et al., 2015). Moreover, some cell types (e.g. certain immune cells) are recalcitrant to DNA transfection-based editing (Schumann et al., 2015). To test whether Nme2Cas9 is functional by RNP delivery, a 6xHis (SEQ ID NO:172)-tagged Nme2Cas9 (fused to three NLSs) was cloned into a bacterial expression construct and the recombinant protein was purified. The recombinant protein was then loaded with T7 RNA polymerase-transcribed sgRNAs targeting three previously validated sites. Electroporation of the Nme2Cas9:sgRNA complex induced successful editing at each of the three target sites in HEK293T cells, as detected by TIDE. See, FIG. 11C. Collectively these results indicate that Nme2Cas9 can be delivered effectively via plasmid or lentivirus, or as an RNP complex, in multiple cell types.

### D. Anti-CRISPR (Acr) Regulation

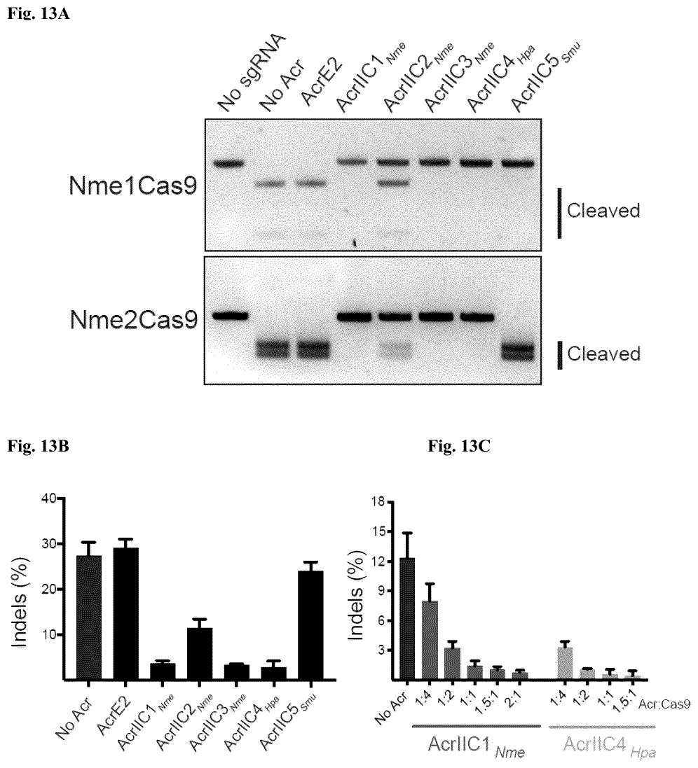

To date, five families of Acr proteins from diverse bacterial species have been shown to inhibit Nme1Cas9 in vitro and in human cells (Pawluk et al., 2016; Lee et al., 2018, submitted). Considering the high sequence identity between Nme1Cas9 and Nme2Cas9, at least some of these Acr families should inhibit Nme2Cas9. To test this, all five families of recombinant Acrs were expressed, purified and tested for Nme2Cas9's ability to cleave a target in vitro in the presence of a member of each family (10:1 Acr:Cas9 molar ratio). An inhibitor was used for the type I-E CRISPR system in E. coli (AcrE2) as a negative control, while Nme1Cas9 was used as a positive control. (Pawluk et al., 2014); (Pawluk et al., 2016). As expected, all 5 families inhibited Nme1Cas9, while AcrE2 failed to do so. See, FIG. 13A, top. AcrIIC1_{Nme}, AcrIIC2_{Nme}, AcrIIC3_{Nme}, and AcrIIC4_{Hpa} completely inhibited Nme2Cas9. Strikingly, however, AcrIIC5_{Smu} which has been previously reported as the most potent of the Nme1Cas9 inhibitors (Lee et al., 2018), did not inhibit Nme2Cas9 in vitro even at a 10-fold molar excess. This suggests that it likely inhibits Nme1Cas9 by interacting with its PID.

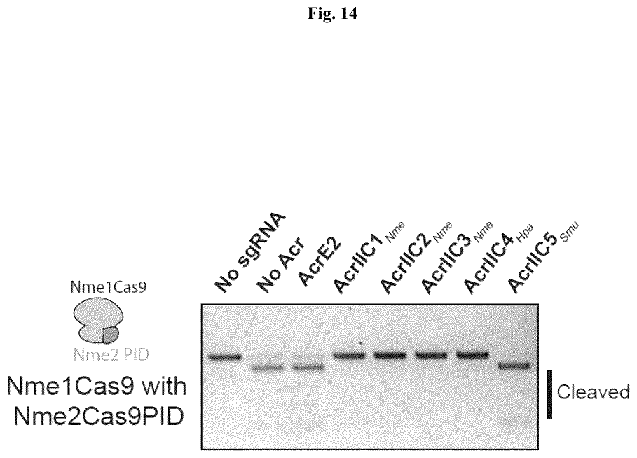

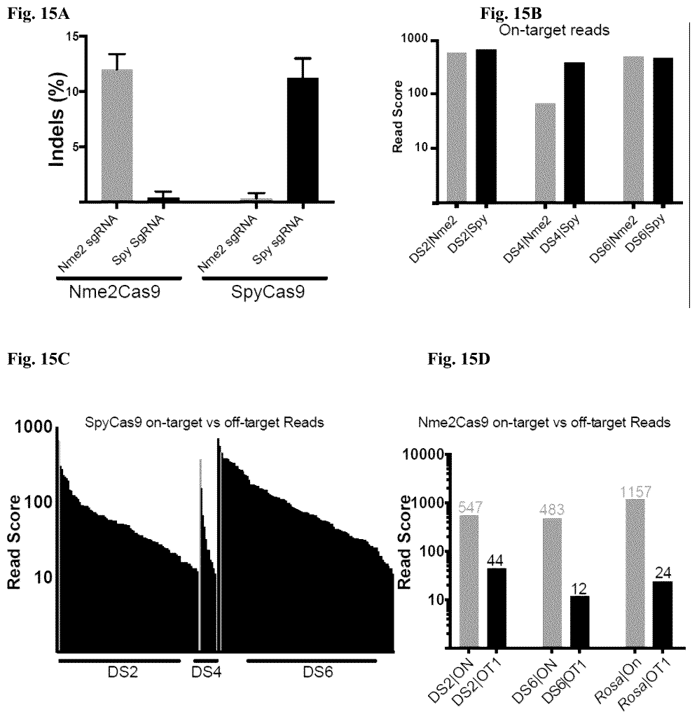

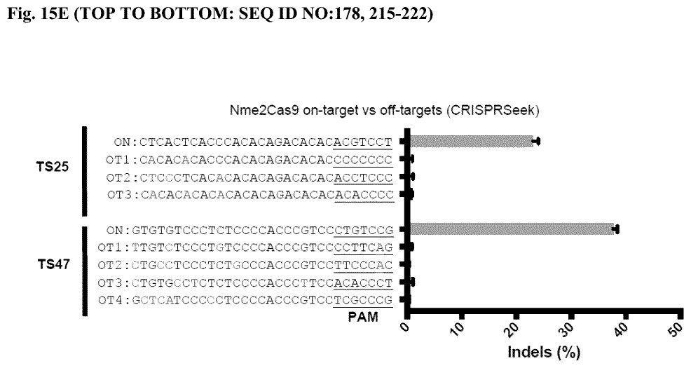

To further test this, a Nme1Cas9/Nme2Cas9 chimera with the PID of Nme2Cas9 was tested. See, FIG. 7D and FIG. 8D. Due to the reduced activity of this hybrid, a ˜30×higher concentration of Cas9 was used to achieve a similar cleavage efficiency while maintaining the 10:1 Cas9:Acr molar ratio. No inhibition was observed by AcrIIC5_{Smu} on this protein chimera. See, FIG. 14. This data provides further evidence that AcrIIC5ₛₘᵤ likely interacts with the PID of Nme1Cas9. Regardless of the mechanistic basis for the differential inhibition by AcrIIC5ₛₘᵤ, these results indicate that Nme2Cas9 is subject to inhibition by the other four type II-C Acr families.

Based on the above in vitro data, it was hypothesized that AcrIIC1_{Nme}, AcrIIC2_{Nme}, AcrIIC3_{Nme}, and AcrIIC4_{Hpa} could be used as off-switches for Nme2Cas9 genome editing. To test this, Nme2Cas9/sgRNA plasmid transfections (150 ng of each plasmid) targeting TS16 were performed in HEK293T cells in the presence or absence of Acr expression plasmids, as it has been reported that most Acrs inhibited Nme1Cas9 at those plasmid ratios (Pawluk et al., 2016). As expected, AcrIIC1_{Nme}, AcrIIC2_{Nme}, AcrIIC3Nₘₑ and AcrIIC4_{Hpa} inhibited Nme2Cas9 genome editing, while AcrIIC5ₛₘᵤ had no effect. See, FIG. 13B. Complete inhibition was observed by AcrIIC3Nₘₑ and AcrIIC4_{Hpa}, suggesting that they have high potency against Nme2Cas9 as compared to AcrIIC1_{Nme} and AcrIIC2_{Nme}. To further compare the potency of AcrIIC1_{Nme} and AcrIIC4_{Hpa}, we repeated the experiments at various ratios of Acr plasmid to Cas9 plasmid. See, FIG. 13C. The data show that the AcrIIC4_{Hpa} plasmid is especially potent against Nme2Cas9. Together, these data suggest that several Acr proteins can be used as off-switches for Nme2Cas9-based applications.

### E. NmeCas9 Gene Editing Efficiency

Nme1Cas9 demonstrates remarkable editing fidelity in cells and mouse models (Lee et al., 2016; Amrani et al., 2018; Ibraheim et al., 2018). Furthermore, the similarity of Nme2Cas9 to Nme1Cas9 over most of its length suggests that it may likewise be hyper-accurate. However, the higher number of sites sampled in the genome as a result of the CC dinucleotide PAM could create more opportunities for Nme2Cas9 off-targeting in comparison with Nme1Cas9 and its less frequently encountered 4-nucleotide PAM.

To assess the off-target profile of Nme2Cas9, genome-wide, unbiased identification of double-stranded breaks enabled by sequencing (GUIDE-seq) was used to identify potential off-target sites empirically and in an unbiased fashion (Tsai et al., 2014). Even the best off-target prediction algorithms are prone to false negatives necessitating empirical target site profiling methods (Bolukbasi et al., 2015b; Tsai and Joung, 2016; Tycko et al., 2016). GUIDE-seq relies on the incorporation of double-stranded oligodeoxynucleotides (dsODNs) into DNA double-stranded break sites throughout the genome. These insertion sites are then detected by amplification and high-throughput sequencing.

Because SpyCas9 is a well-characterized Cas9 ortholog it is useful for multiplexed applications with other Cas9s, and as a benchmark for their editing properties (Jiang and Doudna, 2017; Komor et al., 2017). SpyCas9 and Nme2Cas9 were cloned into identical plasmid backbones, with the same UTRs, linkers, NLSs, and promoters, for parallel transient transfections (along with similarly matched sgRNA-expressing plasmids) into HEK293T cells. First, it was confirmed that the RNA guides for SpyCas9 and Nme2Cas9 are orthogonal, i.e. that Nme2Cas9 sgRNAs do not direct editing by SpyCas9, and vice versa. See, FIG. 15A. This was in contrast to earlier reported results with Nme1 Cas9 (Esvelt et al., 2013; Fonfara et al., 2014).

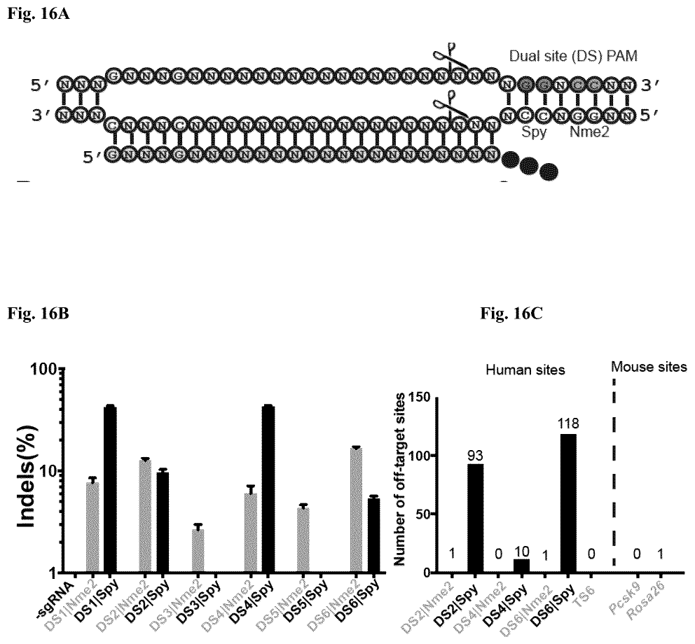

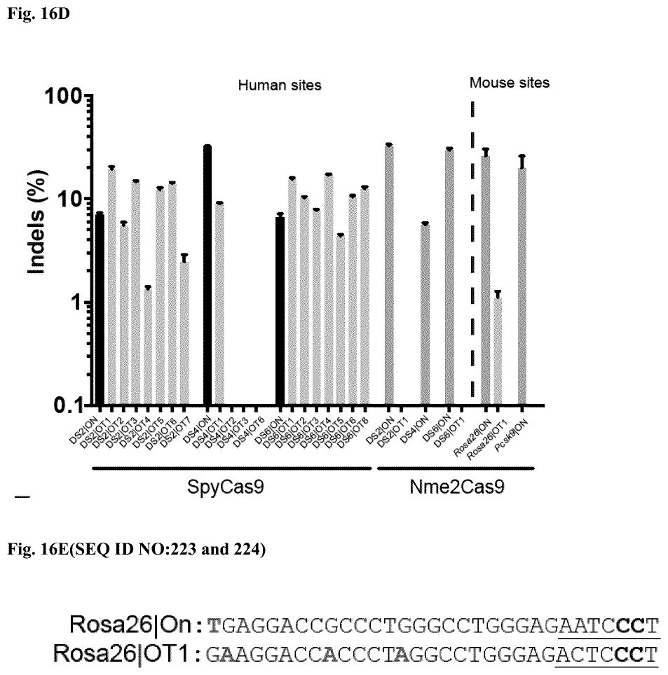

Next, to identify a use of SpyCas9 as a benchmark for GUIDE-seq, because SpyCas9 and Nme2Cas9 have non-overlapping PAMs its can therefore potentially edit any dual site (DS) flanked by a 5-NGGNCC-3′ sequence, which simultaneously fulfills the PAM requirements of both Cas9's. This permits side-by-side comparisons of off-targeting with RNA guides that facilitate an edit of the exact same on-target site. See, FIG. 16A. Six (6) DSs in VEGFA were targeted, each of which also has a G at the appropriate positions 5′ of the PAM such that both SpyCas9 and Nme2Cas9 guides (driven by the U6 promoter) were 100% complementary to the target site. Seventy-two (72) hours after transfection, a TIDE analysis was performed on these sites targeted by each nuclease. Nme2Cas9 induced indels at all six sites, albeit at low efficiencies at two of them, while SpyCas9 induced indels at four of the six sites. See, FIG. 16B. At two of the four sites (DS1 and DS4) at which SpyCas9 was effective, it induced ˜7-fold more indels than Nme2Cas9, while Nme2Cas9 induced a ˜3-fold higher frequency of indels than SpyCas9 at DS6. Both Cas9 orthologs edited DS2 with approximately equal efficiency.

For GUIDE-seq, the DS2, DS4 and DS6 target sites were selected to sample off-target cleavage with Nme2Cas9 guides that direct on-target editing as efficiently, less efficiently, or more efficiently than the corresponding SpyCas9 guides, respectively. In addition to the three dual sites, TS6 was added as it has been observed to be an efficiently edited Nme2Cas9 target sites, having an approximate 30-50% indel efficiency depending on the cell type, See, FIGS. 11A and 12A. Similar data is seen with the mouse Pcsk9 and Rosa26 Nme2Cas9 sites. See, FIG. 1B.

Plasmid transfections were performed for SpyCas9 and Nme2Cas9 along with their cognate sgRNAs and the dsODNs. Subsequently, GUIDE-seq libraries were prepared as described previously (Amrani et al., 2018). A GUIDE-seq analysis revealed efficient on-target editing for both Cas9 orthologs, with relative efficiencies (as reflected by GUIDE-seq read counts) that are similar to those observed by TIDE, FIG. 15A-E; and Tsai et al., 2014; Zhu et al., 2017. For off-target identification, the analysis revealed that the DS2, DS4, and DS6 SpyCas9 sgRNAs appeared to direct editing at 93, 10, and 118 candidate off-target sites, respectively, in the normal range of off-targets when plasmid-based SpyCas9 editing is analyzed by GUIDE-seq (Fu et al., 2014; Tsai et al., 2014), In striking contrast, the DS2. DS4, and DS6 Nme2Cas9 sgRNAs appeared to direct editing at 1, 0, and 1 off-target sites, respectively. FIG. 16C. When compared to the GUIDE-seq read counts for the SpyCas9 off-targets, those of Nme2Cas9 were very low, further suggesting that Nme2Cas9 is highly specific. FIG. 15C cf. FIG. 15D. Nme2Cas9 GUIDE-seq analyses with the TS6, Pcsk9, and Rosa26 yielded similar results (0, 0, and 1 off-target sites, respectively, with a modest read count for the Rosa26-OT1 off-target site). FIG. 15C, and FIG. 16D.

To validate the off-target sites detected by GUIDE-seq, a targeted deep sequencing was performed to measure indel formation at the top off-target loci following GUIDE-seq-independent editing (i.e. without co-transfection of the dsODN). While SpyCas9 showed considerable editing at most off-target sites tested and, in some instances, was more efficient than that at the corresponding on-target site, Nme2Cas9 exhibited no detectable indels at the lone DS2 and DS6 candidate off-target sites. See, FIG. 16D. With the Rosa26 sgRNA, Nme2Cas9 induced ˜1% editing at the Rosa26-OT1 site in Hepa1-6 cells, compared to ˜30% on-target editing. See, FIG. 16D. It is noteworthy that this off-target site has a consensus Nme2Cas9 PAM (ACTCCQT) with only 3 mismatches at the PAM-distal end of the guide-complementary region (i.e. outside of the seed). See, FIG. 16E. These data support and reinforce our GUIDE-seq results indicating a high degree of accuracy for Nme2Cas9 genome editing in mammalian cells.

To further corroborate the above GUIDE-Seq results, CRISPRseek was used to computationally predict potential off-target sites for two active Nme2Cas9 sgRNAs that targeted TS25 and TS47, both of which are also in VEGFA See, FIG. 11A; (Zhu et al., 2014). Three (TS25) or four (TS47) of the most closely matched predicted sites, five with N₄CC PAMs and two with N₄CA PAMs; each had 2-5 mismatches, mostly in their PAM-distal, non-seed regions. See, FIG. 15E. On-vs. off-target editing was compared after Nme2Cas9+ sgRNA plasmid transfections into HEK293T cells by targeted amplification of each locus, followed by TIDE analysis. Consistently, no indels could be detected at those off-target sites for either sgRNA by TIDE, while efficient on-target editing was readily detected in DNA from the same populations of cells. Taken together, our data indicate that Nme2Cas9 is a naturally hyper-accurate genome editing platform in mammalian cells.

### F. Adeno-Associated Virus Nme2Cas9 Delivery

Clustered, regularly interspaced, short, palindromic repeats (CRISPR) along with CRISPR-associated (Cas) proteins constitute bacterial and archaeal adaptive immune pathways against phages and other mobile genetic elements (MGEs) (Barrangou et al., 2007; Brouns et al., 2008; Marraffini and Sontheimer, 2008). In Type II CRISPR systems, CRISPR RNA (crRNA) is bound to a trans-activating crRNA (tracrRNA) and loaded onto a Cas9 effector protein that cleaves MGE nucleic acids complementary to the crRNA (Garneau et al., 2010; Deltcheva et al., 2011; Sapranauskas et al., 2011; Gasiunas et al., 2012; Jinek et al., 2012). The crRNA:tracrRNA hybrid can be fused into a single-guide RNA (sgRNA) (Jinek et al., 2012). The RNA programmability of Cas9 endonucleases has made it a powerful genome editing platform in biotechnology and medicine (Cho et al., 2013; Cong et al., 2013; Hwang et al., 2013; Jiang et al., 2013; Jinek et al., 2013; Mali et al., 2013b).

In addition to sgRNA, Cas9 target recognition is usually associated with a 1-5 nucleotide signature downstream of the complementary DNA sequence, called a protospacer adjacent motif (PAM) (Deveau et al., 2008; Mojica et al., 2009). Cas9 orthologs exhibit considerable diversity in PAM length and sequence. Among Cas9 orthologs that have been characterized, Streptococcus pyogenes Cas9 (SpyCas9) is the most widely used, in part because it recognizes a short NGG PAM (Jinek et al., 2012) (N represents any nucleotide) that affords a high density of targetable sites. Nevertheless, Spy's relatively large size (i.e., 1,368 amino acids) makes this Cas9 difficult to package (along with sgRNA and promoters) into a single recombinant adeno-associated virus (rAAV). This has been shown to be a drawback for therapeutic applications given the promise shown by AAV vectors for in vivo gene delivery (Keeler et al., 2017). Moreover, SpyCas9 and its RNA guides have required extensive characterization and engineering to minimize the tendency to edit near-cognate, off-target sites. (Bolukbasi et al., 2015b; Tsai and Joung, 2016; Tycko et al., 2016; Chen et al., 2017; Casini et al., 2018; Yin et al., 2018). To date, subsequent engineering efforts have not overcome these size limitations.

Several Cas9 orthologs of less than 1,100 amino acids in length obtained from diverse species have been validated for mammalian genome editing, including strains of N. meningitidis (NmeCas9, 1,082 aa) (Esvelt et al., 2013; Hou et al., 2013), Staphylococcus aureus (SauCas9, 1,053 aa) (Ran et al., 2015), Campylobacter jejuni (CjeCas9, 984 aa) (Kim et al., 2017), and Geobacillus stearothermophilus (GeoCas9, 1,089 aa) (Harrington et al., 2017b). NmeCas9, CjeCas9, and GeoCas9 are representatives of type II-C Cas9s (Mir et al., 2018), most of which are <1,100 aa. With the exception of GeoCas9, each of these shorter sequence orthologs has been successfully deployed for in vivo editing via all-in-one AAV delivery (in which a single vector expresses both guide and effector) (Ran et al., 2015; Kim et al., 2017; Ibraheim et al., 2018, submitted). Furthermore, NmeCas9 and CjeCas9 have been shown to be naturally resistant to off-target editing (Lee et al., 2016; Kim et al., 2017; Amrani et al., 2018, submitted). However, the PAMs that are recognized by compact Cas9s are usually longer than that of SpyCas9, substantially reducing the number of targetable sites at or near a given locus; for example, i) N₄GAYW/N₄GYTT/N₄GTCT for NmeCas9 (Esvelt et al., 2013; Hou et al., 2013; Lee et al., 2016; Amrani et al., 2018); ii) N₂GRRT for SauCas9 (Ran et al., 2015); iii) N₄RYAC for CjeCas9 (Kim et al., 2017); and iv) N₄CRAA/N₄GMAA for GeoCas9s (Harrington et al., 2017b) (Y=C, T; R=A, G; M=A, C; W=A, T). A smaller subset of target sites is advantageous for highly accurate and precise gene editing tasks including, but not limited to: i) editing of small targets (e.g. miRNAs); ii) correction of mutations by base editing which alters a very narrow window of bases relative to the PAM (Komor et al., 2016; Gaudelli et al., 2017); or iii) precise editing via homology-directed repair (HDR) which is most efficient when the rewritten bases are close to the cleavage site (Gallagher and Haber, 2018). Because of PAM restrictions, many editing sites cannot be targeted using all-in-one AAV vectors for in vivo delivery even with these shorter Cas9 proteins. For example, A SauCas9 mutant (SauCas9^{KKH}) has been developed that has reduced PAM constraints (N₃RRT), though this increase in targeting range often comes at the cost of reduced on-target editing efficacy, and off-target edits are still observed. (Kleinstiver et al., 2015).

Safe and effective CRISPR-based therapeutic gene editing will be greatly enhanced by Cas9 orthologs and variants that are highly active in human cells, resistant to off-targeting, sufficiently compact for all-in-one AAV delivery, and capable of accessing a high density of genomic sites. In one embodiment, the present invention contemplates a compact, hyper-accurate Cas9 (Nme2Cas9) from a distinct strain of N. meningitidis. In one embodiment, the present invention contemplates a method for single-AAV delivery of Nme2Cas9 and its sgRNA to perform efficient genome editing in vivo and/or ex vivo. Although it is not necessary to understand the mechanism of an invention, it is believed that this ortholog functions efficiently in mammalian cells and recognizes an N₄CC PAM that affords a target site density identical to that of wild-type SpyCas9 (e.g., every 8 bp on average, when both DNA strands are considered).

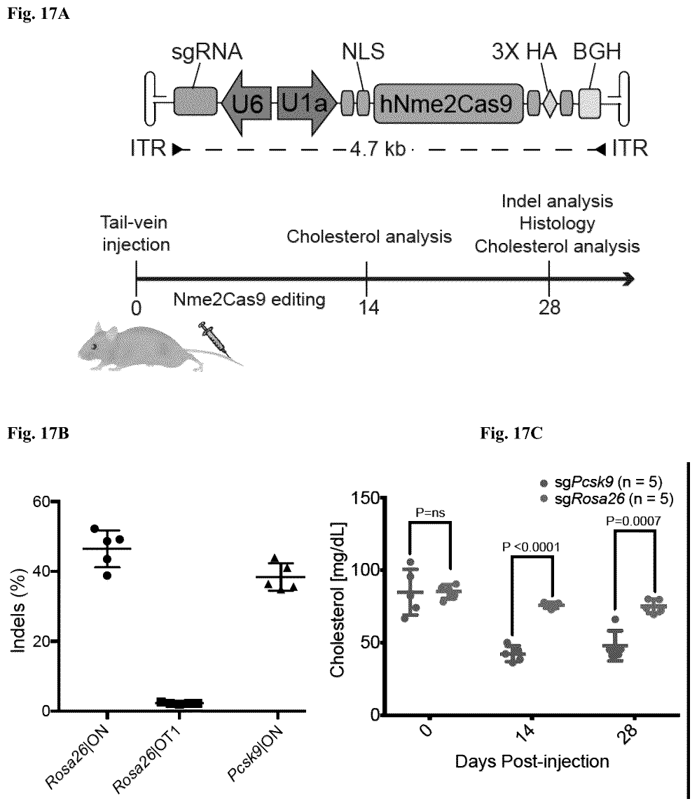

The compact size, small PAM, and high fidelity of Nme2Cas9 offer major advantages for in vivo genome editing using Adeno-Associated Virus (AAV) delivery. To test whether effective Nme2Cas9 genome editing can be achieved via single-AAV delivery, Nme2Cas9 was cloned with its sgRNA and their promoters (U1a and U6, respectively) into an AAV vector backbone. See, FIG. 17A. An all-in-one AAV was prepared with an sgRNA-Nme2Cas9 packaged into a hepatotropic AAV8 capsid to target two genes in the mouse liver: i) Rosa26 (a commonly used safe harbor locus for transgene insertion) (Friedrich and Soriano, 1991) as a negative control; and ii) Pcsk9, a major regulator of circulating cholesterol homeostasis (Rashid et al., 2005), as a phenotypic target.

SauCas9- or Nme1 Cas9-induced indels in Pcsk9 in the mouse liver results and reduced cholesterol levels providing a useful and easy-to-score in vivo benchmark for new editing platforms (Ran et al., 2015; Ibraheim et al., 2018) The Nme2Cas9 RNA guides were the same as those used above. See, FIG. 11B, FIG. 15D, and FIG. 16. As Rosa26-OT1 was the only Nme2Cas9 off-target site that has been validated in cultured mammalian cells, the Rosa26 guide also provided us with an opportunity to assess on-vs. off-target editing in vivo. See, FIGS. 16D-E. The tail veins of two groups of mice (n=5) were injected with 4×10¹¹ AAV8.sgRNA.Nme2Cas9 genome copies (GCs) targeting either Pcsk9 or Rosa26. Serum was collected at 0, 14 and 28 days post-injection for cholesterol level measurement. Mice were sacrificed at 28 days post-injection and liver tissues were harvested. See, FIG. 17A. Targeted deep sequencing of each locus revealed ˜38% and ˜46% indel induction at the Pcsk9 and Rosa26 editing sites, respectively, in the liver. See, FIG. 17B. Because hepatocytes constitute only 65-70% of total cellular content in the adult liver, Nme2Cas9 AAV-induced hepatocyte editing efficiencies with sgPcsk9 and sgRosa were approximately 54-58% and 66-71%, respectively (Racanelli and Rehermann, 2006).

Only 2.25% liver indels overall (˜3-3.5% in hepatocytes) were detected at the Rosa26-OT1 off-target site, comparable to the 1% editing that we observed at this site in transfected Hepa1-6 cells. FIG. 17B cf FIG. 16D, At both 14 and 28 days post-injection, Pcsk9 editing was accompanied by a ˜44% reduction in serum cholesterol levels, whereas mice treated with the sgRosa26-expressing AAV maintained normal level of cholesterol throughout the study. See, FIG. 17C. The ˜44% reduction in serum cholesterol in the Nme2Cas9/sgPcsk9 AAV-treated mice compares well with the ˜40% reduction reported with SauCas9 all-in-one AAV when targeting the same gene (Ran et al., 2015).

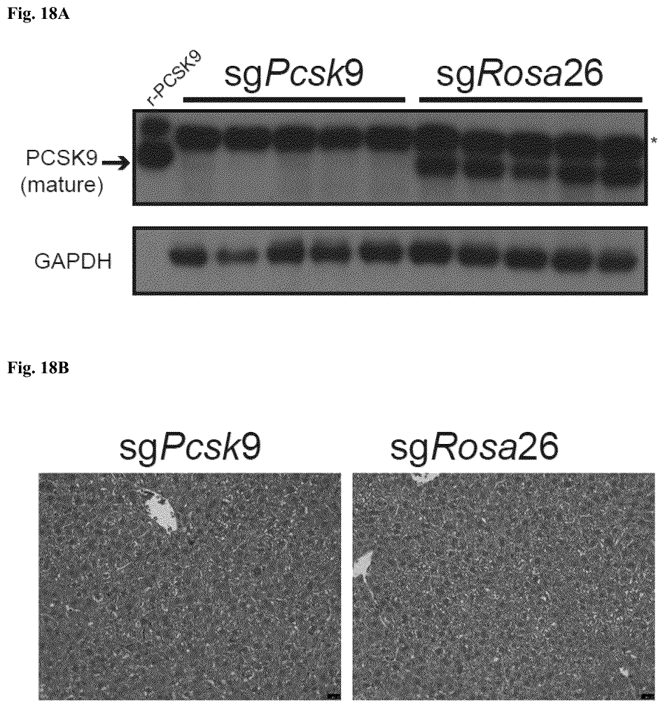

Western blotting was performed using an anti-PCSK9 antibody to estimate PCSK9 protein levels in the livers of mice treated with sgPcsk9 and sgRosa26. Liver PCSK9 was below the detection limit in mice treated with sgPcsk9, whereas sgRosa26-treated mice exhibited normal levels of PCSK9. See, FIG. 18A. Hematoxylin and eosin (H&E) staining and histology revealed no signs of toxicity or tissue damage in either group after Nme2Cas9 expression. See, FIG. 18B. These data validate Nme2Cas9 as a highly effective genome editing system in vivo, including when delivered by single-AAV vectors.

AAV vectors have recently been used for the generation of genome-edited mice, without the need for microinjection or electroporation, simply by soaking the zygotes in culture medium containing AAV vector(s), followed by reimplantation into pseudopregnant females (Yoon et al., 2018). Editing was obtained previously with a dual-AAV system in which SpyCas9 and its sgRNA were delivered in separate vectors (Yoon et al., 2018). To test whether Nme2Cas9 could perform accurate and efficient editing in mouse zygotes with an all-in-one AAV delivery system, Tyrosinase (Tyr) was targeted. A bi-allelic inactivation of Tyr disrupts melanin production resulting in an albino phenotype (Yokoyama et al., 1990).

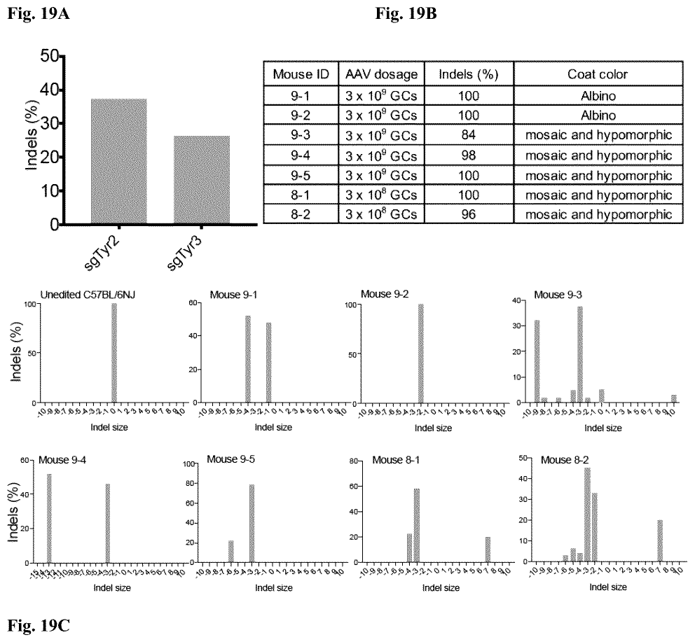

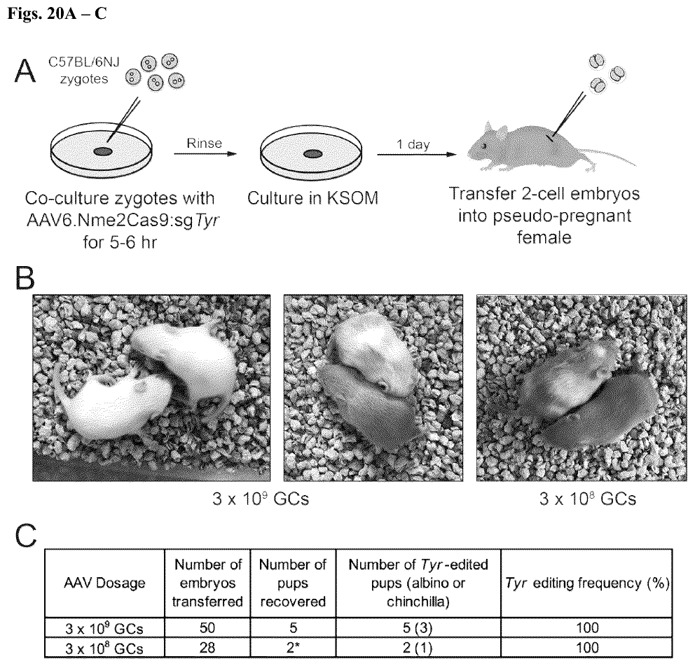

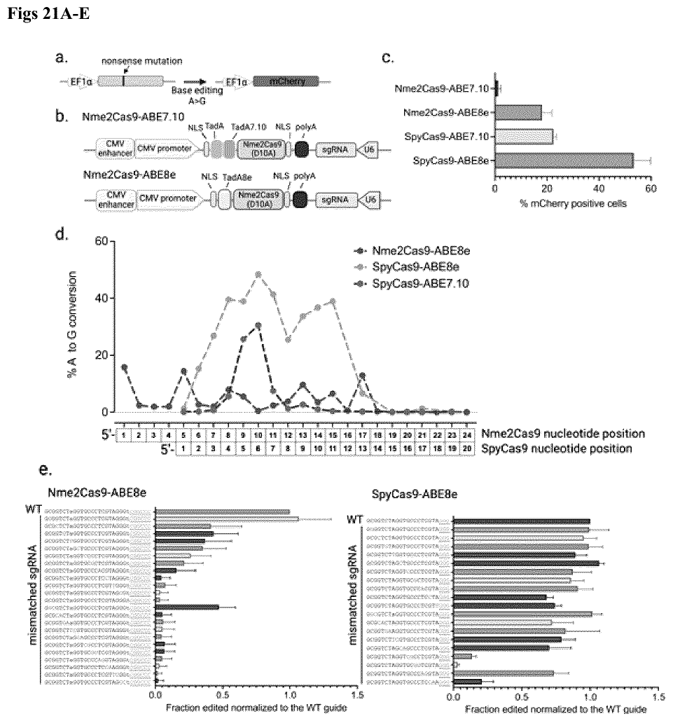

An efficient Tyr sgRNA was validated that cleaves the Tyr locus only seventeen (17) bp from the site of the classic albino mutation in Hepa1-6 cells by transient transfections. See, FIG. 19A, Next, C57BL/6NJ zygotes were incubated for 5-6 hours in culture medium containing 3×10⁹ or 3×10⁸ GCs of an all-in-one AAV6 vector expressing Nme2Cas9 along with the Tyr sgRNA. After overnight culture in fresh media, those zygotes that advanced to the two-cell stage were transferred to the oviduct of pseudopregnant recipients and allowed to develop to term. See, FIG. 20A. Coat color analysis of pups revealed mice that were albino, chinchilla (indicating a hypomorphic allele of Tyrosinase), or that had variegated coat color composed of albino and chinchilla spots but lacking black pigmentation. See, FIGS. 19B-C. These results suggest a high frequency of biallelic mutations since the presence of a wild-type Tyrosinase allele should render black pigmentation. A total of five pups (10%) were born from the 3×10⁹ GCs experiment. All of them carried indels; phenotypically, two were albino, one was chinchilla, and two had variegated pigmentation, indicating mosaicism.

From the 3×10⁸ GCs experiment, four (4) pups (14%) were obtained, two of which died at birth, preventing a coat color or genome analysis. Coat color analysis of the remaining two pups revealed one chinchilla and one mosaic pup. These results indicate that single-AAV delivery of Nme2Cas9 and its guide can be used to generate mutations in mouse zygotes without microinjection or electroporation.

To measure on-target indel formation in the Tyr gene. DNA was isolated from the tails of each mouse, the locus was amplified and upon which a TIDE analysis was performed. All mice had high levels of on-target editing by Nme2Cas9, varying from 84% to 100%. See, FIGS. 19B-C. Most lesions in albino mouse 9-1 were either a 1- or a 4-bp deletion, suggesting either mosaicism or trans-heterozygosity, but albino mouse 9-2 exhibited a uniform 2-bp deletion. See, FIG. 19C.

The data is inconclusive as to whether there was no mosaicism in mouse 9-2, or that additional alleles were absent from mouse 9-1, because only tail samples were sequenced and other tissues could have distinct lesions. Analysis of tail DNA from chinchilla mice revealed the presence of in-frame mutations that are potentially the cause of the chinchilla coat color. The limited mutational complexity suggests that editing occurred early during embryonic development in these mice. These results provide a streamlined route toward mammalian mutagenesis through the application of a single AAV vector, in this case delivering both Nme2Cas9 and its sgRNA.

### G. Cas9 Nucleases As Base Editors

Point mutations represent the largest class of known human pathogenic genetic variants. Base editors (BEs), which comprise a single-guide RNA (sgRNA) loaded onto a Cas9 nickase fused to a deaminase enzyme, enables precise installation of A·T to G·C substitutions, in the case of adenine base editor (ABE), or C·G to T·A substitutions, in the case of cytidine base editor (CBE). In contrast to traditional nuclease-dependent genome editing approaches, base editors do not generate double-stranded DNA breaks (DSBs), do not require a DNA donor template, and are more efficient in editing non-dividing cells, making them attractive agents for in vivo therapeutic genome editing.

While robust editing has been achieved in many cultured mammalian cell systems, safe and effective in vivo delivery of the base editors remains a major challenge. To date, both non-viral and viral delivery methods have been reported to deliver base editors for in vivo therapeutic purposes in rodents and primates which hold great promise. For example, in vivo delivery using adeno-associated virus (AAV) has achieved efficient editing in a wide range of tissue and cell types including liver, heart, muscle, retina, and CNS. However, the large coding sizes (5.2 kb) of most well-characterized Streptococcus pyogenes Cas9 (SpCas9) containing BEs well exceed the packaging limit of AAV (5 kb). Currently, in vivo delivery of base editors by AAV has been approached by splitting the base editors into two AAVs and relying on the use of intein trans-splicing for the reconstitution of the full-length effector. Although effective, this approach requires simultaneous entry of both AAVs in the target cell and successful in trans reconstitution of the two intein halves, which may compromise the on-target efficiency. Furthermore, the requirement of delivering two AAV vectors for each disease target site increases the total viral dosage needed for a treatment, which raises safety concerns and adds burdens to AAV manufacturing.

Compact Cas9 orthologs are ideal candidates for engineering base editors suitable for single-AAV delivery. For example, the Hewitt group has achieved single-AAV delivery of a domain-inlaid Staphylococcus aureus Cas9 (SaCas9) ABE in cultured HEK293 cells. Previously, we characterized Neisseria meningitidis Cas9, Nme2Cas9, for in vivo genome editing.

Nme2Cas9 is a compact, intrinsically accurate Cas9 with a distinct N4CC PAM specificity. In data presented below, N-terminal ABEs fused to a Nme2Cas9 were developed, and their editing efficiencies, editing windows, and off-target activities were defined in comparison with the widely applied SpyCas9-BEs in cultured cells. Next, N-terminal Nme2Cas9-ABE was shown to edit multiple therapeutically relevant loci, including one of the common mutations occurring in Rett syndrome patients that cannot be targeted by the SpCas9-ABEs, because of the PAM restrictions. Lastly, by optimizing the promoter and the nuclear localization signals, we show that Nme2Cas9-ABE can be packaged in a single AAV for in vivo delivery. One systematic administration of the single AAV encoding Nme2Cas9-ABE readily corrects the disease mutation and phenotype in an adult mouse model of hereditary tyrosinemia type 1 (HT1).

First, to quickly evaluate the base editing efficiency, an ABE reporter cell line was developed, where a G-to-A mutation in an mCherry coding sequence generates a nonsense mutation. Adenine base editing can reverse the mutation and recover the red fluorescence, and the editing efficiency can be readily measured by fluorescent-activated cell sorting (FACS). Initially, an Nme2Cas9-ABE7.10 was constructed by linking TadA-TadA7.10 dimer from the SpCas9-ABE7.10 to the N-terminus of the Nme2Cas9 HNH nickase. However, by plasmid transient transfection, Nme2Cas9-ABE7.10 showed very low to no activity in the ABE reporter cell line. Preliminary data comparing NmeCas9 and SypCas9 nucleases with N-terminally fused nucleotide deaminase domains demonstrated several differences between the two constructs. For example, an ABE reporter cell line was constructed to test the gene editing characteristics between the two orthologs. See, FIG. 21A. The NmeCas9 constructs are shown with either an N-terminal fusion of an ABE7.10 control domain, or an N-terminal fusion of an ABE8e domain. Because the evolved TadA8e is highly active and compatible with a wide range of Cas9s, Nme2Cas9-ABE8e was engineered by linking TadA8e to the N-terminus of the Nme2Cas9 HNH nickase. Similar constructs were created that replaced the NmeCas9 with the SpyCas9. See, FIG. 21B. The data show that both Cas9 orthologs had greater gene editing efficiency when N-terminally fused to the ABE8e domain as compared to the ABE7.10 control domain. Further, the SpyCas9 ortholog was observed to have greater gene editing efficiency than the NmeCas9 ortholog. See, FIG. 21C. Next, to define the editing window and editing efficiency of Nme2Cas9-ABE8e, and to compare to those of SpCas9-ABE7.10 and SpCas9-ABE8e, plasmids were transfected expressing the ABE along with sgRNAs targeting 12 human genomic loci for Nme2Cas9-ABE8e (including 8 dual-target sites (target sites followed by NGGNCC PAMs) and 4 Nme2Cas9 specific target sites), and 8 dual-target sites for SpCas9-ABEs. The data shows that Nme2Cas9-ABE8e has an editing window of 2-18 (where position 1 is the first nucleotide of the protospacer and the PAM is at positions 25-30, wider than those of the SpCas9-ABEs, and overall lower efficiency. See, FIG. 21D.

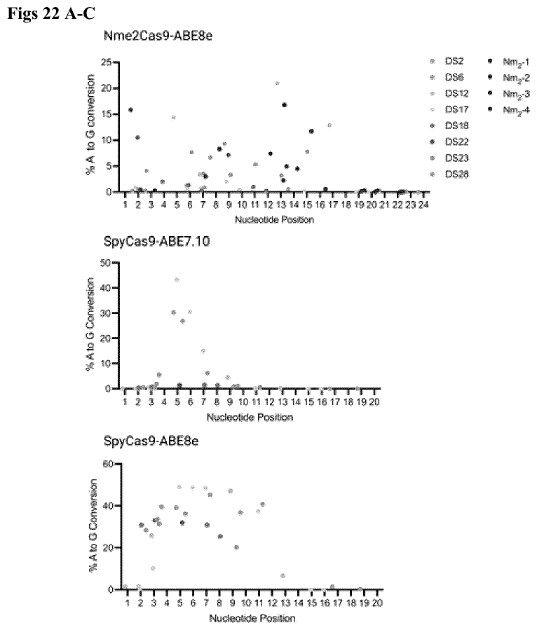

Off-target effects of the Nme2Cas9-ABE8e were then evaluated. It has been shown that the major source of DNA off-target base editing is Cas9-dependent, which is caused by Cas9 binding and unwinding at near-cognate sequences. Because Nme2Cas9 is intrinsically highly accurate, it was hypothesized that Nme2Cas9-ABE8e will show a lower Cas9-dependent off-target effect than SpCas9-ABE8e. However, the overall low on-target efficiency and the limited number of potential genome-wide off-target sites for Nme2Cas9-ABE8e makes it difficult to detect and compare the off-target effect to that of SpCas9-ABE8e. Alternatively, a systematic investigation of the tolerance of nucleotide mismatches between the sgRNAs and the target sequence was undertaken. To do this, panel of guides was designed targeting the ABE reporter with single- or di-nucleotide mismatches to the target sequence for both Nme2Cas9-ABE8e and SpCas9-ABE8e and measured their activities by plasmid transfection and FACS. See, FIG. 21E. In contrast to the gene editing efficiencies, however, the N-terminally fused Nme2Cas9-ABE8e construct had less sgRNA nucleotide mismatches as compared to the SpyCas9 construct. This observation is consistent with the observation that the Nme2Cas9 nucleases, in general, are hyper-accurate as compared to other Cas9 nucleases (infra). See, FIG. 21E. Considering the differences in on-target efficiencies between the two effectors, the activities of the mismatched guides were normalized to the perfectly complementary guides for each effector. The Nme2Cas9-ABE8e has a significantly lower off-target effect than SpCas9-ABE8e: while single-nucleotide mismatches in the seed region (guide nucleotide 17-24 for Nme2Cas9, and 10-20 for SpCas9) and the majority of di-nucleotide mismatches significantly compromised the efficiency of Nme2Cas9-ABE8e, they were mostly well-tolerated by SpCas9-ABE8e. A target-specific summary presentation of these A-G conversion efficiencies confirms these differences. See, FIG. 22.

### II. Therapeutic Applications Of N-Terminal Cas9 Fusion Constructs

### A. Rett Syndrome

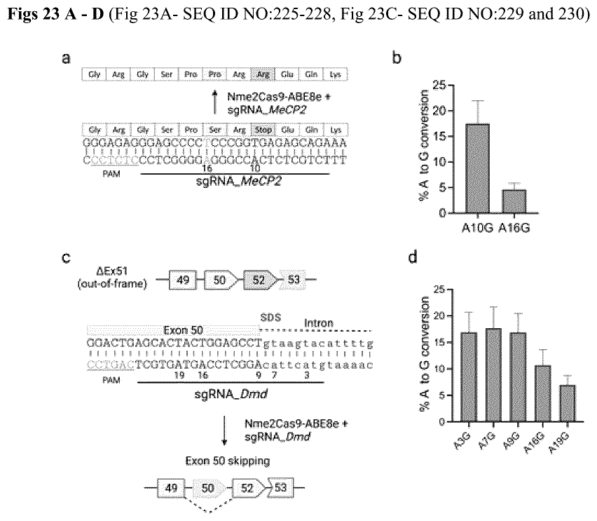

Because of this confirmed hyper-accuracy of the N-terminal Nme2Cas9-ABE8e construct, its ability to revert single base gene mutations that result in genetic diseases was evaluated. For example, an sgRNA was created to guide the N-terminal Nme2Cas9-ABE8e to single base mutations in positions 10 and 16 of the MeCP2 gene, which are known to result in Rett syndrome. An N-terminal Nme2Cas9-ABE8e mRNA was electroporated with a synthetic sgRNA into a Rett syndrome patient-derived fibroblast cell line that possesses this mutation. By amplicon deep sequencing, the data showed that Nme2Cas9-ABE8e generates 17.82±5.07% editing at the target adenine (A10). An inefficient bystander editing (4.6±1.36%) at an upstream adenine (A16) will cause a missense mutation (c.296 T>C; p.S166P). Because S166 has been shown subject to phosphorylation in mice and is conserved from X. laevis to humans, the bystander editing at A16 may impede functional rescue of edited cells. See, FIG. 23A. The data show 15-20% A-G conversion efficiency at position 10 and a 5% A-G conversion efficiency at position 16. See, FIG. 23B.

### B. Duchenne Muscular Dystrophy (DMD)

SgRNAs were also created to guide an N-terminal Nme2Cas9-ABE8e to single base mutations at positions 3, 7, 9, 16 and 19 of the Dmd gene, which are known to result in muscular dystrophy. Also determined was that this gene editing strategy resulted in the skipping of exon 50 to restore the wild type reading frame. A disease-suppressing mutation was generated that has been shown to reverse phenotypes of a validated Duchenne muscular dystrophy (DMD) mouse model (ΔEx51). The ΔEx51 mouse model was generated by deletion of the exon 51 in the Dmd gene, resulting in a downstream premature stop codon in exon 52, causing the production of a nonfunctional truncated dystrophin protein. Previously, it has been shown that the Dmd reading frame can be restored by skipping exon 50 by adenine base editing. However, in vivo base editing in those studies using ABEmax-SpCas9-NG delivered by dual-AAV vectors was limited to local muscle injection due to the high viral dosage required to achieve therapeutic effects. An sgRNA design for an N-terminally fused Nme2Cas9-ABE8e was created to target the adenine (A7) within the splicing donor site of exon 50. See, FIG. 23C. By plasmid transfection in the mouse N2a cell line and amplicon deep sequencing, it was found that N-terminally fused Nme2Cas9-ABE8e can generate 17.67±4.57% editing at A7. While multiple bystander adenines were edited efficiently, these adenines are either within exon 50 or the intron, which will not be expressed. Overall, the data show a range of 5-15% A-G conversion efficiency at these positions. See, FIG. 23D.

### C. AAV Delivery of N-Terminal Cas9 ABE Constructs

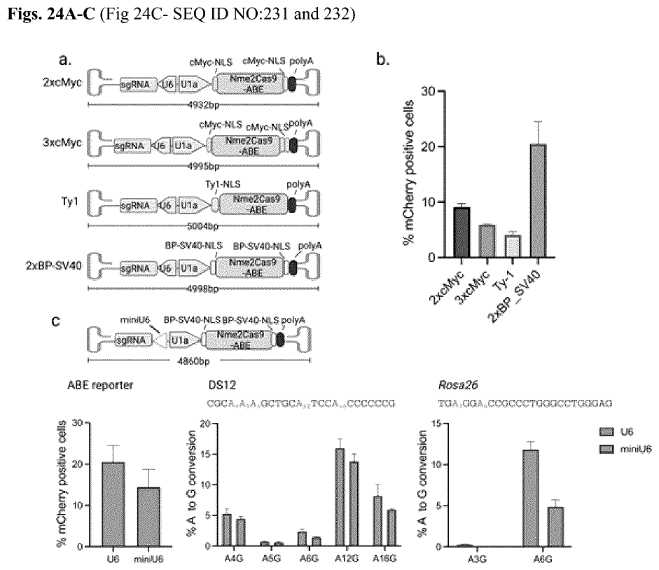

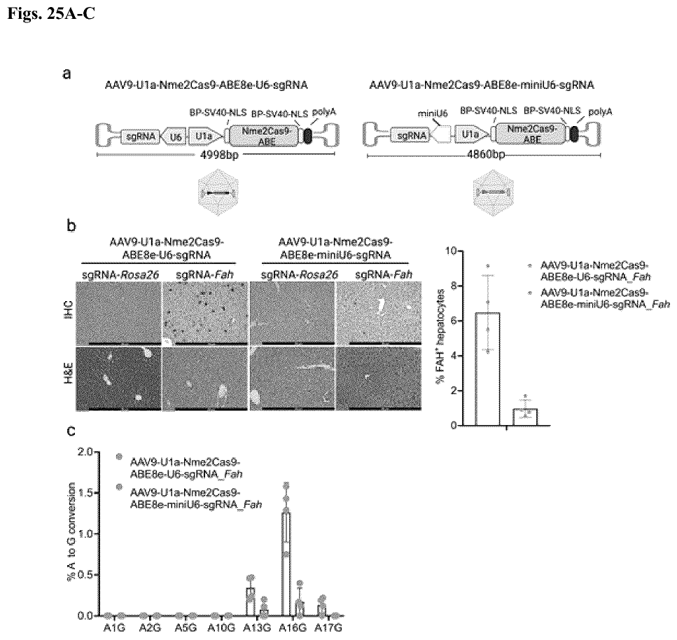

The clinical administration of N-terminal Cas9-ABE8e constructs were evaluated as to their compatibility with adeno-associated virus (AAV) delivery. As detailed further below, AAV delivery of Nme2Cas9 constructs, in particular, have numerous advantages over other Cas9 nuclease orthologs due to their smaller size. Previously, it has been shown that Nme2Cas9 with a sgRNA can be packaged into a single AAV and support efficient editing in vivo. Because of the compact sizes of Nme2Cas9 and TadA8e, Nme2Cas9-ABE8e with a sgRNA can, in theory, be packaged into a single AAV for in vivo base editing. To test this idea, Nme2Cas9 was replaced with an N-terminally fused Nme2Cas9-ABE8e in an all-in-one AAV vector. One cMyc NLS sequence was attached on each terminus of Nme2Cas9-ABE8e while retaining the original promoters for Nme2Cas9-ABE8e and sgRNA expression. By plasmid transient transfection, the single-AAV vector showed 9.12±0.69% editing efficiency in the ABE reporter cell line. To further improve editing efficiency, three different NLS configurations were tested: 1) one cMyc NLS on the N-terminus and two cMyc NLS on the C-terminus; 2) one Ty1 NLS, which derived from the yeast Ty1 retrotransposon that supports robust nuclear localization in dPSPCas13b fusion proteins, on the N-terminus; and 3) one bipartite SV40 NLS (BP_SV40) on each terminus of Nme2Cas9-ABE8e. When transfecting the vector plasmid into the ABE reporter cell line, the construct having BP_SV40 NLS on each terminus showed an editing efficiency of 20.5±3.2%. As the in vivo delivery of AAV plasmid/vector payloads are assisted by appending nuclear localization signal proteins (e.g., NLS), several Nme2Cas9-ABE8e N-terminally fused constructs were created and tested for efficacy using the standard m-Cherry reporter system. See, FIGS. 24A and 24B.

As the 2XBP—SV40 NLS construct was observed to have optimal performance, this construct was chosen to evaluate gene editing efficiency between the U6 and miniU6 promoters at the D12 and Rosa26 test target sites. The total length of the vector construct with the BP—SV40 NLS, hereafter Nme2Cas9-ABE8e-U6, is 4998 bp, just below the packaging limit of AAV. To test if the vector size could be further reduced without significantly compromising the editing efficiency, a “miniU6” promoter, which has been shown to support sgRNA expression to achieve a similar level of CRISPR editing efficiency was compared to the U6 promoter. When replacing the U6 promoter with miniU6 promoter, hereafter Nme2Cas9-ABE8e-miniU6, a vector was generated with a total length of 4860 bp, well below the packaging limit of AAV. (infra). When performing transient plasmid transfection, the construct showed reduced yet significant editing in the ABE reporter cell line. The data show that the U6 promoter consistently showed greater gene editing efficiency. To avoid potential ABE reporter-specific effect, both single-AAV vectors were tested at two endogenous target sites: 1) one of the human dual-target sites, DS12, and 2) a previously reported Nme2Cas9 target site in the mouse Rosa26 gene. By plasmid transfection in human HEK293T or mouse N2a cells, significant editing was observed at these loci by both vectors, although the Nme2Cas9-ABE8e-miniU6 vector was less efficient. See, FIG. 24C. These AAV N-terminally fused Nme2-ABE8e constructs were used to confirm the above Fah gene editing data. See, FIG. 25A-C.

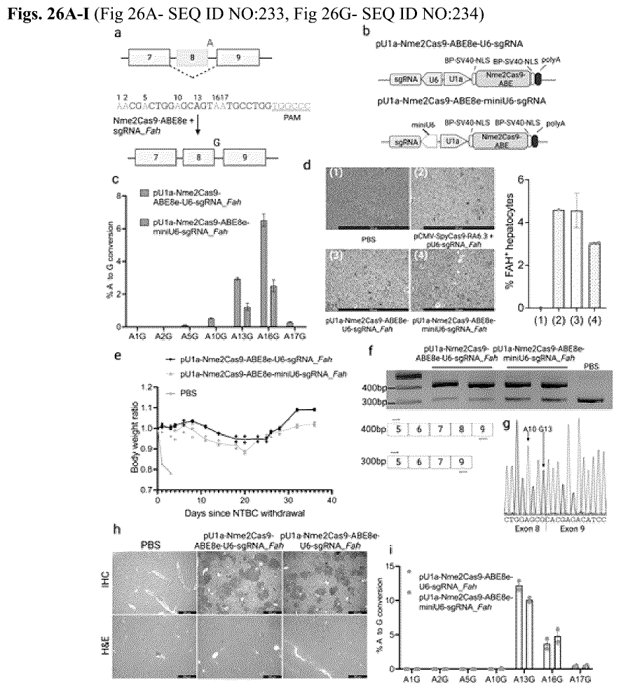

The liver disease HT1 was used to test in vivo editing efficiency and the therapeutic potential of the single-AAV constructs. HT1 is caused by mutations in fumarylacetoacetate hydrolase (Fah) gene, which catalyzes the tyrosine catabolic pathway. FAH deficiency leads to accumulations of toxic fumarylacetoacetate and succinyl acetoacetate, causing liver, kidney, and CNS damage. The Fah^{PM/PM} mouse model possesses a G·C to A·T point mutation in the last nucleotide of exon 8, which causes skipping of exon 8 and FAH deficiency. See, FIG. 26A. Without treatment, an individual will rapidly lose weight and eventually die. The Fah^{PM/PM} mouse can be treated with 2-(2-nitro-4-trifluoromethylbenzoyl)-1,3-cyclohexanedione (NTBC), an inhibitor of an enzyme upstream in the tyrosine degradation pathway, to prevent toxin accumulation. A hepatocyte in which the Fah gene has been repaired has growth advantage and expands after NTBC withdrawal. Repair of 1 in 100,000 hepatocytes was reported to rescue the phenotype of Fah^{PM/PM} mice. Previously, in vivo gene-editing tools have been tested to treat the Fah^{PM/PM} mouse model, including Cas9-directed HDR, base editing, microhomology-directed end joining, and prime editing. Multiple approaches including AAV, lipid nanoparticle (LNP), and hydrodynamic tail vein injection of plasmids have been used to deliver the gene-editing agents into this mouse model. The Fah^{PM/PM} mouse model will facilitate comparisons between different genome-editing platforms.

Exon-skipping strategies was further evaluated using single base mutations in the Fah gene, which is known to cause tyrosinemia. An sgRNA was created that targets the point mutation by electroporation of the single-AAV vector plasmids into the mouse embryonic fibroblasts (MEFs) isolated from the Fah^{PM/PM} mouse. This sgRNA was designed to guide an N-terminal Nme2-Cas9-ABE8e construct to positions 5, 10 13, 16 and 17 within exon 8 of the Fah gene. One construct incorporated a U6 promoter and a second construct incorporated a miniU6 promoter. See, FIG. 26A and FIG. 26B. A low but significant gene editing (2.94±0.11% for N-terminal Nme2Cas9-ABE8e-U6, and 1.23±0.30% for Nme2Cas9-ABE8e-miniU6) was detected at the target adenine at position 13 (A13). Also observed was a significantly higher bystander editing (6.52±0.53% for Nme2Cas9-ABE8e-U6, and 2.51±0.52% for Nme2Cas9-ABE8e-miniU6) at A16. Nevertheless, A16 is within the intron region downstream of the splicing donor and is likely to be harmless. In general, the data show that the constructs with the U6 promoter had higher A-G conversion efficiencies than the miniU6 promoter. See, FIG. 26C.

To test single-AAV vectors of N-terminal Nme2Cas9-ABE8e in vivo, hydrodynamic tail vein injection of the AAV-vector plasmids was performed into 10-week-old HT1 mice, or PBS for the negative control group. Also injected were plasmids expressing SpCas9-RA6.3, which is a codon-optimized SpCas9-ABE with increased efficiency, as a positive control. Seven days post-injection, gene editing efficiency was measured before hepatocyte expansion, and withdrawal NTBC for the rest of the mice for long-term phenotypic study. Before NTBC withdrawal, anti-FAH immunohistochemistry (IHC) staining showed 4.58±1.1% FAH+ hepatocytes from the group that injected with the Nme2Cas9-ABE8e-U6 plasmid, and 3.04±0.07% from the group injected with the Nme2Cas9-ABE8e-miniU6 plasmid. The mouse injected with SpCas9-RA6.3 plasmid showed 4.5% FAH+ hepatocytes, consistent with the reported data. See, FIG. 26D. After NTBC withdrawal, body weight changes were monitored. While the PBS injected group rapidly lost body weight within the first 3 days after NTBC withdrawal and was thus euthanized, the mice that were injected with either the Nme2Cas9-ABE8e-U6 plasmid or the Nme2Cas9-ABE8e-miniU6 plasmid gradually gained body weight over 40 days. See, FIG. 25E. These immunohistochemical analysis and body weight data confirmed mutation reversion by demonstrating the reappearance of Fah⁺ hepatocytes subsequent to gene editing and improved body weight gain in the U6 promoter constructs.

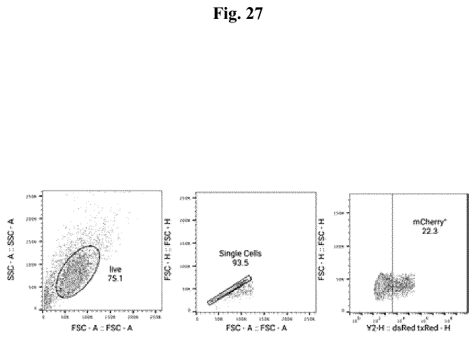

To determine whether the N-terminal Nme2Cas9-ABE8e successfully corrects the Fah gene splicing defect, total RNA was extracted from livers and reverse transcription PCR (RT-PCR) was performed using primers that spanned exons 5 and 9. In contrast to the PBS-injected mice, which only showed a 305 bp PCR band that corresponds to the truncated mRNA lacking exon 8, the majority of the treated mouse livers were observed to have the 405 bp PCR band that contained exon 8. See, FIG. 26F. Sanger sequencing of the 405 bp bands further confirmed the presence of the corrected ‘G’ at position 13. See, FIG. 26G. When performing anti-FAH immunohistochemical (IHC) staining, a significant expansion of FAH+ hepatocytes were observed in the groups that were injected with either of the single-AAV vector plasmids. See, FIG. 25H. Amplicon deep sequencing on the liver genomic DNA from treated mice showed 12.21±1.25% editing for Nme2Cas9-ABE8e-U6 plasmid treated mice, and 10.1±0.647% for Nme2Cas9-ABE8e-miniU6 plasmid treated mice at A13. See, FIG. 26I. These data indicate that N-terminal Nme2Cas9-ABE8e single-AAV vector plasmids successfully corrected the disease genotype and phenotype of the Fah PM/PM mice. These gene editing results for fusion proteins comprising an Nme2Cas9 and an N-terminal adenine deaminase domain were validated by flow cytometry gating. See, FIG. 27.

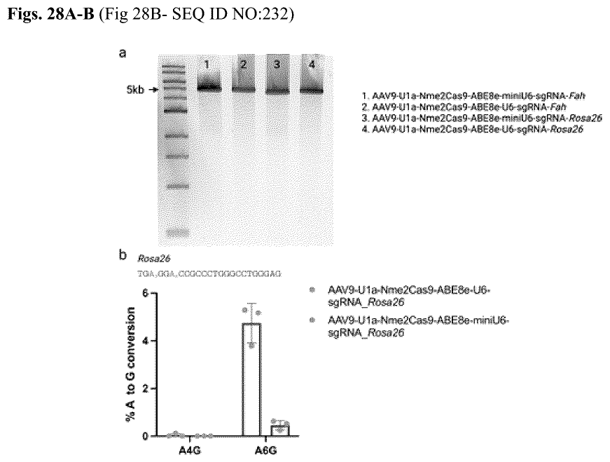

In vivo therapeutic base editing was then assessed using an N-terminal Nme2Cas9-ABE8e delivered by AAV9 in an adult HT1 mouse model. In particular, AAV9 were packaged with the Nme2Cas9-ABE8e-U6 construct or a the Nme2Cas9-ABE8e-miniU6 construct. Next, AAV genome integrity was confirmed by DNA extraction and alkaline gel electrophoresis, where the data did not show any sign of genome truncation. See, FIG. 28A. 8-week-old Fah PM/PM mice were the injected in the tail with 4×10¹³ vg/kg and maintained on NTCB for one month prior to analyzing gene editing efficiency. Because gene expression from the single-strand AAV, unlike from plasmid, requires second-strand synthesis, the mice were kept on NTBC for one month before analyzing the editing efficiency. One month after AAV injection and before NTBC withdrawal, IHC staining was performed using an anti-FAH antibody. The negative control groups injected with AAV9 expressing N-terminal Nme2Cas9-ABE8e and a sgRNA targeting the Rosa26 gene did not show any FAH+ hepatocytes. In contrast, 6.49±2.08% FAH+ hepatocytes were observed in the AAV9-Nme2Cas9-ABE8e-U6-Fah treated group, and 0.98±0.49% FAH+ hepatocytes in AAV9-Nme2Cas9-ABE8e-miniU6-Fah treated group. See, FIG. 28B. The percentage of edited hepatocytes by AAV9-Nme2Cas9-ABE8e-U6-Fah was significantly higher than what has been achieved previously by other genome editing strategies. By targeted deep-sequencing, the editing efficiency at the target adenine (A13) in the AAV9-Nme2Cas9-ABE8e-U6 treated group is 0.34±0.14%, and 0.08±0.09% in AAV9-Nme2Cas9-ABE8e-miniU6 treated group. The reason for the higher frequency of FAH+ hepatocytes than the frequency of editing at the DNA level is because of the polyploidy of the hepatocytes, and the presence of genomic DNA from nonparenchymal cells. Similar results were also observed in previous studies using this mouse model.

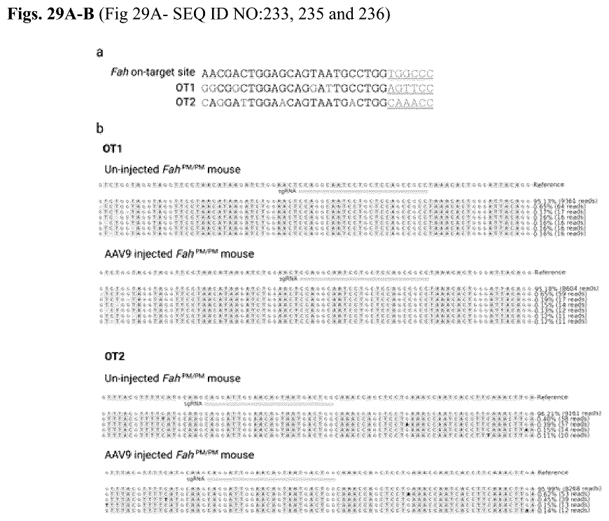

To evaluate potential Cas9-dependent off-target effect in AAV9-injected mice, genome-wide off-target sites for Nme2Cas9 were identified using Cas9-OFFiner, allowing for up to 6 mismatches. Amplicon deep sequencing was performed in AAV9-Nme2Cas9-ABE8e-Fah treated livers (n=8) at the two top-ranking potential off-target sites, each including 5 mismatches. Above-the-background A·T to G·C editing was not detected at these sites. See, FIG. 29.

In conclusion, the data suggest that while a fusion protein comprising an Nme2Cas9 comprising an N-terminal ABE8e domain is capable of hyperaccurate gene editing to revert single base gene mutations and provide therapeutic efficacy, the gene editing efficiency was believed too low to provide a clinically optimal treatment platform.

### III. Nme2Cas9 Single Base Editing

Cas9 is a programmable nuclease that uses a guide RNA to create a double-stranded break at any desired genomic locus. This programmability has been harnessed for biomedical and therapeutic approaches. However, Cas9-induced breaks often lead to imprecise repair by the cellular machinery, hindering its therapeutic application for single-base corrections as well as uniform and precise gene knock-outs. Moreover, it is extremely challenging to combine Cas9-induced DNA double strand breaks and a repair template for homologous directed repair (HDR) for correcting genetic mutations in post-mitotic cells (e.g. neuronal cells).

Single nucleotide base editing is a genome editing approach where a nuclease-dead or-impaired Cas9 (e.g., dead Cas9 (dCas9) or nickase Cas9 (nCas9)) is fused to another enzyme capable of base-editing nucleotides without causing DNA double strand breaks. To date, two broad classes of Cas9 base editors have been developed: i) cytidine deaminase (edits a C·G base pair to a T·A base pair) SpyCas9 fusion protein; and ii) adenosine deaminase (edits a A·T base pair to a G·C base pair) SpyCas9. Liu et al., “Nucleobase editors and uses thereof” US 2017/0121693; and Lui et al., “Fusions of cas9 domains and nucleic acid-editing domains” US 2015/0166980; (both herein incorporated by reference).

In one embodiment, the present invention contemplates a deaminase fusion protein with a compact and hyper-accurate Nme2Cas9 (Neisseria meningitidis spp.). This Nme2Cas9 has 1,082 amino acids as compared to SpyCas9 that has 1,368 amino acids. This Nme2Cas9 ortholog functions efficiently in mammalian cells, recognizes an N₄CC PAM, and is intrinsically hyper-accurate. Edraki et al., Mol Cell. (in preparation). Although it is not necessary to understand the mechanism of an invention, it is believed that the compactness and hyperaccuracy of an NmeCas9 base editor targets single-base mutations that could not be reached previously by other Cas9 platforms currently known in the art. It is further believed that the NmeCas9 base editors contemplated herein target pathogenic mutations that are not feasible via current base editor platforms, and with an increased base editing accuracy.

First generation base editors did include deaminases fused to the N- or C-termini of a Cas domain. Komor et al. Nature. (2016) Programmable editing of a target base in genomic DNA without double-stranded DNA cleavage; and Gaudelli et al. Nature. (2017) Programmable base editing of A·T to G·C in genomic DNA without DNA cleavage. Nature. Newer iterations of base editors have led to improved editing efficiencies and widened or shifted editing windows. These developments relied on efforts to engineer improved deaminases, and/or Cas domains. Richter et al. Nature Biotech. (2020) Phage-assisted evolution of an adenine base editor with improved Cas domain compatibility and activity; Wang et al. Signal Transduction and Targeted Therapy. (2019) BE-PIGS: a base-editing tool with deaminases inlaid into Cas9 PI domain significantly expanded the editing scope; Tran et al. Nature Commun. (2020); Engineering domain-inlaid SaCas9 adenine base editors with reduced RNA off-targets and increased on-target DNA editing; and Chu et al. The Crispr Journal. (2021) Rationally Designed Base Editors for Precise Editing of the Sickle Cell Disease Mutation.

### IV. Inlaid Domain Cas9 Fusion Protein Constructs

Domain-inlaid Nme2Cas9 nucleotide base editors are shown herein to improve editing efficiencies and modulate editing windows. Multiple crystal structures Nme2Cas9 reveal that the N-terminus is on the protein face, opposite that of the edited DNA strand, suggesting that N-terminal deaminase fusions are likely not optimally positioned. Surface loops that are closer to potential paths of the displaced DNA strand were identified and linker-flanked TadA8e were inserted into these internal sites (versions i1-i8). The data shown herein demonstrates that all eight positions of these inlaid domains were active at editing endogenous loci, having editing efficiencies consistently exceeding that of the N-terminal fusion. In particular, the i1, i2, i7 and i8 effectors are consistently 2- to 2.5-fold more active than the N-terminal Nme2-ABE. These results demonstrate that, in general, inlaid domains of Nme2Cas9-ABEs have enhanced editing efficiencies as compared to the N-terminal domain construct.

In one embodiment, the present invention contemplates a fusion protein comprising an NmeCas9 protein and an inlaid nucleotide deaminase protein domain. In one embodiment, the NmeCas9 protein is a Nme2Cas9 protein. In one embodiment, the inlaid nucleotide deaminase protein domain is an inlaid adenine deaminase protein domain. In one embodiment, the inlaid nucleotide deaminase protein domain is an inlaid cytidine deaminase protein domain. In one embodiment, the inlaid adenine deaminase protein domain is an inlaid adenine deaminase8e (ABE8e) protein domain.

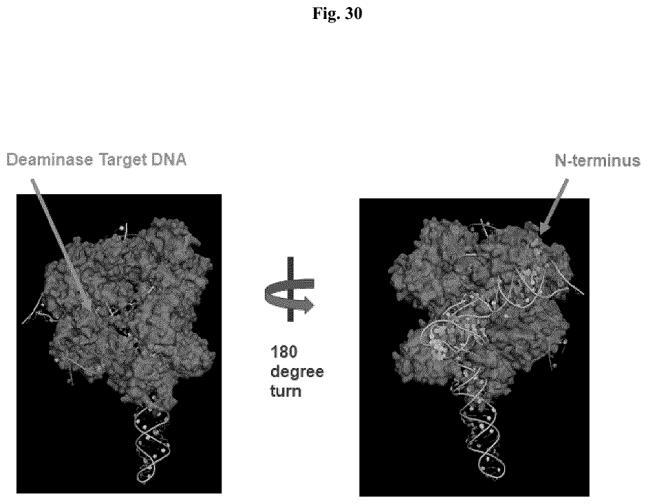

Several approaches to improve the above observed gene editing efficiencies of nucleotide deaminase domains that are N-terminally fused to an NmeCas9 protein may include, but are not limited to: i) move the deaminase domain closer to the R loop; ii) tune the editing window; or iii) increase the deaminase activity. Possible techniques to accomplish these goals may include, but are not limited to: i) use of alternative linkers and location within the Cas9 protein (e.g. shorter/rigid, N-vs. C-termini fusion); iii) alternative deaminase domain insertion into the Cas9 protein; and iii) circular permutation of the Cas9 protein which would create new N- and C-termini. In regards to moving the deaminase domain, both SaCas9 and SpyCas9 have been reported to contain an inlaid adenine deaminase protein domain. Li et al., “Docking Sites Inside Cas9 For Adenine Base Editing Diversification And Off-Target Elimination”; Tran et al., “Engineering Domain-Inlaid SaCas9 Adenine Base Editors With Reduced RNA Off-Targets And Increased On-Target DNA Editing”; Chu et al., “Rationally Designed (Spy) Base Editors (ABE7.10) for Precise Editing of the Sickle Cell Disease Mutation’ The CRISPR Journal (2021). It is envisioned that an inlaid insertion of a nucleotide deaminase protein may three-dimensionally separate the deaminase DNA target site from the Cas9 N-terminus. See, FIG. 30.

These inlaid domain Nme2Cas9 nucleotide base editors enhanced activity and editing window preferences with no significant optimization of the flanking linkers. In one embodiment, the linker flanks each end of the nucleotide base editor. In one embodiment, the linker is flexible and approximately twenty (20) amino acids in length. In one embodiment, linker length may maximize activity gains and improve control over editing windows. For example, a reduced linker length may improve AAV packaging efficiency/titre. In one embodiment, a linker has a length including twenty amino acid linker

The inlaid domain constructs Nme2-ABE i1, i7 and i8 were found to be the most active and exhibited the greatest advantages for favoring PAM-distal (i1) vs PAM-proximal (i7, i8) editing window control.

### A. Inlaid Adenine Base Editors (ABEs)

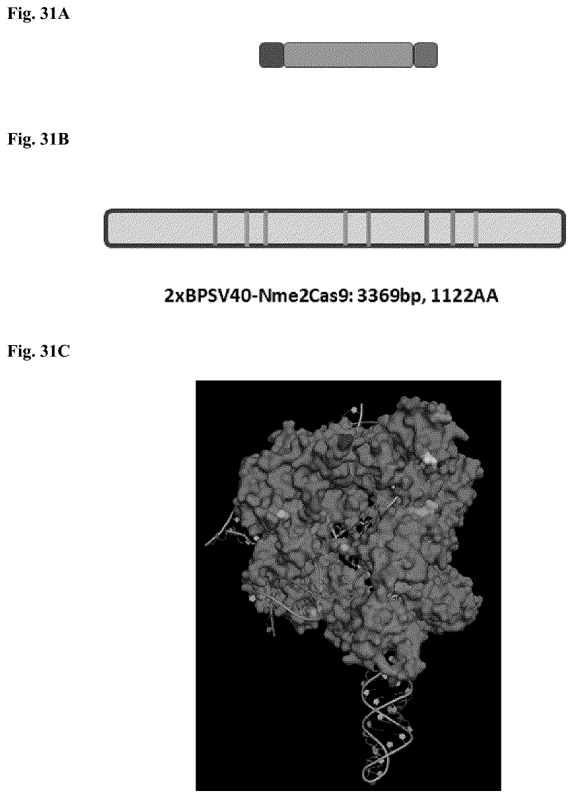

In one embodiment, the present invention contemplates an inlaid adenine deaminase protein domain comprising an adenine deaminase, an N-terminal linker and a C-terminal linker. See, FIG. 31A. For example, the inserted domain may be approximately 206 amino acids in length, wherein the deaminase is TadA8e (166 amino acids in length) and the N-terminal and C-terminal linkers are both about 20 amino acids in length. Oakes (2018) and Chu (2021). Several criteria for inlaid nucleotide deaminase insertion sites were selected including, but not limited to: i) surface exposed amino acids; ii) regions of high conformational flexibility; and iii) apparent proximity to the Non-Target Strand. Based on these selection criteria several potential insertion sites were located along the NmeCas9 protein. See, FIG. 31B. Three dimensional modeling of the Cas9 protein predicted the respective locations of these candidate inlaid insertion locations. See, FIG. 31C.

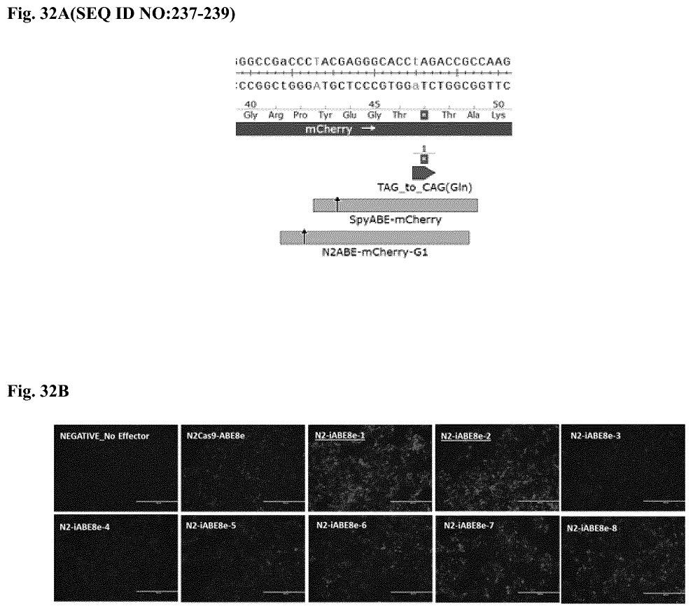

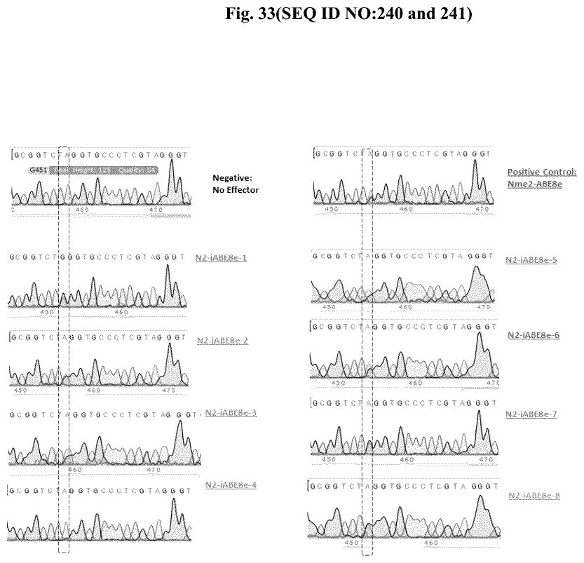

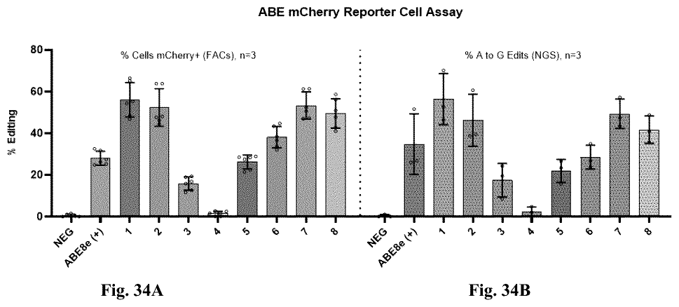

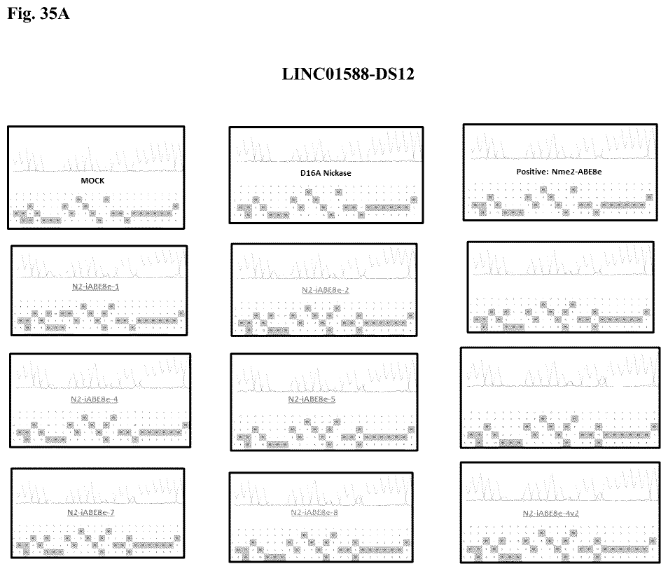

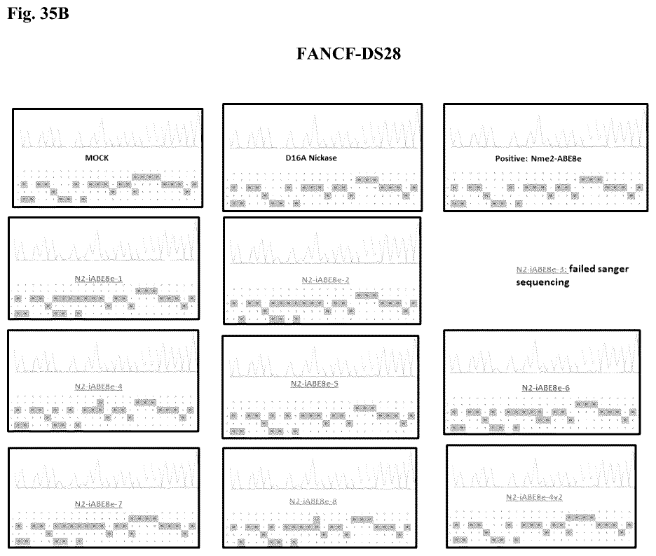

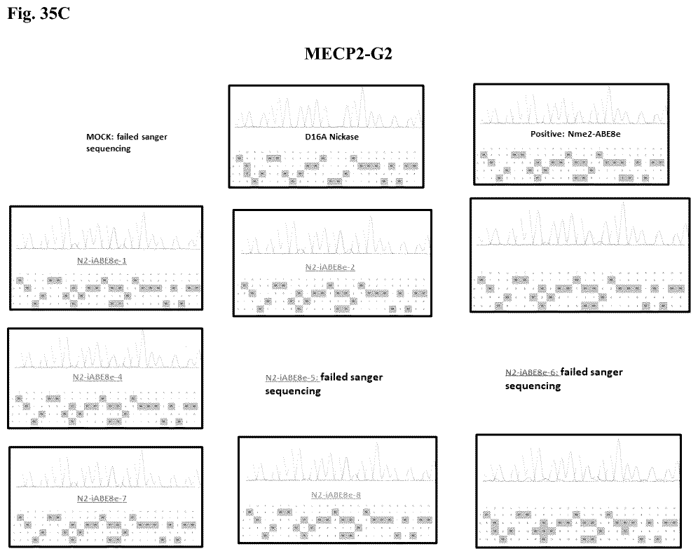

The gene editing capability was determined for each of the eight (8) inlaid locations for the fusion protein comprising an NmeCas9 protein and an inlaid adenine deaminase8e (ABE8e) protein domain shown in in FIGS. 31B and 31C. The assay was performed using a construct comprising the mCherry reporter system. See, FIG. 32A. Briefly, the mCherry reporter system utilizes the following components; i) a premature stop codon inhibits mCherry translation; ii) ABE editing converts an internal STOP codon (e.g., TAG) to a GLN codon (e.g., CAG); iii) the sgRNA targets a distal PAM adenosine position and a proximal PAM bystander adenosine position. The data shows inlaid domain locations having gene editing activity by the appearance of a red fluorescence. See, FIG. 32B. Sanger sequencing was then performed to validate the gene editing activity indicated by the mCherry reporter system analysis. See, FIG. 33. Next, based on the mCherry reporter system data, gene editing efficiencies were then estimated. See, FIGS. 34A and 34B. A general pattern was observed that gene editing was least efficient when the ABE domain was inlaid in the central region of the Cas9 protein. In most cases, the inlaid ABE protein domain had superior gene editing activity as compared to the N-terminally fused ABE protein domain. Gene editing rates were also determined at each of the eight inlaid domain locations at three endogenous gene loci: i) LINC01588-DS12; ii) FANCF-DS28; and iii) MECP2-G2. See, FIGS. 35A, 35B and 35C, respectively.

### B. Therapeutic Applications of Inlaid Domain Nme2-Cas9 Fusion Proteins

### 1. Duchenne's Muscular Dystrophy

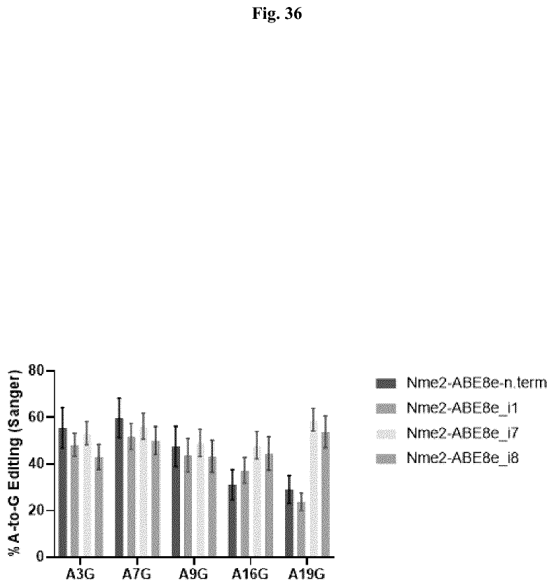

In one embodiment, the present invention contemplates a method to treat DMD with an Nme2Cas9 inlaid ABE construct. For example, several A→G conversion have been successfully converted a mutated DMD gene to wild type. See FIG. 36. These data were collected comparing Nme2Cas9-ABE-nt, i1, −i1, i7 and i8 constructs. The inlaid domain constructs were observed to have a higher editing efficiency than the N-terminal construct for the A16G and A19G conversions.

### 2. Rett Syndrome

In one embodiment, the present invention contemplates a method to treat Rett Syndrome with an Nme2Cas9-ABE construct. In one embodiment, the ABE is a terminal domain. In one embodiment, the ABE is an inlaid domain. For example, Rett syndrome mutations in exon 4 of the MeCP2 gene are targeted by Nme2-ABEs. The empirical nature of this treatment is shown by attempts to correct known Rett syndrome mutations in the HEK293T Rett-PiggyBac cell line by plasmid transfections or editing in Rett-patient derived fibroblasts (PDF) with mRNAs and synthetic gRNA. See, Table 3.

TABLE 3
Representative data showing ABE-conversion of MeCP2 mutations

| | | Patient | | | |
|---|---|---|---|---|---|
| | | Population | | | |
| | Protein | with mutation | Bystander | RETT- | RETT- |
| Mutation | Change | (%) | Adenine's | PiggyBac | PDF |
| c.502C > T | R168X | 7.63 | yes | ✓ | ✓ |
| c.763C > T | R255X | 6.68 | no | ✓ | X |
| c.808C > T | R270X | 5.80 | yes | ✓ | X |
| c.916C > T | R306X | 5.17 | yes | ✓ | ✓ |

✓ = Successful
X = In Progress

The following MeCP2 target site sequences and their respective PAM were tested in attempt to correct Rett syndrome mutations. See, Table 4.

TABLE 4
MeCP2 Target Site Sequences Associated With Rett Syndrome Mutations

SEQ ID NO
54
55
56
57
58
59
60
61
62
63
64
65
66
67
68
69

| Guide | Spacer 5′-3′ | PAM | Target A. | Bystander A's. |
|---|---|---|---|---|
| 502-G6 | GTGGTTTCTGCTCTCACCGGGAGG | GGCTCC | 16 | 22 |
| 502-G7 | TGGTTTCTGCTCTCACCGGGAGGG | GCTCCC | 15 | 21 |
| 502-G8 | TCTGCTCTCACCGGGAGGGGCTCC | CTCTCC | 10 | 16 |
| 509-G9 | AGGTGGTTTCTGCTCTCACCGGGA | GGGGCT | 18 | 24 |
| 502-G10 | GGTTTCTGCTCTCACCGGGAGGGG | CTCCCT | 14 | 20 |
| 763-G1 | GGCCTCAGCTTTTCACTTCCTGCC | GGGGGG | 15 | 7 |
| 763-G2 | CTTTTCACTTCCTGCCGGGGCGTT | TGATCA | 7 | n/a |
| 763-G3 | TTTCACTTCCTGCCGGGGCGTTTG | ATCACC | 5 | n/a |
| 763-G4 | TTCACTTCCTGCCGGGGCGTTTGA | TCACCA | 4 | n/a |
| 808-G1 | CCACACTCCCCGGCTTTCAGCCCC | GTTTCT | 19 | 3, 5 |
| 808-G2 | GGCTTTCAGCCCCGTTTCTTGGGA | ATGGCC | 8 | 24 |
| 808-G3 | GCTTTCAGCCCCGTTTCTTGGGAA | TGGCCT | 7 | 23, 24 |
| 916-G1 | CGGGTCTTGCACTTCTTGATGGGG | AGTACG | 11 | 19 |
| 916-G2 | TCTTGCACTTCTTGATGGGGAGTA | CGGTCT | 7 | 15, 21, 24 |
| 916-G3 | TTGCACTTCTTGATGGGGAGTACG | GTCTCC | 5 | 13, 19, 22 |
| 916-G4 | TGCACTTCTTGATGGGGAGTACGG | TCTCCT | 4 | 12, 19, 23 |

The target adenine position (e.g., Target A) in the spacer sequence is determined by counting from the 5′ terminal base. Bystander adenines are also identified in the same manner. Representative target sites were screened in RETT-PiggyBac cells and Rett-patient derived fibroblasts (PDFs).

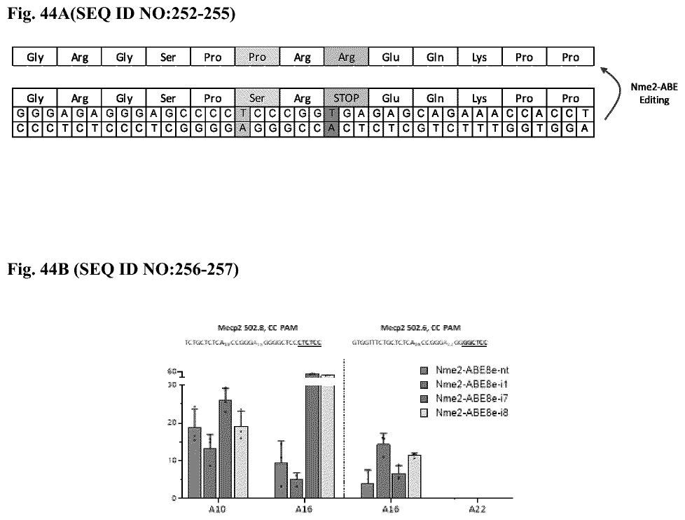

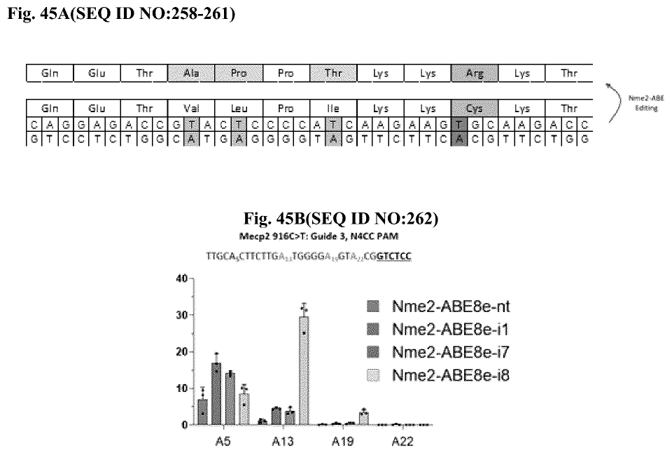

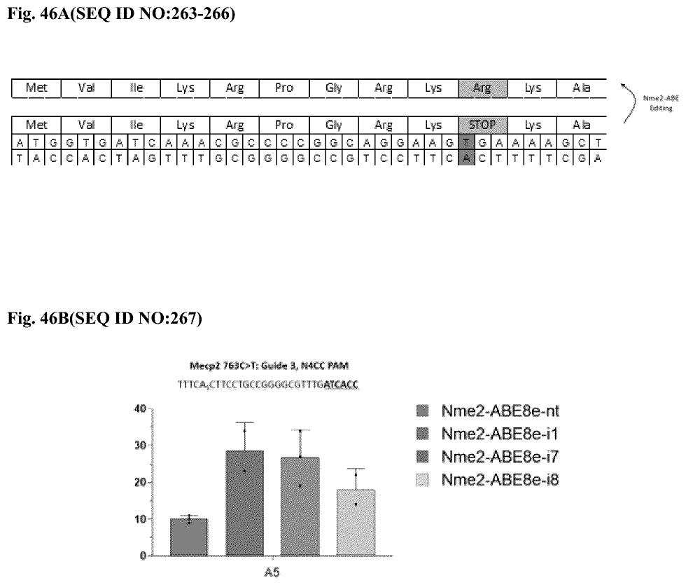

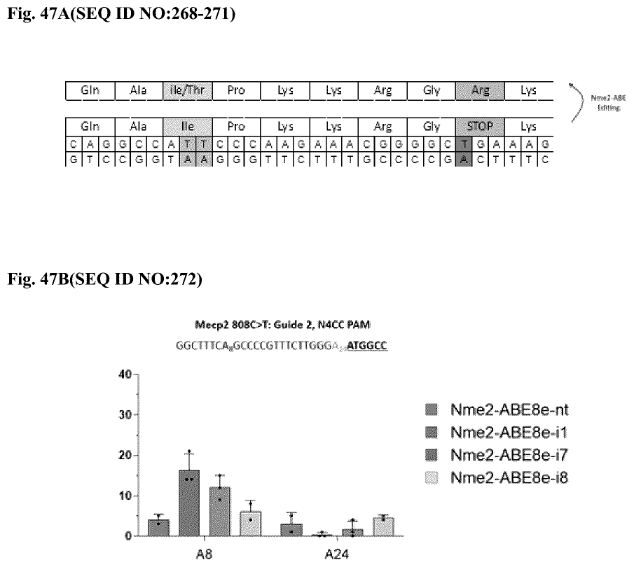

Conversion of four (4) different McCp2 exon 4 mutations have been successfully performed with the presently disclosed Nme2Cas9-ABE constructs. In particular, the N-terminal (-nt) construct was compared to three (3) inlaid domain (i1, i7 and i8) constructs. The inlaid domain constructs demonstrated higher c.502 C>T conversion in most A→G edited sites. See, FIGS. 44A-44B. The inlaid domain constructs demonstrated higher c.916 C>T conversion in some A→G edited sites. See, FIGS. 45A-45B. The inlaid domain constructs demonstrated higher c.763C>T conversion in the A→G edited site. See, FIGS. 46A-46B. The inlaid domain constructs demonstrated higher c.808C>T conversion in some of the A→G edited sites. See, FIGS. 47A-47B.

### 3. Batten Disease

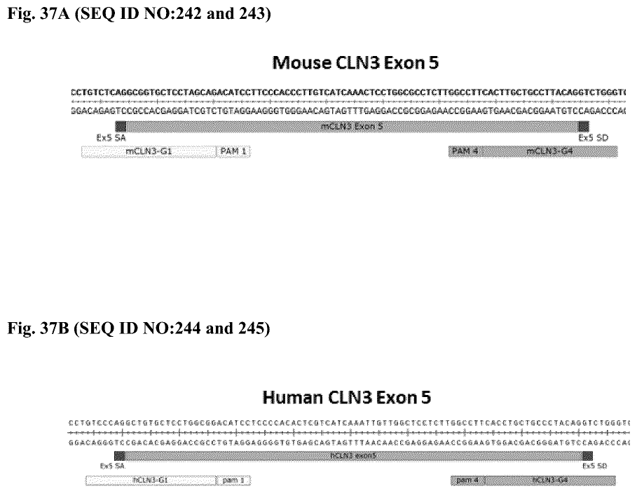

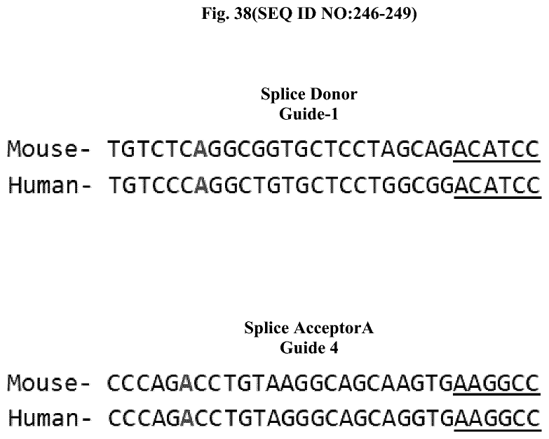

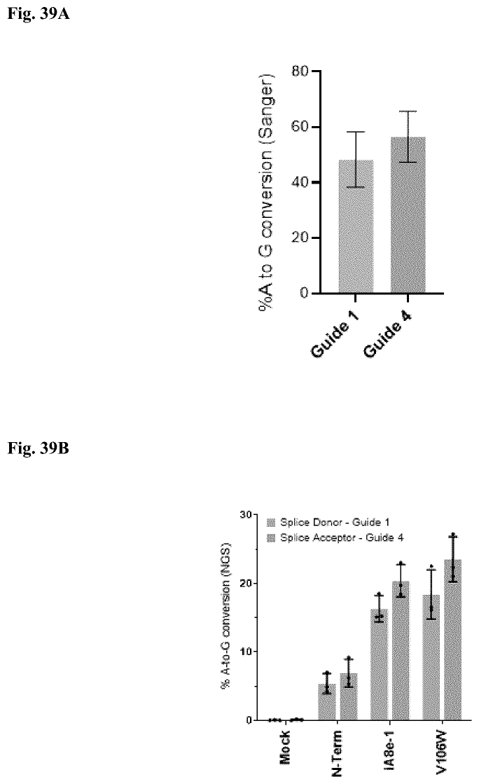

In one embodiment, the present invention contemplates a method of treating Batten disease with Nme2Cas9 base editing of the CLN3 Δex7/8 mutation and concomitant exon 5 skipping. In one embodiment, the method further comprises a guide mRNA to target the CLN3 mutation See, FIGS. 37A, 37B and 38. Significant gene editing activity was observed with inlaid domain constructs using these mRNA sequences, and were superior to that seen with N-terminal constructs. See, FIGS. 39A and 39B.

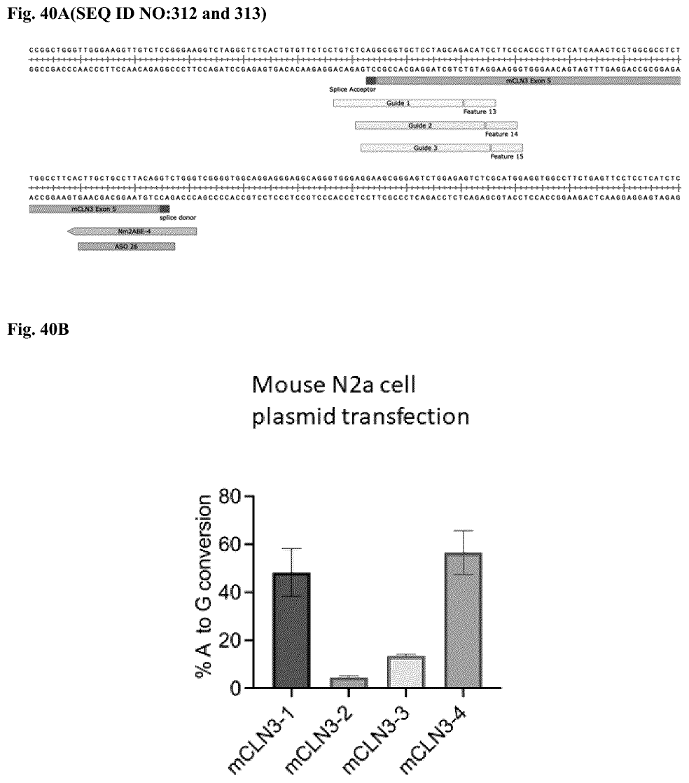

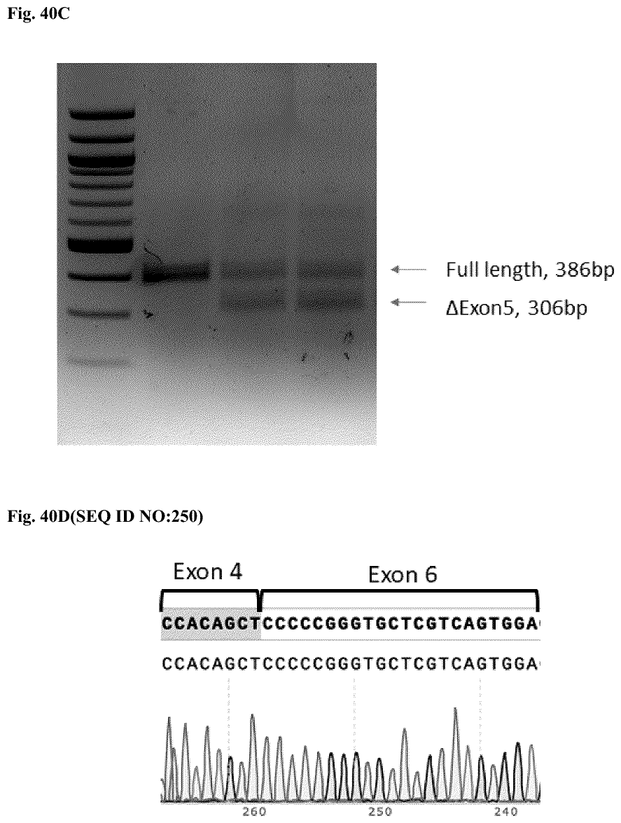

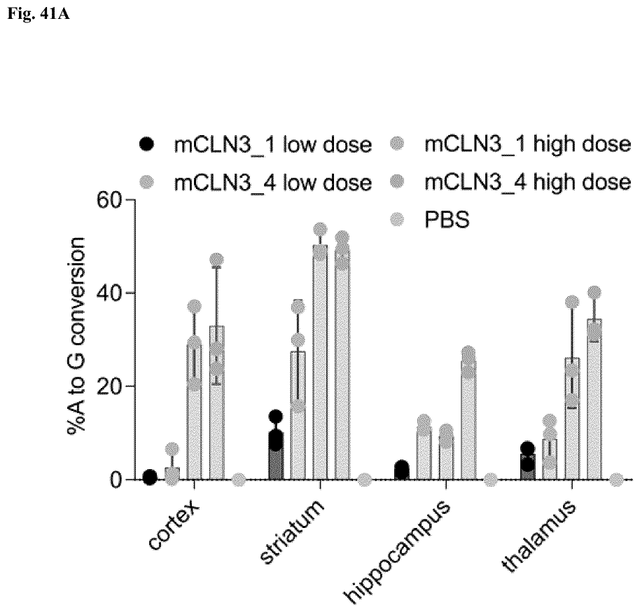

Exon 5 skipping was observed. See, FIGS. 40A-40D. ASO-induced exon 5 skipping of mouse CLN3 transiently restores reading frame in Cln3Δex7/8 mice and ameliorates Batten disease phenotypes. Nme2-ABE editing of exon 5 splice sites could potentially induce long-term exon skipping. The above data demonstrates the targeting of the WT allele in mouse Neuro2a cells, which express Cln3, using Nme2-ABE-i1 and candidate guide RNAs. These guide mRNAs yielded efficient editing at each splice site (acceptor site and receptor site). Splice site mutations can sometimes lead to activation of nearby cryptic splice sites rather than exon skipping. To assess this, RT-PCR analysis of total RNA was performed using primers for exons 4 and 6. A smaller gel band appeared in the edited samples. See, FIG. 40C. Sanger sequencing confirmed its identity as an exon-5-skipped species. See, FIG. 40D. These results confirm that inlaid domain Nme2-ABEs can induce exon skipping via splice site editing.

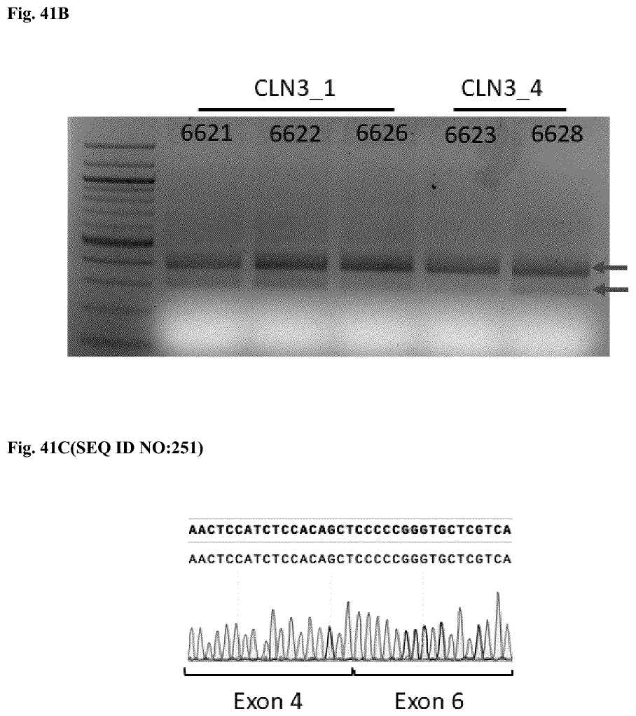

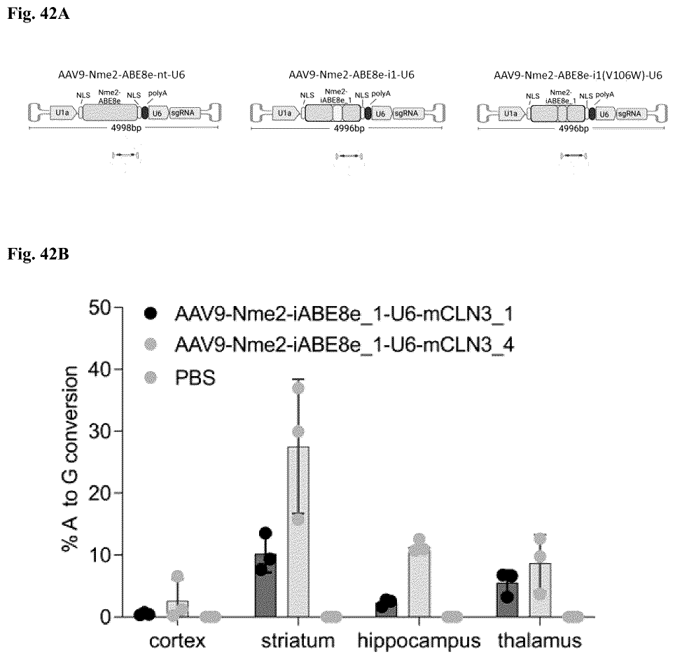

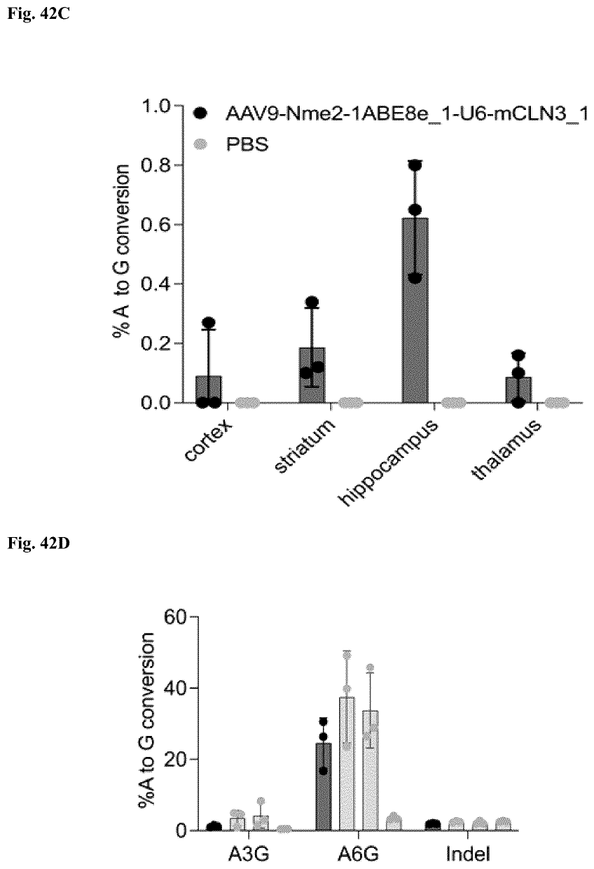

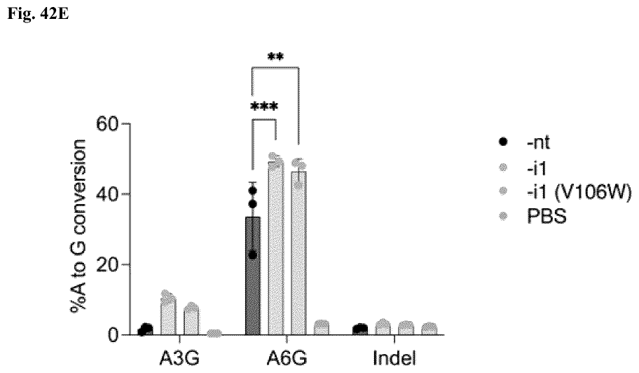

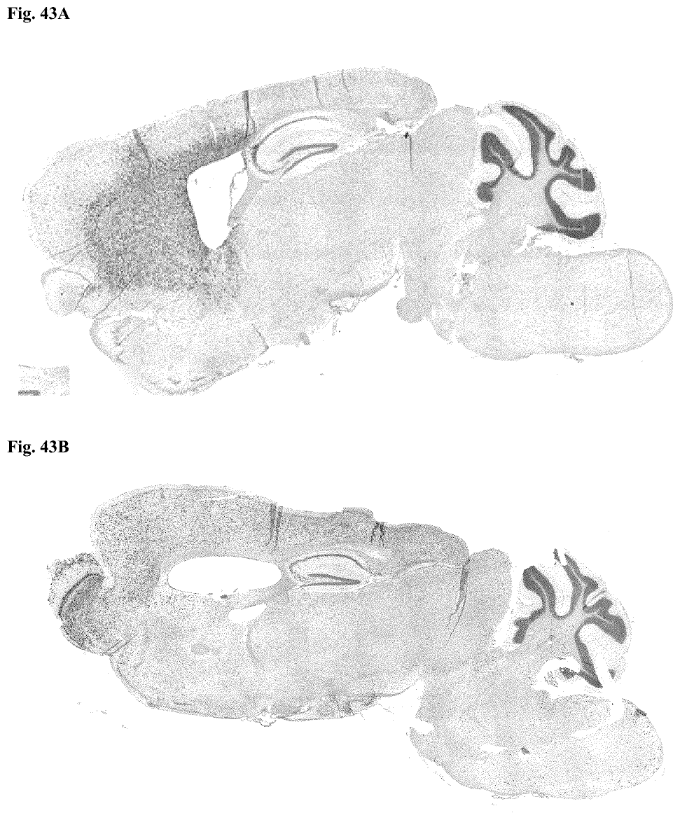

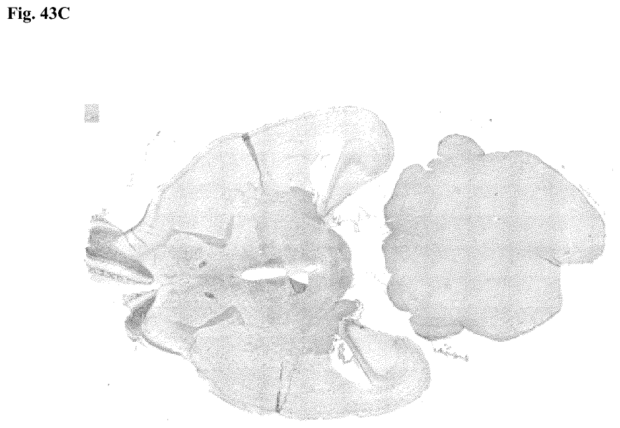

Gene editing and exon skipping was also observed in brain structures such as cortex, striatum, hippocampus and thalamus using AAV delivery of the inlaid domain and N-terminal domain Nme2-Cas9 constructs. See, FIGS. 41A-41C. CLN3 gene editing was observed in specific brain regions of both adults and neonates, as well as in liver. See, FIGS. 42A-42E. The regional distribution of CLN3 mutation conversions was documented by brain slice mRNA transcript imaging. See, FIG. 43A-43C.

Batten disease is an autosomal recessive fatal neurological disorder caused by mutations in the CLN3 gene. Patients are often blind by later childhood and gradually develop seizure and movement abnormalities, and most patients only live into early adulthood. Approximately 85% of Batten disease patients carry a 1.02 kb deletion spanning exons 7 and 8 of CLN3 61, which results in a premature termination codon in exon 9 and loss of a C-terminal lysosomal targeting sequence. The homozygous Cln3Δex7/8 mouse model (JAX #017895) exhibits pathological changes. Administering AAVrh.10 expressing human wild-type CLN3 protein to newborn Cln3Δex7/8 mice partially corrects neurological lysosomal storage defects. Homozygous Cln3Δex7/8 mice can also be treated by skipping exon 5 of the Cln3 gene via splice-switching antisense oligos (ASOs) to bring the C-terminus of the CLN3 ORF back in frame, suggesting that BE-induced CLN3 exon 5 skipping could have durable therapeutic value. Kyttälä et al., “Two motifs target Batten disease protein CLN3 to lysosomes in transfected nonneuronal and neuronal cells” Mol Biol Cell 2004 March; 15(3):1313-1323; Pontikis et al., “Late onset neurodegeneration in the Cln3−/− mouse model of juvenile neuronal ceroid lipofuscinosis is preceded by low level glial activation” Brain Res 2004 Oct. 15; 1023(2):231-242; Osbrio et al., “Neurodevelopmental delay in the Cln3Deltaex7/8 mouse model for Batten disease” Genes Brain Behav2009 April; 8(3):337-345; Burkovetskaya et al., “Evidence for aberrant astrocyte hemichannel activity in Juvenile Neuronal Ceroid Lipofuscinosis (JNCL)” PLoS One2014 Apr. 15; 9(4):e95023; and Sondhi et al., “Partial correction of the CNS lysosomal storage defect in a mouse model of juvenile neuronal ceroid lipofuscinosis by neonatal CNS administration of an adeno-associated virus serotype rh.10 vector expressing the human CLN3 gene” Hum Gene Ther2014 March; 25(3):223-239.

### 4. Amyotrophic Lateral Sclerosis (ALS)

In one embodiment, the present invention contemplates a method of treating ALS with Nme2Cas9 base editing of a SOD1 mutation.

ALS is a neurodegenerative disease in which loss of motor neurons results in progressive muscle weakness, paralysis, and death, typically within 2-5 years of onset. Only two FDA-approved drugs are available, with modest delays (˜three months) in ALS progression. There is an unmet clinical need to develop treatments for ALS. Approximately 90% of ALS cases are sporadic and 10% of cases are familial. Mutations in the free-radical scavenger gene SOD1 (Cu—Zn superoxide dismutase 1) are the second most common genetic cause of ALS. ALS-associated, dominant SOD1 mutations destabilize the protein, causing aberrant misfolding and aggregation that likely contribute to cell death. A SOD1G93A (a gain-of-function mutation) transgenic mouse model exhibits motor neuron loss and a shortened lifespan (5 to 6 months). SOD1G37R mice carrying another toxic SOD1 mutation develop similar ALS symptoms. Sod1−/− knockout mice are normal and healthy, while in rare instances humans devoid of SOD1 exhibit neurodevelopmental defects. These findings point to the therapeutic potential of depleting mutant SOD1 to treat ALS in adults. Gurney et al., “Motor neuron degeneration in mice that express a human Cu, Zn superoxide dismutase mutation” Science 1994 Jun. 17; 264(5166):1772-1775; Wong et al., “An adverse property of a familial ALS-linked SOD1 mutation causes motor neuron disease characterized by vacuolar degeneration of mitochondria” Neuron 1995 June; 14(6):1105-1116; Taylor et al., “Decoding ALS: from genes to mechanism” Nature 2016 Nov. 10; 539(7628):197-206; Liu et al., “Toxicity of familial ALS-linked SOD1 mutants from selective recruitment to spinal mitochondria” Neuron 2004 Jul. 8; 43(1):5-17; Park et al., “SOD1 deficiency: a novel syndrome distinct from amyotrophic lateral sclerosis” Brain 2019 Aug. 1; 142(8):2230-2237; and Ezer et al., “Infantile SOD1 deficiency syndrome caused by a homozygous SOD1 variant with absence of enzyme activity” Brain 2022 Apr. 29; 145(3):872-878. Previous studies demonstrated that repressing SOD1 by RNAi ameliorates ALS in animal models and increases survival, and potential clinical benefit of SOD1 suppression has been reported in humans. Borel et al., “Therapeutic rAAVrh10 Mediated SOD1 Silencing in Adult SOD1(G93A) Mice and Nonhuman Primates” Hum Gene Ther 2016 January; 27(1):19-31; Miller et al., “Phase 1-2 Trial of Antisense Oligonucleotide Tofersen for SOD1 ALS” N Engl J Med 2020 Jul. 9; 383(2):109-119; and Mueller et al., “SOD1 Suppression with Adeno-Associated Virus and MicroRNA in Familial ALS. N Engl J Med 2020 Jul. 9; 383(2):151-158.

A Cas9 strategy for targeting SOD1 is appealing because of the ability to cause permanent genetic alteration, eliminating the need for repeated dosing. It has been shown that AAV9 delivery of Cas9 and guide RNA in vivo can deplete mutant SOD1 and prolong survival in SOD1G93A mice, though DSB-induced editing efficiencies were very low in these studies. Lee et al., “Imaging Net Retrograde Axonal Transport In Vivo: A Physiological Biomarker” Ann Neurol. 2022 May; 91(5):716-729; and Gaj et al., “In vivo genome editing improves motor function and extends survival in a mouse model of ALS” Sci Adv 2017 December; 3(12):eaar3952.

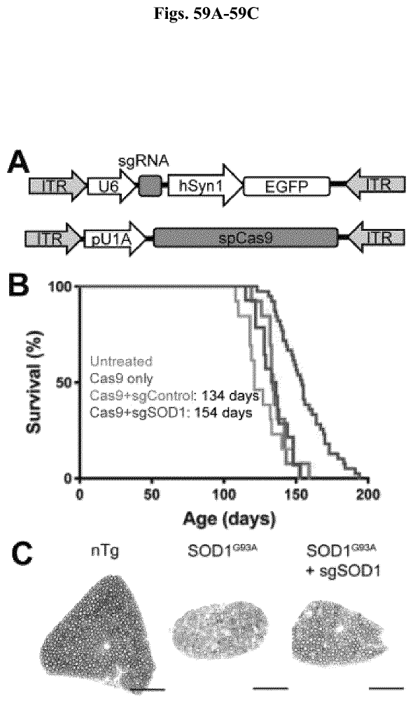

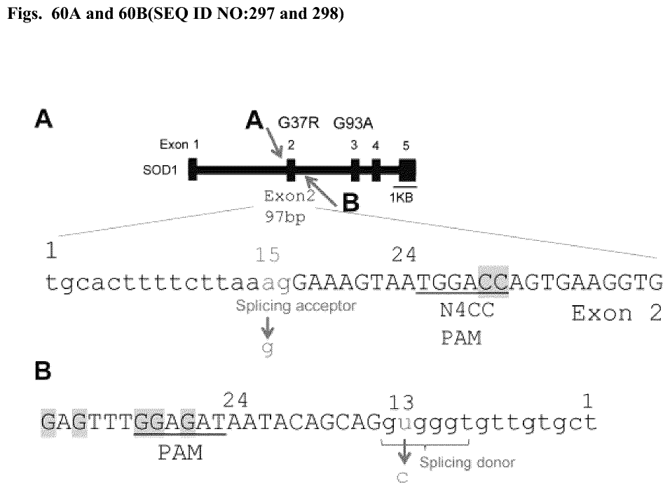

In one embodiment, the present invention contemplates a method comprising an inlaid domain Nm32Cas9-ABE to perform SOD1 gene editing. Previous data show that AAV-Cas9 treatment prolongs survival in SOD1G93A mice with a SOD1 exonic sgRNA targeting both WT and G93A human alleles. Consequently, two AAV9 vectors, clinically validated for motor neuron transduction were generated: i) AAV9.sgSOD1 expressing a guide RNA, and ii) AAV9.Cas9 expressing SpyCas9. See, FIG. 59A; and Mendell et al., “Single-Dose Gene-Replacement Therapy for Spinal Muscular Atrophy” N Engl J Med 2017 Nov. 2; 377(18). In HEK293T cells, dual plasmid backbone transfections reduced SOD1 protein levels. In addition, sgSOD1+Cas9 mice showed significant improvement in rotarod performance. Lee et al., “Imaging Net Retrograde Axonal Transport In Vivo: A Physiological Biomarker” Ann Neurol 2022 May; 91(5):716-729. To test editing in vivo, the dual AAV9 to transgenic SOD1G93A were delivered to mouse P1 neonates via ICV injections. The mice were monitored throughout their lifespan for ALS onset and symptoms. Compared to all control groups, AAV9 sgSOD1+Cas9 mice exhibited increased survival. See, FIG. 59B. To determine if sgSOD1 preserves axonal architecture of the ventral spinal root in ALS mice, L5 dorsal root axon cross sections were analyzed. sgSOD1 mice had significantly more intact axons than sgControl as measured by distributions of axon area. See, FIG. 59C. Together, these data indicate that dual AAV-Cas9 treatment has therapeutic benefits for ALS conditions.

In one embodiment, the present invention contemplates a method to induce SOD1 exon 2 skipping to treat ALS. Both N₄CC and N₄CD PAMs can be considered for disrupting splice sites and inducing SOD1 exon 2 skipping. Exon 2 skipping leads to a frameshift and loss of SOD1 function leading to at least two SOD1 mutations, e.g., G37R and G93A. An Nme2-ABE sgRNA targeting the intron 1 splice acceptor has been identified. See, FIG. 60A. For Nme2Cas9-ABEs, the A15 position of the protospacer (e.g., target A) is the “AG” splice acceptor and targets a dual-C PAM. For Nme2Cas9^{Smu}-ABEs, the A14 position (e.g., target B) of the protospacer is the “AG” splice acceptor and targets a single-C PAM. Similarly, the intron 2 splicing donor is targeted, including single-G (C on the opposite strand) PAMs. See, FIG. 60B.

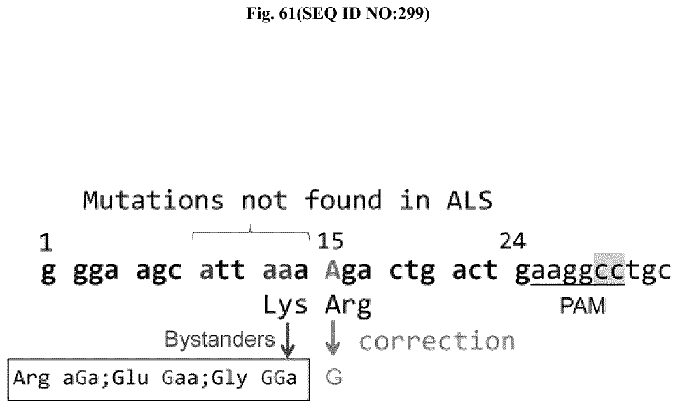

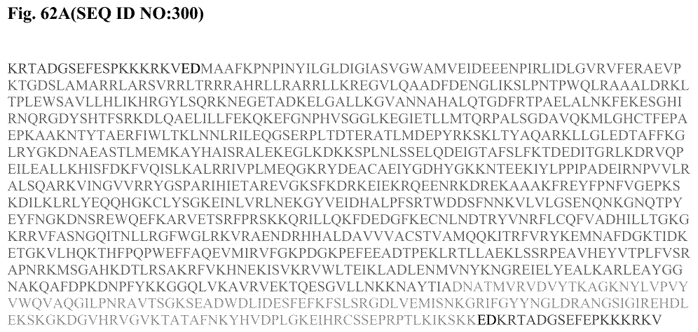

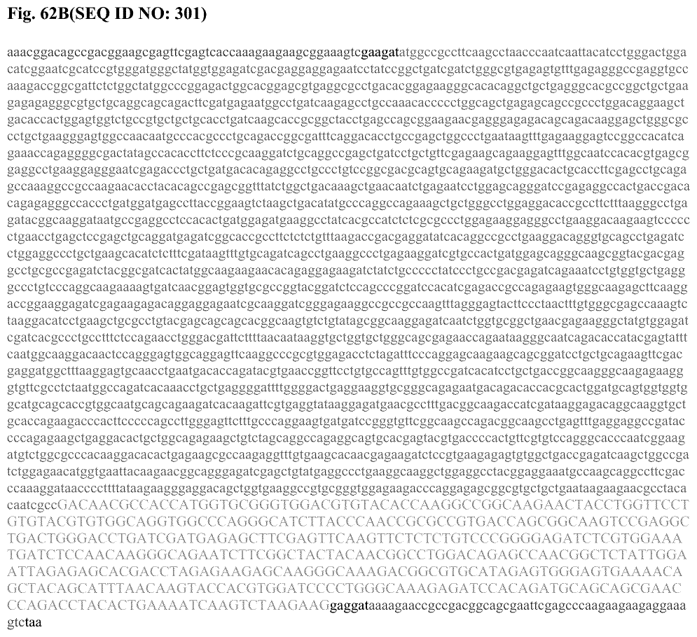

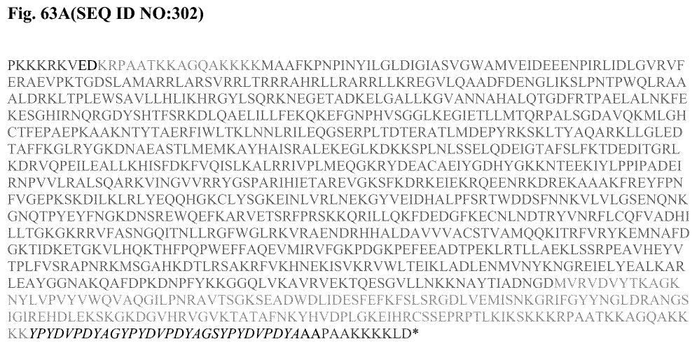

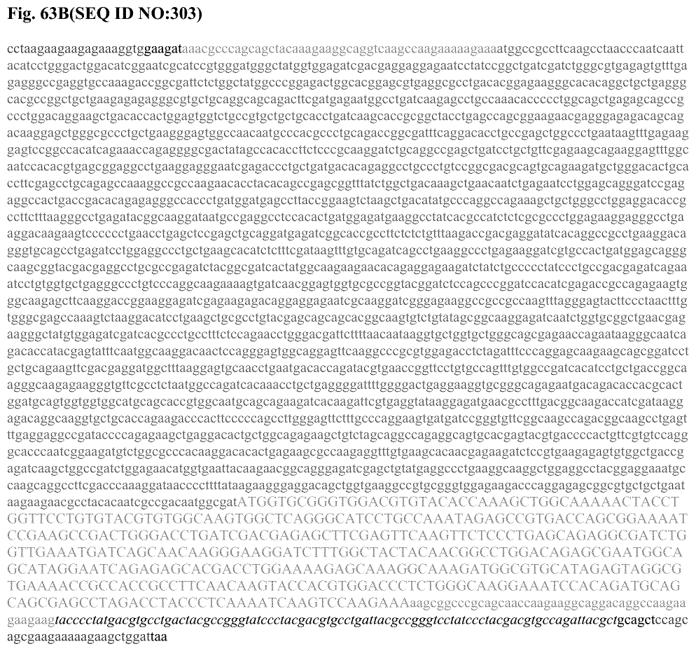

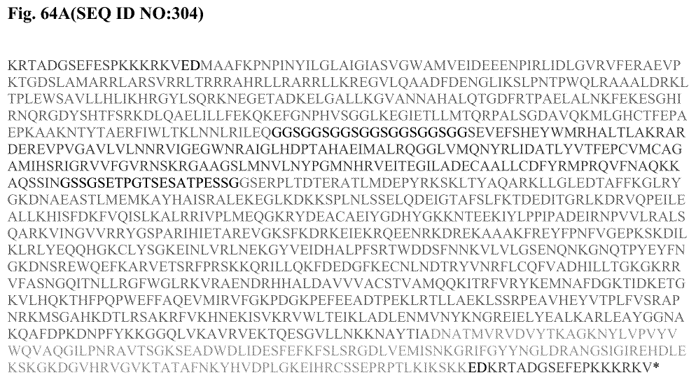

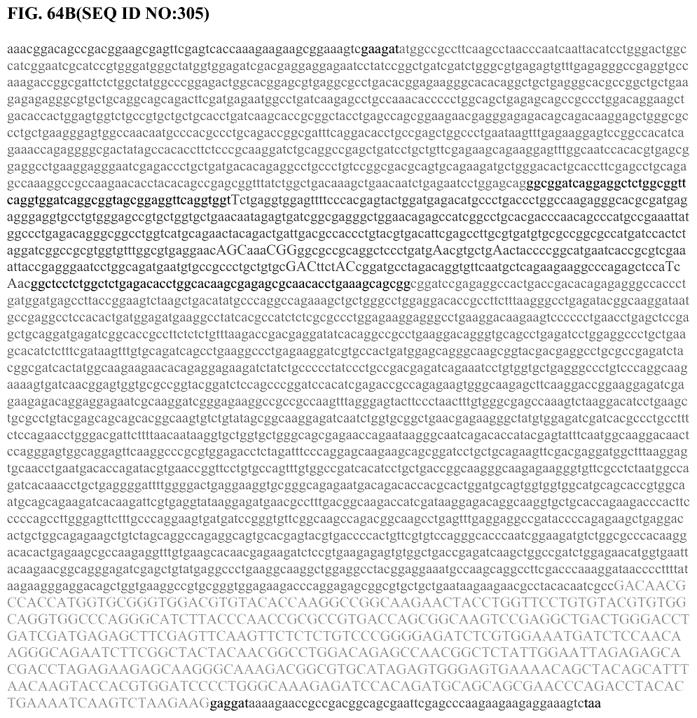

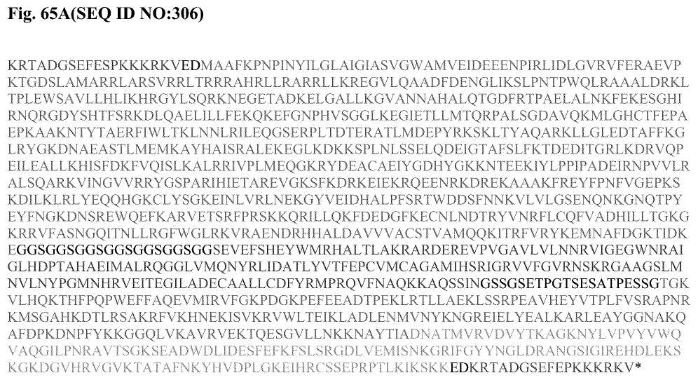

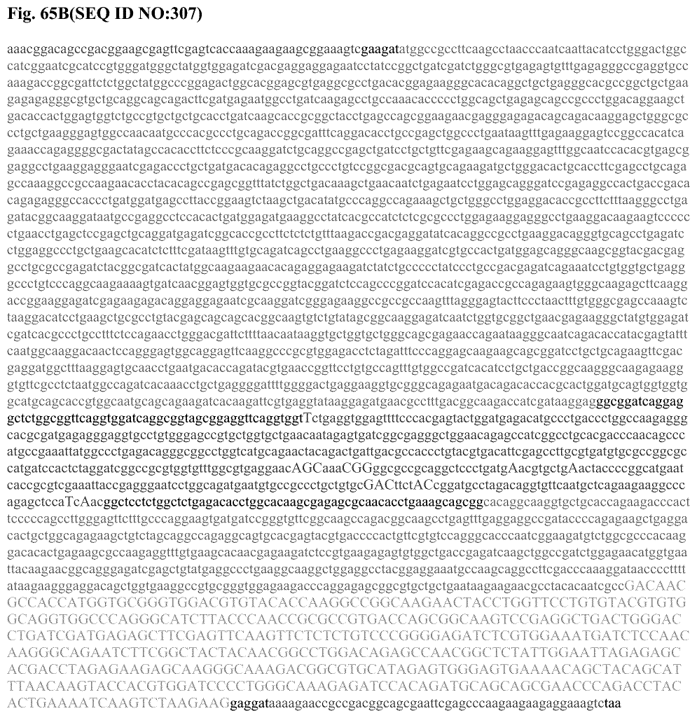

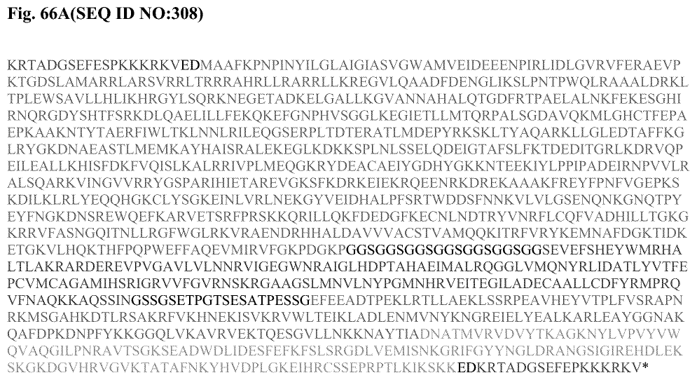

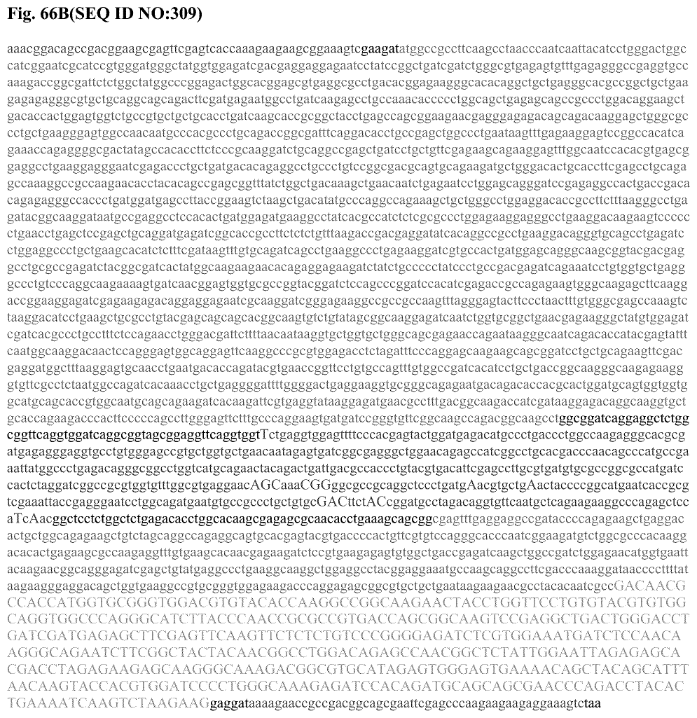

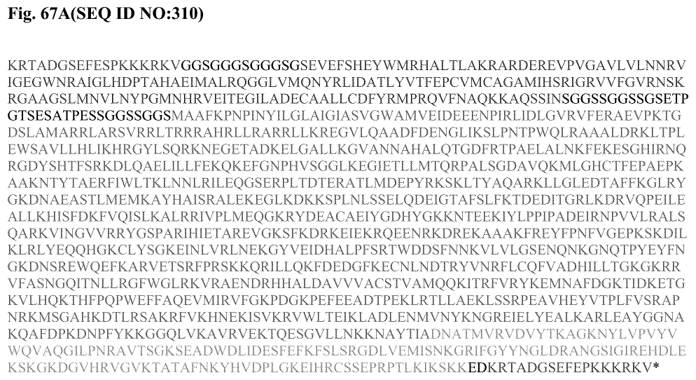

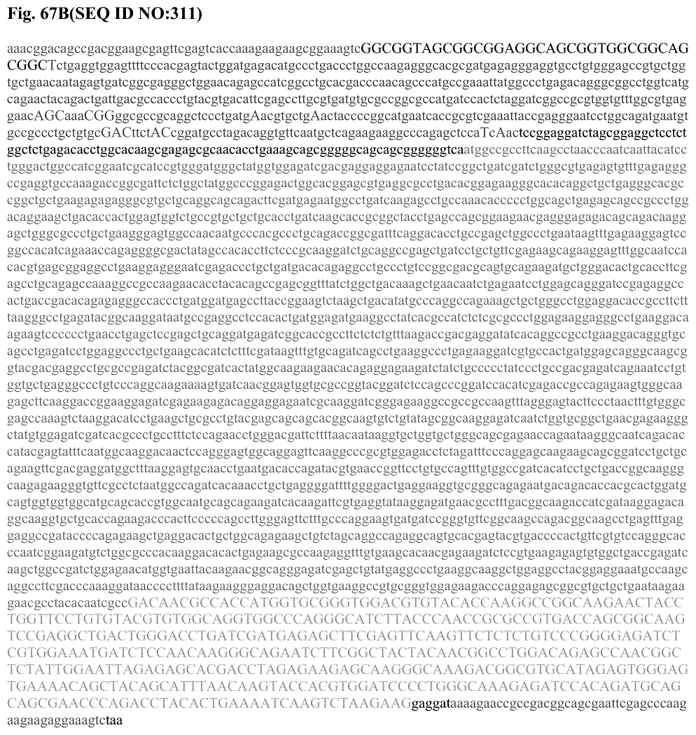

In one embodiment, the present invention contemplates an inlaid domain Nme2Cas9-ABE to correct a mutated SOD1 G37R allele and treat ALS symptoms, Although splice site disruption and exon 2 skipping has potential therapeutic benefit, base editors are more efficient for correction of disease-causing SNPs, such as the common SOD1^{G37R} G-to-A mutation. 17; and FIG. 61. Several missense mutations may occur in the SOD1. See, FIG. 61 (blue nts). While it is not yet known if mutations in the residues cause ALS, their tolerance for mutation (though possible) cannot be assumed. There are three PAMs (one N₄CC and two N₄CD) within this loci that could be targeted with inlaid Nme2-ABEs and Nme2^{Smu}-ABEs, respectively. The N₄CC PAM places the target at A15 (red).

### C. AAV Nme2Cas9 Inlaid Domain Constructs

Inlaid domain Nme2Cas9-ABE constructs are shown herein to be compatible with an in vivo single-AAV delivery platform. The all-in-one AAV vector for N-terminal Nme2-ABE was validated with the U1a and U6 promoters driving effector and sgRNA expression (respectively), is 4998 bp including the ITRs. Two analogous AAV9 versions (both 4996 nts) were generated with an inlaid deaminase domain at the i1 site: a first construct comprised the TadA8e domain, while the second construct comprised a TadA8e^{V106W} mutant that greatly reduces unintended A-to-G conversion in RNA molecules while minimally affecting on-target DNA deamination activity 69,70. All three vectors were targeted a common site in Rosa26 and administered via tail vein injection of AAV9 at 4×10¹¹ vg (vector genomes) in adult mice.

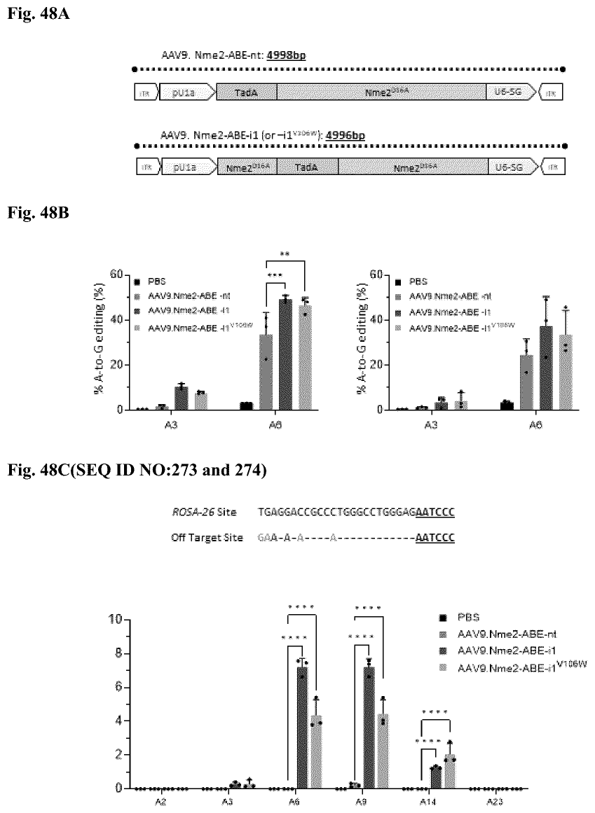

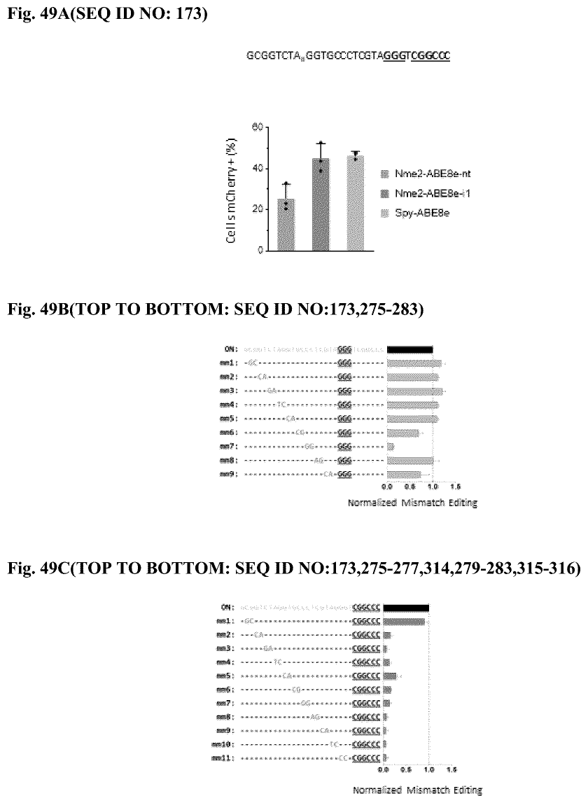

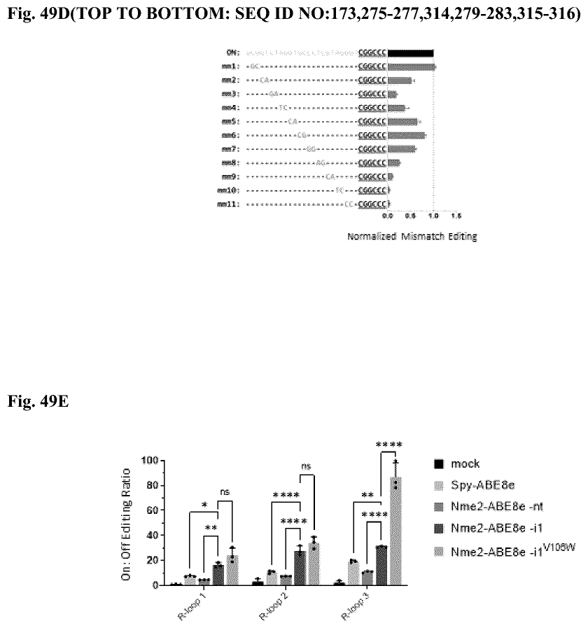

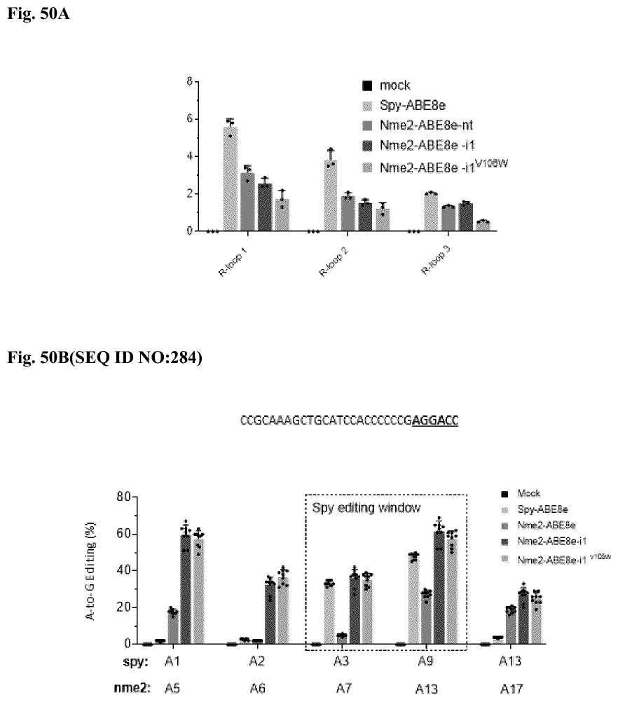

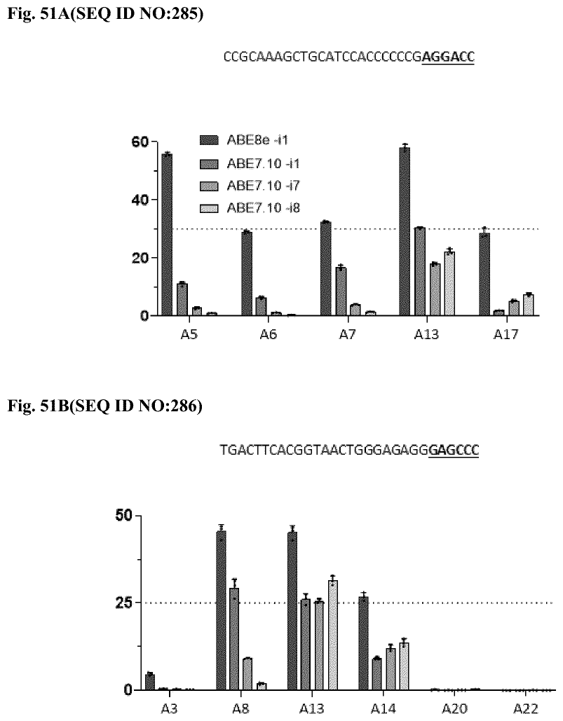

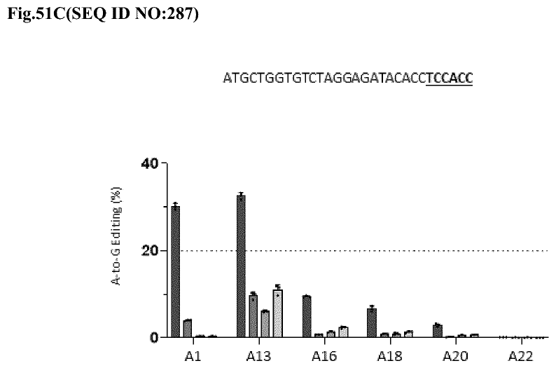

The data presented herein demonstrates significant adenine editing activity at Rosa26 mutations with the editing with AAV9 integration the Nme2Cas9-ABE-nt, i1 and i1^{V106W} constructs as compared to a physiological buffer saline (PBS) control. See, FIGS. 48A-48C. These results demonstrate the in vivo efficacy of single-AAV delivery systems for enhanced-efficiency, domain-inlaid Nme2-ABEs.

### D. Inlaid Cytosine Base Editors (CBEs)

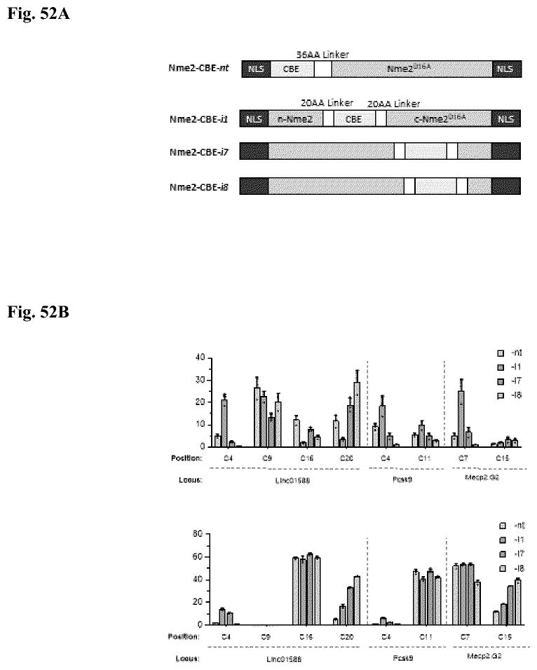

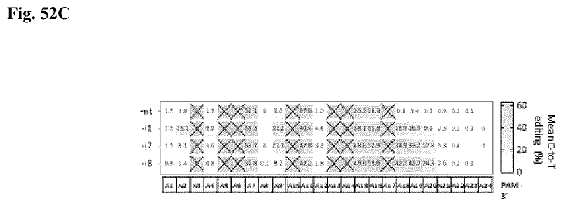

In one embodiment, the present invention contemplates an Nme2Cas9-CBE construct. In one embodiment, the Nme2Cas9-CBE construct comprises an N-terminal CBE. In one embodiment, the Nme2Cas9-CBE construct comprises an inlaid domain CBE. In one embodiment, the inlaid domain includes, but is not limited to Nme2Cas9-CBE-(i1), Nme2Cas9-CBE-(i7) and Nme2Cas9-CBE-(i8). In one embodiment, the CBE is a cytidine deaminase. In one embodiment, the cytidine deaminase includes, but is not limited to, evoFERNY or rAPOBEC1. See, FIG. 52A.

The data presented herein shows that inlaid domain Nme2Cas9-CBE constructs have cytidine editing activity that is either comparable to, or superior to, the N-terminal domain construct. See, FIG. 52B-52C.

### V. Inter-Cas9 Protospacer Interacting Domain (PID) Swapping

The therapeutic promise of base editing systems is believed to hinge upon improvements in editing efficiency, limiting bystander edits (or their consequences), maximizing PAM-dependent targeting scope, and minimizing immunogenicity, toxicity, and prolonged deaminase expression (which can compromise editing efficiency and lead to safety risks such as hepatotoxicity and the accumulation of unwanted edits). Advances to overcome these issues are encompassed by the presently discloses chimeric Cas9 nucleases which encompass a cross-species PID. It is believed that these improved base editing constructs have increased effectiveness, targeting scope, utility, and safety.

Gene editing using CRISPR-Cas9 technologies has advanced genetic research and promises to revolutionize gene therapy. For efficient editing to occur, a Cas9 recognizes a sequence motif, called a protospacer adjacent motif (PAM), adjacent to the target site. Different Cas9 homologs have distinct PAM sequences; most are 2-5 nucleotides long. The shorter the PAM, the less restrictive the PAM sequence requirement for editing, and the higher the density of candidate target sites. One example discussed above, is a compact Type II-C Cas9 ortholog from Neisseria meningitidis (Nme2Cas9). Nme2Cas9 exhibits a unique DNA targeting motif (PAM of N₄CC), high accuracy, and the ability to mediate efficient ex vivo and in vivo gene editing in mammalian cells. Edraki et al., Mol Cell (2019) “A Compact, High-Accuracy Cas9 with a Dinucleotide PAM for In Vivo Genome Editing”.

Also as disclosed herein, base editors (BEs) can be fused to Nme2Cas9 either as an N-terminal or inlaid domain. Zhang et al., GEN Biotechnology (2022) “Adenine Base Editing In Vivo with a Single Adeno-Associated Virus Vector”. Base editors include, but are not limited to adenine base editors (ABEs) or cytidine base editors (CBEs) facilitated by Nme2Cas9 fusion with an adenosine deaminase (ABE8e) or a cytosine deaminase. Richter et al., Nature Biotechnology (2020) “Phage-assisted evolution of an adenine base editor with improved Cas domain compatibility and activity”; and Chatterjee et al. Nature Commun. (2020) “A Cas9 with PAM recognition for adenine dinucleotides”. These Nme2Cas9-ABE/CBE constructs comprise a wild-type PAM-interacting domain (PID) that recognizes a CC dinucleotide. For example if a potential editing site is not positioned an appropriate distance from a CC dinucleotide, these Nme2Cas9 base editors are unable to bind and cleave at that site.

In one embodiment, the present invention contemplates an Nme2_Cas9-ABE?CBE that has undergone a protospacer interacting domain (PID)-swapping that alters the PAM specificity of the Nme2Cas9 protein. In one embodiment, a PID is removed from a first Cas9 nuclease and inserted into a second Cas9 nuclease. Closely related type II-C Cas9 orthologs have been reported to recognize diverse PAMs. doi.org/10.7554/eLife.77825.

The general concept of PID-swapping was demonstrated between closely related Neisseria meningitidis orthologues. (Edraki et al., 2019). Distantly related Cas9 orthologues were also shown to be tolerant of PID swapping. Hu et al. Nucleic Acids Res. (2021) “Discovery and engineering of small SlugCas9 with broad targeting range and high specificity and activity”; and Schmidt et al. The SmuCas9 PAM (N₄C) of SmuCas9 was characterized using in vitro cleavage assays. Lee et al., mBio (2018) Potent Cas9 Inhibition in Bacterial and Human Cells by AcrIIC4 and AcrIIC5 Anti-CRISPR Proteins. More recently, SmuCas9's N₄C PAM was confirmed using a cell-based assay and an Nsp2Cas9-SmuCas9 PID hybrid nuclease was developed. An Nme2Cas9-SmuCas9 PID hybrid nuclease was also reported. Wei et al., eLife (2022) 11, e77825.

In one embodiment, the present invention contemplates a chimeric Nme2Cas9 fusion protein comprising an SmuCas9 protospacer interacting domain (PID) and a nucleotide deaminase protein. In one embodiment, the nucleotide deaminase protein is an adenosine deaminase. In one embodiment, the adenosine deaminase is ABE8e. In one embodiment, the nucleotide deaminase protein is a cytosine deaminase. (Huang et al., Nature Biotechnology 2022; dx.doi.org/10.1038/s41587-022-01410-2. In one embodiment, the nucleotide deaminase protein is an N-terminal domain of the chimeric Nme2^{Smu}Cas9 fusion protein. In one embodiment, the nucleotide deaminase protein is an inlaid domain of the chimeric Nme2^{Smu}Cas9 fusions protein. In one embodiment, the SmuCas9 PID replaces a wild type Nme2Cas9 PID. Although it is not necessary to understand the mechanism of the invention, it is believed that Nme2^{Smu}Cas9 chimeric proteins as disclosed herein have a predicted DNA targeting motif (e.g., an N₄C PAM) and can mediate gene editing with N₄C PAM-targeting guide RNAs.

In one embodiment, the present invention contemplates a composition comprising a chimeric Nme2^{Smu}Cas9-ABE8e fusion proteins and an sgRNA. In one embodiment, the sgRNA targets a human MeCP2 gene mutation. In one embodiment, the MeCP2 gene causes Rett syndrome.

In one embodiment, the present invention contemplates a method of treating Rett syndrome comprising administering to a patient a composition comprising a chimeric Nme2^{Smu}Cas9-ABE8e fusion protein and an sgRNA, wherein the sgRNA targets an MeCP2 gene mutation. In one embodiment, the composition converts the MeCP2 gene mutation into a wild type sequence. For example, MeCP2 gene mutation-directed sgRNAs were administered with a chimeric Nme2^{Smu}Cas9-ABE83 fusion protein to a HEK293T cell line (Rett-PiggyBac) and/or Rett patient derived fibroblast cells (Rett-PDFs) harboring pathogenic MeCP2 mutant alleles including, but not limited to: i) c.502 C>T; p.R168X with four (4) sgRNAs; ii) c.763 C>T; p.R255X with two (2) sgRNAs; iii) c. 808 C>T; p.R270X with two (2) sgRNAs; and/or iv) c.916 C>T; p.R306C with two (2) sgRNAs.

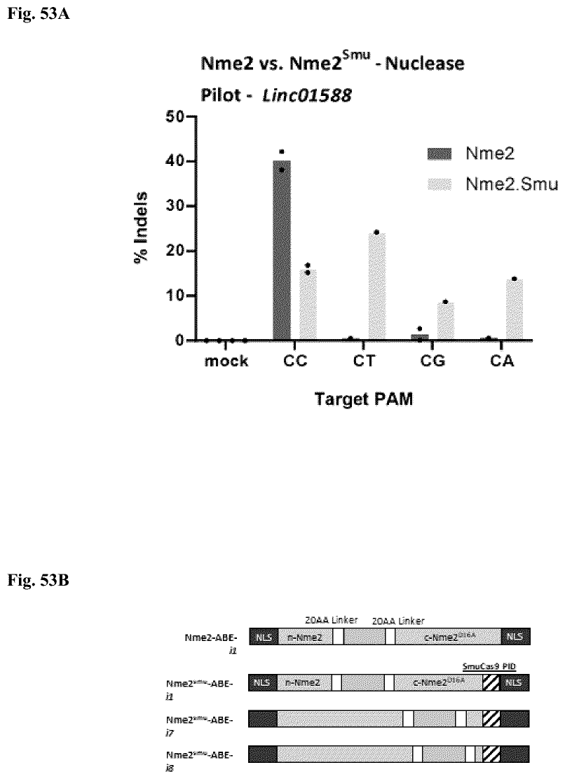

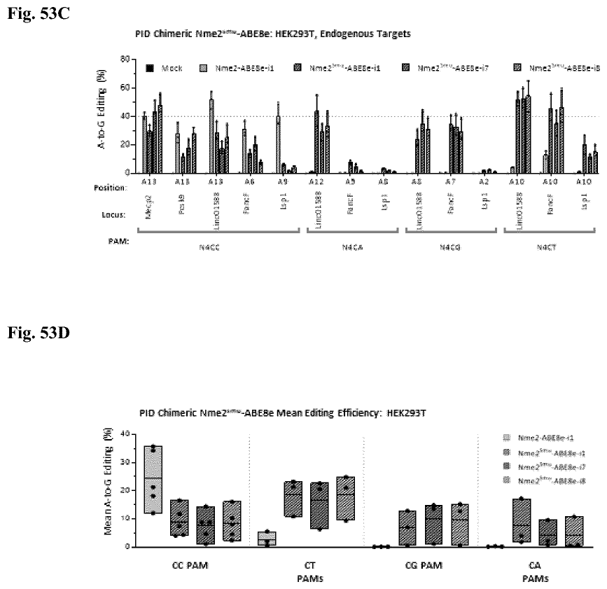

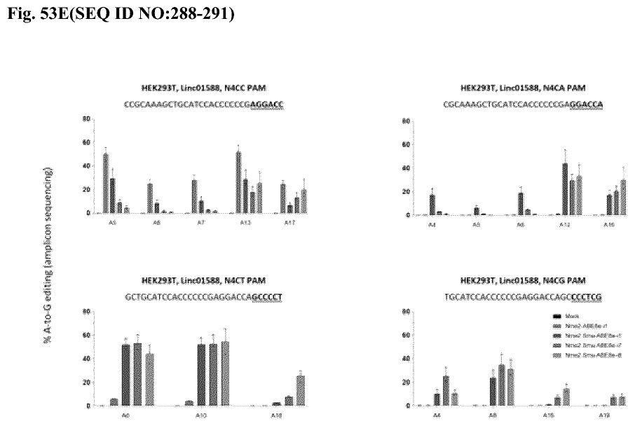

Although it is not necessary to understand the mechanism of an invention, it is believed that PID chimeric Nme2Cas9 base editors comprising a cross-species (e.g., exogenous) PID have an expanded targeting scope as compared to an Nme2Cas9 base editor with a wild type (e.g., endogenous) PID. See, FIG. 53A. For example, several chimeric Nme2Cas9^{Smu} constructs were created that also contain inlaid domains of a nucleotide base editor. See, FIG. 53B. These Nme2Cas9^{Smu} constructs were shown to have significant base editing activity and efficiency. See, FIG. 53C and FIG. 53D. Gene editing activity was observed the at Linc01588 endogenous HEK293T genomic loci targeted to N₄CN PAMs. See, FIG. 53D.

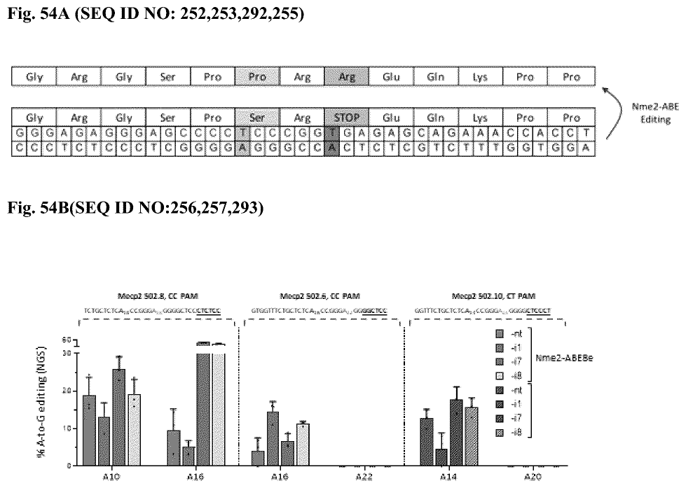

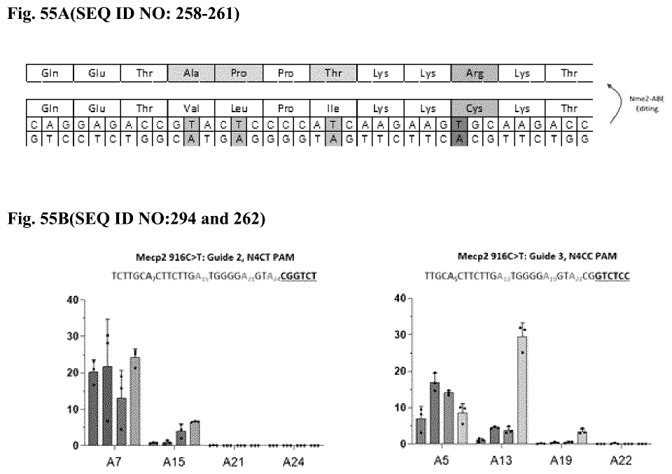

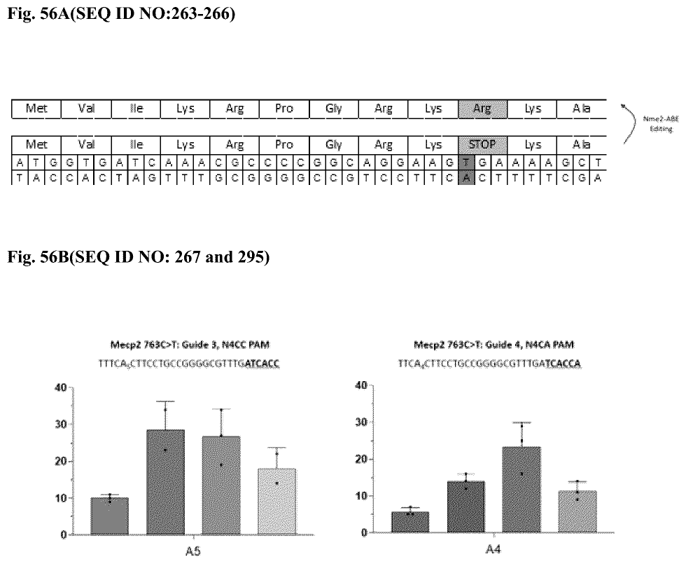

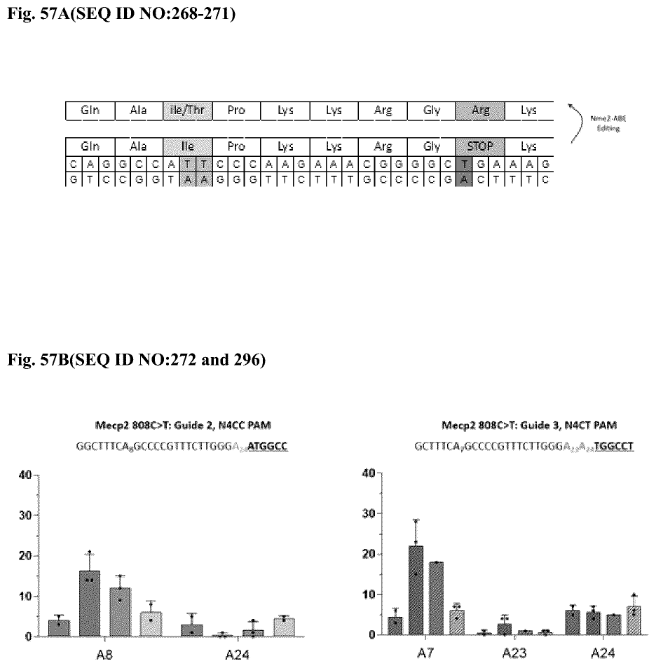

Conversion of the Rett syndrome mutations were observed with the chimeric Nme2Cas9^{Smu} constructs: i) c.502 C>T (RETT-PDF); See, FIGS. 54A and 54B; ii) c.916 C>T (RETT-PDF); See, FIGS. 55A and 55B; iii) c.763C>T (RETT-PiggyBac); See, FIGS. 56A and 56B; and iv) c.808C>T (RETT-PiggyBac); See, FIGS. 57A and 57B.

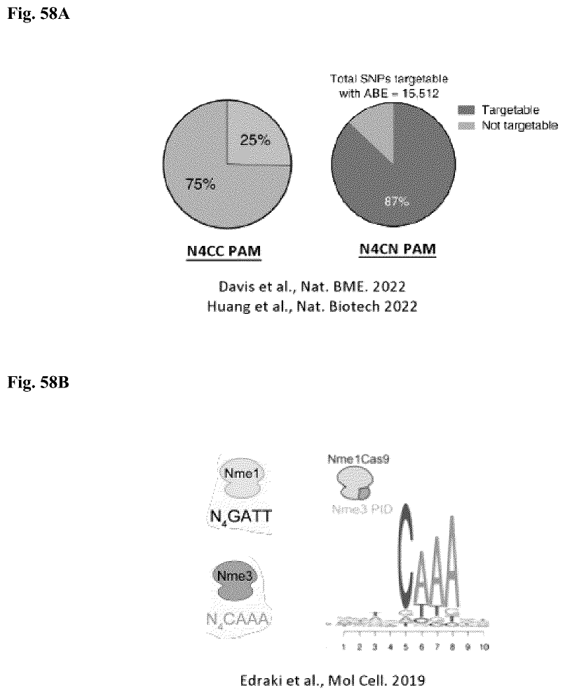

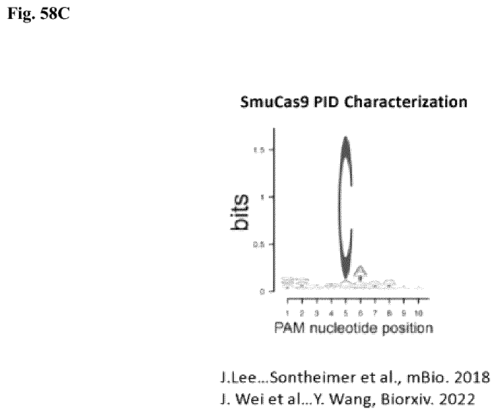

Although it is not necessary to understand the mechanism of an invention, it is believed that an expanded PAM scope for chimeric Cas9^{Smu} nucleases concomitantly increase the number of candidate targets as compared to a wild type Cas9 nuclease. See, FIG. 58A. It has been reported that PID replacement (e.g., swap) concomitantly changes the Cas9 nuclease PAM preference. See, FIG. 58B. It has been reported that the SmuCas9 PID does interact with a PAM comprising a single C nucleotide. See, FIG. 58C.

It is believed that no compact base editing platforms with single-nt PAMs have been previously reported. SpyCas9 and its BEs have been engineered to SpyCas9-NG, SpRY, and other versions with reduced PAM requirements that (on average) enable targeting every 2-4 nts. Nonetheless, size and off-targeting remain issues with those platforms. SmuCas9 was identified with an apparent single-nt (N4C) PAM requirement, but its native tracrRNA sequence has not been available, and its activity with tracrRNA sequences from related Cas9s was poor. Lee et al., “Potent Cas9 Inhibition in Bacterial and Human Cells by AcrIIC4 and AcrIIC5 Anti-CRISPR Proteins” 2018 Dec. 4; 9(6):e02321-18. However, further exploration of this single-nt PAM capability to fully exploit its usefulness is necessary, as described below. For example, PAM minimization not only allows more sites to be accessed but can also enable target nts to be edited by multiple guides. This can allow the definition of a guide register within the local sequence that avoids bystander editing.

### A. A Nme2Cas9 Nuclease With A Single-Cytidine PAM Compatibility

Nme2Cas9 has been found to be effective at N₄CC PAMs across a broad editing window (nts ˜2 to ˜19 of the 24 nt protospacer), with maximal activity between nts 6-17. Zhang et al., “Adenine Base Editing In Vivo with a Single Adeno-Associated Virus Vector” GEN Biotechnol 2022 Jun. 1; 1(3):285-299; and Davis et al., “Efficient in vivo base editing via single adeno-associated viruses with size-optimized genomes encoding compact adenine base editors” Nat Biomed Eng. 2022 Jul. 28; 1-12.

A relatively wide editing window imparts a trade-off: more editing sites can be accessed, but bystander editing can also be exacerbated. Anzalone et al., “Genome editing with CRISPR-Cas nucleases, base editors, transposases and prime editors” Nat Biotechnol 2020 July; 38(7):824-844; and Rees et al., “Base editing: precision chemistry on the genome and transcriptome of living cells” Nat Rev Genet 2018 December; 19(12):770-788. The ability to control Nme2-ABE bystander editing would be very useful for therapeutic and other applications. Finally, although the N₄CC PAM enables Nme2-ABE to access target sites that other AAV-validated compact Cas9s cannot, targeting range improvement via PAM minimization would be tremendously beneficial, including for minimizing bystander editing. The data presented herein address these three areas—efficiency improvement, bystander modulation, and PAM minimization—and indicate that all are achievable.

In one embodiment, the present invention contemplates Nme2-ABEs with an inserted SmuCas9 PAM-interacting domain (PID) that replaces the wild type Nme2 PID and enables editing of sites via N₄C PAMs. An in vitro analyses showed that SmuCas9 has strong PAM preference of a single cytidine residue. Lee et al., “Tissue-restricted genome editing in vivo specified by microRNA-repressible anti-CRISPR proteins” RNA 2019 November; 25(11):1421-1431. Further, PIDs from other Cas9 homologs, besides Smu can be transplanted into Nme1Cas9 (98% identical to Nme2Cas9 outside of the PID) to functionally reprogram PAM requirements. Edraki et al., “A Compact, High-Accuracy Cas9 with a Dinucleotide PAM for In Vivo Genome Editing” Mol Cell 2019 Feb. 21; 73(4):714-726.e4.

Results confirm that inserting a SmuCas9 PID into Nme2Cas9 reduces its PAM requirement from two cytidines to one cytidine. While Nme2-ABEs are largely inactive at N₄CA, N₄CG, and N₄CT PAMs, the i1, i7, and i8 inlaid Nme2Smu-ABEs efficiently edit many such sites. FIG. 53C. These results confirm that Nme2-ABEs remain the platforms of choice at N4CC PAM sites, but that Nme2Smu-ABEs can expand the range of targetable sites to N4CD (D=not C) PAMs.

### B. A Single-AAV Nme2Cas9^{Smu}-ABE Construct

As shown above, the Nme2^{Smu}-ABE construct is functionally validated, but the SmuCas9 PID is 8 aa larger than the native Nme2Cas9 PID, increasing vector size by 24 nts (5,020 nts total). It is possible that even a modest 24 nt increase in cargo size might compromise packaging efficiency and integrity as well as delivery efficiency.

In one embodiment, the single AAV Nme2Cas9^{Smu}-ABE construct comprises an EFS promoter (212 bp). Data has been reported the EFS promoter is effective in driving expression from both Nme2-ABE and Nme2Smu-ABE vectors (4,957 and 4,981 nts, respectively). Davis et al., “Efficient in vivo base editing via single adeno-associated viruses with size-optimized genomes encoding compact adenine base editors” Nat Biomed Eng 2022 Jul. 28; 1-12.

In one embodiment, an all-in-one AAV9 construct includes, but is not limited to, Nme2^{Smu}-ABE [-i1(V106W), −i7(V106W), and −i8(V106W)] that target mouse N₄C PAM sites and measure editing activity in multiple tissues in vivo.

### VI. Cas9 Therapeutic Plasmid/Vector Constructs

Although compact Cas9 orthologs have been previously validated for genome editing, including via single-AAV delivery, their longer PAMs have restricted therapeutic development due to target site frequencies that are lower than that of the more widely adopted SpyCas9. In addition, SauCas9 and its KKH variant with relaxed PAM requirements (Kleinstiver et al., 2015) are prone to off-target editing with some sgRNAs (Friedland et al., 2015; Kleinstiver et al., 2015). These limitations are exacerbated with target loci that require editing within a narrow sequence window, or that require precise segmental deletion.

As described above, Nme2Cas9 has been identified as a compact and highly accurate Cas9 with a less restrictive dinucleotide PAM for genome editing by AAV delivery in vivo. The development of Nme2Cas9 greatly expands the genomic scope of in vivo editing, especially via viral vector delivery. The Nme2Cas9 all-in-one AAV delivery platform, can in principle, be used to target as wide a range of sites as SpyCas9 (due to the identical densities of optimal N₄CC and NGG PAMs), but without the need to deliver two separate vectors to the same target cells. The availability of a catalytically dead version of Nme2Cas9 (dNme2Cas9) also promises to expand the scope of applications such as CRISPRi, CRISPRa, base editing, and related approaches (Dominguez et al., 2016; Komor et al., 2017). Moreover, Nme2Cas9's hyper-accuracy enables precise editing of target genes, potentially ameliorating safety issues resulting from off-target activities. Perhaps counterintuitively, the higher target site density of Nme2Cas9 (compared to that of Nme1Cas9) does not lead to a relative increase in off-target editing for the former. Similar results have been reported recently with SpyCas9 variants evolved to have shorter PAMs (Hu et al., 2018). Type II-C Cas9 orthologs are generally slower nucleases in vitro than SpyCas9 (Ma et al., 2015; Mir et al., 2018); interestingly, enzymological principles indicate that a reduced apparent k_{cat} (within limits) can improve on-vs. off-target specificity for RNA-guided nucleases (Bisaria et al., 2017).

The discovery of Nme2Cas9 and Nme3Cas9 hinged on unexplored Cas9s that are highly related (outside of the PID) to an ortholog that was previously validated for human genome editing (Esvelt et al., 2013; Hou et al., 2013; Lee et al., 2016; Amrani et al., 2018). The relatedness of Nme2Cas9 and Nme3Cas9 to Nme1Cas9 brought an added benefit, namely that they use the exact same sgRNA scaffold, circumventing the need to identify and validate functional tracrRNA sequences for each. In the context of natural CRISPR immunity, the accelerated evolution of novel PAM specificities could reflect selective pressure to restore targeting of phages and MGEs that have escaped interference through PAM mutations (Deveau et al., 2008; Paez-Espino et al., 2015). Representative examples of plasmids/vectors and associated sequences that are compatible with NmeCas9 constructs are shown below. See, Table 5 and Table 6.

TABLE 5
Exemplary Plasmids For NmeCas9 Constructs

| Plasmid | | Insert | | | | SEQ |
|---|---|---|---|---|---|---|
| # | Name | description | Backbone | Purpose | Insert Sequence | ID NO |
| 1 | pAE70 | Nme3Cas9 | pMCSG7 | Bacterial expression | See examples herein. | |
| | | PID on | | of Nme1Cas9 with | | |
| | | Nme1Cas9 | | Nme3Cas9 PID | | |
| 2 | pAE71 | Nme2Cas9 | pMCSG7 | Bacterial expression | See examples herein. | |
| | | PID on | | of Nme1Cas9 with | | |
| | | Nme1Cas9 | | Nme2Cas9 PID | | |
| 3 | pAE113 | Nme2TLR1 | pLKO | Targeting TLR2.0 | GTCACCTGCCTCGT | 70 |
| | | | | with Nme2Cas9 | GGAATACGG | |
| 4 | pAE114 | Nme2TLR2 | pLKO | Targeting TLR2.0 | GCACCTGCCTCGTG | 71 |
| | | | | with Nme2Cas9 | GAATACGGT | |
| 5 | pAE115 | Nme2TLR5 | pLKO | Targeting TLR2.0 | GTTCAGCGTGTCCG | 72 |
| | | | | with Nme2Cas9 | GCTTTGGC | |
| 6 | pAE116 | Nme2TLR11 | pLKO | Targeting TLR2.0 | GTGGTGAGCAAGG | 73 |
| | | | | with Nme2Cas9 | GCGAGGAGCTG | |
| 7 | pAE117 | Nme2TLR12 | pLKO | Targeting TLR2.0 | GGGCGAGGAGCTG | 74 |
| | | | | with Nme2Cas9 | TTCACCGGGGT | |
| 8 | pAE118 | Nme2TLR13 | pLKO | Targeting TLR2.0 | GTGAACTTGTGGCC | 75 |
| | | | | with Nme2Cas9 | GTTTACGTCG | |
| 9 | pAE119 | Nme2TLR14 | pLKO | Targeting TLR2.0 | GCGTCCAGCTCGAC | 76 |
| | | | | with Nme2Cas9 | CAGGATGGGC | |
| 10 | pAE120 | Nme2TLR15 | pLKO | Targeting TLR2.0 | GCGGTGAACAGCT | 77 |
| | | | | with Nme2Cas9 | CCTCGCCCTTG | |
| 11 | pAE121 | Nme2TLR16 | pLKO | Targeting TLR2.0 | GGGCACCACCCCG | 78 |
| | | | | with Nme2Cas9 | GTGAACAGCTC | |
| 12 | pAE122 | Nme2TLR17 | pLKO | Targeting TLR2.0 | GGCACCACCCCGGT | 79 |
| | | | | with Nme2Cas9 | GAACAGCTCC | |
| 13 | pAE123 | Nme2TLR18 | pLKO | Targeting TLR2.0 | GGGATGGGCACCA | 80 |
| | | | | with Nme2Cas9 | CCCCGGTGAAC | |
| 14 | pAE124 | Nme2TLR19 | pLKO | Targeting TLR2.0 | GCGTGTCCGGCTTT | 81 |
| | | | | with Nme2Cas9 | GGCGAGACAA | |
| 15 | PAE125 | Nme2TLR20 | pLKO | Targeting TLR2.0 | GTCCGGCTTTGGCG | 82 |
| | | | | with Nme2Cas9 | AGACAAATCA | |
| 16 | pAE126 | Nme2TLR21 | pLKO | Targeting TLR2.0 | GATCACCTGCCTCG | 83 |
| | | | | with Nme2Cas9 | TGGAATACGG | |
| 17 | pAE149 | Nme2TLR22 | pLKO | Targeting TLR2.0 | GACGCTGAACTTGT | 84 |
| | | | | with Nme2Cas9 | GGCCGTTTAC | |
| 18 | pAE150 | Nme2TLR23 | pLKO | Targeting TLR2.0 | GCCAAAGCCGGAC | 85 |
| | | | | with Nme2Cas9 | ACGCTGAACTT | |
| 19 | PAE193 | Nme2TLR13 | pLKO | Targeting TLR2.0 | GGAACTTGTGGCCG | 86 |
| | | with 23 nt | | with Nme2Cas9 | TTTACGTCG | |
| | | spacer | | | | |
| 20 | pAE194 | Nme2TLR13 | pLKO | Targeting TLR2.0 | GAACTTGTGGCCGT | 87 |
| | | with 22 nt | | with Nme2Cas9 | TTACGTCG | |
| | | spacer | | | | |
| 21 | PAE195 | Nme2TLR13 | pLKO | Targeting TLR2.0 | GACTTGTGGCCGTT | 88 |
| | | with 21 nt | | with Nme2Cas9 | TACGTCG | |
| | | spacer | | | | |
| 22 | pAE196 | Nme2TLR13 | pLKO | Targeting TLR2.0 | GCTTGTGGCCGTTT | 89 |
| | | with 20 nt | | with Nme2Cas9 | ACGTCG | |
| | | spacer | | | | |
| 23 | pAE197 | Nme2TLR13 | pLKO | Targeting TLR2.0 | GTTGTGGCCGTTTA | 90 |
| | | with 19 nt | | with Nme2Cas9 | CGTCG | |
| | | spacer | | | | |
| 24 | pAE213 | Nme2TLR21 | pLKO | Targeting TLR2.0 | GTCACCTGCCTCGT | 70 |
| | | with G22 | | with Nme2Cas9 | GGAATACGG | |
| | | spacer | | | | |
| 25 | PAE214 | Nme2TLR21 | pLKO | Targeting TLR2.0 | GCACCTGCCTCGTG | 91 |
| | | with G21 | | with Nme2Cas9 | GAATACGG | |
| | | spacer | | | | |
| 26 | pAE215 | Nme2TLR21 | pLKO | Targeting TLR2.0 | GACCTGCCTCGTGG | 92 |
| | | with G20 | | with Nme2Cas9 | AATACGG | |
| | | spacer | | | | |
| 27 | pAE216 | Nme2TLR21 | pLKO | Targeting TLR2.0 | GCCTGCCTCGTGGA | 93 |
| | | with G19 | | with Nme2Cas9 | ATACGG | |
| | | spacer | | | | |
| 28 | pAE90 | Nme2TS1 | pLKO | Targeting AAVS1 | GGTTCTGGGTACTT | 3 |
| | | | | with Nme2Cas9 | TTATCTGTCC | |
| 29 | pAE93 | Nme2TS4 | pLKO | Targeting AAVS1 | GTCTGCCTAACAGG | 4 |
| | | | | with Nme2Cas9 | AGGTGGGGGT | |
| 30 | pAE94 | Nme2TS5 | pLKO | Targeting AAVS1 | GAATATCAGGAGA | 5 |
| | | | | with Nme2Cas9 | CTAGGAAGGAG | |
| 31 | pAE129 | Nme2TS6 | pLKO | Targeting LINC01588 | GCCTCCCTGCAGGG | 6 |
| | | | | with Nme2Cas9 | CTGCTCCC | |
| 32 | PAE130 | Nme2TS10 | pLKO | Targeting AAVS1 | GAGCTAGTCTTCTT | 7 |
| | | | | with Nme2Cas9 | CCTCCAACCC | |
| 33 | pAE131 | Nme2TS11 | pLKO | Targeting AAVS1 | GATCTGTCCCCTCC | 8 |
| | | | | with Nme2Cas9 | ACCCCACAGT | |
| 34 | pAE132 | Nme2TS12 | pLKO | Targeting AAVS1 | GGCCCAAATGAAA | 9 |
| | | | | with Nme2Cas9 | GGAGTGAGAGG | |
| 35 | pAE133 | Nme2TS13 | pLKO | Targeting AAVS1 | GCATCCTCTTGCTT | 10 |
| | | | | with Nme2Cas9 | TCTTTGCCTG | |
| 36 | pAE136 | Nme2TS16 | pLKO | Targeting LINC01588 | GGAGTCGCCAGAG | 11 |
| | | | | with Nme2Cas9 | GCCGGTGGTGG | |
| 37 | pAE137 | Nme2TS17 | pLKO | Targeting LINC01588 | GCCCAGCGGCCGG | 12 |
| | | | | with Nme2Cas9 | ATATCAGCTGC | |
| 38 | pAE138 | Nme2TS18 | pLKO | Targeting CYBB with | GGAAGGGAACATA | 13 |
| | | | | Nme2Cas9 | TTACTATTGC | |
| 39 | pAE139 | Nme2TS19 | pLKO | Targeting CYBB with | GTGGAGTGGCCTGC | 14 |
| | | | | Nme2Cas9 | TATCAGCTAC | |
| 40 | pAE140 | Nme2TS20 | pLKO | Targeting CYBB with | GAGGAAGGGAACA | 15 |
| | | | | Nme2Cas9 | TATTACTATTG | |
| 41 | pAE141 | Nme2TS21 | pLKO | Targeting CYBB with | GTGAATTCTCATCA | 16 |
| | | | | Nme2Cas9 | GCTAAAATGC | |
| 42 | pAE144 | Nme2TS25 | pLKO | Targeting VEGFA | GCTCACTCACCCAC | 17 |
| | | | | with Nme2Cas9 | ACAGACACAC | |
| 43 | pAE145 | Nme2TS26 | pLKO | Targeting CFTR with | GGAAGAATTTCATT | 18 |
| | | | | Nme2Cas9 | CTGTTCTCAG | |
| 44 | pAE146 | Nme2TS27 | pLKO | Targeting CFTR with | GCTCAGTTTTCCTG | 19 |
| | | | | Nme2Cas9 | GATTATGCCT | |
| 45 | pAE152 | Nme2TS31 | pLKO | Targeting VEGFA | GCGTTGGAGCGGG | 20 |
| | | | | with Nme2Cas9 | GAGAAGGCCAG | |
| 46 | pAE153 | Nme2TS34 | pLKO | Targeting LINC01588 | GGGCCGCGGAGAT | 21 |
| | | | | with Nme2Cas9 | AGCTGCAGGGC | |
| 47 | pAE154 | Nme2TS35 | pLKO | Targeting LINC01588 | GCCCACCCGGCGG | 22 |
| | | | | with Nme2Cas9 | CGCCTCCCTGC | |
| 48 | pAE155 | Nme2TS36 | pLKO | Targeting LINC01588 | GCGTGGCAGCTGAT | 23 |
| | | | | with Nme2Cas9 | ATCCGGCCGC | |
| 49 | pAE156 | Nme2TS37 | pLKO | Targeting LINC01588 | GCCGCGGCGCGAC | 24 |
| | | | | with Nme2Cas9 | GTGGAGCCAGC | |
| 50 | pAE157 | Nme2TS38 | pLKO | Targeting LINC01588 | GTGCTCCCCAGCCC | 25 |
| | | | | with Nme2Cas9 | AAACCGCCGC | |
| 51 | pAE159 | Nme2TS41 | pLKO | Targeting AGA with | GTCAGATTGGCTTG | 26 |
| | | | | Nme2Cas9 | CTCGGAATTG | |
| 52 | pAE185 | Nme2TS44 | pLKO | Targeting VEGFA | GCTGGGTGAATGG | 27 |
| | | | | with Nme2Cas9 | AGCGAGCAGCG | |
| 53 | pAE186 | Nme2TS45 | pLKO | Targeting VEGFA | GTCCTGGAGTGACC | 28 |
| | | | | with Nme2Cas9 | CCTGGCCTTC | |
| 54 | pAE187 | Nme2TS46 | pLKO | Targeting VEGFA | GATCCTGGAGTGAC | 29 |
| | | | | with Nme2Cas9 | CCCTGGCCTT | |
| 55 | pAE188 | Nme2TS47 | pLKO | Targeting VEGFA | GTGTGTCCCTCTCC | 30 |
| | | | | with Nme2Cas9 | CCACCCGTCC | |
| 56 | pAE189 | Nme2TS48 | pLKO | Targeting VEGFA | GTTGGAGCGGGGA | 31 |
| | | | | with Nme2Cas9 | GAAGGCCAGGG | |
| 57 | pAE190 | Nme2TS49 | pLKO | Targeting VEGFA | GCGTTGGAGCGGG | 20 |
| | | | | with Nme2Cas9 | GAGAAGGCCAG | |
| 58 | pAE191 | Nme2TS50 | pLKO | Targeting AGA with | GTACCCTCCAATAA | 32 |
| | | | | Nme2Cas9 | TTTGGCTGGC | |
| 59 | pAE192 | Nme2TS51 | pLKO | Targeting AGA with | GATAATTTGGCTGG | 33 |
| | | | | Nme2Cas9 | CAATTCCGAG | |
| 60 | PAE232 | TS64_FancJ1 | pLKO | Targeting FANCJ | GAAAATTGTGATTT | 40 |
| | | | | with Nme2Cas9 | CCAGATCCAC | |
| 61 | PAE233 | TS65_FancJ2 | pLKO | Targeting FANCJ | GAGCAGAAAAAAT | 41 |
| | | | | with Nme2Cas9 | TGTGATTTCC | |
| 62 | pAE200 | Nme2TS58 | pLKO | Targeting DS in | GCAGGGGCCAGGT | 34 |
| | | (Nme2DS1) | | VEGFA with | GTCCTTCTCTG | |
| | | | | Nme2Cas9 | | |
| 63 | pAE201 | Nme2TS59 | pLKO | Targeting DS in | GAATGGCAGGCGG | 35 |
| | | (Nme2DS2) | | VEGFA with | AGGTTGTACTG | |
| | | | | Nme2Cas9 | | |
| 64 | pAE202 | Nme2TS60 | pLKO | Targeting DS in | GAGTGAGAGAGTG | 36 |
| | | (Nme2DS3) | | VEGFA with | AGAGAGAGACA | |
| | | | | Nme2Cas9 | | |
| 65 | pAE203 | Nme2TS61 | pLKO | Targeting DS in | GTGAGCAGGCACC | 37 |
| | | (Nme2DS4) | | VEGFA with | TGTGCCAACAT | |
| | | | | Nme2Cas9 | | |
| 66 | pAE204 | Nme2TS62 | pLKO | Targeting DS in | GCGTGGGGGCTCC | 38 |
| | | (Nme2DS5) | | VEGFA with | GTGCCCCACGC | |
| | | | | Nme2Cas9 | | |
| 67 | PAE205 | Nme2TS63 | pLKO | Targeting DS in | GCATGGGCAGGGG | 39 |
| | | (Nme2DS6) | | VEGFA with | CTGGGGTGCAC | |
| | | | | Nme2Cas9 | | |
| 68 | pAE207 | SpyDS1 | pLKO | Targeting DS in | GGGCCAGGTGTCCT | 94 |
| | | | | VEGFA with | TCTCTG | |
| | | | | SpyCas9 | | |
| 69 | PAE208 | SpyDS2 | pLKO | Targeting DS in | GGCAGGCGGAGGT | 95 |
| | | | | VEGFA with | TGTACTG | |
| | | | | SpyCas9 | | |
| 70 | PAE209 | SpyDS3 | pLKO | Targeting DS in | GAGAGAGTGAGAG | 96 |
| | | | | VEGFA with | AGAGACA | |
| | | | | SpyCas9 | | |
| 71 | PAE210 | SpyDS4 | pLKO | Targeting DS in | GCAGGCACCTGTGC | 97 |
| | | | | VEGFA with | CAACAT | |
| | | | | SpyCas9 | | |
| 72 | pAE211 | SpyDS5 | pLKO | Targeting DS in | GGGGGCTCCGTGCC | 98 |
| | | | | VEGFA with | CCACGC | |
| | | | | SpyCas9 | | |
| 73 | pAE212 | SpyDS6 | pLKO | Targeting DS in | GGGCAGGGGCTGG | 99 |
| | | | | VEGFA with | GGTGCAC | |
| | | | | SpyCas9 | | |
| 74 | pAE169 | hDeCas9 Wt | AAV | Nme2Cas9 all-in-one | See examples herein. | |
| | | in AAV | | AAV expression with | | |
| | | backbone | | sgRNA cassette | | |
| 75 | pAE217 | backbone | pMCSG7 | wildtype Nme2Cas9 | See examples herein. | |
| | | hDeCas9 wt | | for bacterial | | |
| | | in pMSCG7 | | expression | | |
| 76 | pAE107 | 2xNLS | pCdest | Nme2Cas9 CMV- | See examples herein. | |
| | | Nme2Cas9 | | driven expression | | |
| | | with HA | | plasmid | | |
| 77 | pAE127 | hDemonCas9 | pMSCG7 | Targeting | See examples herein. | |
| | | 3X NLS in | | endogenous loci with | | |
| | | pMSCG7 | | Nme2Cas9 | | |
| 78 | pAM172 | hNme2Cas9 | pCVL | Lentivector | See examples herein. | |
| | | 4X NLS with | | containing UCOE, | | |
| | | 3XHA (SEQ | | SFFV driven | | |
| | | ID NO: 171) | | Nme2Cas9 and Puro | | |
| 79 | pAM174 | nickase | pCVL | Lentivector | See examples herein. | |
| | | hNme2Cas9 | | containing UCOE, | | |
| | | D16A 4X | | SFFV driven | | |
| | | NLS with | | Nme2Cas9 and Puro | | |
| | | 3XHA (SEQ | | | | |
| | | ID NO: 171) | | | | |
| 80 | pAM175 | nickase | pCVL | Lentivector | See examples herein. | |
| | | hNme2Cas9 | | containing UCOE, | | |
| | | H588A 4X | | SFFV driven | | |
| | | NLS with | | Nme2Cas9 and Puro | | |
| | | 3XHA (SEQ | | | | |
| | | ID NO: 171) | | | | |
| 81 | pAM177 | dead | pCVL | Lentivector | See examples herein. | |
| | | hNme2Cas9 | | containing UCOE, | | |
| | | 4X NLS with | | SFFV driven | | |
| | | 3XHA (SEQ | | Nme2Cas9 and Puro | | |
| | | ID NO: 171) | | | | |

TABLE 6
Exemplary Oligonucleotide Sequences For NmeCas9 Constructs

| | | SEQ | | |
|---|---|---|---|---|
| Number | Name | ID NO | Sequence | Purpose |
| 1 | AAVS1_TIDE1_FW | 42 | TGGCTTAGCACCTCTCCAT | TIDE analysis |
| 2 | LINC01588_TIDE_FW | 43 | AGAGGAGCCTTCTGACTGCTGCAGA | TIDE analysis |
| 3 | AAVS1_TIDE2_FW | 44 | TCCGTCTTCCTCCACTCC | TIDE analysis |
| 4 | NTS55_TIDE_FW | 45 | TAGAGAACTGGGTAGTGTG | TIDE analysis |
| 5 | VEGF_TIDE3_FW | 46 | GTACATGAAGCAACTCCAGTCCCA | TIDE analysis |
| 6 | hCFTR_TIDE1_FW | 47 | TGGTGATTATGGGAGAACTGGAGC | TIDE analysis |
| 7 | AGA_TIDE1_FW | 48 | GGCATAAGGAAATCGAAGGTC | TIDE analysis |
| 8 | VEGF_TIDE4_FW | 49 | ACACGGGCAGCATGGGAATAGTC | TIDE analysis |
| 9 | VEGF_TIDE5_FW | 100 | CCTGTGTGGCTTTGCTTTGGTCG | TIDE analysis |
| 10 | VEGF_TIDE6_FW | 51 | GGAGGAAGAGTAGCTCGCCGAGG | TIDE analysis |
| 11 | VEGF_TIDE7_FW | 52 | AGGGAGAGGGAAGTGTGGGGAAGG | TIDE analysis |
| 12 | AAVS1_TIDE1_RV | 101 | AGAACTCAGGACCAACTTATTCTG | TIDE analysis |
| 13 | LINC01588_TIDE_RV | 102 | ATGACAGACACAACCAGAGGGCA | TIDE analysis |
| 14 | AAVS1_TIDE2_RV | 103 | TAGGAAGGAGGAGGCCTAAG | TIDE analysis |
| 15 | NTS55_TIDE_RV | 104 | CCAATATTGCATGGGATGG | TIDE analysis |
| 16 | VEGF_TIDE3_RV | 105 | ATCAAATTCCAGCACCGAGCGC | TIDE analysis |
| 17 | hCFTR_TIDE1_RV | 106 | ACCATTGAGGACGTTTGTCTCAC | TIDE analysis |
| 18 | AGA_TIDE1_RV | 107 | CATGTCCTCAAGTCAAGAACAAG | TIDE analysis |
| 19 | VEGF_TIDE4_RV | 108 | GCTAGGGGAGAGTCCCACTGTCCA | TIDE analysis |
| 20 | VEGF_TIDE5_RV | 109 | GTAGGGTGTGATGGGAGGCTAAGC | TIDE analysis |
| 21 | VEGF_TIDE6_RV | 110 | AGACCGAGTGGCAGTGACAGCAAG | TIDE analysis |
| 22 | VEGF_TIDE7_RV | 111 | GTCTTCCTGCTCTGTGCGCACGAC | TIDE analysis |
| 23 | Random PAM_FW | 112 | TAGCGGCCGCTCATGCGCGGCGCAT | Protospacer with |
| | | | TACCTTTACNNNNNNNNNNGGATCC | randomized |
| | | | TCTAGAGTCG | PAM |
| 24 | Random PAM_RV | 113 | ACAGGAAACAGCTATGACCATGAAA | Protospacer with |
| | | | GCTTGCATGCCTGCAGGTCGACTCTA | randomized |
| | | | GAGGATC | PAM |
| 25 | DS2_ON_FW1 | 114 | ctacacgacgctcttccgatctCCTGGAGCGTGT | Targeted Deep |
| | | | ACGTTGG | Seq |
| 26 | SpyDS2_OT1_FW1 | 115 | ctacacgacgctcttccgatctCCTGTGGTCCCA | Targeted Deep |
| | | | GCTACTTG | Seq |
| 27 | SpyDS2_OT2_FW1 | 116 | ctacacgacgctcttccgatctATCTGCGATGTC | Targeted Deep |
| | | | CTCGAGG | Seq |
| 28 | SpyDS2_OT3_FW1 | 117 | ctacacgacgctcttccgatctTGGTGTGCGCCT | Targeted Deep |
| | | | CTAACG | Seq |
| 29 | SpyDS2_OT4_FW1 | 118 | ctacacgacgctcttccgatctGGAGTCTTGCTTT | Targeted Deep |
| | | | GTCACTCAGA | Seq |
| 30 | SpyDS2_OT5_FW1 | 119 | ctacacgacgctcttccgatctAGCCTAGACCCA | Targeted Deep |
| | | | GTCCCAT | Seq |
| 31 | SpyDS2_OT6_FW1 | 120 | ctacacgacgctcttccgatctGCTGGGCATAGT | Targeted Deep |
| | | | AGTGGACT | Seq |
| 32 | SpyDS2_OT7_FW1 | 121 | ctacacgacgctcttccgatctTGGGGAGGCTGA | Targeted Deep |
| | | | GACACGA | Seq |
| 33 | SpyDS2_OT8_FW1 | 122 | ctacacgacgctcttccgatctCTTGGGAGGCTG | Targeted Deep |
| | | | AGGCAAG | Seq |
| 34 | DS2_ON_RV1 | 123 | agacgtgtgctcttccgatctCAGGAGGATGAG | Targeted Deep |
| | | | AGCCAGG | Seq |
| 35 | SpyDS2_OT1_RV1 | 124 | agacgtgtgctcttccgatctCAGGGTCTCACTC | Targeted Deep |
| | | | TATCACCCA | Seq |
| 36 | SpyDS2_OT2_RV1 | 125 | agacgtgtgctcttccgatctACTGAATGGGTTG | Targeted Deep |
| | | | AACTTGGC | Seq |
| 37 | SpyDS2_OT3_RV1 | 126 | agacgtgtgctcttccgatctGAGACAGAATCTT | Targeted Deep |
| | | | GCTCTGTCTCC | Seq |
| 38 | SpyDS2_OT4_RV1 | 127 | agacgtgtgctcttccgatctTCCCAGCTACTTG | Targeted Deep |
| | | | GGAGGC | Seq |
| 39 | SpyDS2_OT5_RV1 | 128 | agacgtgtgctcttccgatctCCTGCCCAAATAG | Targeted Deep |
| | | | GGAAGCAG | Seq |
| 40 | SpyDS2_OT6_RV1 | 129 | agacgtgtgctcttccgatctTGGCGCCTTAGTC | Targeted Deep |
| | | | TCTGCTAC | Seq |
| 41 | SpyDS2_OT7_RV1 | 130 | agacgtgtgctcttccgatctGCATGAGACACAG | Targeted Deep |
| | | | TTTCACTCTG | Seq |
| 42 | SpyDS2_OT8_RV1 | 131 | agacgtgtgctcttccgatctGAGAGAGTCTCAC | Targeted Deep |
| | | | TGCGTTGC | Seq |
| 43 | DS4_ON_FW3 | 132 | ctacacgacgctcttccgatctTCTCTCACCCACT | Targeted Deep |
| | | | GGGCAC | Seq |
| 44 | DS4_ON_RV3 | 133 | agacgtgtgctcttccgatctGCTTCCAGACGAG | Targeted Deep |
| | | | TGCAGA | Seq |
| 45 | SpyDS4_OT1_FW1 | 134 | ctacacgacgctcttccgatctAAGTTTTCAAAC | Targeted Deep |
| | | | CAGAAGAACTACGAC | Seq |
| 46 | SpyDS4_OT2_FW1 | 135 | ctacacgacgctcttccgatctCCGGTATAAGTC | Targeted Deep |
| | | | CTGGAGCG | Seq |
| 47 | SpyDS4_OT3_FW1 | 136 | ctacacgacgctcttccgatctGCCAGGGAGCAA | Targeted Deep |
| | | | TGGCAG | Seq |
| 48 | SpyDS4_OT6_FW1 | 137 | ctacacgacgctcttccgatctCCTCGAATTCCA | Targeted Deep |
| | | | CGGGGTT | Sea |
| 49 | DS16_ON_FW1 | 138 | ctacacgacgctcttccgatctGTTGGTGGGAGG | Targeted Deep |
| | | | GAAGTGAG | Seq |
| 50 | SpyDS6_OT1_FW1 | 139 | ctacacgacgctcttccgatctGATGGCGGTTGT | Targeted Deep |
| | | | AGCGGC | Seq |
| 51 | SpyDS6_OT2_FW1 | 140 | ctacacgacgctcttccgatctCACATAAACCTA | Targeted Deep |
| | | | TGTTTCAGCAGA | Seq |
| 52 | SpyDS6_OT3_FW1 | 141 | ctacacgacgctcttccgatctGCTAGTTGGATT | Targeted Deep |
| | | | GAAGCAGGGT | Seq |
| 53 | SpyDS6_OT4_FW1 | 142 | ctacacgacgctcttccgatctTTGAGTGCGGCA | Targeted Deep |
| | | | GCTTCC | Seq |
| 54 | SpyDS6_OT6_FW1 | 143 | ctacacgacgctcttccgatctATAACCCTCCCA | Targeted Deep |
| | | | GGCAAAGTC | Seq |
| 55 | SpyDS6_OT7_FW1 | 144 | ctacacgacgctcttccgatctAGCCTGCACATC | Targeted Deep |
| | | | TGAGCTC | Seq |
| 56 | SpyDS6_OT8_FW1 | 145 | ctacacgacgctcttccgatctGGAGCATTGAAG | Targeted Deep |
| | | | TGCCTGG | Seq |
| 57 | DeDS6_ON_RV1 | 146 | agacgtgtgctcttccgatctCAGCCTGGGACCA | Targeted Deep |
| | | | CTGA | Seq |
| 58 | SpyDS6_OT1_RV1 | 147 | agacgtgtgctcttccgatctCATCCTCGACAGT | Targeted Deep |
| | | | CGCGG | Seq |
| 59 | SpyDS6_OT2_RV1 | 148 | agacgtgtgctcttccgatctGACTGATCAAGTA | Targeted Deep |
| | | | GAATACTCATGGG | Seq |
| 60 | SpyDS6_OT3_RV1 | 149 | agacgtgtgctcttccgatctCCCTGCCAGCACT | Targeted Deep |
| | | | GAAGC | Seq |
| 61 | SpyDS6_OT4_Rv1 | 150 | agacgtgtgctcttccgatctGGTTCCTATCTTTC | Targeted Deep |
| | | | TAGACCAGGAGT | Seq |
| 62 | SpyDS6_OT6_RV1 | 151 | agacgtgtgctcttccgatctAGTGTGGAGGGCT | Targeted Deep |
| | | | CAGGG | Seq |
| 63 | SpyDS6_OT7_RV1 | 152 | agacgtgtgctcttccgatctGATGGGCAGAGG | Targeted Deep |
| | | | AAGGCAA | Seq |
| 64 | SpyDS6_OT8_RV1 | 153 | agacgtgtgctcttccgatctTCACTCTCATGAG | Targeted Deep |
| | | | CGTCCCA | Seq |
| 65 | Nme2DS2_OT1_FW1 | 154 | ctacacgacgctcttccgatctAAGGTTCCTTGC | Targeted Deep |
| | | | GGTTCGC | Seq |
| 66 | Nme2DS2_OT1_RV1 | 155 | agacgtgtgctcttccgatctCGCTGCCATTGCT | Targeted Deep |
| | | | CCCT | Seq |
| 67 | Nme2DS6_OT1_FW1 | 156 | ctacacgacgctcttccgatctTCTCGCACATTCT | Targeted Deep |
| | | | TCACGTCC | Seq |
| 68 | Nme2DS6_OT1_RV1 | 157 | agacgtgtgctcttccgatctAGGAACCTTCCCG | Targeted Deep |
| | | | ACTTAGGG | Seq |
| 69 | Rosa26_ON_FW1 | 158 | ctacacgacgctcttccgatctCCCGCCCATCTTC | Targeted Deep |
| | | | TAGAAAGAC | Seq |
| 70 | Rosa26_OT1_FW1 | 159 | ctacacgacgctcttccgatctTGCCAGGTGAGG | Targeted Deep |
| | | | GACTGG | Seq |
| 71 | Rosa26_ON_RV1 | 160 | agacgtgtgctcttccgatctTCTGGGAGTTCTC | Targeted Deep |
| | | | TGCTGCC | Seq |
| 72 | Rosa26_OT1_RV1 | 161 | agacgtgtgctcttccgatctTGCCCAACCTTAG | Targeted Deep |
| | | | CAAGGAG | Seq |
| 73 | pCSK9_ON_FW2 | 162 | ctacacgacgctcttccgatcttaccttggagcaac | Targeted Deep |
| | | | ggcg | Seq |
| 74 | PCSK9_ON_RV2 | 163 | agacgtgtgctcttccgatctcccaggacgaggatg | Targeted Deep |
| | | | gag | Seq |
| 75 | Tyr_500_FW3 | 164 | GATAGTCACTCCAGGGGTTG | TIDE analysis |
| 76 | Tyr_500_RV3 | 165 | GTGGTGAACCAATCAGTCCT | TIDE analysis |

### EXPERIMENTAL

### Example I

Discovery of Cas9 Orthologs with Differentially Diverged PIDs

Nme1Cas9 peptide sequence was used as a query in BLAST searches to find all Cas9 orthologs in Neisseria meningitidis species. Orthologs with >80% identity to Nme1Cas9 were selected for the remainder of this study. The PIDs were then aligned with that of Nme1Cas9 (residues 820-1082) using ClustalW2 and those with clusters of mutations in the PID were selected for further analysis. An unrooted phylogenetic tree of NmeCas9 orthologs was constructed using FigTree (tree.bio.ed.ac.uk/software/figtree/).

### Example II

### In Vitro PAM Discovery Assay

A dsDNA target library with randomized PAM sequences was generated by overlapping PCR, with the forward primer containing the 10-nt randomized PAM region. The library was gel-purified and subjected to in vitro cleavage reaction by purified Cas9 along with T7-transcribed sgRNAs. 300 nM Cas9:sgRNA complex was used to cleave 300 nM of the target fragment in 1× NEBuffer 3.1 (NEB) at 37° C. for 1 hr. The reaction was then treated with proteinase K at 50° C. for 10 minutes and run on a 4% agarose/1×TAE gel. The cleavage product was excised, eluted, and cloned using a previously described protocol (Zhang et al., 2012), with modifications. Briefly, DNA ends were repaired, non-templated 2′-deoxyadenosine tails were added, and Y-shaped adapters were ligated. After PCR, the product was quantitated with KAPA Library Quantification Kit and sequenced using a NextSeq 500 (Illumina) to obtain 75 nt paired-end reads. Sequences were analyzed with custom scripts and R.

### Example IV

### Transfections and Mammalian Genome Editing

Human codon-optimized Nme2Cas9 was cloned by Gibson Assembly into the pCDest2 plasmid backbone previously used for Nme1Cas9 and SpyCas9 expression (Pawluk et al., 2016; Amrani et al., 2018). Transfection of HEK293T and HEK293T-TLR2.0 cells was performed as previously described (Amrani et al., 2018). For Hepa1-6 transfections, Lipofectamine LTX was used to transfect 500 ng of all-in-one AAV.sgRNA.Nme2Cas9 plasmid in 24-well plates (˜10⁵ cells/well), using cells that had been cultured 24 hours before transfection. For K562 cells stably expressing Nme2Cas9 delivered via lentivector (see below), 50,000-150,000 cells were electroporated with 500 ng sgRNA plasmid using 10 μL Neon tips. To measure indels in all cells 72 hr after transfections, cells were harvested and genomic DNA was extracted using the DNaesy Blood and Tissue kit (Qiagen). The targeted locus was amplified by PCR, Sanger-sequenced (Genewiz), and analyzed by TIDE (Brinkman et al., 2014) using the Desktop Genetics web-based interface (tide.deskgen.com).

### Example V

### Lentiviral Transduction of K562 Cells to Stably Express Nme2Cas9

K562 cells stably expressing Nme2Cas9 were generated as previously described for Nme1Cas9 (Amrani et al., 2018). For lentivirus production, the lentiviral vector was co-transfected into HEK293T cells along with the packaging plasmids (Addgene 12260 & 12259) in 6-well plates using TransIT-LT1 transfection reagent (Mirus Bio). After 24 hours, culture media was aspirated from the transfected cells and replaced with 1 mL of fresh DMEM. The next day, the supernatant containing the virus was collected and filtered through a 0.45 μm filter. 10 uL of the undiluted supernatant along with 2.5 ug of Polybrene was used to transduce ˜10⁶ K562 cells in 6-well plates. The transduced cells were selected using media supplemented with 2.5 μg/mL puromycin.

### Example VI

### RNP Delivery for Mammalian Genome Editing

For RNP experiments, the Neon electroporation system was used exactly as described (Amrani et al., 2018). Briefly, 40 picomoles of 3xNLS-Nme2Cas9 along with 50 picomoles of T7-transcribed sgRNA was assembled in buffer R and electroporated using 10 μL Neon tips. After electroporation, cells were plated in pre-warmed 24-well plates containing the appropriate culture media without antibiotics. Electroporation parameters (voltage, width, number of pulses) were 1150 V, 20 ms, 2 pulses for HEK293T cells; 1000 V, 50 ms, 1 pulse for K562 cells.

### Example VII

### GUIDE-Seq

GUIDE-seq experiments were performed as described previously (Tsai et al., 2014), with minor modifications (Bolukbasi et al., 2015a). Briefly, HEK293T cells were transfected with 200 ng of Cas9 plasmid, 200 ng of sgRNA plasmid, and 7.5 pmol of annealed GUIDE-seq oligonucleotides using Polyfect (Qiagen). Alternatively, Hepa1-6 cells were transfected as described above. Genomic DNA was extracted with a DNeasy Blood and Tissue kit (Qiagen) 72 h after transfection according to the manufacturer's protocol. Library preparation and sequencing were performed exactly as described previously (Bolukbasi et al., 2015a). For analysis, all sequences with up to ten mismatches with the target site, as well as a C in the fifth PAM position (N₄CN), were considered potential off-target sites. Data were analyzed using the Bioconductor package GUIDEseq version 1.1.17 (Zhu et al., 2017).

### Example VIII

### Targeted Deep Sequencing and Analysis

We used targeted deep sequencing to confirm the results of GUIDE-seq and to measure indel rates with maximal accuracy. We used two-step PCR amplification to produce DNA fragments for each on- and off-target site. For SpyCas9 editing at DS2 and DS6, we selected the top off-target sites based on GUIDE-seq read counts. For SpyCas9 editing at DS4, fewer candidate off-target sites were identified by GUIDE-seq, and only those with NGG (DS4|OT1, DS4|OT3, DS4|OT6) or NGC (DS4|OT2) PAMs were examined by sequencing. In the first step, we used locus-specific primers bearing universal overhangs with ends complementary to the adapters. In the first step, 2× PCR master mix (NEB) was used to generate fragments bearing the overhangs. In the second step, the purified PCR products were amplified with a universal forward primer and indexed reverse primers. Full-size products (˜250 bp) were gel-purified and sequenced on an Illumina MiSeq in paired-end mode. MiSeq data analysis was performed as previously described (Pinello et al., 2016; Ibraheim et al., 2018).

### Example IX

### Off-Target Analysis Using CRISPRseek

Global off-target predictions for TS25 and TS47 were performed using the Bioconductor package CRISPRseek. Minor changes were made to accommodate characteristics of Nme2Cas9 not shared with SpyCas9. Specifically, we used the following changes to: gRNA.size=24, PAM=“NNNNCC”, PAM.size=6, RNA.PAM.pattern=“NNNNCN”, and candidate off-target sites with fewer than 6 mismatches were collected. The top potential off-target sites based on the numbers and positions of mismatches were selected. Genomic DNA from cells targeted by each respective sgRNA was used to amplify each candidate off-target locus and then analyzed by TIDE.

### Example X

### Mouse Strains and Embryo Collection

All animal experiments were conducted under the guidance of the Institutional Animal Care and Use Committee (IACUC) of the University of Massachusetts Medical School. C57BL/6NJ (Stock No. 005304). Mice were obtained from The Jackson Laboratory. All animals were maintained in a 12 h light cycle. The middle of the light cycle of the day when a mating plug was observed was considered embryonic day 0.5 (E0.5) of gestation. Zygotes were collected at E0.5 by tearing the ampulla with forceps and incubation in M2 medium containing hyaluronidase to remove cumulus cells.

### Example XI

In Vivo AAV8.Nme2Cas9+ sgRNA Delivery and Liver Tissue Processing

For the AAV8 vector injections, 8-week-old female C57BL/6NJ mice were injected with 4×10¹¹ genome copies per mouse via tail vein, with the sgRNA targeting a validated site in either Pcsk9 or Rosa26. Mice were sacrificed 28 days after vector administration and liver tissues were collected for analysis. Liver tissues were fixed in 4% formalin overnight, embedded in paraffin, sectioned and stained with hematoxylin and eosin (H&E). Blood was drawn from the facial vein at 0, 14 and 28 days post injection, and serum was isolated using a serum separator (BD, Cat. No. 365967) and stored at −80° C. until assay. Serum cholesterol level was measured using the Infinity™ colorimetric endpoint assay (Thermo-Scientific) following the manufacturer's protocol and as previously described (Ibraheim et al., 2018). For the anti-PCSK9 Western blot, 40 μg of protein from tissue or 2 ng of Recombinant Mouse PCSK9 Protein (R&D Systems, 9258-SE-020) were loaded onto a MiniPROTEAN® TGX™ Precast Gel (Bio-Rad). The separated bands were transferred onto a PVDF membrane and blocked with 5% Blocking-Grade Blocker solution (Bio-Rad) for 2 hours at room temperature. Next, the membrane was incubated with rabbit anti-GAPDH (Abcam ab9485, 1:2,000) or goat anti-PCSK9 (R&D Systems AF3985, 1:400) antibodies overnight. Membranes were washed in TBST and incubated with horseradish peroxidase (HRP)-conjugated goat anti-rabbit (Bio-Rad 1706515, 1:4,000), and donkey anti-goat (R&D Systems HAF109, 1:2,000) secondary antibodies for 2 hours at room temperature. The membranes were washed again in TBST and visualized using Clarity™ western ECL substrate (Bio-Rad) using an M35A XOMAT Processor (Kodak).

### Example XII

### Ex Vivo AAV6.Nme2Cas9 Delivery in Mouse Zygotes

Zygotes were incubated in 15 μl drops of KSOM (Potassium-Supplemented Simplex Optimized Medium, Millipore, Cat. No. MR-106-D) containing 3×10⁹ or 3×10⁸ GCs of AAV6.Nme2Cas9.sgTyr vector for 5-6 h (4 zygotes in each drop). After incubation, zygotes were rinsed in M2 and transferred to fresh KSOM for overnight culture. The next day, the embryos that advanced to 2-cell stage were transferred into the oviduct of pseudopregnant recipients and allowed to develop to term.

### Example XIII

### Ribonucleoprotein (RNP) Cas9 Delivery

For RNP experiments, the Neon electroporation system was used exactly as described (Amrani et al., 2018). Briefly, 40 picomoles of 3xNLS-Nme2Cas9 along with 50 picomoles of T7-transcribed sgRNA was assembled in buffer R and electroporated using 10 μL Neon tips. After electroporation, cells were plated in pre-warmed 24-well plates containing the appropriate culture media without antibiotics. Electroporation parameters (voltage, width, number of pulses) were 1150 V, 20 ms, 2 pulses for HEK293T cells; 1000 V, 50 ms, 1 pulse for K562 cells.

### Example IX

Nme2Cas9 Plasmid Insert Construction

MAAFKPNPINYILGLDIGIASVGWAMVEIDEEENPIRLIDLGVRVFERAEVPKTGDSLAMARRLA
RSVRRLTRRRAHRLLRARRLLKREGVLQAADFDENGLIKSLPNTPWQLRAAALDRKLTPLEWSA
VLLHLIKHRGYLSQRKNEGETADKELGALLKGVANNAHALQTGDFRTPAELALNKFEKESGHIR
NQRGDYSHTFSRKDLQAELILLFEKQKEFGNPHVSGGLKEGIETLLMTQRPALSGDAVQKMLG
HCTFEPAEPKAAKNTYTAERFIWLTKLNNLRILEQGSERPLTDTERATLMDEPYRKSKLTYAQAR
KLLGLEDTAFFKGLRYGKDNAEASTLMEMKAYHAISRALEKEGLKDKKSPLNLSSELQDEIGTA
FSLFKTDEDITGRLKDRVQPEILEALLKHISFDKFVQISLKALRRIVPLMEQGKRYDEACAEIYGD
HYGKKNTEEKIYLPPIPADEIRNPVVLRALSQARKVINGVVRRYGSPARIHIETAREVGKSFKDRK
EIEKRQEENRKDREKAAAKFREYFPNFVGEPKSKDILKLRLYEQQHGKCLYSGKEINLVRLNEK
GYVEIDHALPFSRTWDDSFNNKVLVLGSENQNKGNQTPYEYFNGKDNSREWQEFKARVETSRF
PRSKKQRILLQKFDEDGFKECNLNDTRYVNRFLCQFVADHILLTGKGKRRVFASNGQITNLLRG
FWGLRKVRAENDRHHALDAVVVACSTVAMQQKITRFVRYKEMNAFDGKTIDKETGKVLHQKT
HFPQPWEFFAQEVMIRVFGKPDGKPEFEEADTPEKLRTLLAEKLSSRPEAVHEYVTPLFVSRAP
NRKMSGAHKDTLRSAKRFVKHNEKISVKRVWLTEIKLADLENMVNYKNGREIELYEALKARLEA
YGGNAKQAFDPKDNPFYKKGGQLVKAVRVEKTQESGVLLNKKNAYTIADNGDMVRVDVFCKV
DKKGKNQYFIVPIYAWQVAENILPDIDCKGYRIDDSYTFCFSLHKYDLIAFQKDEKSKVEFAYYI
NCDSSNGRFYLAWHDKGSKEQQFRISTQNLVLIQKYQVNELGKEIRPCRLKKRPPVRGTGGPK
KKRKVYPYDVPDYAGYPYDVPDYAGSYPYDVPDYAGSAAPAAKKKKLDFESG* (SEQ ID NO: 166)
SV40 NLS (yellow-BOLD); 3X-HA-Tag (green-(underlined/bold); cMyc-like NLS (teal-plain); Linker (magenta-bold italics) and Nme2Cas9 (italics).

### Example X

Nme2Cas9 AAV Insert Construction

MVPKKKRKVEDKRPAATKKAGQAKKKKMAAFKPNPINYILGLDIGIASVGWAMVEIDEEE
NPIRLIDLGVRVFERAEVPKTGDSLAMARRLARSVRRLTRRRAHRLLRARRLLKREGVLQAADFD
ENGLIKSLPNTPWQLRAAALDRKLTPLEWSAVLLHLIKHRGYLSQRKNEGETADKELGALLKGV
ANNAHALQTGDFRTPAELALNKFEKESGHIRNQRGDYSHTFSRKDLQAELILLFEKQKEFGNP
HVSGGLKEGIETLLMTQRPALSGDAVQKMLGHCTFEPAEPKAAKNTYTAERFIWLTKLNNLRIL
EQGSERPLTDTERATLMDEPYRKSKLTYAQARKLLGLEDTAFFKGLRYGKDNAEASTLMEMKA
YHAISRALEKEGLKDKKSPLNLSSELQDEIGTAFSLFKTDEDITGRLKDRVQPEILEALLKHISFD
KFVQISLKALRRIVPLMEQGKRYDEACAEIYGDHYGKKNTEEKIYLPPIPADEIRNPVVLRALSQA
RKVINGVVRRYGSPARIHIETAREVGKSFKDRKEIEKRQEENRKDREKAAAKFREYFPNFVGEP
KSKDILKLRLYEQQHGKCLYSGKEINLVRLNEKGYVEIDHALPFSRTWDDSFNNKVLVLGSENQ
NKGNQTPYEYFNGKDNSREWQEFKARVETSRFPRSKKQRILLQKFDEDGFKECNLNDTRYVNR
FLCQFVADHILLTGKGKRRVFASNGQITNLLRGFWGLRKVRAENDRHHALDAVVVACSTVAMQ
QKITRFVRYKEMNAFDGKTIDKETGKVLHQKTHFPQPWEFFAQEVMIRVFGKPDGKPEFEEA
DTPEKLRTLLAEKLSSRPEAVHEYVTPLFVSRAPNRKMSGAHKDTLRSAKRFVKHNEKISVKRV
WLTEIKLADLENMVNYKNGREIELYEALKARLEAYGGNAKQAFDPKDNPFYKKGGQLVKAVRV
EKTQESGVLLNKKNAYTIADNGDMVRVDVFCKVDKKGKNQYFIVPIYAWQVAENILPDIDCKG
YRIDDSYTFCFSLHKYDLIAFQKDEKSKVEFAYYINCDSSNGRFYLAWHDKGSKEQQFRISTQNL
VLIQKYQVNELGKEIRPCRLKKRPPVREDKRPAATKKAGQAKKKKYPYDVPDYAGYPYDV
PDYAGSYPYDVPDYAAAPAAKKKKLD* (SEQ ID NO: 167)
SV40 NLS (yellow-BOLD); 3X-HA-Tag (green-(underlined/bold); Nucleoplasmin-like NLS (red-underline); c-myc NLS (teal-plain); Linker (magenta-bold italics) and Nme2Cas9 (italics).

### Example XI

Recombinant Nme2Cas9 Construction

PKKKRKVNAMAAFKPNPINYILGLDIGIASVGWAMVEIDEEENPIRLIDLGVRVFERAEVPKTG
DSLAMARRLARSVRRLTRRRAHRLLRARRLLKREGVLQAADFDENGLIKSLPNTPWQLRAAALD
RKLTPLEWSAVLLHLIKHRGYLSQRKNEGETADKELGALLKGVANNAHALQTGDFRTPAELALN
KFEKESGHIRNORGDYSHTFSRKDLQAELILLFEKQKEFGNPHVSGGLKEGIETLLMTQRPALS
GDAVQKMLGHCTFEPAEPKAAKNTYTAERFIWLTKLNNLRILEQGSERPLTDTERATLMDEPYR
KSKLTYAQARKLLGLEDTAFFKGLRYGKDNAEASTLMEMKAYHAISRALEKEGLKDKKSPLNLS
SELQDEIGTAFSLFKTDEDITGRLKDRVQPEILEALLKHISFDKFVQISLKALRRIVPLMEQGKRY
DEACAEIYGDHYGKKNTEEKIYLPPIPADEIRNPVVLRALSQARKVINGVVRRYGSPARIHIETAR
EVGKSFKDRKEIEKRQEENRKDREKAAAKFREYFPNFVGEPKSKDILKLRLYEQQHGKCLYSG
KEINLVRLNEKGYVEIDHALPFSRTWDDSFNNKVLVLGSENONKGNQTPYEYFNGKDNSREWQ
EFKARVETSRFPRSKKQRILLQKFDEDGFKECNLNDTRYVNRFLCQFVADHILLTGKGKRRVFA
SNGQITNLLRGFWGLRKVRAENDRHHALDAVVVACSTVAMQQKITRFVRYKEMNAFDGKTIDK
ETGKVLHQKTHFPQPWEFFAQEVMIRVFGKPDGKPEFEEADTPEKLRTLLAEKLSSRPEAVHE
YVTPLFVSRAPNRKMSGAHKDTLRSAKRFVKHNEKISVKRVWLTEIKLADLENMVNYKNGREIE
LYEALKARLEAYGGNAKQAFDPKDNPFYKKGGQLVKAVRVEKTQESGVLLNKKNAYTIADNGD
MVRVDVFCKVDKKGKNQYFIVPIYAWQVAENILPDIDCKGYRIDDSYTFCFSLHKYDLIAFQKD
EKSKVEFAYYINCDSSNGRFYLAWHDKGSKEQQFRISTQNLVLIQKYQVNELGKEIRPCRLKKRP
PVRGGGGSGGGGSGGGGSPAAKKKKLDGGGSKRPAATKKAGQAKKKK* (SEQ ID NO: 168)
SV40 NLS (yellow-BOLD); Nucleoplasmin-like NLS (red-underline); Linker (magenta-bold italics) and Nme2Cas9 (italics).

### Example XII

RNP-Recombinant Nme2Cas9 Construction

PKKKRKVNAMAAFKPNPINYILGLDIGIASVGWAMVEIDEEENPIRLIDLGVRVFERAEVPKTG
DSLAMARRLARSVRRLTRRRAHRLLRARRLLKREGVLQAADFDENGLIKSLPNTPWQLRAAALD
RKLTPLEWSAVLLHLIKHRGYLSQRKNEGETADKELGALLKGVANNAHALQTGDFRTPAELALN
KFEKESGHIRNQRGDYSHTFSRKDLQAELILLFEKQKEFGNPHVSGGLKEGIETLLMTQRPALS
GDAVQKMLGHCTFEPAEPKAAKNTYTAERFIWLTKLNNLRILEQGSERPLTDTERATLMDEPYR
KSKLTYAQARKLLGLEDTAFFKGLRYGKDNAEASTLMEMKAYHAISRALEKEGLKDKKSPLNLS
SELQDEIGTAFSLFKTDEDITGRLKDRVQPEILEALLKHISFDKFVQISLKALRRIVPLMEQGKRY
DEACAEIYGDHYGKKNTEEKIYLPPIPADEIRNPVVLRALSQARKVINGVVRRYGSPARIHIETAR
EVGKSFKDRKEIEKRQEENRKDREKAAAKFREYFPNFVGEPKSKDILKLRLYEQQHGKCLYSG
KEINLVRLNEKGYVEIDHALPFSRTWDDSFNNKVLVLGSENONKGNQTPYEYFNGKDNSREWQ
EFKARVETSRFPRSKKQRILLQKFDEDGFKECNLNDTRYVNRFLCQFVADHILLTGKGKRRVFA
SNGQITNLLRGFWGLRKVRAENDRHHALDAVVVACSTVAMQQKITRFVRYKEMNAFDGKTIDK
ETGKVLHQKTHFPQPWEFFAQEVMIRVFGKPDGKPEFEEADTPEKLRTLLAEKLSSRPEAVHE
YVTPLFVSRAPNRKMSGAHKDTLRSAKRFVKHNEKISVKRVWLTEIKLADLENMVNYKNGREIE
LYEALKARLEAYGGNAKQAFDPKDNPFYKKGGQLVKAVRVEKTQESGVLLNKKNAYTIADNGD
MVRVDVFCKVDKKGKNQYFIVPIYAWQVAENILPDIDCKGYRIDDSYTFCFSLHKYDLIAFQKD
EKSKVEFAYYINCDSSNGRFYLAWHDKGSKEQQFRISTQNLVLIQKYQVNELGKEIRPCRLKKRP
PVRGGGGSGGGGSGGGGSPAAKKKKLDGGGSKRPAATKKAGOAKKKK* (SEQ ID NO: 168)
SV40 NLS (yellow-BOLD); Nucleoplasmin-like NLS (red-underline); Linker (magenta-bold italics) and Nme2Cas9 (italics).

### REFERENCES

Each is herein incorporated by reference in their entirety:
Amrani, N., Gao, X. D., Liu, P., Edraki, A., Mir, A., Ibraheim, R., Gupta, A., Sasaki, K. E., Wu, T., Donohoue, P. D., et al. (2018). NmeCas9 is an intrinsically high-fidelity genome editing platform. BioRxiv, doi.org/10.1101/172650.
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709-1712.
Bisaria, N., Jarmoskaite, I., and Herschlag, D. (2017). Lessons from Enzyme Kinetics Reveal Specificity Principles for RNA-Guided Nucleases in RNA Interference and CRISPR-Based Genome Editing. Cell Syst. 4, 21-29.
Bolukbasi, M. F., Gupta, A., Oikemus, S., Derr, A. G., Garber, M., Brodsky, M. H., Zhu, L. J., and Wolfe, S. A. (2015a). DNA-binding-domain fusions enhance the targeting range and precision of Cas9. Nat. Methods 12, 1150-1156.
Bolukbasi, M. F., Gupta, A., and Wolfe, S. A. (2015b). Creating and evaluating accurate CRISPR-Cas9 scalpels for genomic surgery. Nat. Methods 13, 41-50.
Brinkman, E. K., Chen, T., Amendola, M., and van Steensel, B. (2014). Easy quantitative assessment of genome editing by sequence trace decomposition. Nucleic Acids Res. 42, e168.
Brouns, S. J., Jore, M. M., Lundgren, M., Westra, E. R., Slijkhuis, R. J., Snijders, A. P., Dickman, M. J., Makarova, K. S., Koonin, E. V., and van der Oost, J. (2008). Small CRISPR RNAs guide antiviral defense in prokaryotes. Science 321, 960-964.
Casini, A., Olivieri, M., Petris, G., Montagna, C., Reginato, G., Maule, G., Lorenzin, F., Prandi, D., Romanel, A., Demichelis, F., et al. (2018). A highly specific SpCas9 variant is identified by in vivo screening in yeast. Nat. Biotechnol. 36, 265-271.
Certo, M. T., Ryu, B. Y., Annis, J. E., Garibov, M., Jarjour, J., Rawlings, D. J., and Scharenberg, A. M. (2011). Tracking genome engineering outcome at individual DNA breakpoints. Nat. Methods 8, 671-676.
Chen, J. S., Dagdas, Y. S., Kleinstiver, B. P., Welch, M. M., Sousa, A. A., Harrington, L. B., Sternberg, S. H., Joung, J. K., Yildiz, A., and Doudna, J. A. (2017). Enhanced proofreading governs CRISPR-Cas9 targeting accuracy. Nature 550, 407-410.
Cho, S. W., Kim, S., Kim, J. M., and Kim, J. S. (2013). Targeted genome engineering in human cells with the Cas9 RNA-guided endonuclease. Nat. Biotechnol. 31, 230-232.
Cho, S. W., Kim, S., Kim, Y., Kweon, J., Kim, H. S., Bae, S., and Kim, J. S. (2014). Analysis of off-target effects of CRISPR/Cas-derived RNA-guided endonucleases and nickases. Genome Res. 24, 132-141.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
Deltcheva, E., Chylinski, K., Sharma, C. M., Gonzales, K., Chao, Y., Pirzada, Z. A., Eckert, M. R., Vogel, J., and Charpentier, E. (2011). CRISPR RNA maturation by trans-encoded small RNA and host factor RNase III. Nature 471, 602-607.
Deveau, H., Barrangou, R., Garneau, J. E., Labonte, J., Fremaux, C., Boyaval, P., Romero, D. A., Horvath, P., and Moineau, S. (2008). Phage response to CRISPR-encoded resistance in Streptococcus thermophilus. J. Bacteriol. 190, 1390-1400.
Dominguez, A. A., Lim, W. A., and Qi, L. S. (2016). Beyond editing: repurposing CRISPR-Cas9 for precision genome regulation and interrogation. Nat. Rev. Mol. Cell Biol. 17, 5-15.
Dong, Guo, M., Wang, S., Zhu, Y., Wang, S., Xiong, Z., Yang, J., Xu, Z., and Huang, Z. (2017). Structural basis of CRISPR-SpyCas9 inhibition by an anti-CRISPR protein. Nature 546, 436-439.
Esvelt, K. M., Mali, P., Braff, J. L., Moosburner, M., Yaung, S. J., and Church, G. M. (2013). Orthogonal Cas9 proteins for RNA-guided gene regulation and editing. Nat. Methods 10, 1116-1121.
Fonfara, I., Le Rhun, A., Chylinski, K., Makarova, K. S., Lecrivain, A. L., Bzdrenga, J., Koonin, E. V., and Charpentier, E. (2014). Phylogeny of Cas9 determines functional exchangeability of dual-RNA and Cas9 among orthologous type II CRISPR-Cas systems. Nucleic Acids Res. 42, 2577-2590.
Friedland, A. E., Baral, R., Singhal, P., Loveluck, K., Shen, S., Sanchez, M., Marco, E., Gotta, G. M., Maeder, M. L., Kennedy, E. M., et al. (2015). Characterization of Staphylococcus aureus Cas9: a smaller Cas9 for all-in-one adeno-associated virus delivery and paired nickase applications. Genome Biol. 16, 257.
Friedrich, G., and Soriano, P. (1991). Promoter traps in embryonic stem cells: a genetic screen to identify and mutate developmental genes in mice. Genes Dev. 5, 1513-1523.
Fu, Y., Sander, J. D., Reyon, D., Cascio, V. M., and Joung, J. K. (2014). Improving CRISPR-Cas nuclease specificity using truncated guide RNAs. Nat. Biotechnol. 32, 279-284.
Gallagher, D. N., and Haber, J. E. (2018). Repair of a Site-Specific DNA Cleavage: Old-School Lessons for Cas9-Mediated Gene Editing. ACS Chem. Biol. 13, 397-405.
Garneau, J. E., Dupuis, M. E., Villion, M., Romero, D. A., Barrangou, R., Boyaval, P., Fremaux, C., Horvath, P., Magadan, A. H., and Moineau, S. (2010). The CRISPR/Cas bacterial immune system cleaves bacteriophage and plasmid DNA. Nature 468, 67-71.
Gasiunas, G., Barrangou, R., Horvath, P., and Siksnys, V. (2012). Cas9-crRNA ribonucleoprotein complex mediates specific DNA cleavage for adaptive immunity in bacteria. Proc. Natl. Acad. Sci. USA 109, E2579-2586.
Gaudelli, N. M., Komor, A. C., Rees, H. A., Packer, M. S., Badran, A. H., Bryson, D. I., and Liu, D. R. (2017). Programmable base editing of A*T to G*C in genomic DNA without DNA cleavage. Nature 551, 464-471.
Ghanta, K., Dokshin, G., Mir, A., Krishnamurthy, P., Gneid, H., Edraki, A., Watts, J., Sontheimer, E., and Mello, C. (2018). 5′ Modifications Improve Potency and Efficacy of DNA Donors for Precision Genome Editing. Biorxiv 354480.
Gorski, S. A., Vogel, J., and Doudna, J. A. (2017). RNA-based recognition and targeting: sowing the seeds of specificity. Nat. Rev. Mol. Cell Biol. 18, 215-228.
Harrington, L. B., Doxzen, K. W., Ma, E., Liu, J. J., Knott, G. J., Edraki, A., Garcia, B., Amrani, N., Chen, J. S., Cofsky, J. C., et al. (2017a). A Broad-Spectrum Inhibitor of CRISPR-Cas9. Cell 170, 1224-1233.
Harrington, L. B., Paez-Espino, D., Staahl, B. T., Chen, J. S., Ma, E., Kyrpides, N. C., and Doudna, J. A. (2017b). A thermostable Cas9 with increased lifetime in human plasma. Nat. Commun. 8, 1424.
Hou, Z., Zhang, Y., Propson, N. E., Howden, S. E., Chu, L. F., Sontheimer, E. J., and Thomson, J. A. (2013). Efficient genome engineering in human pluripotent stem cells using Cas9 from Neisseria meningitidis. Proc. Natl. Acad. Sci. USA 110, 15644-15649.
Hu, J. H., Miller, S. M., Geurts, M. H., Tang, W., Chen, L., Sun, N., Zeina, C. M., Gao, X., Rees, H. A., Lin, Z., et al. (2018). Evolved Cas9 variants with broad PAM compatibility and high DNA specificity. Nature 556, 57-63.
Hwang, W. Y., Fu, Y., Reyon, D., Maeder, M. L., Tsai, S. Q., Sander, J. D., Peterson, R. T., Yeh, J. R., and Joung, J. K. (2013). Efficient genome editing in zebrafish using a CRISPR-Cas system. Nat. Biotechnol. 31, 227-229.
Hynes, A. P., Rousseau, G. M., Lemay, M.-L., Horvath, P., Romero, D. A., Fremaux, C., and Moineau, S. (2017). An anti-CRISPR from a virulent streptococcal phage inhibits Streptococcus pyogenes Cas9. Nat. Microbiol. 2, 1374-1380.
Ibraheim, R., Song, C.-Q., Mir, A., Amrani, N., Xue, W., and Sontheimer, E. J. (2018). All-in-One Adeno-associated Virus Delivery and Genome Editing by Neisseria meningitidis Cas9 in vivo. BioRxiv, doi.org/10.1101/295055.
Jiang, F., and Doudna, J. A. (2017). CRISPR-Cas9 Structures and Mechanisms. Annu. Rev. Biophys. 46, 505-529.
Jiang, W., Bikard, D., Cox, D., Zhang, F., and Marraffini, L. A. (2013). RNA-guided editing of bacterial genomes using CRISPR-Cas systems. Nat. Biotechnol. 31, 233-239.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., East, A., Cheng, A., Lin, S., Ma, E., and Doudna, J. (2013). RNA-programmed genome editing in human cells. eLife 2, e00471.
Karvelis, T., Gasiunas, G., Young, J., Bigelyte, G., Silanskas, A., Cigan, M., and Siksnys, V. (2015). Rapid characterization of CRISPR-Cas9 protospacer adjacent motif sequence elements. Genome Biol. 16, 253.
Keeler, A. M., ElMallah, M. K., and Flotte, T. R. (2017). Gene Therapy 2017: Progress and Future Directions. Clin. Transl. Sci. 10, 242-248.
Kim, E., Koo, T., Park, S. W., Kim, D., Kim, K.-E., Kim, K., Cho, H.-Y., Song, D. W., Lee, K. J., Jung, M. H., et al. (2017). In vivo genome editing with a small Cas9 ortholog derived from Campylobacter jejuni. Nat. Commun. 8, 14500.
Kim, S., Kim, D., Cho, S. W., Kim, J., and Kim, J. S. (2014). Highly efficient RNA-guided genome editing in human cells via delivery of purified Cas9 ribonucleoproteins. Genome Res. 24, 1012-1019.
Kim, B., Komor, A., Levy, J., Packer, M., Zhao, K., and Liu, D. (2017). Increasing the genome-targeting scope and precision of base editing with engineered Cas9-cytidine deaminase fusions. Nature Biotechnology 35.
Kleinstiver, B. P., Prew, M. S., Tsai, S. Q., Nguyen, N. T., Topkar, V. V., Zheng, Z., and Joung, J. K. (2015). Broadening the targeting range of Staphylococcus aureus CRISPR-Cas9 by modifying PAM recognition. Nat. Biotechnol. 33, 1293-1298.
Kluesner, M., Nedveck, D., Lahr, W., Garbe, J., Abrahante, J., Webber, B., and Moriarity, B. (2018). EditR: A Method to Quantify Base Editing from Sanger Sequencing. The CRISPR Journal 1, 239-250.
Koblan, L., Doman, J., Wilson, C., Levy, J., Tay, T., Newby, G., Maianti, J., Raguram, A., and Liu, D. (2018). Improving cytidine and adenine base editors by expression optimization and ancestral reconstruction. Nat Biotechnol 36, 843.
Komor, A. C., Badran, A. H., and Liu, D. R. (2017). CRISPR-Based Technologies for the Manipulation of Eukaryotic Genomes. Cell 168, 20-36.
Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
Lee, C. M., Cradick, T. J., and Bao, G. (2016). The Neisseria meningitidis CRISPR-Cas9 system enables specific genome editing in mammalian cells. Mol. Ther. 24, 645-654.
Lee, J., Mir, A., Edraki, A., Garcia, B., Amrani, N., Lou, H. E., Gainetdinov, I., Pawluk, A., Ibraheim, R., Gao, X. D., et al. (2018). Potent Cas9 inhibition in bacterial and human cells by new anti-CRISPR protein families. BioRxiv, biorxiv.org/content/early/2018/2006/2020/350504.
Ma, E., Harrington, L. B., O'Connell, M. R., Zhou, K., and Doudna, J. A. (2015). Single-Stranded DNA Cleavage by Divergent CRISPR-Cas9 Enzymes. Mol. Cell 60, 398-407.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nat. Biotechnol. 31, 833-838.
Mali, P., Yang, L., Esvelt, K. M., Aach, J., Guell, M., DiCarlo, J. E., Norville, J. E., and Church, G. M. (2013b). RNA-guided human genome engineering via Cas9. Science 339, 823-826.
Marraffini, L. A., and Sontheimer, E. J. (2008). CRISPR interference limits horizontal gene transfer in staphylococci by targeting DNA. Science 322, 1843-1845.
Mir, A., Edraki, A., Lee, J., and Sontheimer, E. J. (2018). Type II-C CRISPR-Cas9 biology, mechanism and application. ACS Chem. Biol. 13, 357-365.
Mojica, F. J., Diez-Villasenor, C., Garcia-Martinez, J., and Almendros, C. (2009). Short motif sequences determine the targets of the prokaryotic CRISPR defence system. Microbiology 155, 733-740.
Paez-Espino, D., Sharon, I., Morovic, W., Stahl, B., Thomas, B. C., Barrangou, R., and Banfield, J. F. (2015). CRISPR immunity drives rapid phage genome evolution in Streptococcus thermophilus. mBio 6.
Pawluk, A., Amrani, N., Zhang, Y., Garcia, B., Hidalgo-Reyes, Y., Lee, J., Edraki, A., Shah, M., Sontheimer, E. J., Maxwell, K. L., et al. (2016). Naturally occurring off-switches for CRISPR-Cas9. Cell 167, 1829-1838 e1829.
Pawluk, A., Bondy-Denomy, J., Cheung, V. H., Maxwell, K. L., and Davidson, A. R. (2014). A new group of phage anti-CRISPR genes inhibits the type I-E CRISPR-Cas system of Pseudomonas aeruginosa. mBio 5, e00896.
Pinello, L., Canver, M. C., Hoban, M. D., Orkin, S. H., Kohn, D. B., Bauer, D. E., and Yuan, G. C. (2016). Analyzing CRISPR genome-editing experiments with CRISPResso. Nat. Biotechnol. 34, 695-697.
Racanelli, V., and Rehermann, B. (2006). The liver as an immunological organ. Hepatology 43, S54-62.
Ran, F. A., Cong, L., Yan, W. X., Scott, D. A., Gootenberg, J. S., Kriz, A. J., Zetsche, B., Shalem, O., Wu, X., Makarova, K. S., et al. (2015). In vivo genome editing using Staphylococcus aureus Cas9. Nature 520, 186-191.
Ran, F. A., Hsu, P. D., Lin, C. Y., Gootenberg, J. S., Konermann, S., Trevino, A. E., Scott, D. A., Inoue, A., Matoba, S., Zhang, Y., et al. (2013). Double nicking by RNA-guided CRISPR Cas9 for enhanced genome editing specificity. Cell 154, 1380-1389.
Rashid, S., Curtis, D. E., Garuti, R., Anderson, N. N., Bashmakov, Y., Ho, Y. K., Hammer, R. E., Moon, Y. A., and Horton, J. D. (2005). Decreased plasma cholesterol and hypersensitivity to statins in mice lacking Pcsk9. Proc. Natl. Acad. Sci. USA 102, 5374-5379.
Rauch, B. J., Silvis, M. R., Hultquist, J. F., Waters, C. S., McGregor, M. J., Krogan, N. J., and Bondy-Denomy, J. (2017). Inhibition of CRISPR-Cas9 with Bacteriophage Proteins. Cell 168, 150-158 e110.
Sapranauskas, R., Gasiunas, G., Fremaux, C., Barrangou, R., Horvath, P., and Siksnys, V. (2011). The Streptococcus thermophilus CRISPR/Cas system provides immunity in Escherichia coli. Nucleic Acids Res. 39, 9275-9282.
Schumann, K., Lin, S., Boyer, E., Simeonov, D. R., Subramaniam, M., Gate, R. E., Haliburton, G. E., Ye, C. J., Bluestone, J. A., Doudna, J. A., et al. (2015). Generation of knock-in primary human T cells using Cas9 ribonucleoproteins. Proc. Natl. Acad. Sci. USA 112, 10437-10442.
Shin, J., Jiang, F., Liu, J. J., Bray, N. L., Rauch, B. J., Baik, S. H., Nogales, E., Bondy-Denomy, J., Corn, J. E., and Doudna, J. A. (2017). Disabling Cas9 by an anti-CRISPR DNA mimic. Sci. Adv. 3, e1701620.
Tsai, S. Q., and Joung, J. K. (2016). Defining and improving the genome-wide specificities of CRISPR-Cas9 nucleases. Nat. Rev. Genet. 17, 300-312.
Tsai, S. Q., Zheng, Z., Nguyen, N. T., Liebers, M., Topkar, V. V., Thapar, V., Wyvekens, N., Khayter, C., Iafrate, A. J., Le, L. P., et al. (2014). GUIDE-seq enables genome-wide profiling of off-target cleavage by CRISPR-Cas nucleases. Nat. Biotechnol. 33, 187-197.
Tycko, J., Myer, V. E., and Hsu, P. D. (2016). Methods for optimizing CRISPR-Cas9 genome editing specificity. Mol. Cell 63, 355-370.
Yang, H., and Patel, D. J. (2017). Inhibition Mechanism of an Anti-CRISPR Suppressor AcrIIA4 Targeting SpyCas9. Mol Cell 67, 117-127 e115.
Yin, H., Song, C. Q., Suresh, S., Kwan, S. Y., Wu, Q., Walsh, S., Ding, J., Bogorad, R. L., Zhu, L. J., Wolfe, S. A., et al. (2018). Partial DNA-guided Cas9 enables genome editing with reduced off-target activity. Nat. Chem. Biol. 14, 311-316.
Yokoyama, T., Silversides, D. W., Waymire, K. G., Kwon, B. S., Takeuchi, T., and Overbeek, P. A. (1990). Conserved cysteine to serine mutation in tyrosinase is responsible for the classical albino mutation in laboratory mice. Nucleic Acids Res. 18, 7293-7298.
Yoon, Y., Wang, D., Tai, P. W. L., Riley, J., Gao, G., and Rivera-Perez, J. A. (2018). Streamlined ex vivo and in vivo genome editing in mouse embryos using recombinant adeno-associated viruses. Nat. Commun. 9, 412.
Zhang, Y., Heidrich, N., Ampattu, B. J., Gunderson, C. W., Seifert, H. S., Schoen, C., Vogel, J., and Sontheimer, E. J. (2013). Processing-independent CRISPR RNAs limit natural transformation in Neisseria meningitidis. Mol. Cell 50, 488-503.
Zhang, Y., Rajan, R., Seifert, H. S., Mondragón, A., and Sontheimer, E. J. (2015). DNase H activity of Neisseria meningitidis Cas9. Mol. Cell 60, 242-255.
Zhang, Z., Theurkauf, W. E., Weng, Z., and Zamore, P. D. (2012). Strand-specific libraries for high throughput RNA sequencing (RNA-Seq) prepared without poly(A) selection. Silence 3, 9.
Zhu, L. J., Holmes, B. R., Aronin, N., and Brodsky, M. H. (2014). CRISPRseek: a bioconductor package to identify target-specific guide RNAs for CRISPR-Cas9 genome-editing systems. PLoS One 9, e108424.
Zhu, L. J., Lawrence, M., Gupta, A., Pag6s, H., Kucukural, A., Garber, M., and Wolfe, S. A. (2017). GUIDEseq: a bioconductor package to analyze GUIDE-Seq datasets for CRISPR-Cas nucleases. BMC Genomics 18, 379.
Zuris, J. A., Thompson, D. B., Shu, Y., Guilinger, J. P., Bessen, J. L., Hu, J. H., Maeder, M. L., Joung, J. K., Chen, Z.-Y., and Liu, D. R. (2015). Cationic lipid-mediated delivery of proteins enables efficient protein-based genome editing in vitro and in vivo. Nat. Biotechnol. 33, 73-80.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biological control, biochemistry, molecular biology, entomology, plankton, fishery systems, and fresh water ecology, or related fields are intended to be within the scope of the following claims.

1. A fusion protein comprising an Nme2Cas9 protein and an inlaid nucleotide base editor (NBE) domain.

2. The fusion protein of claim 1, wherein the inlaid NBE domain is an adenine base editor (ABE) domain.

3. The fusion protein of claim 2, wherein the inlaid ABE domain is an inlaid adenosine deaminase protein domain.

4. The fusion protein of claim 3, wherein the inlaid adenosine deaminase protein domain is an adenosine deaminase8e protein domain (ABE8e).

5. The fusion protein of claim 1, wherein the inlaid NBE domain is a cytidine base editor (CBE) domain.

6. The fusion protein of claim 5, wherein the inlaid CBE domain is an inlaid cytosine deaminase protein domain.

7. The fusion protein of claim 6, wherein the cytosine deaminase protein domain is selected from the group consisting of evoFERNY and rAPOBEC1.

8. The fusion protein of claim 1, wherein the Nme2Cas9 protein comprises a protospacer adjacent motif interacting domain (PID) having a peptide that hybridizes with an N₄CC nucleotide sequence or an N₄C nucleotide sequence.

9. (canceled)

10. The fusion protein of claim 1, wherein the fusion protein further comprises:
a nuclear localization signal (NLS) protein selected from the group consisting of nucleoplasmin NLS, SV40 NLS, and C-myc NLS; or
a uracil glycosylase inhibitor.

11. (canceled)

12. The fusion protein of claim 1, wherein the Nme2Cas9 protein comprises a mutation.

13-14. (canceled)

15. The fusion protein of claim 1, wherein the Nme2Cas9 protein comprises a linker that flanks at least one inlaid domain protein.

16-18. (canceled)

19. An adeno-associated virus (AAV) comprising a vector encoding the fusion protein of claim 1.

20-27. (canceled)

28. The AAV of claim 19, wherein the AAV is an adeno-associated virus 8 or an adeno-associated virus 6.

29-38. (canceled)

39. A method comprising:
a) providing;
i) a patient exhibiting at least one symptom of a genetic disease; and
ii) an adeno-associated virus (AAV) comprising a vector encoding a fusion protein comprising an Nme2Cas9 protein and an inlaid nucleotide base editor (NBE) domain; and
b) treating the patient with the adeno-associated virus under conditions such that the at least one symptom of the genetic disease is reduced.

40. The method of claim 39, wherein the genetic disease is caused by a gene with a mutated single base, wherein the gene is flanked by an N₄CC nucleotide sequence or an N₄C nucleotide sequence.

41. (canceled)

42. The method of claim 40, wherein the treating replaces the mutated single base with a wild type single base.

43-61. (canceled)

62. The method of claim 39, wherein the genetic disease is selected from the group consisting of tyrosinemia, muscular dystrophy, Rett syndrome, Batten disease, or amyotrophic lateral sclerosis (ALS).

63. A method comprising:
a) providing;
i) a patient comprising a gene with a mutated single base, wherein the gene is flanked by an N₄CC nucleotide sequence or an N₄C nucleotide sequence; and
ii) an adeno-associated virus (AAV) comprising a vector encoding a fusion protein comprising an Nme2Cas9 protein and an inlaid nucleotide deaminase protein domain; and
b) treating the patient with the adeno-associated virus under conditions such that the mutated single base is replaced with a wild type single base and a genetic disease does not develop.

64. The method of claim 63, wherein the genetic disease is selected from the group consisting of tyrosinemia, muscular dystrophy, Rett syndrome, Batten disease, and amyotrophic lateral sclerosis (ALS).

65-81. (canceled)

82. The method of claim 63, wherein the gene is selected from the group consisting of a Fah gene, a Dmd gene, a MeCP2 gene, a CLN3 gene, and an SOD1 gene.
